(19) United States
(12) Patent Application Publication
(10) Pub. No.: US 2023/0016424 A1
(43) Pub. Date: Jan. 19, 2023

(54) HYBRID AMIDE DERIVATIVES OF AMPHOTERICIN B

(71) Applicant: The Board of Trustees of the University of Illinois, Urbana, IL (US)

(72) Inventors: Martin D. Burke, Champaign, IL (US); Arun Maji, Champaign, IL (US); Jiabao Zhang, Urbana, IL (US); Su Yan, Champaign, IL (US); Yong Nian, Urbana, IL (US)

(21) Appl. No.: 17/633,254

(22) PCT Filed: Aug. 10, 2020
(86) PCT No.: PCT/US20/45566

§ 371 Date: Feb. 7, 2022

Related U.S. Application Data
(60) Provisional application No. 62/951,753, filed on Dec. 20, 2019.
(60) Provisional application No. 62/884,464, filed on Aug. 8, 2019.

(51) Int. Cl. C07D 493/08; A61P 31/10

(52) U.S. Cl. CPC C07D 493/08; A61P 31/10

(57) ABSTRACT

Disclosed are C16 amide derivatives of C2′-epi-amphotericin B (C2′epiAmB) and amphotericin B (AmB), characterized by improved clinical efficacy with reduced toxicity compared to AmB. Also disclosed are pharmaceutical compositions comprising either type of the C16 amide derivatives, and therapeutic methods of using either type of the C16 amide derivatives; and methods of making the C16 amide derivatives of C2′-epi-amphotericin B.

### CROSS-REFERENCE TO RELATED APPLICATIONS

This application claims the benefit of priority to U.S. Provisional Patent Application No. 62/884,464, filed Aug. 8, 2019, and U.S. Provisional Patent Application No. 62/951,753, filed Dec. 20, 2019. The content of these applications is incorporated herein by reference in its entirety.

### GOVERNMENT SUPPORT

This invention was made with government support under grants GM118185 and AI135812 awarded by the National Institutes of Health. The government has certain rights in the invention.

### BACKGROUND OF THE INVENTION

Morbidity and mortality from invasive fungal infections are significant, and largely caused by two genera of fungal pathogens: Candida and Aspergillus. Candida species are the 4th most common pathogen isolated in all bloodstream infections. Treatment for invasive candidiasis has a limited (50-70%) success rate, and this is typically only in the healthiest patients. Attributable mortality for invasive candidiasis is substantial (20-30%). The incidence of invasive aspergillosis due to A. fumigatus has increased three-fold in the last decade and its mortality has risen by over 300%. Moreover, current therapy for invasive aspergillosis has a lower 40-50% treatment success rate. Invasive aspergillosis is consistently a leading killer in immunocompromised patients, and moreover, whereas invasive mold infections (fusariosis, scedosporosis, and mucromycosis) have even higher mortality rates and no effective therapeutic options. The current guideline-recommended first line therapeutic for invasive aspergillosis, as well as most other invasive mold infections, is the triazole antifungal voriconazole. However, pan-triazole resistance in Aspergillus is as high as 30% in some locations and amongst certain high-risk patient groups. Recognizing this lack of effective treatments, the Infectious Diseases Society of America highlighted A. fumigatus as one of only six pathogens where a “substantive breakthrough is urgently needed.”

Amphotericin B (AmB) is an exceptionally promising starting point, because this drug has potent and dose-dependent fungicidal activity against a broad range of fungal pathogens and has evaded resistance for over half a century The fungicidal, as opposed to fungistatic, activity of AmB is essential in immunocompromised patients which lack a robust immune system to help clear an infection. Broad antifungal activity is especially important in critically ill patients when the identity of the pathogen is unknown and immediate empirical therapy is required. An international expert panel recently mandated that novel therapeutic approaches centered around AmB, with no resistance issues, are required. The problem is that AmB is exceptionally toxic, which limits its use to low-dose protocols that often fail to eradicate disease.

A new, paradigm-shifting mechanistic understanding of AmB that evaded the field for half a century was achieved. Previous studies report AmB binding to sterols, which was such thought to primarily drive formation of membrane-permeabilizing pores to kill both fungal and human cells. After 10 years of intensive synthesis-enabled atomistic interrogations of this natural product and frontier SSNMR experiments, it is alternatively discovered that AmB primarily kills both fungal and human cells by forming a cytocidal extramembranous sterol sponge. This large aggregate sits on the surface of lipid bilayers and rapidly extracts membrane sterols, which leads to cell death. Membrane permeabilization is not required. Based on this new mechanism and increasingly refined structural information, it is proposed that a small molecule-based ligand-selective allosteric effect could enable selective binding of ergosterol over cholesterol. Guided by this model, the elimination of cholesterol binding and thus mammalian toxicity in the form of a new derivative, C2′epiAmB, was achieved.

A limitation with C2′epiAmB, however, is lack of potency against a number of clinically relevant yeast and molds. An AmB derivative that retains potent, broad spectrum, and resistance-evasive fungicidal activity but lacks dose-limiting toxicities would enable a new high dose treatment paradigm with improved clinical efficacy.

### SUMMARY OF THE INVENTION

In certain aspects, provided are compounds of Formula (I):

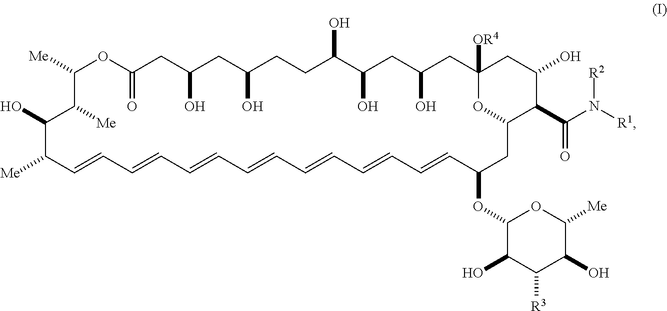

or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

In certain aspects, provided are compounds of Formula (II):

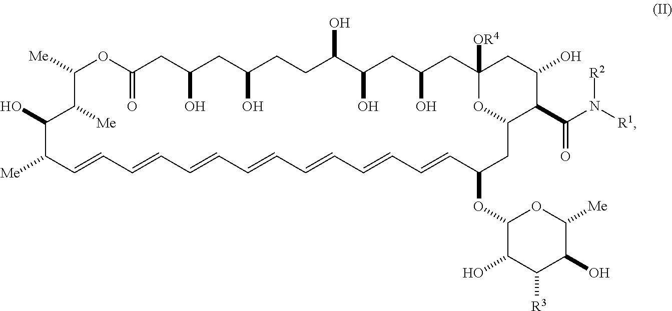

or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

In certain aspects, provided are pharmaceutical compositions, comprising a compound provided herein; and a pharmaceutically acceptable carrier.

In certain aspects, provided are methods of treating a fungal infection, comprising administering to a subject in need thereof a therapeutically effective amount of a compound provided herein, thereby treating the fungal infection.

In certain aspects, provided are methods of making a C16 amide of C2′-epi-amphotericin B according to the transformation shown in Scheme 1:

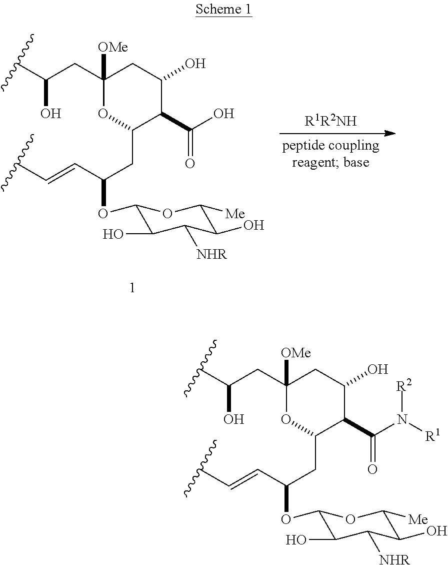

wherein:
1 represents

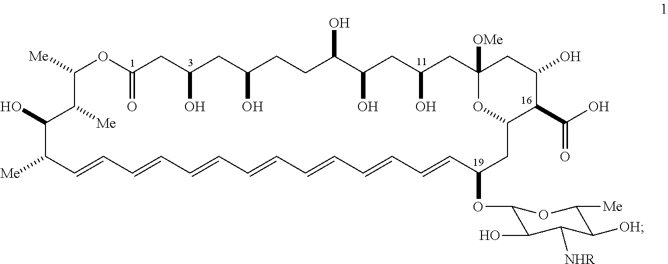

base is a tertiary amine (e.g., a trialkylamine [such as Et₃N]);
peptide coupling reagent is a peptide coupling reagent used in solid phase peptide synthesis (e.g., PyBOP, BOP, HATU, HBTU, DEPBT, DCC, or EDCI);
R is H or an amine protecting group (e.g., a carbamate protecting group selected from the group consisting of Fmoc, t-Boc, alloc, and Cbz); and
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or R¹ and R², together with the nitrogen to which they are attached, form a substituted or unsubstituted 3- to 10-membered heterocyclyl.

### BRIEF DESCRIPTION OF THE FIGURES

FIG. 1A represents chemical structures of amphotericin B, the primary fungal sterol-ergosterol, and the primary human sterol-cholesterol.

FIG. 1B depicts a two-step “Sterol Sponge” model for the cytocidal action of AmB.

FIG. 2A represents chemical structures and biophysical activities of AmB, AmdeB, C2′deOAmB, and C2′epiAmB.

FIG. 2B represents biophysical activities of AmB, AmdeB, C2′deOAmB, and C2′epiAmB in primary human renal epithelial cells.

FIG. 2C represents ergosterol and cholesterol activities of AmB, AmdeB, C2′deOAmB, and C2′epiAmB.

FIG. 3A is an X-ray crystal structure of N-iodoacyl AmB.

FIG. 3B depicts a proposed structural model for AmB-Erg complex. A similar model is proposed for cholesterol.

FIG. 4 represents the 11-step synthesis of C2′epiAmB from AmB.

FIG. 5A depicts sterol binding. Sterol sponges formed in vitro from AmB were titrated with ergosterol and analyzed by UV-Vis spectroscopy.

FIG. 5B depicts sterol binding. Sterol sponges formed in vitro from AmB were titrated with cholesterol and analyzed by UV-Vis spectroscopy.

FIG. 5C depicts sterol binding. Sterol sponges formed in vitro from C2′epiAmB were titrated with ergosterol and analyzed by UV-Vis spectroscopy.

FIG. 5D depicts sterol binding. Sterol sponges formed in vitro from C2′epiAmB were titrated with cholesterol and analyzed by UV-Vis spectroscopy.

FIG. 6 represents toxicity data of AmB-deoxycholate and C2′epiAmB-doxycholate in mice.

FIG. 7 represents toxicity data of AmBisome® compared directly with C2′epiAmB, as judged by renal genotoxicity biomarkers.

FIG. 8A depicts in vitro antifungal activity of AmB and C2′epiAmB against a broad range of fungal pathogens in a panel of Candida and Aspergillus isolates.

FIG. 8B depicts in vitro antifungal activity of AmB and C2′epiAmB against a broad range of fungal pathogens in a panel of Aspergillus isolates.

FIG. 8C depicts in vitro antifungal activity of AmB and C2′epiAmB against a broad range of fungal pathogens in a panel of clinically relevant invasive molds.

FIG. 9 depicts the MICs of AmB and C2′epiAmB against C. albicans with and without pre-complexation with ergosterol.

FIG. 10 represents the efficacy of AmB and C2′epiAmB in a mouse model of invasive candidiasis.

FIG. 11 is a graph depicting the killing kinetics for AmB and C16 amide AmB (AmBHEA) towards C. albicans SN250.

FIG. 12 is a scheme depicting the rational design of C16 amide C2′epiAmB with both potency and reduced toxicity.

FIG. 13A is a UV-Vis graph depicting AmB binding to cholesterol.

FIG. 13B is a UV-Vis graph depicting C2′epiAmB binding to cholesterol.

FIG. 13C is a UV-Vis graph depicting C16 amide C2′epiAmB (C2′epiAmBHEA; the amide formed from (2-hydroxyethyl)amine and C2′epiAmB) binding to cholesterol.

FIG. 14 depicts using an AmBisome®-like formulation to increase the solubility of C2′epiAmB and C16 amide C2′epiAmB.

FIG. 15 describes the mice study for AmB, C2′epiAmB, and C16 amide C2′epiAmB (C2′epiAmB-L-His) in AmBisome®-like formulation.

FIG. 16 is a graph depicting the impact on activity of the size of the aliphatic ring and the aliphatic acyclic chain at the C16 position.

FIG. 17 is graph depicting the impact on activity of polar functional groups at the C16 position.

FIG. 18 is a graph depicting the pharmacokinetics of compound BA as compared to amphotericin B.

FIG. 19 is a graph depicting the changes in expression of biomarkers of renal injury by RTPCR upon treatment with fungizone, ambisome, or Sfu-AM220.

FIG. 20 is a graph depicting the efficacy of compound BA, C2′epiAmB and AmBisome, as defined in terms of colony forming units (CFUs).

FIG. 21A-21D depict in vitro and in vivo safety data for Compound BA.

FIG. 22 depicts in vivo mouse pharmacokinetic data for Compound BA.

### DETAILED DESCRIPTION OF THE INVENTION

Amphotericin B (AmB) is a polyene macrolide with a mycosamine appendage, the complete compound having the following structure:

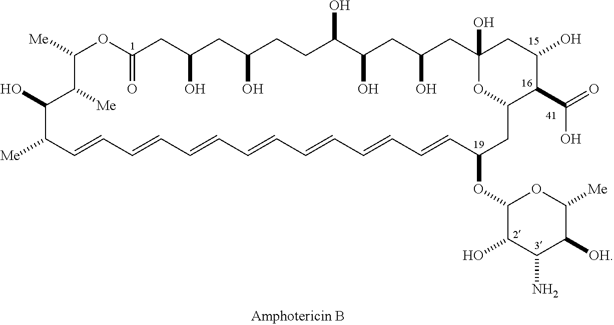

AmB is generally obtained from a strain of Streptomyces nodosus. It is currently approved for clinical use in the United States for the treatment of progressive, potentially life-threatening fungal infections, including such infections as systemic or deep tissue candidiasis, aspergillosis, cryptococcosis, blastomycosis, coccidioidomycosis, histoplasmosis, and mucormycosis, among others. It is generally formulated for intravenous injection. Amphotericin B is commercially available, for example, as Fungizone® (Squibb), Amphocin® (Pfizer), Abelcet® (Enzon), and Ambisome® (Astellas). Due to its undesirable toxic side effects, dosing is generally limited to a maximum of about 1.0 mg/kg/day and total cumulative doses not to exceed about 3 g in humans.

AmB kills both fungal and human cells by forming a cytocidal extramembranous sterol sponge. Anderson, T. M. et al., Nat Chem Biol 2014, 10 (5), 400-6. This large aggregate sits on the surface of lipid bilayers and rapidly extracts membrane sterols, which leads to cell death. Membrane permeabilization is not required. Based on this mechanism, a small molecule-based ligand-selective allosteric effect would enable selective binding of ergosterol over cholesterol and would eliminate the mammalian toxicity of AmB (in the form of C2′epiAmB). See Wilcock, B. C. et al., J Am Chem Soc 2013, 135 (23), 8488-91. The present invention discloses the K_{D}s for the binding of both ergosterol and cholesterol to the AmB sterol sponge, which provides a quantitative and mechanistically-grounded biophysical parameter to guide rational optimization of the therapeutic index of this clinically significant natural product.

The present invention relates, at least in part, to the discovery by the inventors of further derivatives of AmB which also are characterized by improved therapeutic index compared to AmB. The various derivatives, i.e., compounds of the invention, can be semi-synthetic or fully synthetic. An aspect of the invention is the development of a new synthetic derivative of AmB that retains potent binding of ergosterol but shows no detectable binding of cholesterol. This derivative retains fungicidal potency against many yeasts and molds but shows no detectable mammalian toxicity. This demonstrates that differential binding of ergosterol over cholesterol is possible and provides a non-toxic variant of AmB that preserves desirable antifungal properties. Compounds of the invention enable a new high-dose treatment strategy to eradicate life-threatening invasive fungal infections with a significantly improved safety profile.

Compounds of the invention and pharmaceutical compositions of the invention are useful for inhibiting the growth of a fungus. In one embodiment, an effective amount of a compound of the invention is contacted with a fungus, thereby inhibiting growth of the fungus. In one embodiment, a compound of the invention, or a pharmaceutically acceptable salt thereof, is added to or included in tissue culture medium.

Compounds of the invention and pharmaceutical compositions of the invention are useful for the treatment of fungal infections in a subject. In one embodiment, a therapeutically effective amount of a compound of the invention, or a pharmaceutically acceptable salt thereof, is administered to a subject in need thereof, thereby treating the fungal infection.

Yeasts are eukaryotic organisms classified in the kingdom Fungi. Fungi include yeasts, molds, and larger organisms including mushrooms. Yeasts and molds are of clinical relevance as infectious agents. Yeasts are typically described as budding forms of fungi. Of particular importance in connection with the invention are species of yeast that can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic yeasts include, without limitation, various species of the genus Candida, as well as of Cryptococcus. Of particular note among pathogenic yeasts of the genus Candida are C. albicans, C. tropicalis, C. stellatoidea, C. glabrata, C. krusei, C. parapsilosis, C. guilliermondii, C. viswanathii, and C. lusitaniae. The genus Cryptococcus specifically includes Cryptococcus neoformans. Yeast can cause infections of mucosal membranes, for example oral, esophageal, and vaginal infections in humans, as well as infections of bone, blood, urogenital tract, and central nervous system. This list is exemplary and is not limiting in any way.

A number of fungi (apart from yeast) can cause infections in mammalian hosts. Such infections most commonly occur in immunocompromised hosts, including hosts with compromised barriers to infection (e.g., burn victims) and hosts with compromised immune systems (e.g., hosts receiving chemotherapy or immune suppressive therapy, and hosts infected with HIV). Pathogenic fungi (apart from yeast) include, without limitation, species of Aspergillus, Rhizopus, Mucor, Histoplasma, Coccidioides, Blastomyces, Trichophyton, Microsporum, and Epidermophyton. Of particular note among the foregoing are A. fumigatus, A. flavus, A. niger, H. capsulatum, C. immitis, and B. dermatitidis. Fungi can cause systemic and deep tissue infections in lung, bone, blood, urogenital tract, and central nervous system, to name a few. Some fungi are responsible for infections of the skin and nails.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley &amp; Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPFC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. F. Eliel, Ed., Univ. of Notre Dame Press, Notre Dame, Ind. 1972).

The invention additionally encompasses compounds described herein as individual isomers substantially free of other isomers, and alternatively, as mixtures of various isomers.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present invention. When describing the invention, which may include compounds, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), isobutyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), i-Pr (—CH(CH₃)₂), n-Pr (—CH₂CH₂CH₃), n-Bu (—CH₂CH₂CH₂CH₃), or i-Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)═CHCH₂—, —CH═C(CH₃)CH₂—, —CH═CHCH(CH₃)—, —CH═CHC(CH₃)₂—, —CH(CH₃)—CH═CH—, —C(CH₃)₂—CH═CH—, —CH₂—C(CH₃)═CH—, —CH₂—CH═C(CH₃)—), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and/or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆ alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and for 2 heteroatoms (“heteroC₂₋₄ alkenyl”). In some embodiments, a heteroalkenyl group has 2 to 3 carbon atoms, at least one double bond, and 1 heteroatom (“heteroC₂₋₃ alkenyl”). In some embodiments, a heteroalkenyl group has 2 to 6 carbon atoms, at least one double bond, and 1 or 2 heteroatoms (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆ alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and for 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “alkylene,” “alkenylene,” “alkynylene,” “heteroalkylene,” “heteroalkenylene,” and “heteroalkynylene,” refer to a divalent radical of an alkyl, alkenyl, alkynyl group, heteroalkyl, heteroalkenyl, and heteroalkynyl group respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon divalent chain. “Alkylene,” “alkenylene,” “alkynylene,” “heteroalkylene,” “heteroalkenylene,” and “heteroalkynylene” groups may be substituted or unsubstituted with one or more substituents as described herein.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁₋₈ alkyl, C₁₋₈ haloalkyl, cyano, hydroxy, C₁₋₈ alkoxy, and amino.

Examples of representative substituted aryls include the following

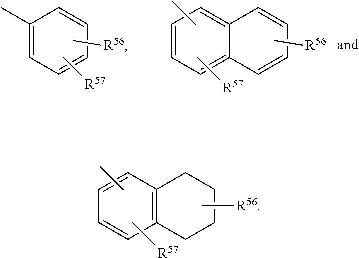

wherein one of R⁵⁶ and R⁵⁷ may be hydrogen and at least one of R⁵⁶ and R⁵⁷ is each independently selected from C₁₋₈ alkyl, C₁₋₈ haloalkyl, 4- to 10-membered heterocyclyl, alkanoyl, C₁₋₈ alkoxy, heteroaryloxy, alkylamino, arylamino, heteroarylamino, NR⁵⁸COR⁵⁹, NR⁵⁸SOR⁵⁹ NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁₋₈ alkyl, C₁₋₄ haloalkyl, C₃₋₁₀ carbocyclyl, 4- to 10-membered heterocyclyl, C₆₋₁₀ aryl, substituted C₆₋₁₀ aryl, 5-10 membered heteroaryl, or substituted 5- to 10-membered heteroaryl.

Other representative aryl groups having a fused heterocyclyl group include the following:

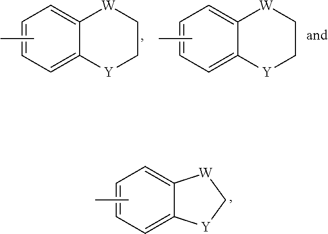

wherein each W is selected from C(R⁶⁶)₂, NR⁶⁶, O, and S; and each Y is selected from carbonyl, NR⁶⁶, O and S; and R⁶⁶ is independently hydrogen, C₁₋₈ alkyl, C₃₋₁₀ carbocyclyl, 4- to 10-membered heterocyclyl, C₆₋₁₀ aryl, and 5- to 10-membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5- to 10-membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5- to 10-membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5- to 10-membered aromatic ring system having ring carbon atoms and 1-4 ring heteroatoms provided in the aromatic ring system, wherein each heteroatom is independently selected from nitrogen, oxygen, and sulfur (“5- to 10-membered heteroaryl”). In some embodiments, a heteroaryl group is a 5- to 8-membered aromatic ring system having ring carbon atoms and 1-4 ring heteroatoms provided in the aromatic ring system, wherein each heteroatom is independently selected from nitrogen, oxygen, and sulfur (“5- to 8-membered heteroaryl”). In some embodiments, a heteroaryl group is a 5- to 6-membered aromatic ring system having ring carbon atoms and 1-4 ring heteroatoms provided in the aromatic ring system, wherein each heteroatom is independently selected from nitrogen, oxygen, and sulfur (“5- to 6-membered heteroaryl”). In some embodiments, the 5-6 membered heteroaryl has 1-3 ring heteroatoms selected from nitrogen, oxygen, and sulfur. In some embodiments, the 5- to 6-membered heteroaryl has 1-2 ring heteroatoms selected from nitrogen, oxygen, and sulfur. In some embodiments, the 5- to 6-membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5- to 14-membered heteroaryl. In certain embodiments, the heteroaryl group is substituted 5- to 14-membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

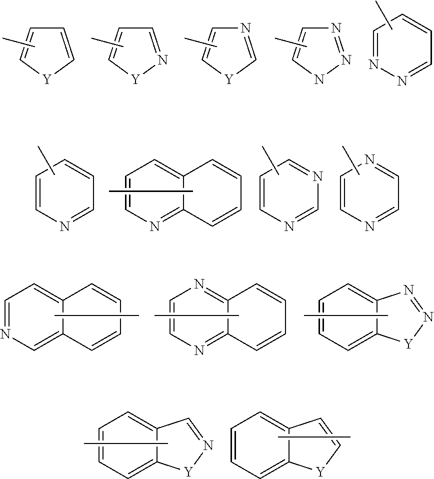

wherein each Y is selected from carbonyl, N, NR⁶⁵, O, and S; and R⁶⁵ is independently hydrogen, C₁₋₈ alkyl, C₃₋₁₀ carbocyclyl, 4-10 membered heterocyclyl, C₆₋₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Examples of C₅₋₆ carbocyclyl groups include cyclopentyl (C₅) and cyclohexyl (C₅). Examples of C₃₋₆ carbocyclyl groups include the aforementioned C₅₋₆ carbocyclyl groups as well as cyclopropyl (C₃) and cyclobutyl (C₄). Examples of C₃₋₈ carbocyclyl groups include the aforementioned C₃₋₆ carbocyclyl groups as well as cycloheptyl (C₇) and cyclooctyl (C₈). Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is substituted C₃₋₁₀ carbocyclyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3- to 10-membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3- to 10-membered heterocyclyl. In certain embodiments, the heterocyclyl group is substituted 3- to 10-membered heterocyclyl.

In some embodiments, a heterocyclyl group is a 5- to 10-membered non-aromatic ring system having ring carbon atoms and 1-4 ring heteroatoms, wherein each heteroatom is independently selected from nitrogen, oxygen, sulfur, boron, phosphorus, and silicon (“5- to 10-membered heterocyclyl”). In some embodiments, a heterocyclyl group is a 5- to 8-membered non-aromatic ring system having ring carbon atoms and 1-4 ring heteroatoms, wherein each heteroatom is independently selected from nitrogen, oxygen, and sulfur (“5- to 8-membered heterocyclyl”). In some embodiments, a heterocyclyl group is a 5- to 6-membered non-aromatic ring system having ring carbon atoms and 1-4 ring heteroatoms, wherein each heteroatom is independently selected from nitrogen, oxygen, and sulfur (“5- to 6-membered heterocyclyl”). In some embodiments, the 5-6 membered heterocyclyl has 1-3 ring heteroatoms selected from nitrogen, oxygen, and sulfur. In some embodiments, the 5- to 6-membered heterocyclyl has 1-2 ring heteroatoms selected from nitrogen, oxygen, and sulfur. In some embodiments, the 5- to 6-membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

Particular examples of heterocyclyl groups are shown in the following illustrative examples:

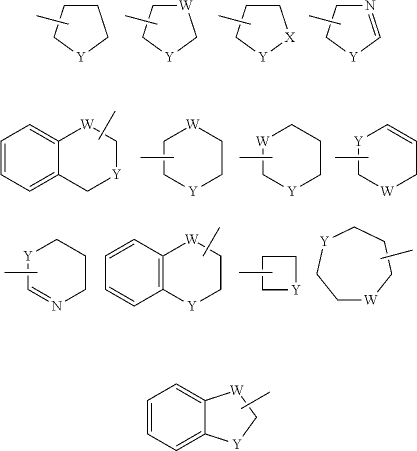

wherein each W is selected from CR⁶⁷, C(R⁶⁷)₂, NR⁶⁷, O, and S; and each Y is selected from NR⁶⁷, O, and S; and R⁶⁷ is independently hydrogen, C₁₋₈ alkyl, C₃₋₁₀ carbocyclyl, 4- to 10-membered heterocyclyl, C₆₋₁₀ aryl, 5- to 10-membered heteroaryl. These heterocyclyl rings may be optionally substituted with one or more groups selected from the group consisting of acyl, acylamino, acyloxy, alkoxy, alkoxycarbonyl, alkoxycarbonylamino, amino, substituted amino, aminocarbonyl (carbamoyl or amido), aminocarbonylamino, aminosulfonyl, sulfonylamino, aryl, aryloxy, azido, carboxyl, cyano, carbocyclyl, halogen, hydroxy, keto, nitro, thiol, —S-alkyl, —S-aryl, —S(O)-alkyl, —S(O)-aryl, —S(O)₂-alkyl, and —S(O)₂-aryl. Substituting groups include carbonyl or thiocarbonyl which provide, for example, lactam and urea derivatives.

“Hetero” when used to describe a compound or a group present on a compound means that one or more carbon atoms in the compound or group have been replaced by a nitrogen, oxygen or sulfur heteroatom. Hetero may be applied to any of the hydrocarbyl groups described above such as alkyl, e.g., heteroalkyl, carbocyclyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(O)—C₁₋₈ alkyl, —C(O)—(CH₂)ₜ(C₆₋₁₀ aryl), —C(O)—(CH₂)ₜ(5- to 10-membered heteroaryl), —C(O)—(CH₂)ₜ(C₃₋₁₀ carbocyclyl), and —C(O)—(CH₂)ₜ(4- to 10-membered heterocyclyl), wherein t is an integer from 0 to 4. In certain embodiments, R is C₁₋₈ alkyl, substituted with halo or hydroxy; or C₃₋₁₀ carbocyclyl, 4- to 10-membered heterocyclyl, C₆₋₁₀ aryl, arylalkyl, 5- to 10-membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Acylamino” refers to a radical —NR²²C(O)R²³, where each instance of R²² and R²³ is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein, or R²² is an amino protecting group. Exemplary “acylamino” groups include, but are not limited to, formylamino, acetylamino, cyclohexylcarbonylamino, cyclohexylmethyl-carbonylamino, benzoylamino and benzylcarbonylamino. Particular exemplary “acylamino” groups are —NR²⁴C(O)—C₁₋₈ alkyl, —NR²⁴C(O)—(CH₂)ₜ(C₆₋₁₀ aryl), —NR²⁴C(O)—(CH₂)ₜ(5- to 10-membered heteroaryl), —NR²⁴C(O)—(CH₂)ₜ(C₃₋₁₀ carbocyclyl), and —NR²⁴C(O)—(CH₂)ₜ(4- to 10-membered heterocyclyl), wherein t is an integer from 0 to 4, and each R²⁴ independently represents H or C₁₋₈ alkyl. In certain embodiments, R²⁵ is H, C₁₋₈ alkyl, substituted with halo or hydroxy; C₃₋₁₀ carbocyclyl, 4- to 10-membered heterocyclyl, C₆₋₁₀ aryl, arylalkyl, 5-10 membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; and R²⁶ is H, C₁₋₈ alkyl, substituted with halo or hydroxy; C₃₋₁₀ carbocyclyl, 4-10 membered heterocyclyl, C₆₋₁₀ aryl, arylalkyl, 5-10 membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxyl; provided at least one of R²⁵ and R²⁶ is other than H.

“Acyloxy” refers to a radical —OC(O)R²⁷, where R²⁷ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. Representative examples include, but are not limited to, formyl, acetyl, cyclohexylcarbonyl, cyclohexylmethylcarbonyl, benzoyl and benzylcarbonyl. In certain embodiments, R²⁸ is C₁₋₈ alkyl, substituted with halo or hydroxy; C₃₋₁₀ carbocyclyl, 4- to 10-membered heterocyclyl, C₆₋₁₀ aryl, arylalkyl, 5- to 10-membered heteroaryl or heteroarylalkyl, each of which is substituted with unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆₋₁₀ aryl, aryloxy, carboxyl, cyano, C₃₋₁₀ carbocyclyl, 3- to 10-membered heterocyclyl, halogen, 5- to 10-membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆₋₁₀) aryl), —O—(CH₂)ₜ(5- to 10-membered heteroaryl), —O—(CH₂)ₜ(C₃₋₁₀) carbocyclyl), and —O—(CH₂)ₜ(4- to 10-membered heterocyclyl), wherein t is an integer from 0 to 4 and any aryl, heteroaryl, carbocyclyl or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁₋₈ alkyl, C₃₋₈ alkenyl, C₃₋₈ alkynyl, C₆₋₁₀ aryl, 5- to 10-membered heteroaryl, 4- to 10-membered heterocyclyl, or C₃₋₁₀ carbocyclyl; or C₁₋₈ alkyl, substituted with halo or hydroxy; C₃₋₈ alkenyl, substituted with halo or hydroxy; C₃₋₈ alkynyl, substituted with halo or hydroxy, or —(CH₂)ₜ(C₆₋₁₀ aryl), —(CH₂)ₜ(5- to 10-membered heteroaryl), —(CH₂)ₜ(C₃₋₁₀ carbocyclyl), or —(CH₂)ₜ(4- to 10-membered heterocyclyl), wherein t is an integer between 0 and 8, each of which is substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; or both R groups are joined to form an alkylene group.

Exemplary “substituted amino” groups include, but are not limited to, —NR³⁹—C₁₋₈ alkyl, —NR³⁹—(CH₂)ₜ(C₆₋₁₀ aryl), —NR³⁹—(CH₂)ₜ(5-10 membered heteroaryl), —NR³⁹—(CH₂)ₜ(C₃₋₁₀ carbocyclyl), and —NR³⁹—(CH₂)ₜ(4-10 membered heterocyclyl), wherein t is an integer from 0 to 4, for instance 1 or 2, each R³⁹ independently represents H or C₁₋₈ alkyl; and any alkyl groups present, may themselves be substituted by halo, substituted or unsubstituted amino, or hydroxy; and any aryl, heteroaryl, carbocyclyl, or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Azido” refers to the radical —N₃.

“Carbamoyl” or “amido” refers to the radical —C(O)NH₂.

“Substituted carbamoyl” or “substituted amido” refers to the radical —C(O)N(R⁶²)₂ wherein each R⁶² is independently hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R⁶² is not a hydrogen. In certain embodiments, R⁶² is selected from H, C₁₋₈ alkyl, C₃₋₁₀ carbocyclyl, 4- to 10-membered heterocyclyl, C₆₋₁₀ aryl, aralkyl, 5- to 10-membered heteroaryl, and heteroaralkyl; or C₁₋₈ alkyl substituted with halo or hydroxy; or C₃₋₁₀ carbocyclyl, 4- to 10-membered heterocyclyl, C₆₋₁₀ aryl, aralkyl, 5- to 10-membered heteroaryl, or heteroaralkyl, each of which is substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy; provided that at least one R⁶² is other than H.

Exemplary “substituted carbamoyl” groups include, but are not limited to, —C(O)NR⁶⁴—C₁₋₈ alkyl, —C(O)NR⁶⁴—(CH₂)ₜ(C₆₋₁₀ aryl), —C(O)N⁶⁴—(CH₂)ₜ(5- to 10-membered heteroaryl), —C(O)NR⁶⁴—(CH₂)ₜ(C₃₋₁₀ carbocyclyl), and —C(O)NR⁶⁴—(CH₂)ₜ(4- to 10-membered heterocyclyl), wherein t is an integer from 0 to 4, each R⁶⁴ independently represents H or C₁₋₈ alkyl and any aryl, heteroaryl, carbocyclyl or heterocyclyl groups present, may themselves be substituted by unsubstituted C₁₋₄ alkyl, halo, unsubstituted C₁₋₄ alkoxy, unsubstituted C₁₋₄ haloalkyl, unsubstituted C₁₋₄ hydroxyalkyl, or unsubstituted C₁₋₄ haloalkoxy or hydroxy.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Carbocyclylalkyl” refers to an alkyl radical in which the alkyl group is substituted with a carbocyclyl group. Typical carbocyclylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Cycloalkenyl” refers to substituted or unsubstituted carbocyclyl group having from 3 to 10 carbon atoms and having a single cyclic ring or multiple condensed rings, including fused and bridged ring systems and having at least one and particularly from 1 to 2 sites of olefinic unsaturation. Such cycloalkenyl groups include, by way of example, single ring structures such as cyclohexenyl, cyclopentenyl, cyclopropenyl, and the like.

“Fused cycloalkenyl” refers to a cycloalkenyl having two of its ring carbon atoms in common with a second aliphatic or aromatic ring and having its olefinic unsaturation located to impart aromaticity to the cycloalkenyl ring.

“Ethylene” refers to substituted or unsubstituted —(C—C)—.

“Ethenyl” refers to substituted or unsubstituted —(C═C)—.

“Ethynyl” refers to —(C═C)—.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —ON(R^{bb})₂, —N(R^{bb})₂, —N(R^{bb})-TXk-N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb} SO₂R^{aa}, —NR^{bb} SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP (═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{CC})₂, —P(R^{CC})₃, —OP(R^{cc})₂, —OP(R^{cc})₃, —B(R^{aa})₂, —B(OR⁰⁰)₂, —BRₐₐ(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{CC};

each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3- to 14-membered heterocyclyl, C₆₋₁₄ aryl, and 5- to 14-membered heteroaryl, or two R^{aa} groups are joined to form a 3-14 membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —N(R^{CC})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{CC})₂, —SO₂R^{cc}, —SO₂OR^{cc}, —SOR^{aa}, —C(═S)N(R^{cc})₂, —C(═O)SR^{cc}, —C(═S) SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3- to 10-membered heterocyclyl, C₆₋₁₀ aryl, 5- to 10-membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 3- to 10-membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen C₁₋₆ alkyl, C₁₋₆ perhaloalkyl, C₂₋₆ alkenyl, C₂₋₆ alkynyl, C₃₋₁₀ carbocyclyl, 3- to 10-membered heterocyclyl, C₆₋₁₀ aryl and 5- to 10-membered heteroaryl, or two R^{ff} groups are joined to form a 3-14 membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₂⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆alkyl)₂, —OP(═O)(C₁₋₆alkyl)₂, —OP(═O)(OC₁₋₆alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, 5- to 10-membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄²⁻ sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxy late ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR′, —N(R^{CC})₂, —CN, —C(═O)R^{aa}, —C(═O)N(R^{CC})₂, —CO₂R^{aa}, —SO₂R^{aa}, —C(—NR^{bb})R^{aa}, —C(═NR^{cc})OR^{aa}, —C(═NR^{CC})N(R^{CC})₂, —SO₂N(R^{CC})₂, —SO₂R^{CC}, —SO₂OR⁰⁰, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{CC})₂, —P(═O)(NR^{CC})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3- to 14-membered heterocyclyl, C₆₋₁₄ aryl, and 5- to 14-membered heteroaryl, or two R^{cc} groups attached to a nitrogen atom are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

These and other exemplary substituents are described in more detail in the Detailed Description, Examples, and claims. The invention is not intended to be limited in any manner by the above exemplary listing of substituents.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, chlorobenzenesulfonic acid, 2-naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium potassium, calcium, magnesium, ammonium, tetraalkylammonium, and the like; and when the compound contains a basic functionality, salts of nontoxic organic or inorganic acids, such as hydrochloride, hydrobromide, tartrate, mesylate, acetate, maleate, oxalate and the like.

“Pharmaceutically acceptable cation” refers to an acceptable cationic counterion of an acidic functional group. Such cations are exemplified by sodium, potassium, calcium, magnesium, ammonium, tetraalkylammonium cations, and the like (see, e. g., Berge, et al., J. Pharm. Sci. 66 (1):1-79 (January 77).

“Pharmaceutically acceptable vehicle” refers to a diluent, adjuvant, excipient or carrier with which a compound of the invention is administered.

“Pharmaceutically acceptable metabolically cleavable group” refers to a group which is cleaved in vivo to yield the parent molecule of the structural formula indicated herein. Examples of metabolically cleavable groups include —COR, —COOR, —CONRR and —CH₂OR radicals, where R is selected independently at each occurrence from alkyl, trialkylsilyl, carbocyclic aryl or carbocyclic aryl substituted with one or more of alkyl, halogen, hydroxy or alkoxy. Specific examples of representative metabolically cleavable groups include acetyl, methoxycarbonyl, benzoyl, methoxymethyl and trimethylsilyl groups.

“Prodrugs” refers to compounds, including derivatives of the compounds of the invention, which have cleavable groups and become by solvolysis or under physiological conditions the compounds of the invention which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides and anhydrides derived from acidic groups pendant on the compounds of this invention are particular prodrugs. In some cases it is desirable to prepare double ester type prodrugs such as (acyloxy)alkylesters or (alkoxycarbonyl)oxy)alkylesters. Particularly the C₁₋₈ alkyl, C₂₋₈ alkenyl, C₂₋₈ alkynyl, aryl, C₇₋₁₂ substituted aryl, and C₇₋₁₂ arylalkyl esters of the compounds of the invention.

“Solvate” refers to forms of the compound that are associated with a solvent or water (also referred to as “hydrate”), usually by a solvolysis reaction. This physical association includes hydrogen bonding. Conventional solvents include water, ethanol, acetic acid and the like. The compounds of the invention may be prepared e.g., in crystalline form and may be solvated or hydrated. Suitable solvates include pharmaceutically acceptable solvates, such as hydrates, and further include both stoichiometric solvates and non-stoichiometric solvates. In certain instances, the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “subject” to which administration is contemplated includes, but is not limited to, humans (i.e., a male or female of any age group, e.g., a pediatric subject (e.g, infant, child, adolescent) or adult subject (e.g., young adult, middle aged adult or senior adult) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

An “effective amount” means the amount of a compound that, when administered to a subject for treating or preventing a disease, is sufficient to effect such treatment or prevention. The “effective amount” can vary depending on the compound, the disease and its severity, and the age, weight, etc., of the subject to be treated. A “therapeutically effective amount” refers to the effective amount for therapeutic treatment. A “prophylactically effective amount” refers to the effective amount for prophylactic treatment.

“Preventing” or “prevention” or “prophylactic treatment” refers to a reduction in risk of acquiring or developing a disease or disorder (i.e., causing at least one of the clinical symptoms of the disease not to develop in a subject not yet exposed to a disease-causing agent, or predisposed to the disease in advance of disease onset.

“Prophylaxis” is related to “prevention,” and refers to a measure or procedure the purpose of which is to prevent, rather than to treat or cure a disease. Non limiting examples of prophylactic measures may include the administration of vaccines; the administration of low molecular weight heparin to hospital patients at risk for thrombosis due, for example, to immobilization, and the administration of an anti-malarial agent such as chloroquine, in advance of a visit to a geographical region where malaria is endemic or the risk of contracting malaria is high.

“Treating” or “treatment” or “therapeutic treatment” of any disease or disorder refers, in one embodiment, to ameliorating the disease or disorder (i.e., arresting the disease or reducing the manifestation, extent or severity of at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In a further embodiment, “treating” or “treatment” relates to slowing the progression of the disease.

As used herein, the term “isotopic variant” refers to a compound that contains unnatural proportions of isotopes at one or more of the atoms that constitute such compound. For example, an “isotopic variant” of a compound can contain one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be “²H/D, any carbon may be ¹³C, or any nitrogen may be ¹⁵N, and that the presence and placement of such atoms may be determined within the skill of the art. Likewise, the invention may include the preparation of isotopic variants with radioisotopes, in the instance for example, where the resulting compounds may be used for drug and/or substrate tissue distribution studies. The radio-active isotopes tritium, i.e., ³H, and carbon-14, i.e., ¹⁴C, are particularly useful for this purpose in view of their ease of incorporation and ready means of detection. Further, compounds may be prepared that are substituted with positron emitting isotopes, such as ¹¹C, ¹⁸F, ¹⁵O and ¹³N, a N, and would be useful in Positron Emission Topography (PET) studies for examining substrate receptor occupancy. All isotopic variants of the compounds provided herein, radioactive or not, are intended to be encompassed within the scope of the invention.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either individual enantiomer or as a mixture thereof. A mixture containing equal proportions of the enantiomers is called a “racemic mixture”.

“Tautomers” refer to compounds that are interchangeable forms of a particular compound structure, and that vary in the displacement of hydrogen atoms and electrons. Thus, two structures may be in equilibrium through the movement of it electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base. Tautomeric forms may be relevant to the attainment of the optimal chemical reactivity and biological activity of a compound of interest.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

As used herein and unless otherwise indicated, the term “enantiomerically pure R-compound” refers to at least about 95% by weight R-compound and at most about 5% by weight S-compound, at least about 99% by weight R-compound and at most about 1% by weight S-compound, or at least about 99.9% by weight R-compound and at most about 0.1% by weight S-compound. In certain embodiments, the weights are based upon total weight of compound.

As used herein and unless otherwise indicated, the term “enantiomerically pure 5-compound” or “S-compound” refers to at least about 95% by weight S-compound and at most about 5% by weight R-compound, at least about 99% by weight S-compound and at most about 1% by weight R-compound or at least about 99.9% by weight S-compound and at most about 0.1% by weight R-compound. In certain embodiments, the weights are based upon total weight of compound.

In the compositions provided herein, an enantiomerically pure compound or a pharmaceutically acceptable salt, solvate, hydrate or prodrug thereof can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

The compounds of this invention may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

One having ordinary skill in the art of organic synthesis will recognize that the maximum number of heteroatoms in a stable, chemically feasible heterocyclic ring, whether it is aromatic or non-aromatic, is determined by the size of the ring, the degree of unsaturation and the valence of the heteroatoms. In general, a heterocyclic ring may have one to four heteroatoms so long as the heteroaromatic ring is chemically feasible and stable.

### Compounds of the Invention

In certain aspects, provided are compounds of Formula (I):

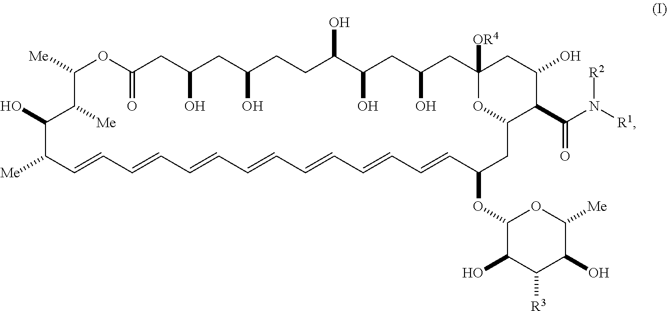

or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

In certain embodiments,
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

In certain embodiments,
R¹ and R² independently are hydrogen, unsubstituted C₁₋₆ alkyl, alkoxy C₁₋₆ alkyl, halo C₁₋₆ alkyl, amino C₁₋₆ alkyl, heterocyclyl C₁₋₆ alkyl, unsubstituted C₂₋₆ alkynyl, unsubstituted C₃₋₁₀ carbocyclyl, amino C₃₋₁₀ carbocyclyl, unsubstituted 3- to 10-membered heterocyclyl, or hydroxyl 3- to 10-membered heterocyclyl.

In certain embodiments, at least one of R¹ and R² is hydrogen.

In certain embodiments, at least one of R¹ and R² is hydrogen and R¹ and R² are not both hydrogen.

In certain embodiments,
R¹ and R², together with the nitrogen to which they are attached, form a substituted or unsubstituted 3- to 10-membered heterocyclyl.

In certain embodiments,
R¹ and R², together with the nitrogen to which they are attached, form an unsubstituted 3- to 10-membered heterocyclyl, amino 3- to 10-membered heterocyclyl, hydroxyl 3- to 10-membered heterocyclyl, or heterocyclyl 3- to 10-membered heterocyclyl.

In certain embodiments, R³ is —NR⁵R⁶, wherein
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein
R^{f} is selected from the group consisting of 2-alken-1-yl, tert-butyl, benzyl and fluorenylmethyl; or
R⁵ and R⁶, together with the nitrogen to which they are attached, form a substituted or unsubstituted 3- to 10-membered heterocyclyl.

In certain embodiments,
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein

In certain embodiments, R⁵ and R⁶ independently are hydrogen or C(O)OR^{f}.

In certain embodiments, R^{f} is fluorenylmethyl.

In certain embodiments, at least one of R⁵ and R⁶ is hydrogen.

In certain embodiments, R⁵ and R⁶ are both hydrogen.

In certain embodiments, R⁴ is hydrogen, substituted or unsubstituted C₁₋₆ alkyl, or substituted or unsubstituted C₂₋₆ alkenyl.

In certain embodiments, R⁴ is hydrogen, halo C₁₋₆ alkyl, or unsubstituted C₂₋₆ alkenyl.

In certain embodiments, R⁴ is hydrogen.

In certain aspects, provided are compounds of Formula (II):

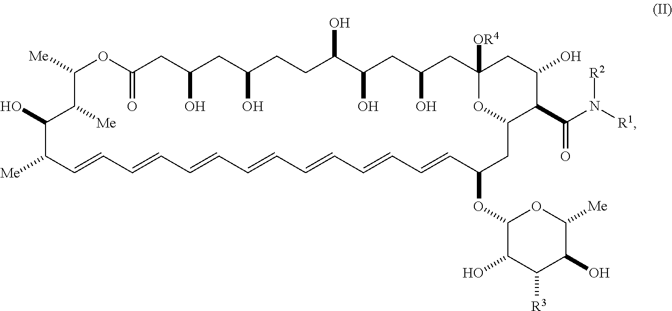

or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

In certain embodiments,
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

In certain embodiments,
R¹ and R² independently are hydrogen, unsubstituted C₁₋₆ alkyl, hydroxyl C₁₋₆ alkyl, amino C₁₋₆ alkyl, unsubstituted C₃₋₁₀ carbocyclyl.

In certain embodiments, at least one of R¹ and R² is hydrogen.

In certain embodiments, at least one of R¹ and R² is hydrogen and R¹ and R² are not both hydrogen.

In certain embodiments, R³ is —NR⁵R⁶, wherein
R⁵ and R⁶ independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, together with the nitrogen to which they are attached, form a substituted or unsubstituted 3- to 10-membered heterocyclyl;

In certain embodiments,
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein
R^{f} is selected from the group consisting of 2-alken-1-yl, tert-butyl, benzyl and fluorenylmethyl.

In certain embodiments, R⁵ and R⁶ independently are hydrogen or C(O)OR^{f}.

In certain embodiments, R^{f} is fluorenylmethyl.

In certain embodiments, at least one of R⁵ and R⁶ is hydrogen.

In certain embodiments, R⁵ and R⁶ are both hydrogen.

In certain embodiments, R⁴ is hydrogen, substituted or unsubstituted C₁₋₆ alkyl, or substituted or unsubstituted C₂₋₆ alkenyl.

In certain embodiments, R⁴ is hydrogen, halo C₁₋₆ alkyl, or unsubstituted C₂₋₆ alkenyl.

In certain embodiments, R⁴ is hydrogen.

In certain aspects, provided is a compound selected from the group consisting of:

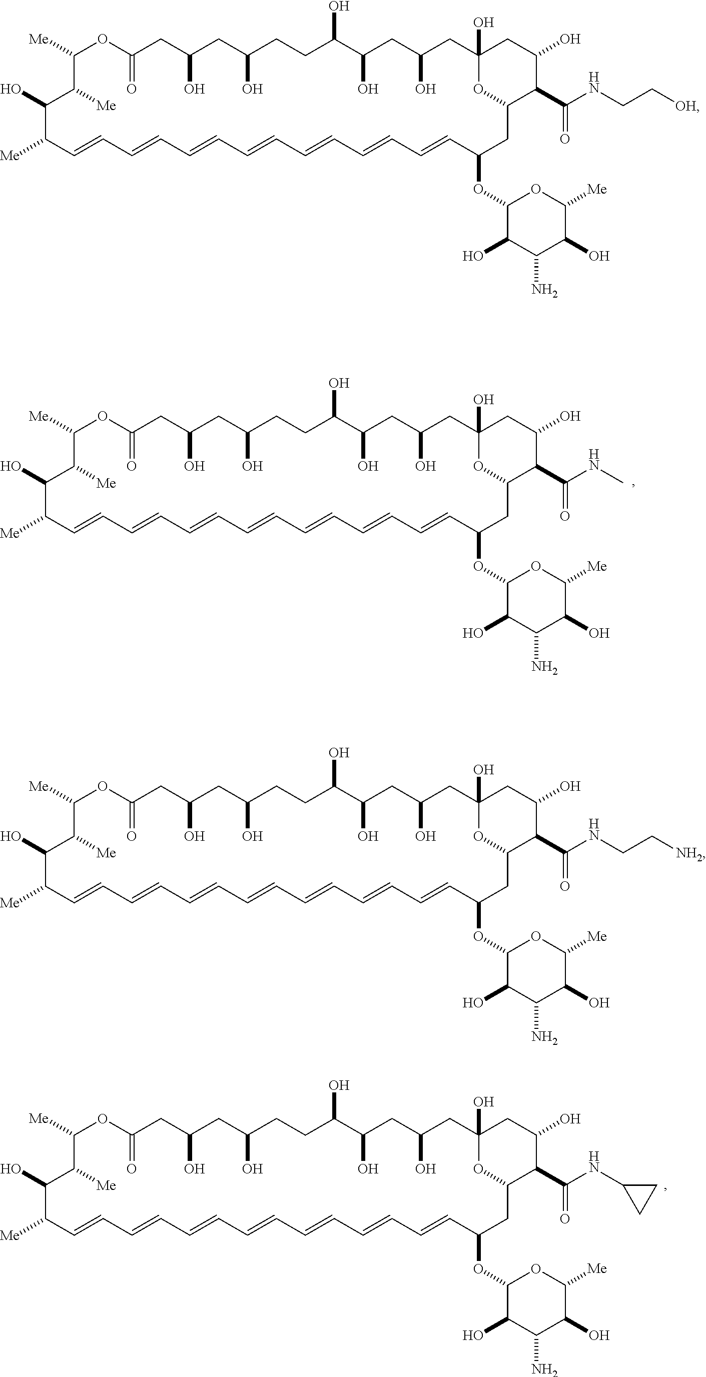

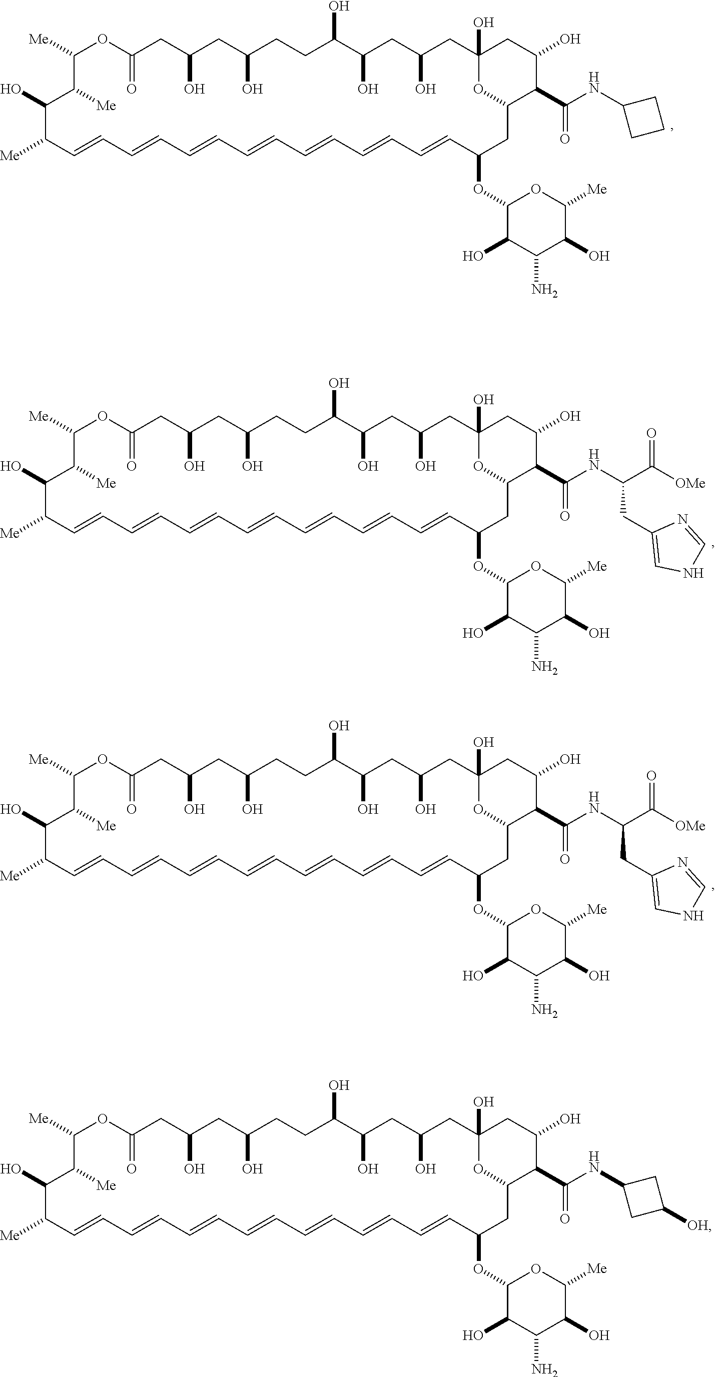

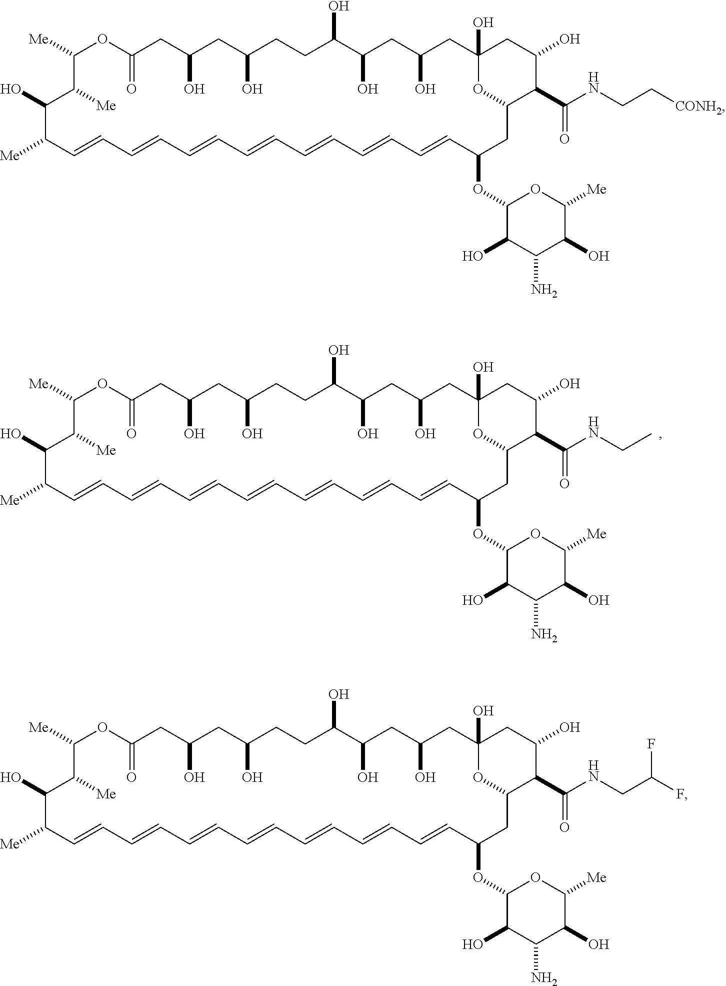

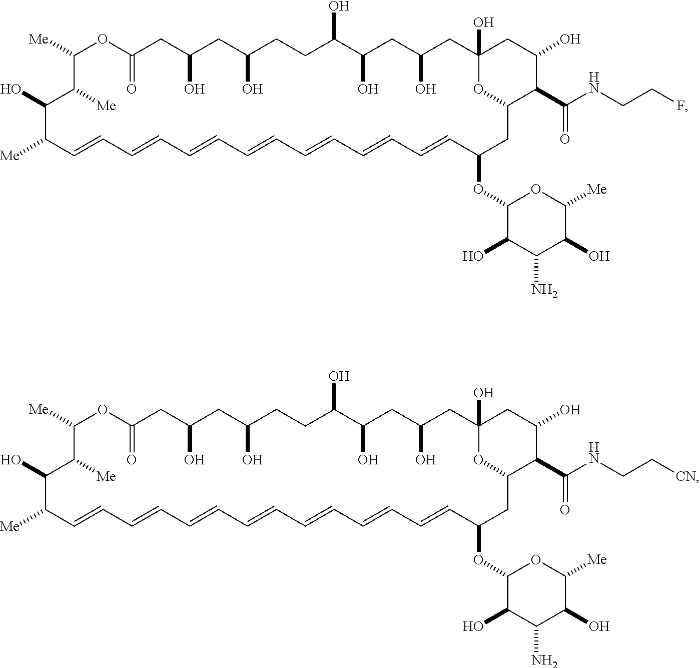

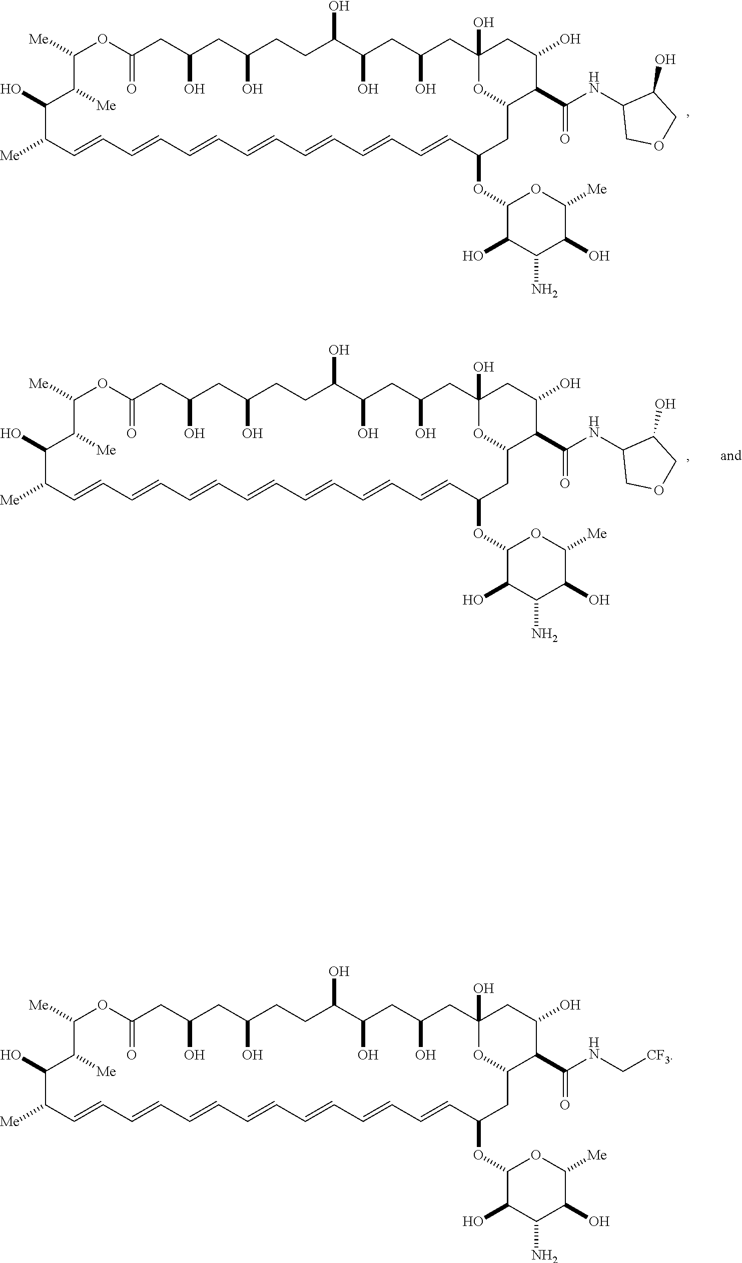

In certain aspects, provided is a compound selected from the group consisting of:

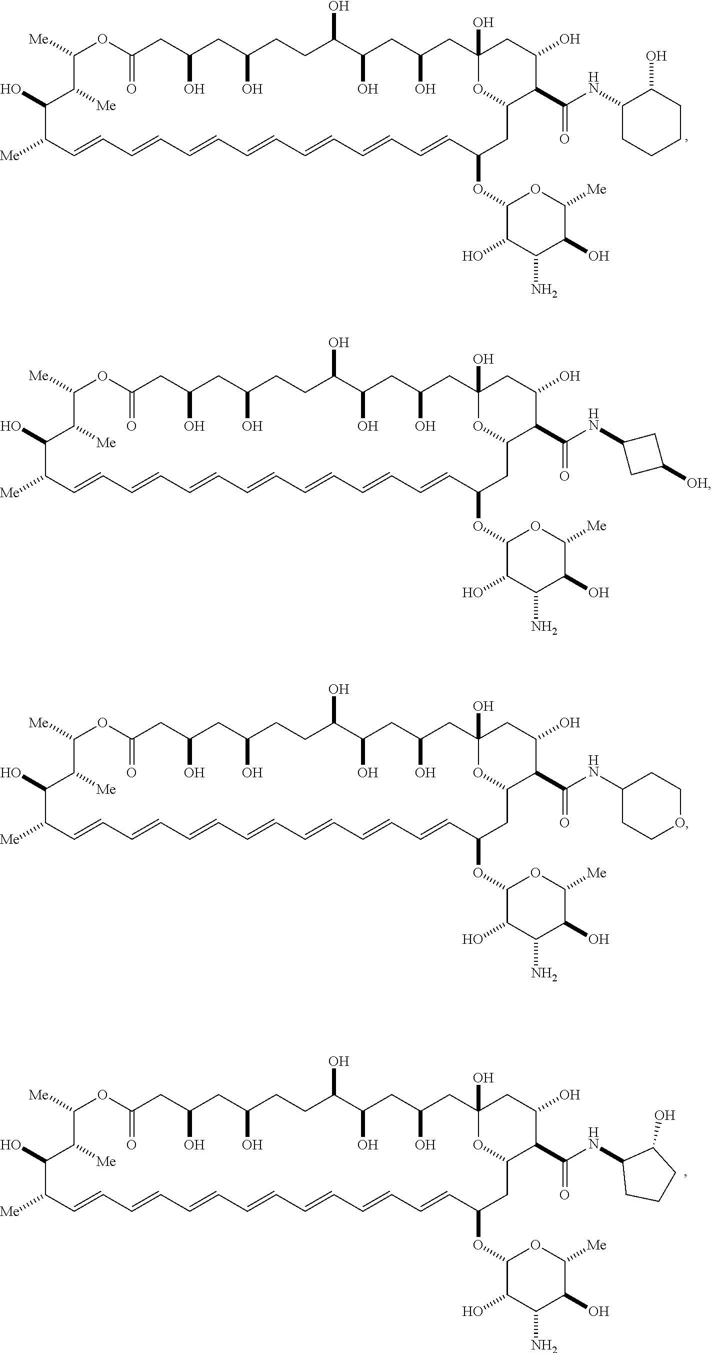

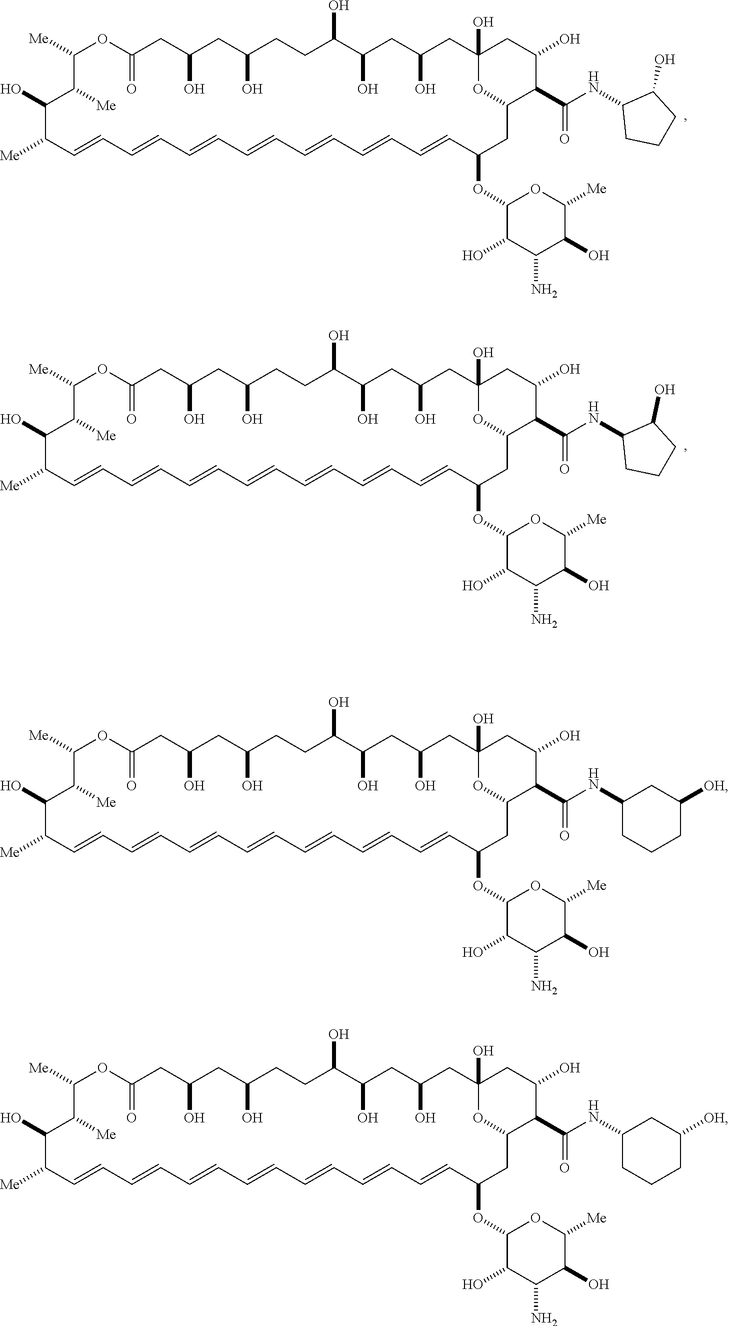

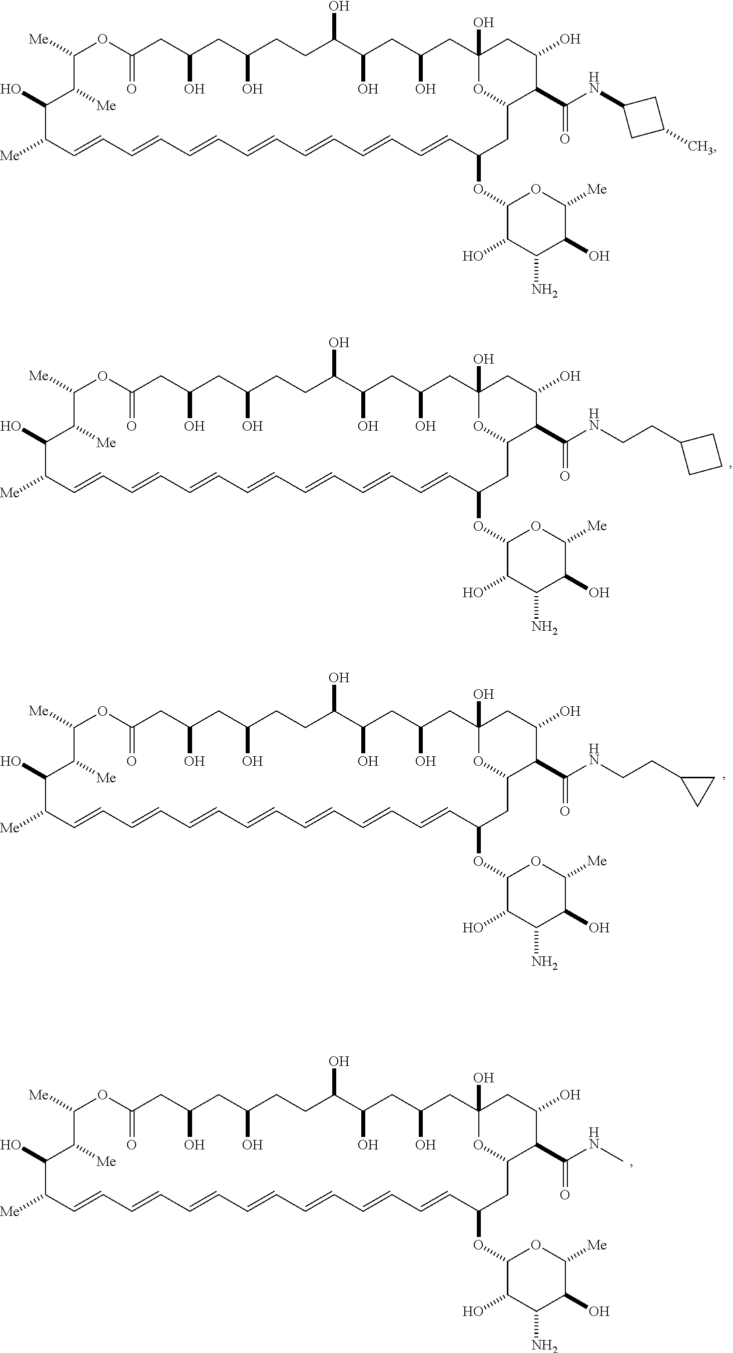

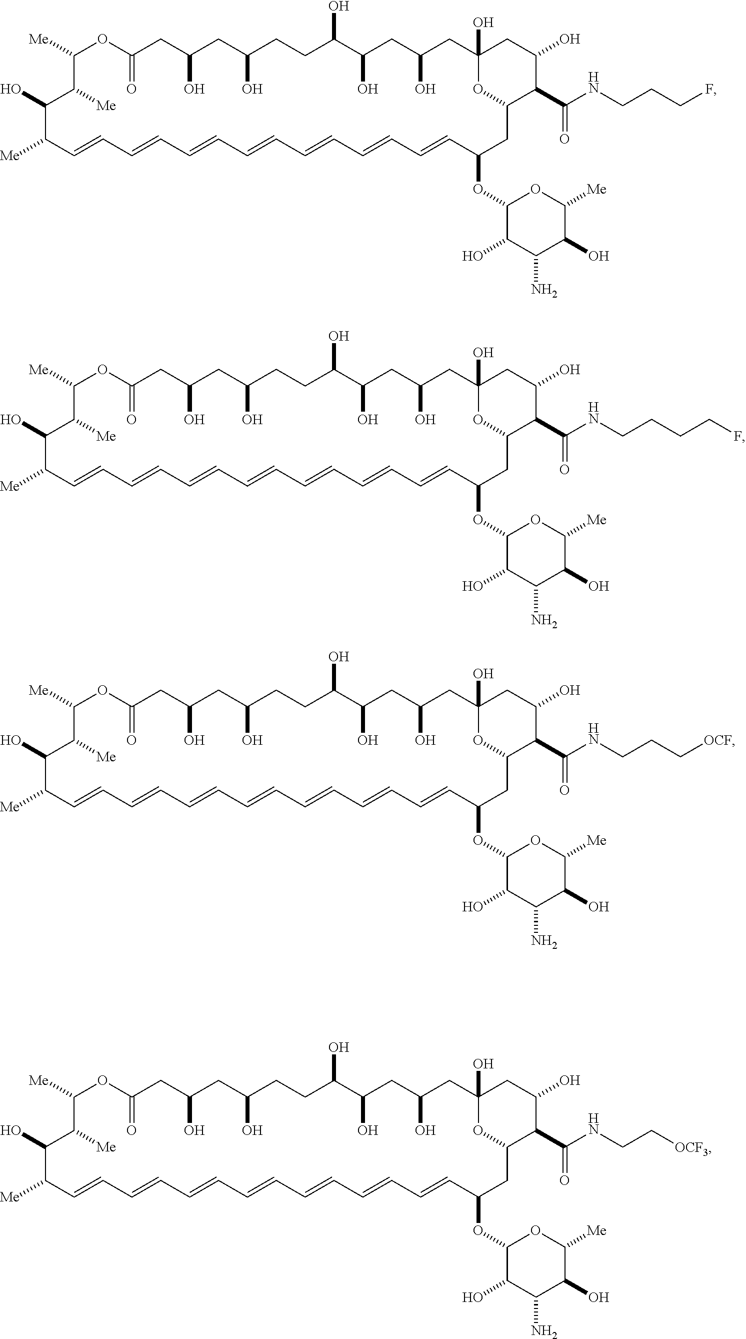

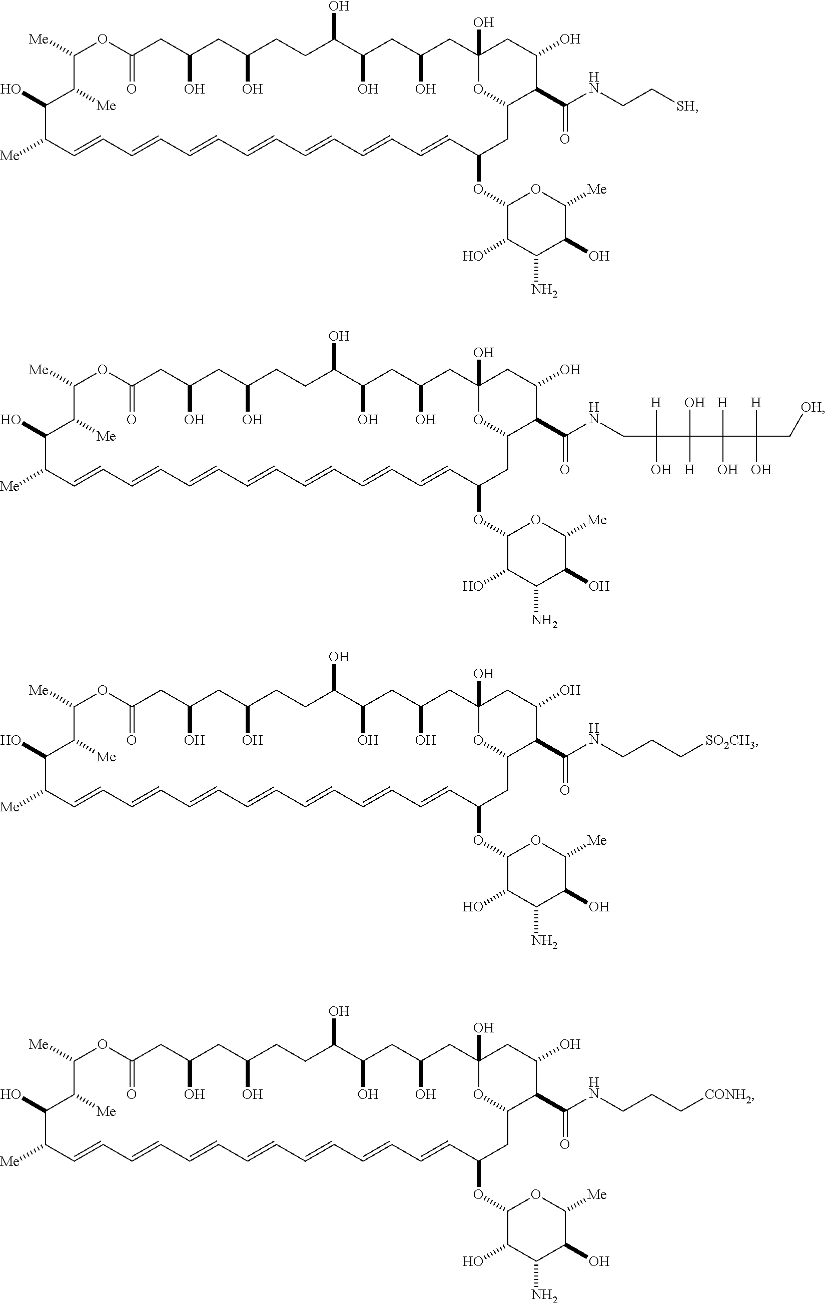

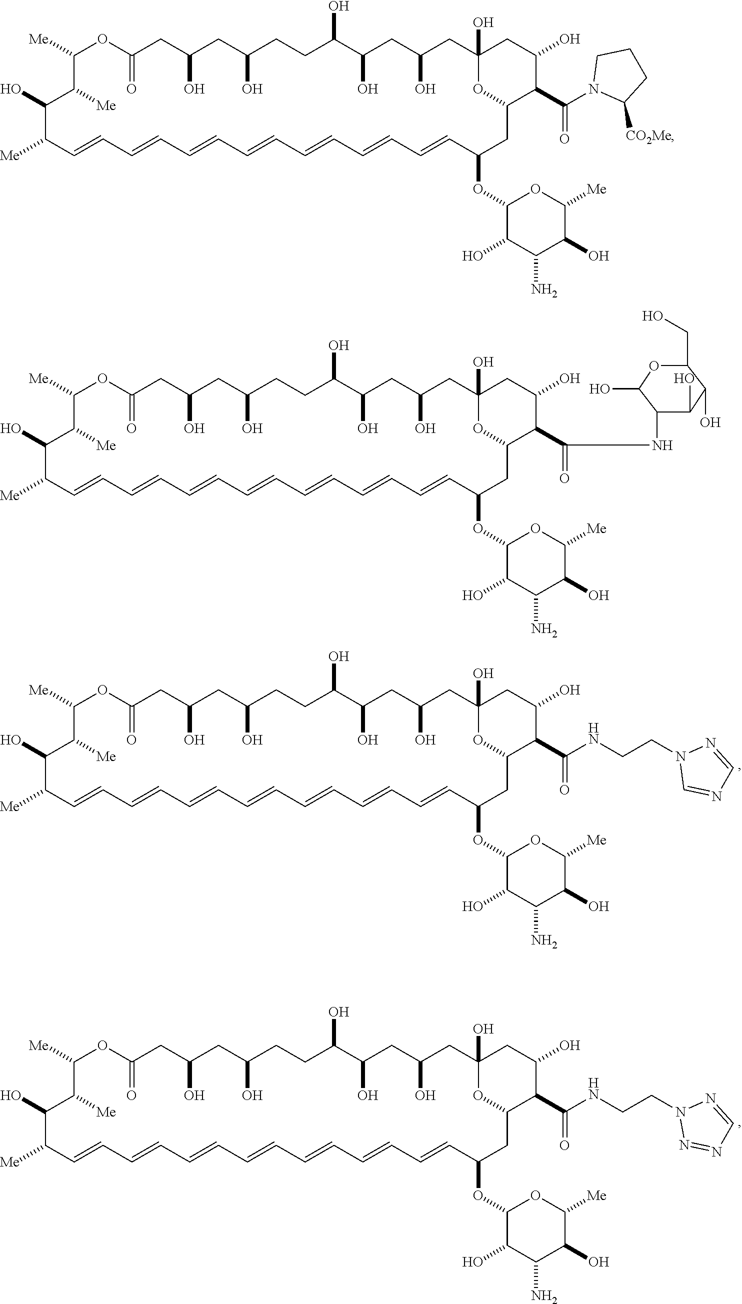

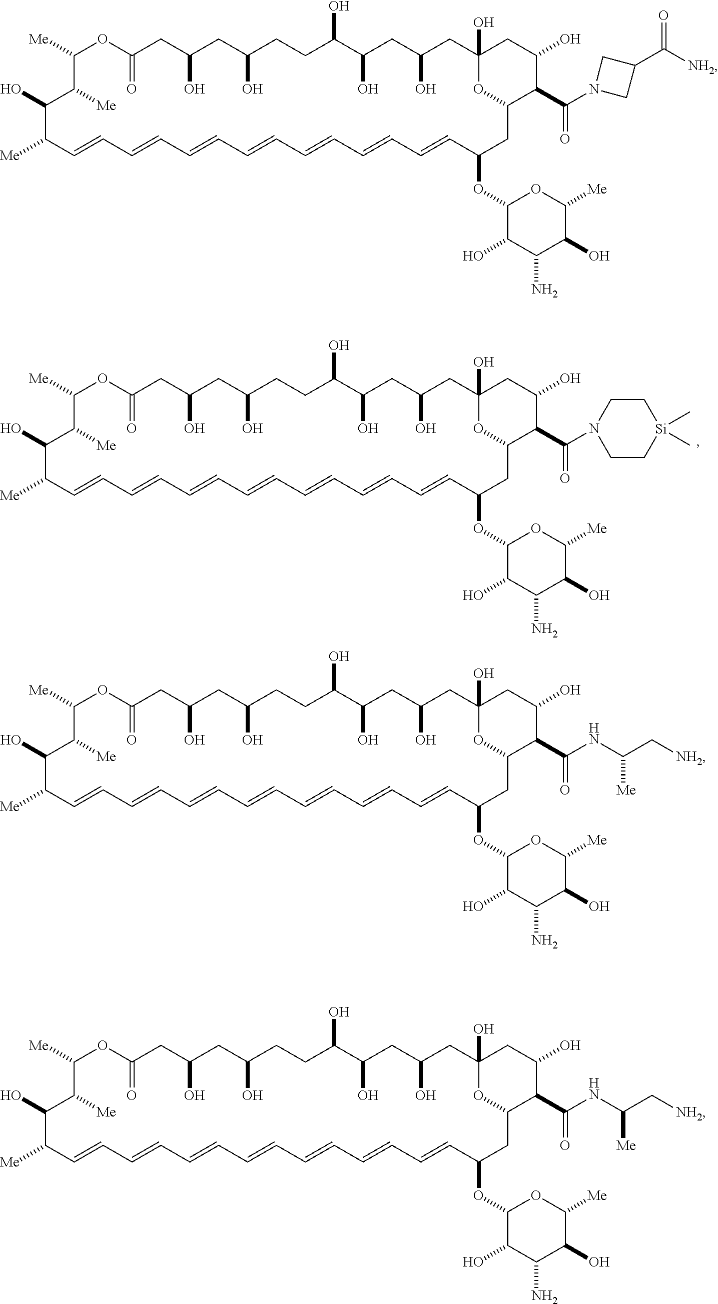

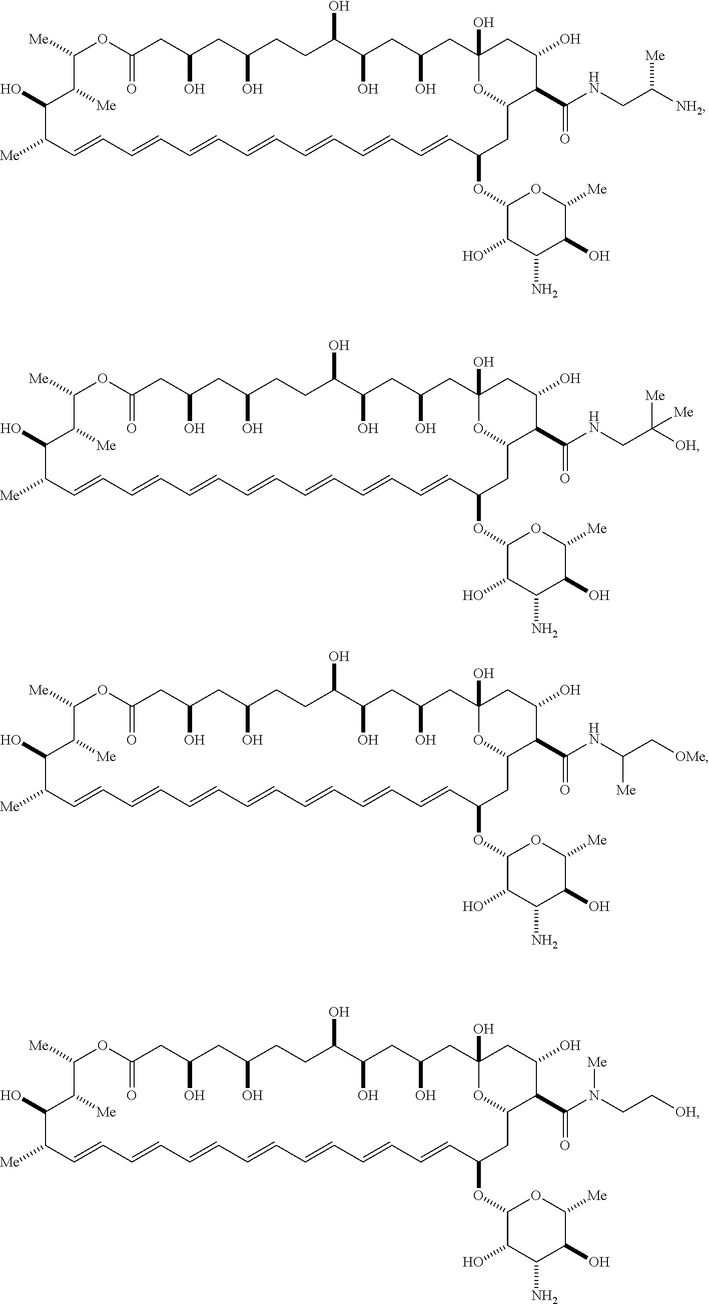

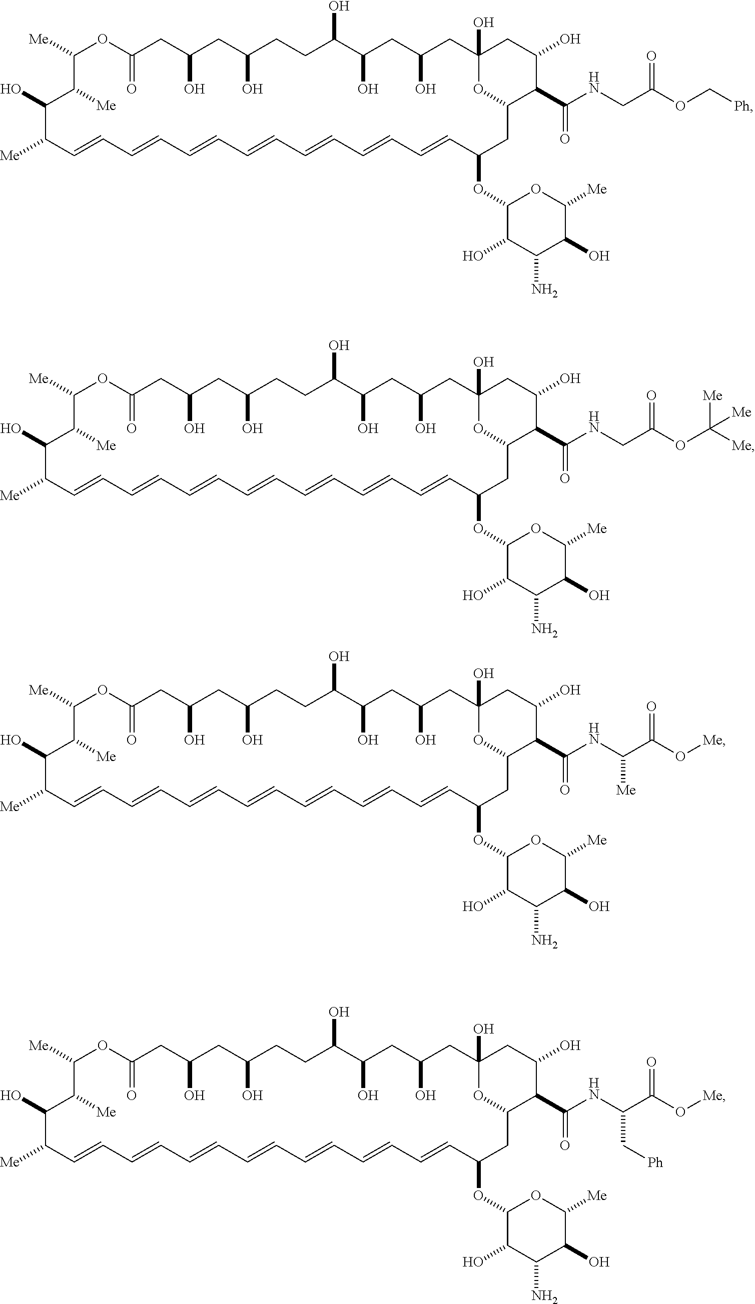

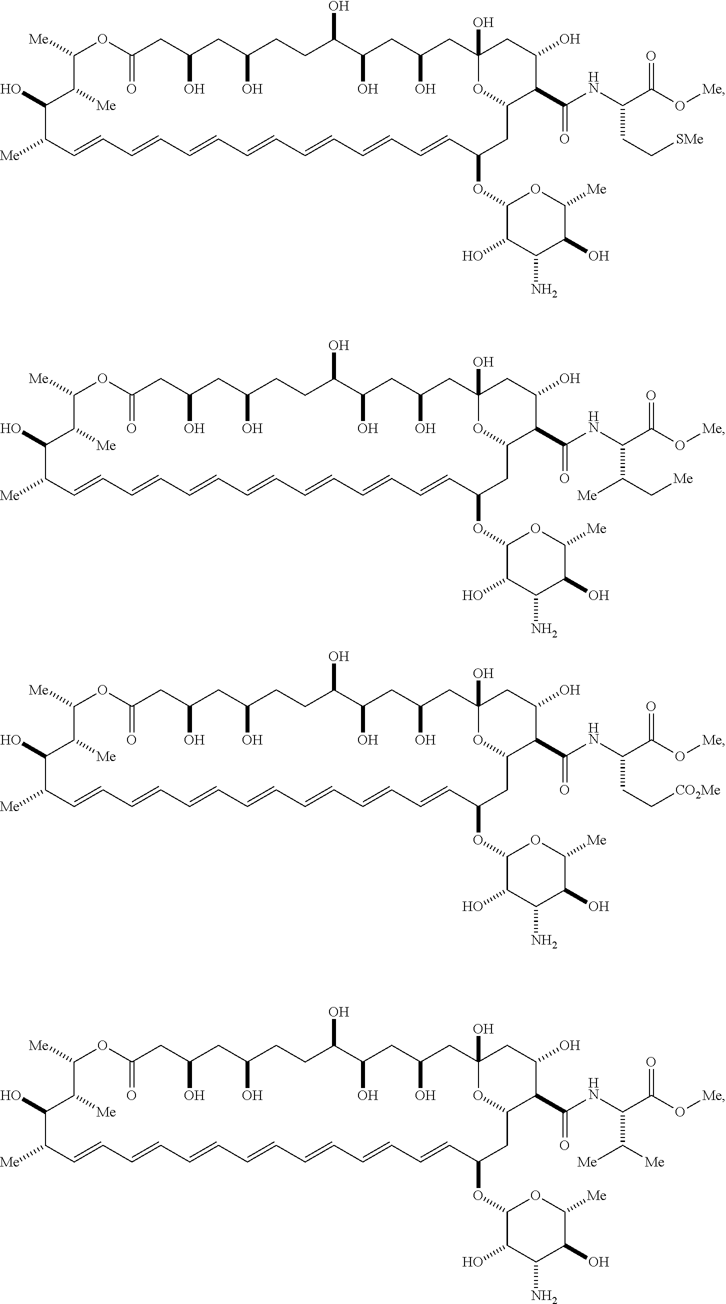

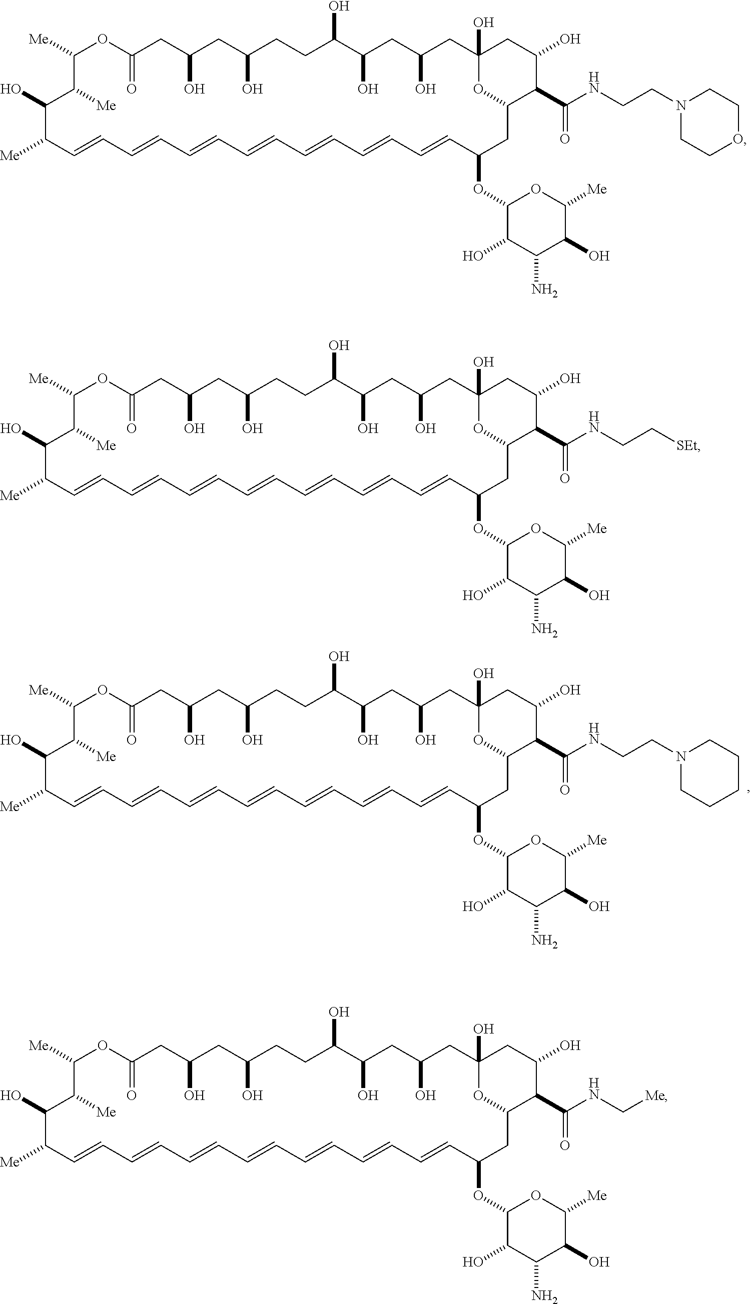

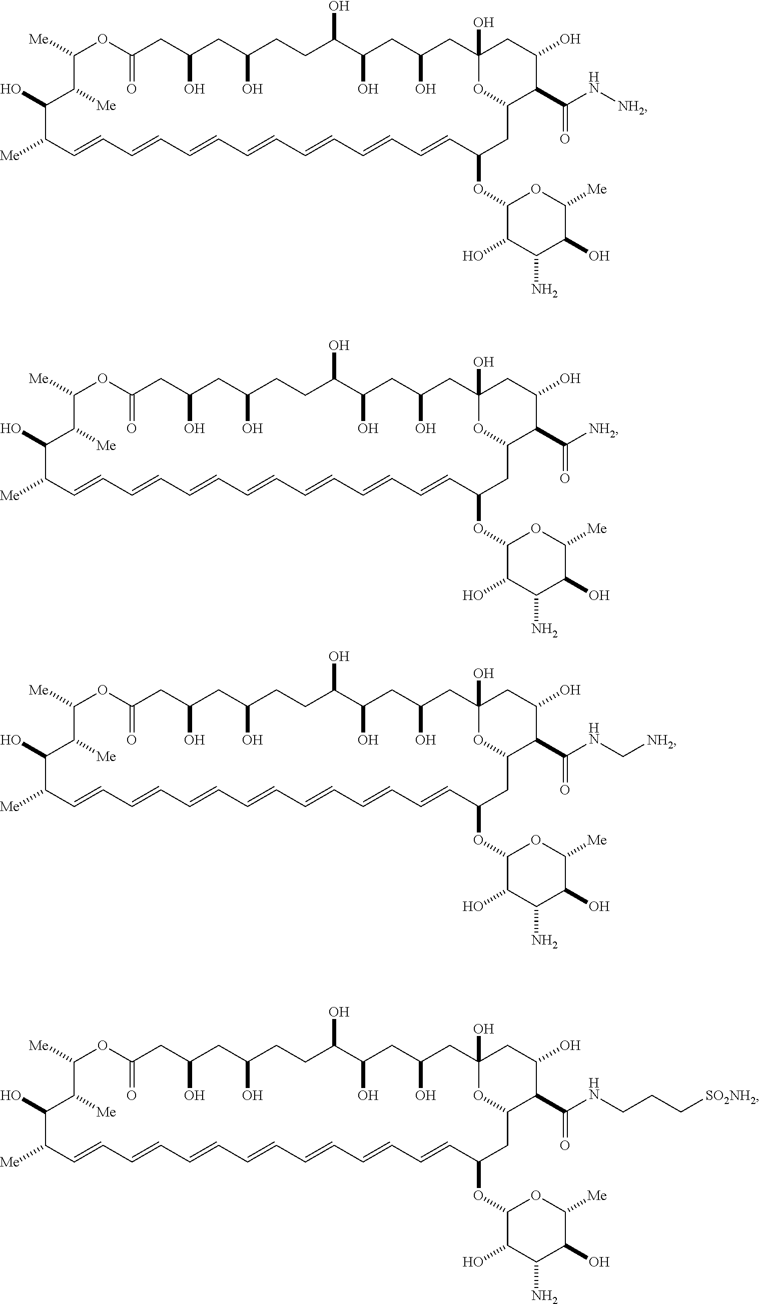

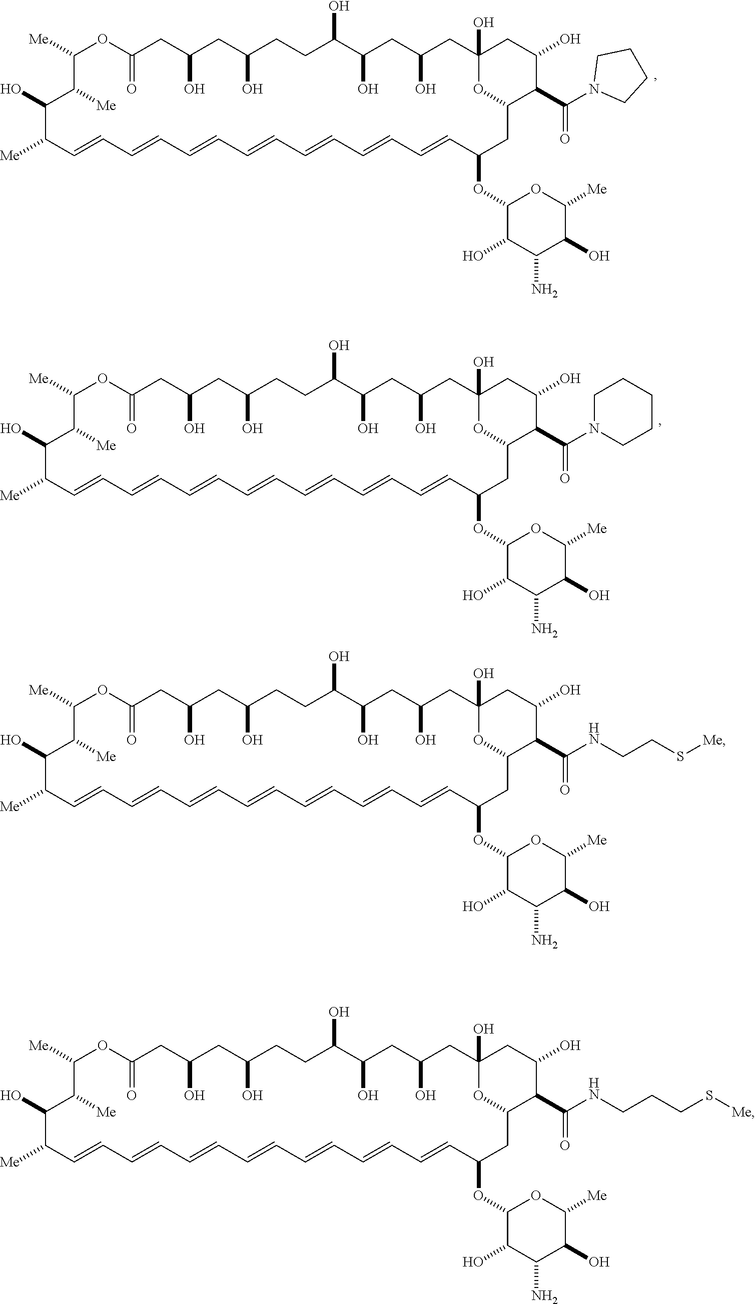

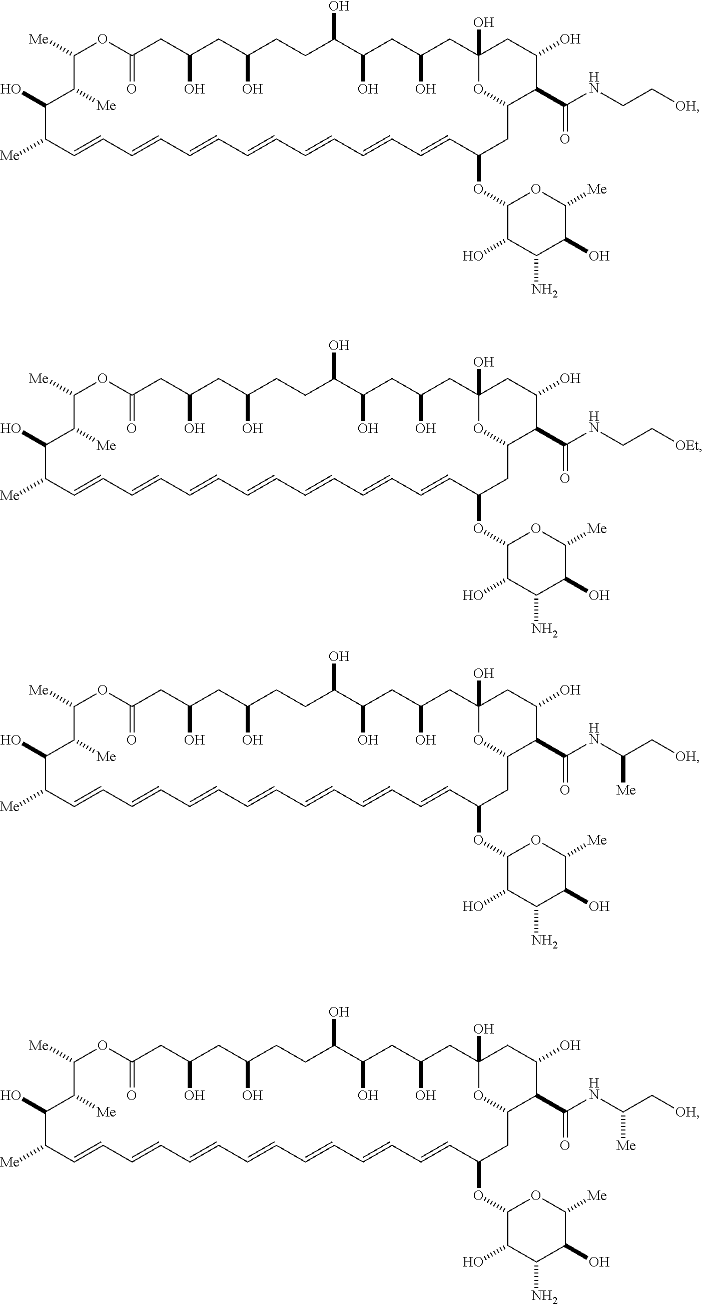

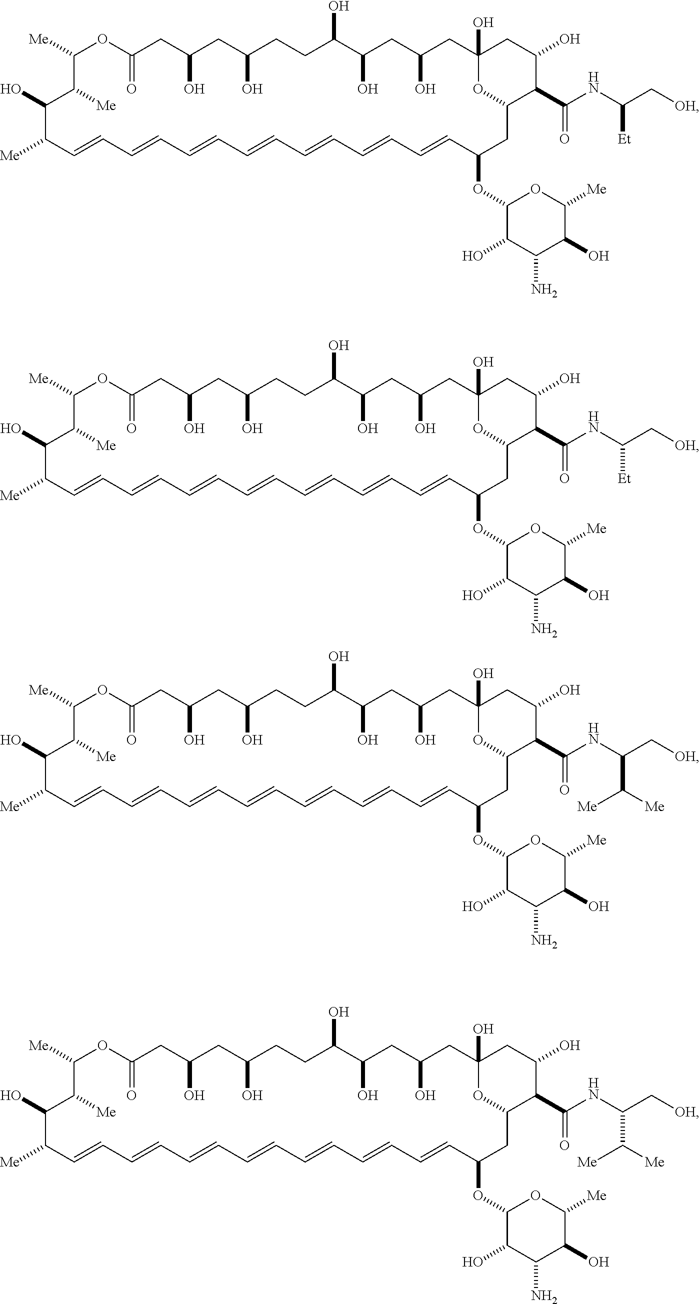

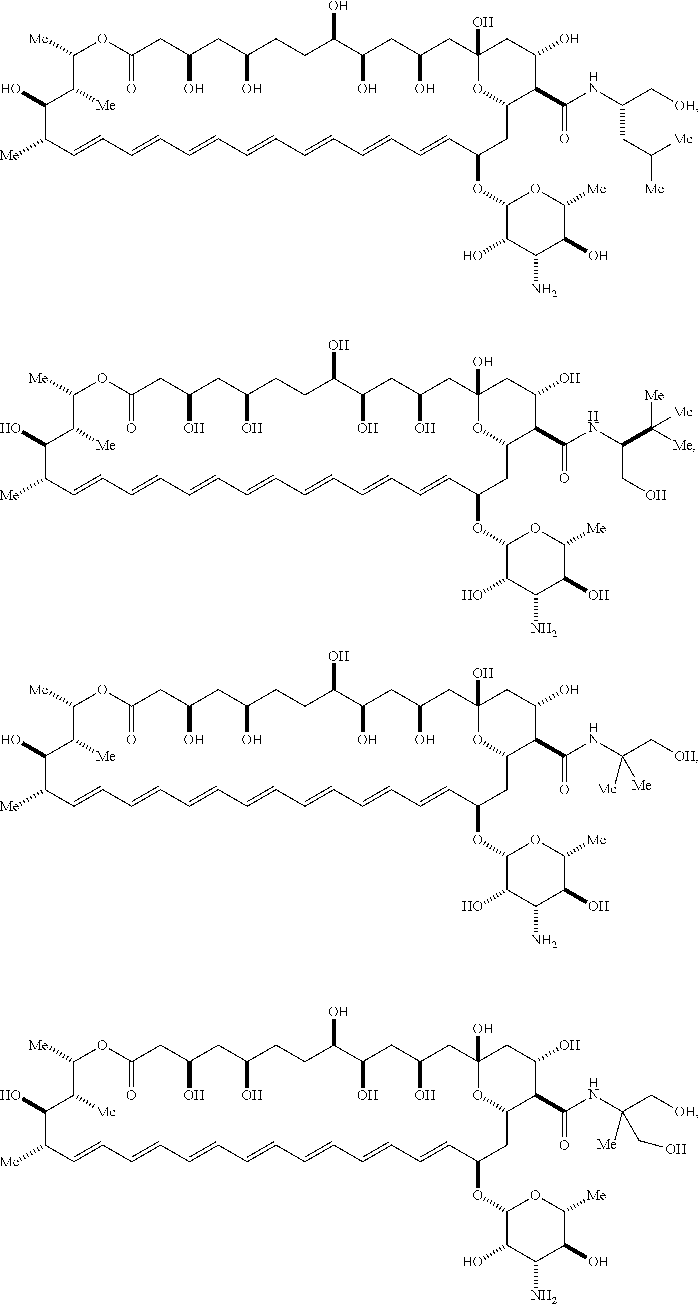

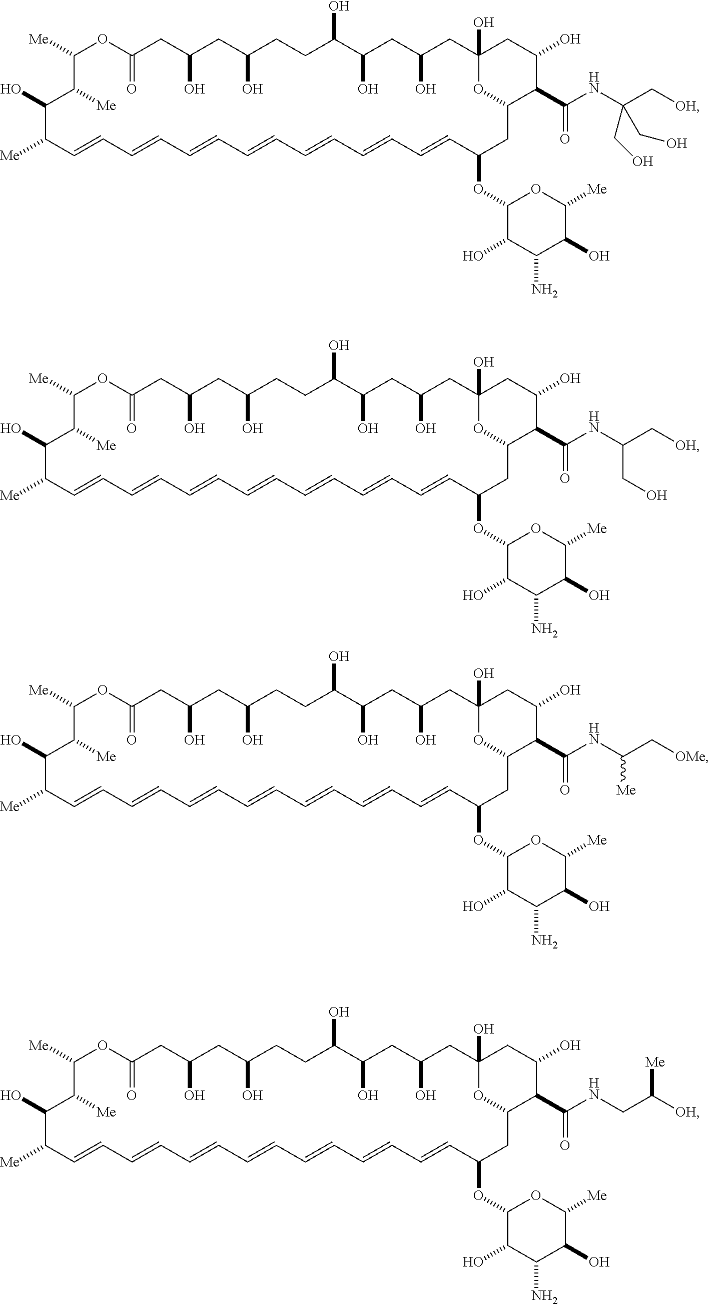

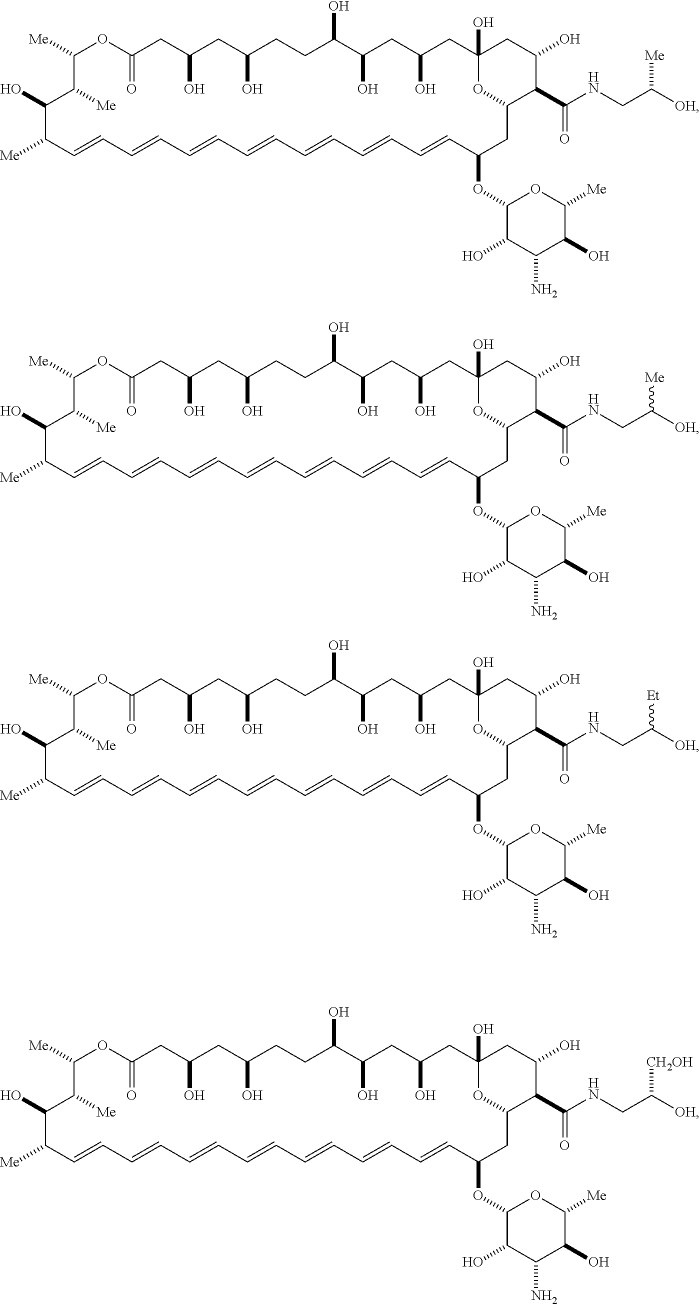

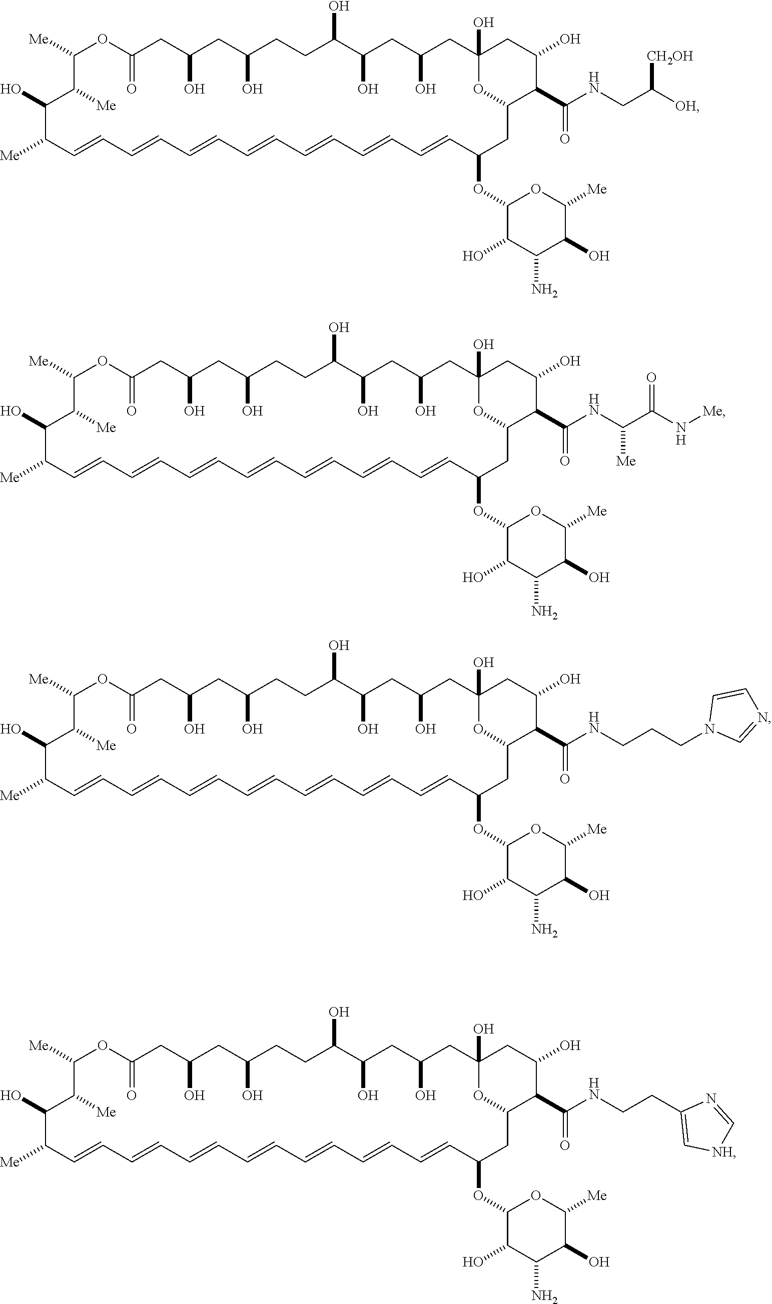

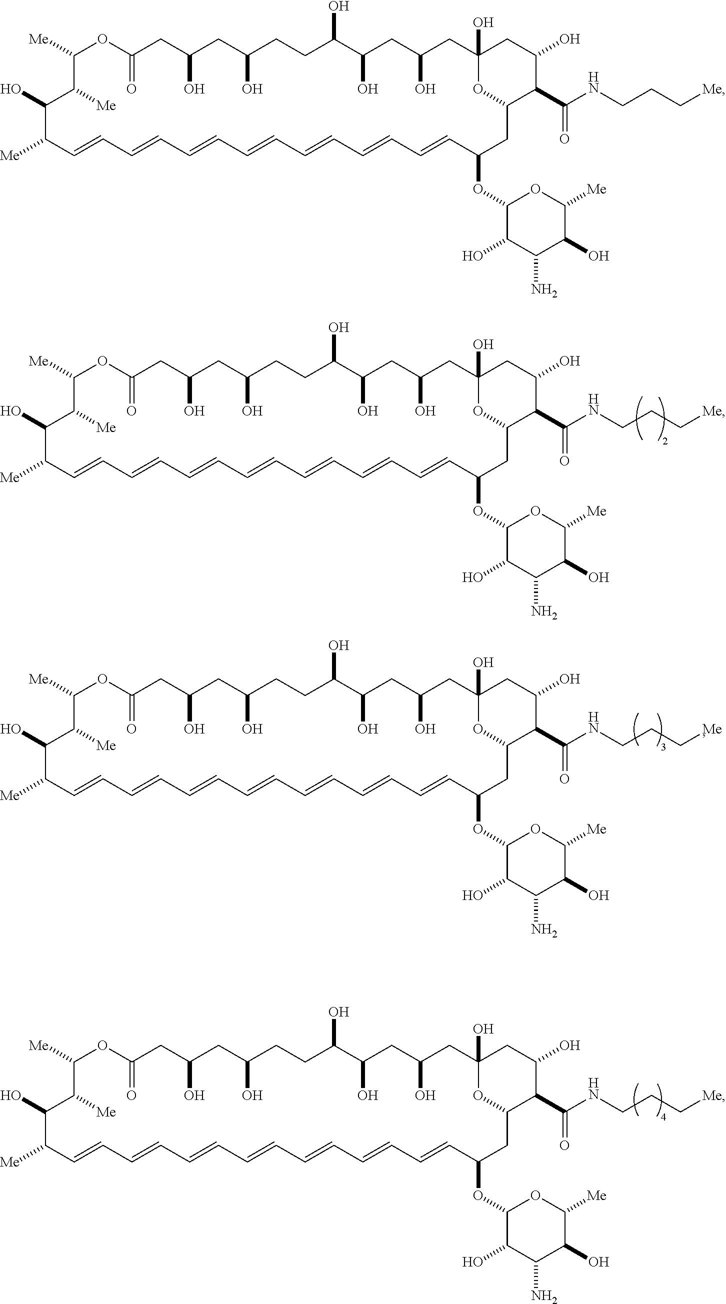

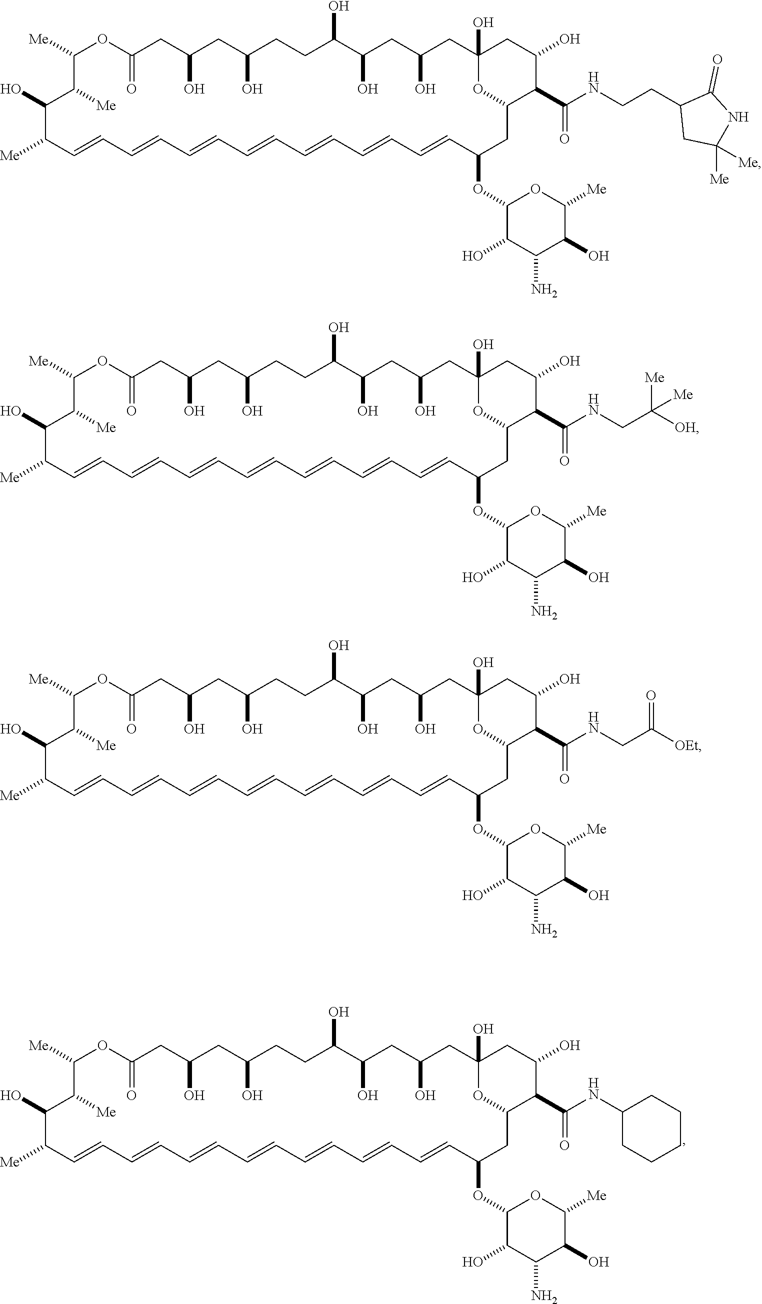

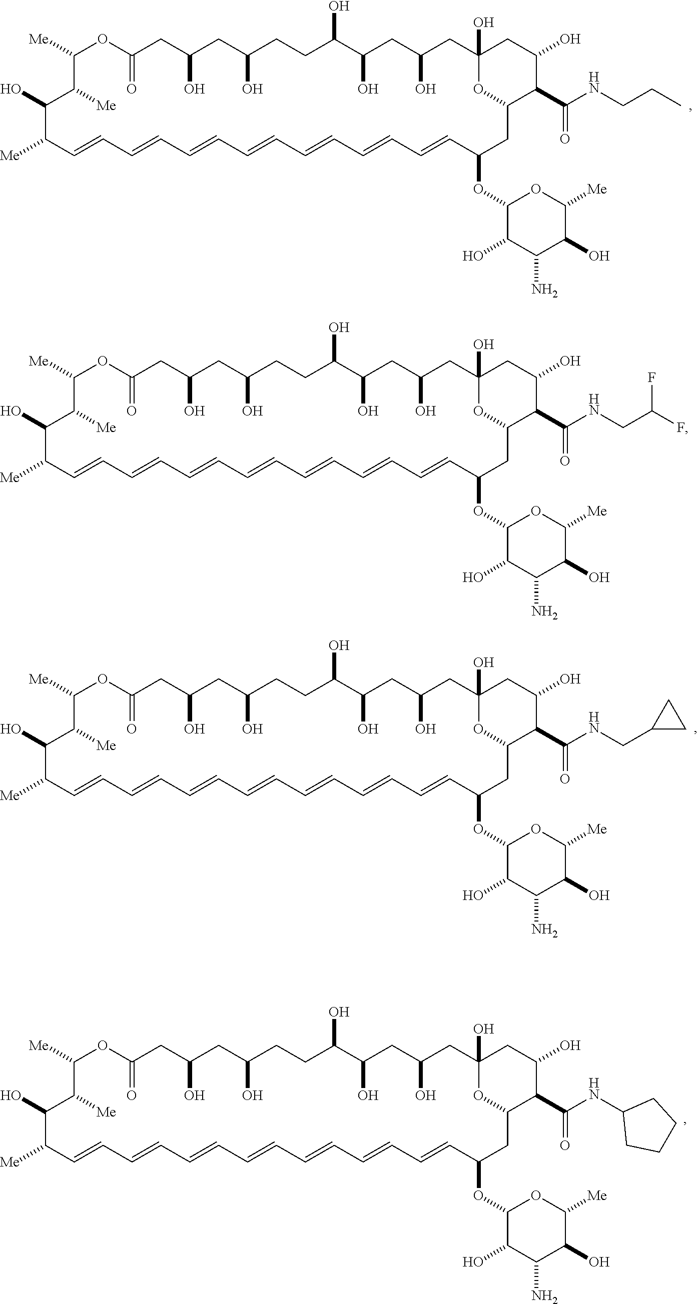

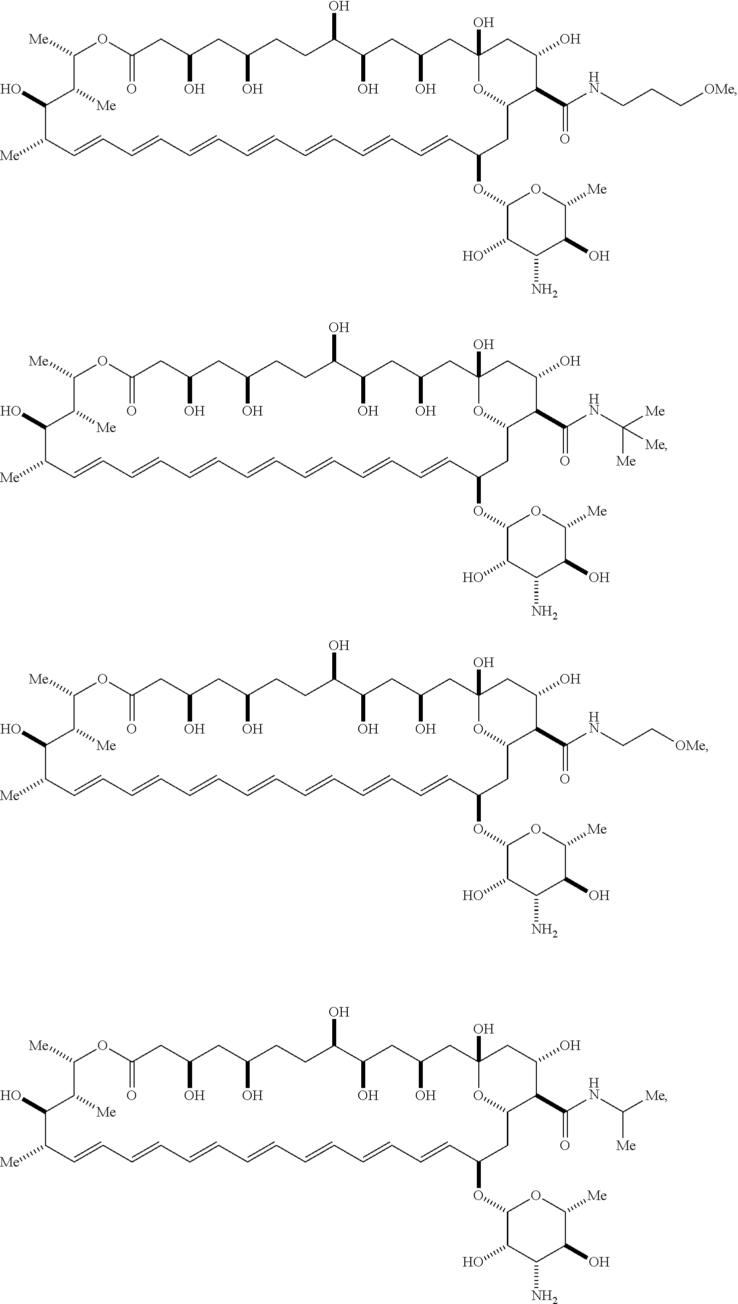

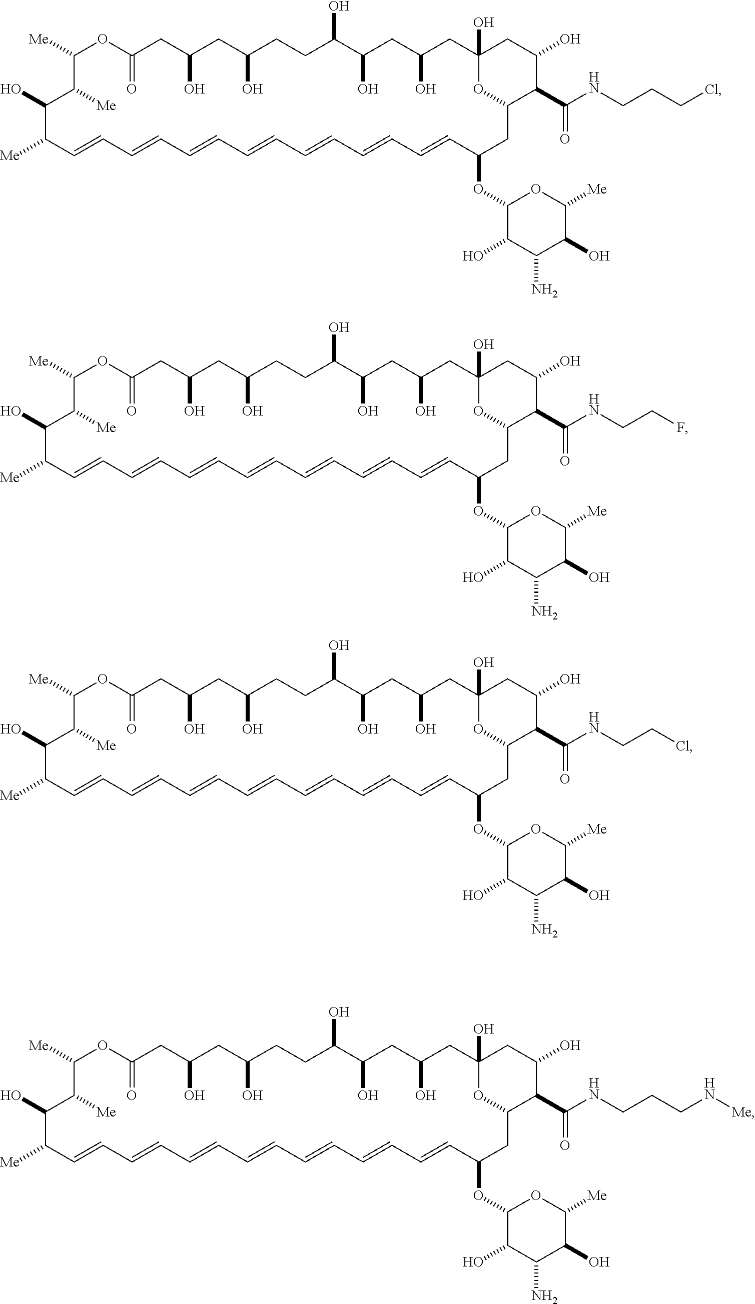

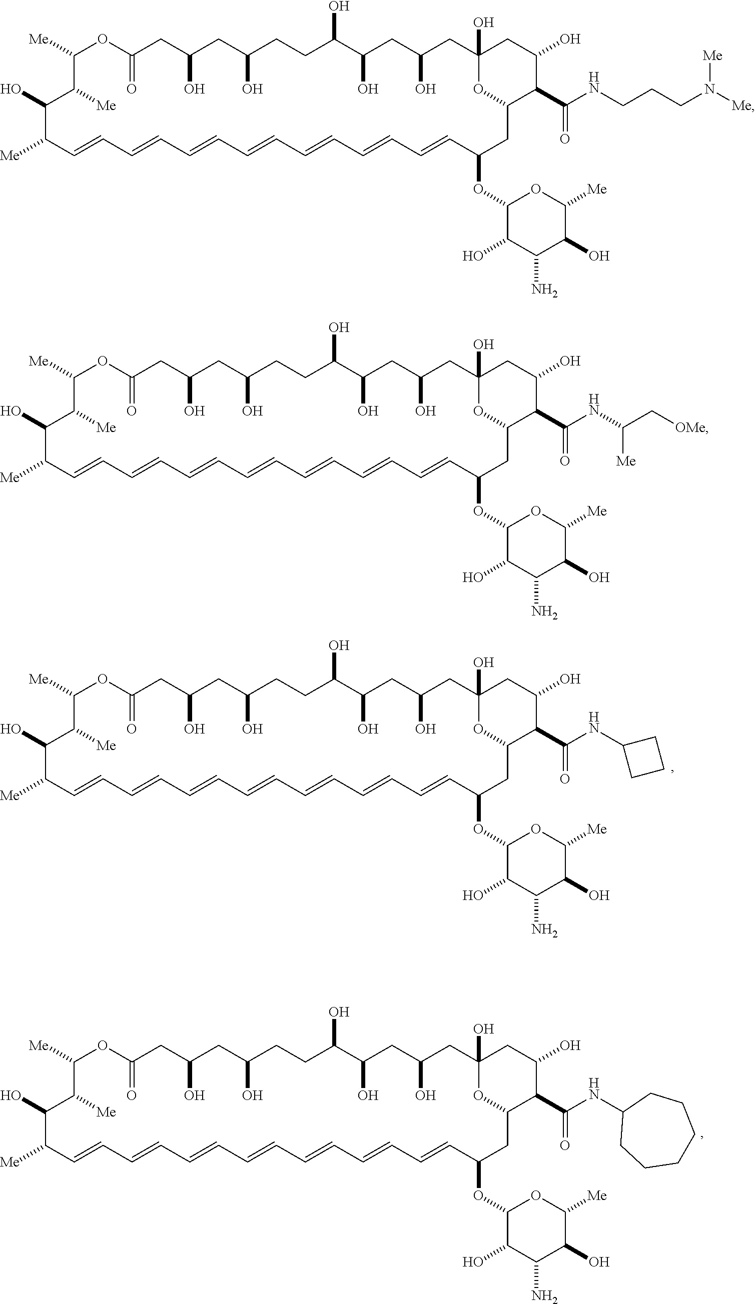

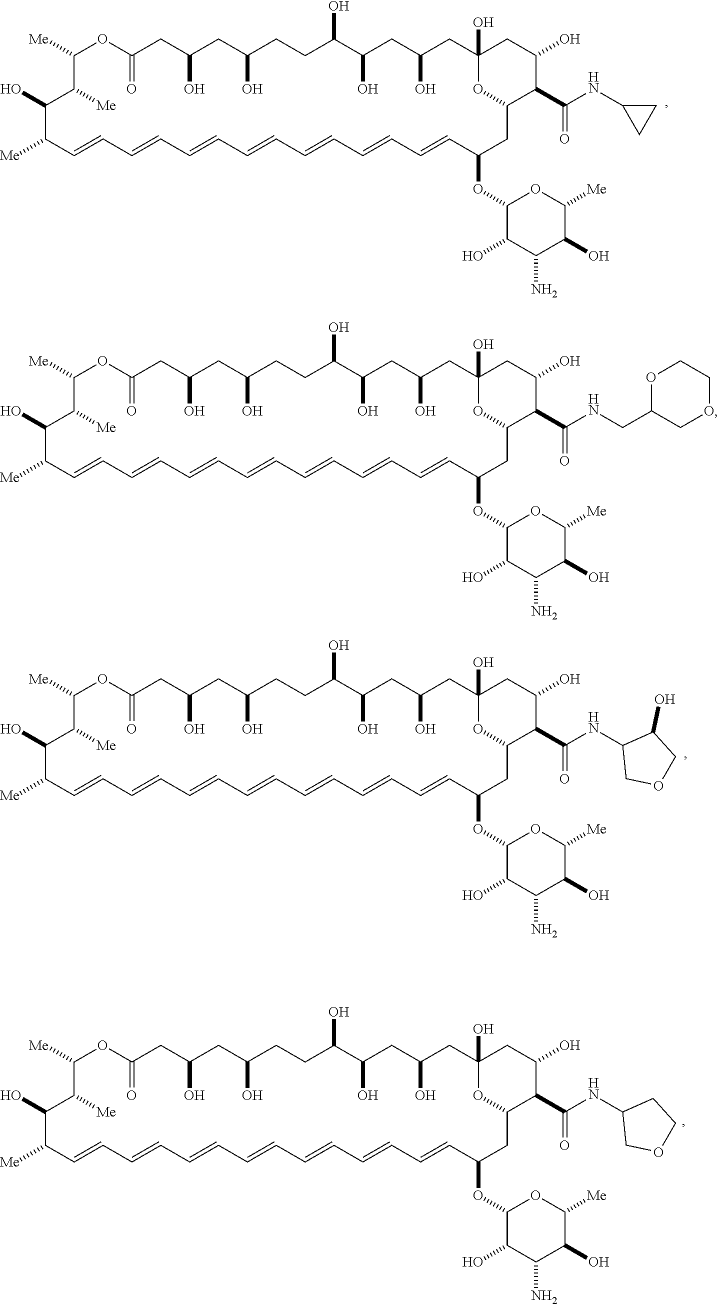

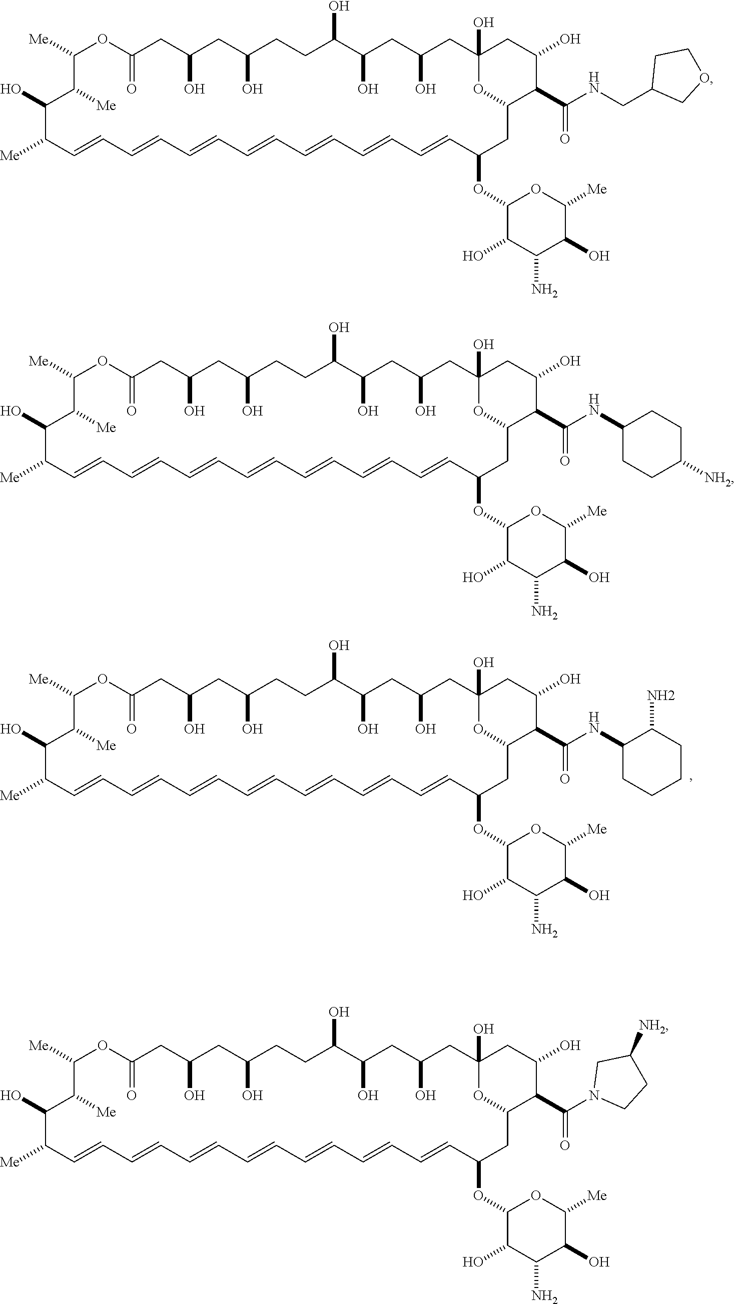

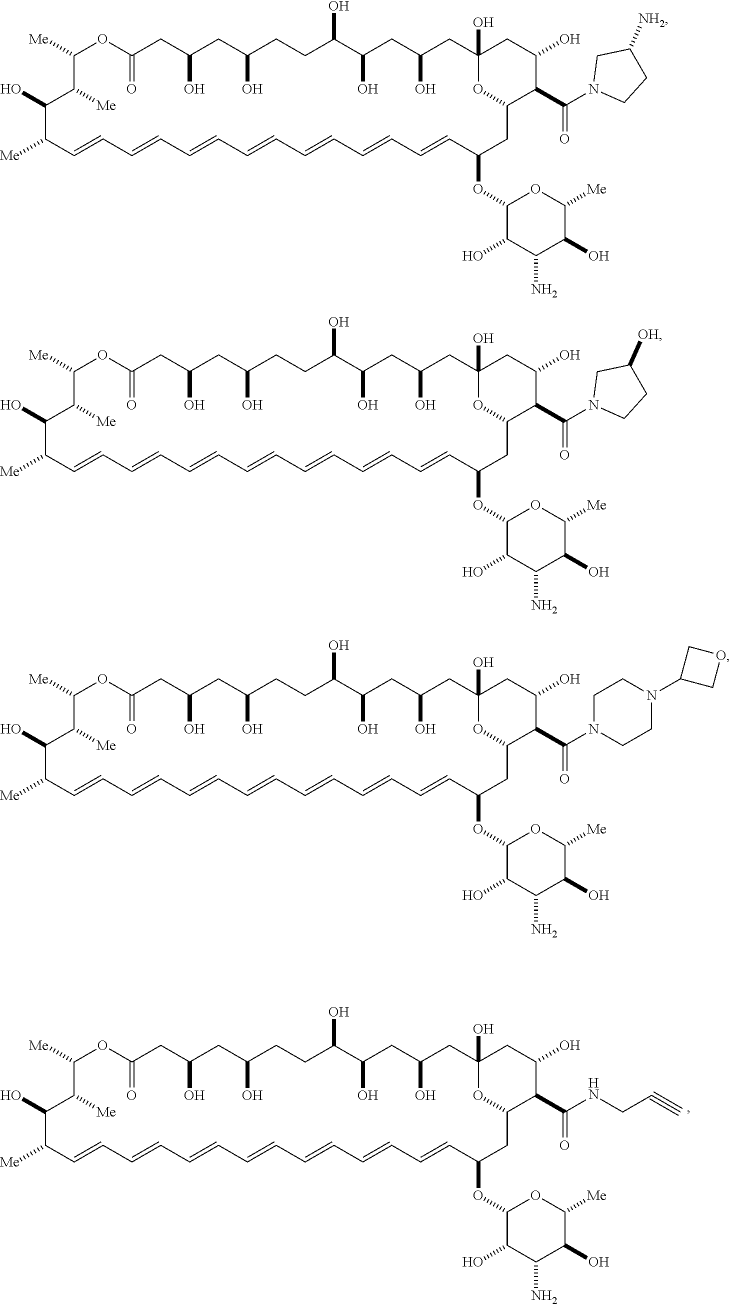

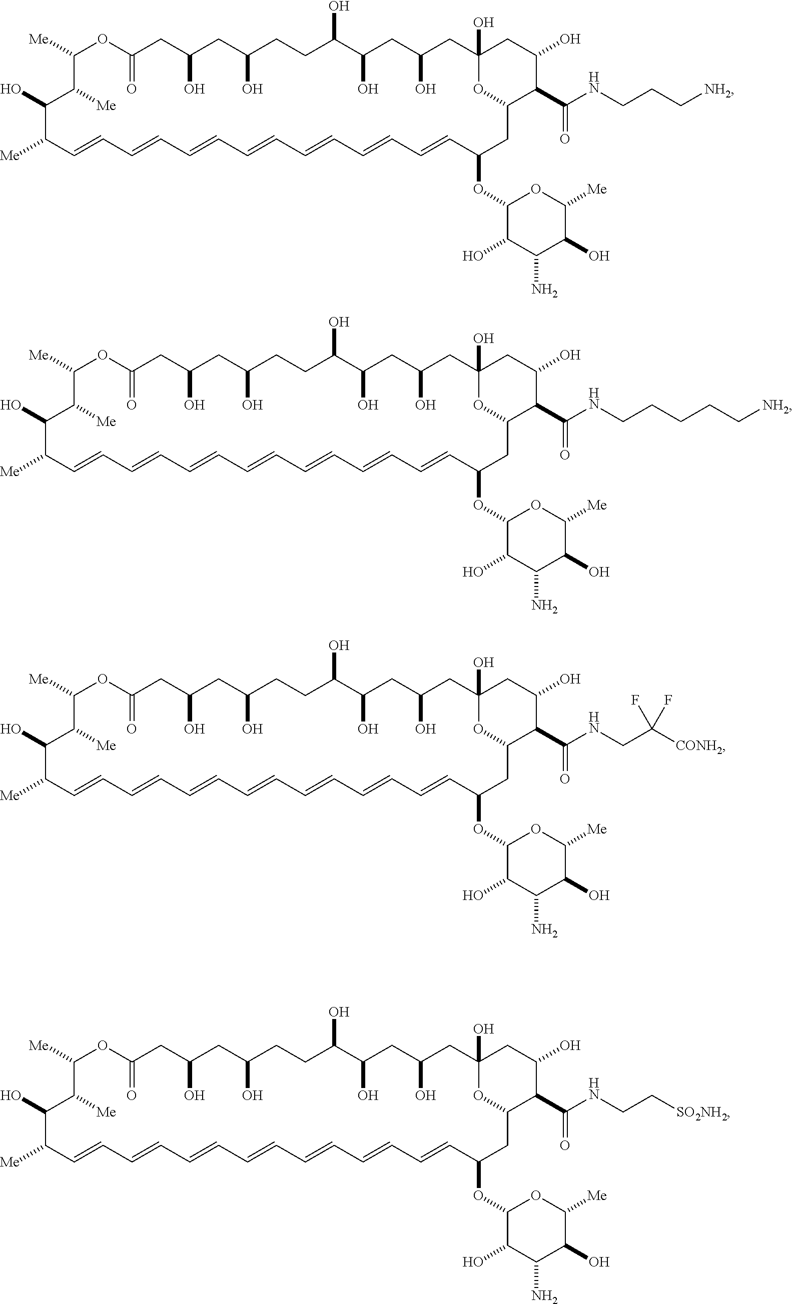

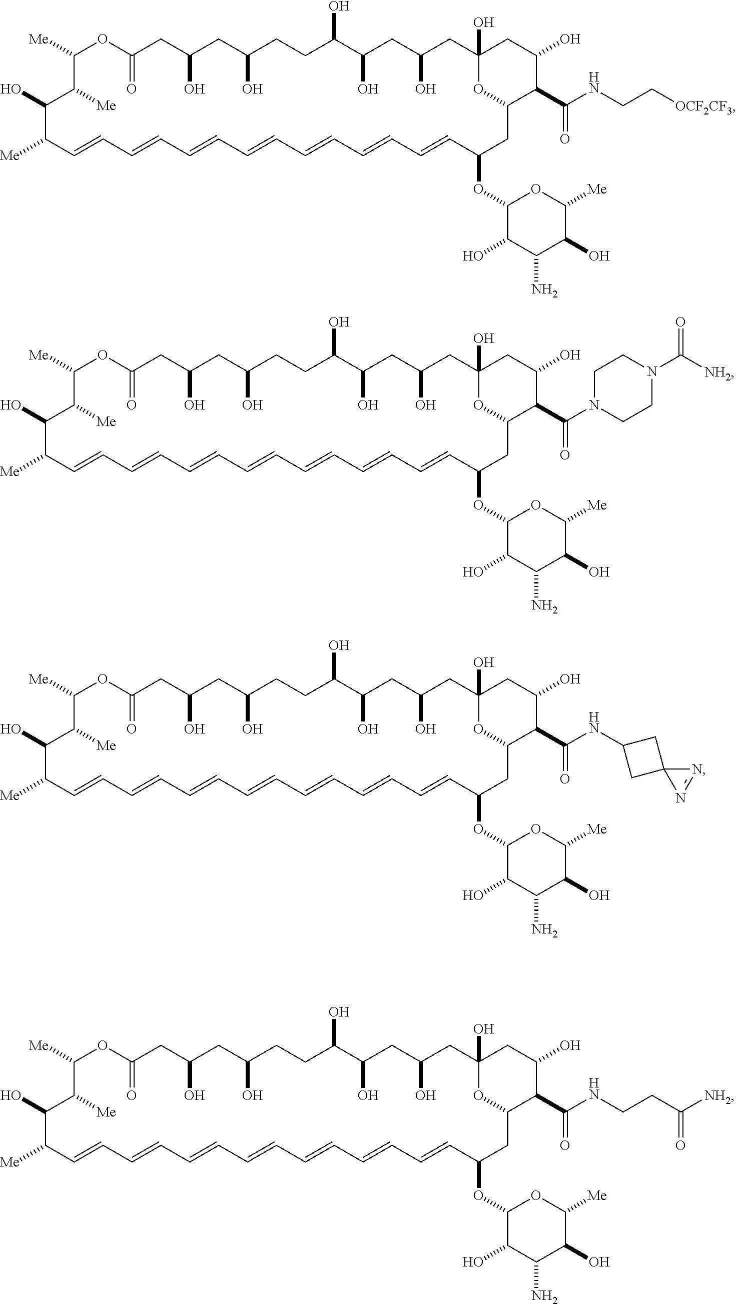

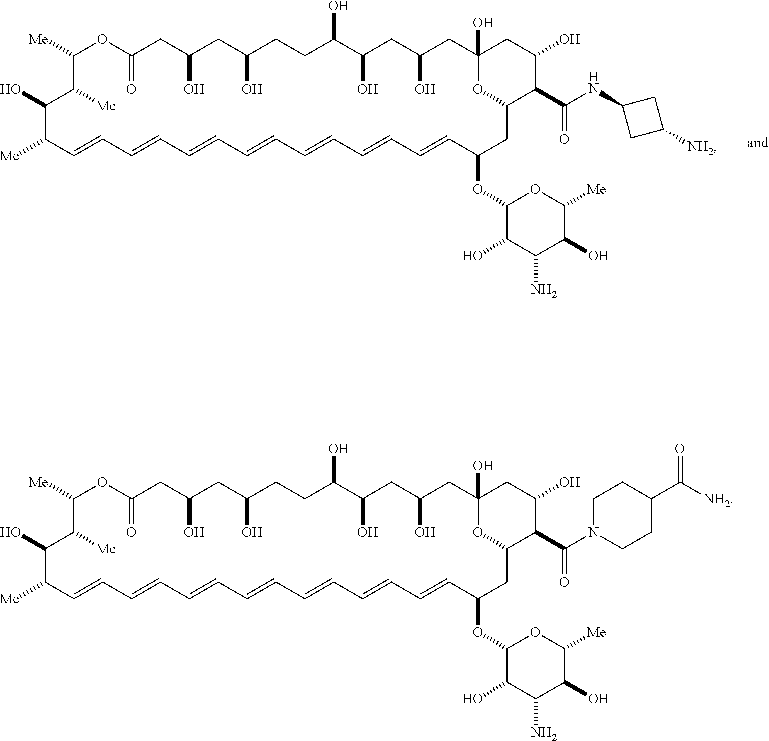

In certain aspects, provided are pharmaceutical compositions, comprising a compound provided herein; and a pharmaceutically acceptable carrier.

In certain embodiments, the pharmaceutical composition is an intravenous dosage form.

In certain embodiments, the pharmaceutical composition is an oral dosage form.

In certain aspects, provided are methods of treating a fungal infection, comprising administering to a subject in need thereof a therapeutically effective amount of a compound provided herein, thereby treating the fungal infection.

In certain embodiments, the compound is administered intravenously.

In certain embodiments, the compound is administered orally.

In certain aspects, provided are methods of making a C16 amide of C2′-epi-amphotericin B according to the transformation shown in Scheme 1:

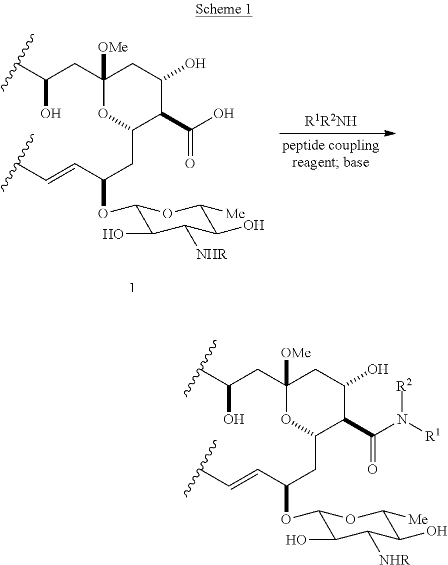

wherein:
1 represents

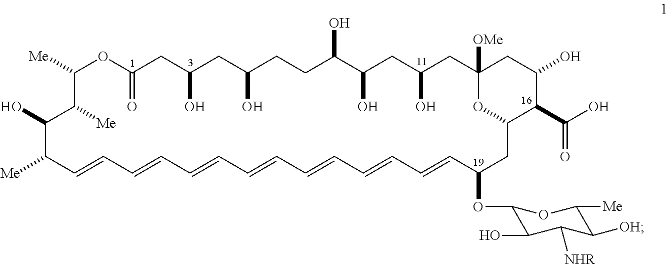

base is a tertiary amine (e.g., a trialkylamine [such as Et₃N]);
peptide coupling reagent is a peptide coupling reagent used in solid phase peptide synthesis (e.g., PyBOP, BOP, HATU, HBTU, DEPBT, DCC, or EDCI);
R is H or an amine protecting group (e.g., a carbamate protecting group selected from the group consisting of Fmoc, t-Boc, alloc, and Cbz); and
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or R¹ and R², together with the nitrogen to which they are attached, form a substituted or unsubstituted 3- to 10-membered heterocyclyl.

In certain embodiments, R is H.

In certain embodiments, R is a carbamate protecting group selected from the group consisting of Fmoc, t-Boc, alloc, and Cbz.

In certain embodiments, the base is a trialkylamine.

In certain embodiments, the base is Et₃N.

In certain embodiments, the peptide coupling reagent is PyBOP, BOP, HATU, HBTU, DEPBT, DCC, or EDCI.

### Pharmaceutical Compositions

The invention also provides pharmaceutical compositions and methods for making same.

An aspect of the invention is a pharmaceutical composition comprising a compound of the invention; and a pharmaceutically acceptable carrier. In certain embodiments, the invention is a pharmaceutical composition, comprising a compound of the invention, or a pharmaceutically acceptable salt thereof, and a pharmaceutically acceptable carrier. The term “pharmaceutically acceptable carrier” means one or more compatible solid or liquid filler, diluent, or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficacy.

In certain embodiments, the pharmaceutical composition is an intravenous dosage form.

In certain embodiments, the pharmaceutical composition is an oral dosage form.

In certain embodiments, the pharmaceutical composition is a lyophilized preparation of a liposome-intercalated or liposome-encapsulated active compound.

In certain embodiments, the pharmaceutical composition is a lipid complex of the compound in aqueous suspension.

The foregoing embodiments of pharmaceutical compositions of the invention are meant to be exemplary and are not limiting.

Also provided is a method for making such pharmaceutical compositions. The method comprises placing a compound of the invention, or a pharmaceutically acceptable salt thereof, in a pharmaceutically acceptable carrier.

### Methods of the Invention

Compounds of the invention are useful for inhibiting growth of fungi and yeast, including, in particular, fungi and yeast of clinical significance as pathogens. Advantageously, the compounds of the invention have improved therapeutic indices compared to AmB, thereby providing agents with improved efficacy and reduced toxicity as compared to AmB. Compounds of the invention are useful in methods of treating fungal and yeast infections, including, in particular, systemic fungal and yeast infections. Compounds of the invention are also useful in the manufacture of medicaments for treating fungal and yeast infections, including, in particular, systemic fungal and yeast infections. The invention further provides the use of compounds of the invention for the treatment of fungal and yeast infections, including, in particular, systemic fungal and yeast infections.

An aspect of the invention is a method of treating a fungal infection, comprising administering to a subject in need thereof a therapeutically effective amount of a compound of the invention, thereby treating the fungal infection.

As used herein, “inhibit” or “inhibiting” means reduce by an objectively measureable amount or degree compared to control. In one embodiment, inhibit or inhibiting means reduce by at least a statistically significant amount compared to control. In one embodiment, inhibit or inhibiting means reduce by at least 5 percent compared to control. In various individual embodiments, inhibit or inhibiting means reduce by at least 10, 15, 20, 25, 30, 33, 40, 50, 60, 67, 70, 75, 80, 90, or 95 percent (%) compared to control.

As used herein, the terms “treat” and “treating” refer to performing an intervention that results in (a) preventing a condition or disease from occurring in a subject that may be at risk of developing or predisposed to having the condition or disease but has not yet been diagnosed as having it; (b) inhibiting a condition or disease, e.g., slowing or arresting its development; or (c) relieving or ameliorating a condition or disease, e.g., causing regression of the condition or disease. In one embodiment the terms “treating” and “treat” refer to performing an intervention that results in (a) inhibiting a condition or disease, e.g., slowing or arresting its development; or (b) relieving or ameliorating a condition or disease, e.g., causing regression of the condition or disease. For example, in one embodiment the terms “treating” and “treat” refer to performing an intervention that results in (a) inhibiting a fungal infection, e.g., slowing or arresting its development; or (b) relieving or ameliorating a fungal infection, e.g., causing regression of the fungal infection.

A “fungal infection” as used herein refers to an infection in or of a subject with a fungus as defined herein. In one embodiment the term “fungal infection” includes a yeast infection. A “yeast infection” as used herein refers to an infection in or of a subject with a yeast as defined herein.

As used herein, a “subject” refers to a living mammal. In various embodiments a subject is a non-human mammal, including, without limitation, a mouse, rat, hamster, guinea pig, rabbit, sheep, goat, cat, dog, pig, horse, cow, or non-human primate. In one embodiment a subject is a human.

As used herein, a “subject having a fungal infection” refers to a subject that exhibits at least one objective manifestation of a fungal infection. In one embodiment a subject having a fungal infection is a subject that has been diagnosed as having a fungal infection and is in need of treatment thereof. Methods of diagnosing a fungal infection are well known and need not be described here in any detail.

As used herein, a “subject having a yeast infection” refers to a subject that exhibits at least one objective manifestation of a yeast infection. In one embodiment a subject having a yeast infection is a subject that has been diagnosed as having a yeast infection and is in need of treatment thereof. Methods of diagnosing a yeast infection are well known and need not be described here in any detail.

In certain embodiments, the compound is administered intravenously.

In certain embodiments, the compound is administered orally.

In certain embodiments, the compound is administered systemically.

In certain embodiments, the compound is administered parenterally.

In certain embodiments, the compound is administered intraperitoneally.

In certain embodiments, the compound is administered enterally.

In certain embodiments, the compound is administered intraocularly.

In certain embodiments, the compound is administered topically.

Additional routes of administration of compounds of the invention are contemplated by the invention, including, without limitation, intravesicularly (urinary bladder), pulmonary, and intrathecally.

As used herein, the phrase “effective amount” refers to any amount that is sufficient to achieve a desired biological effect.

As used herein, the phrase “therapeutically effective amount” refers to an amount that is sufficient to achieve a desired therapeutic effect, e.g., to treat a fungal or yeast infection.

For any compound described herein, a therapeutically effective amount can, in general, be initially determined from in vitro studies, animal models, or both in vitro studies and animal models. In vitro methods are well known and can include determination of minimum inhibitory concentration (MIC), minimum fungicidal concentration (MFC), concentration at which growth is inhibited by 50 percent (IC₅₀), concentration at which growth is inhibited by 90 percent (IC₉₀), and the like. A therapeutically effective amount can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described herein and other methods as are well-known in the art is well within the capabilities of the ordinarily skilled artisan.

For any compound described herein, a therapeutically effective amount for use in human subjects can be initially determined from in vitro studies, animal models, or both in vitro studies and animal models. A therapeutically effective amount for use in human subjects can also be determined from human data for compounds of the invention which have been tested in humans and for compounds which are known to exhibit similar pharmacological activities, such as other related active agents (e.g., AmB). Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

Dosing and Formulation

Compounds of the invention can be combined with other therapeutic agents. The compound of the invention and other therapeutic agent may be administered simultaneously or sequentially. When the other therapeutic agents are administered simultaneously, they can be administered in the same or separate formulations, but they are administered substantially at the same time. The other therapeutic agents are administered sequentially with one another and with compound of the invention, when the administration of the other therapeutic agents and the compound of the invention is temporally separated. The separation in time between the administration of these compounds may be a matter of minutes or it may be longer.

Examples of other therapeutic agents include other antifungal agents, including AmB, as well as other antibiotics, anti-viral agents, anti-inflammatory agents, immunosuppressive agents, and anti-cancer agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. It is preferred generally that a maximum dose be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of active compounds will be, for human subjects, from about 0.01 milligrams/kg per day to 1000 milligrams/kg per day. It is expected that oral doses in the range of 0.5 to 50 milligrams/kg, in one or several administrations per day, will yield the desired results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of compounds.

In one embodiment, intravenous administration of a compound of the invention may typically be from 0.1 mg/kg/day to 20 mg/kg/day. Intravenous dosing thus may be similar to, or advantageously, may exceed maximal tolerated doses of AmB. Intravenous dosing also may be similar to, or advantageously, may exceed maximal tolerated daily doses of AmB. Intravenous dosing also may be similar to, or advantageously, may exceed maximal tolerated cumulative doses of AmB.

Intravenous dosing also may be similar to, or advantageously, may exceed maximal recommended doses of AmB. Intravenous dosing also may be similar to, or advantageously, may exceed maximal recommended daily doses of AmB. Intravenous dosing also may be similar to, or advantageously, may exceed maximal recommended cumulative doses of AmB.

For any compound described herein the therapeutically effective amount can be initially determined from animal models. A therapeutically effective dose can also be determined from human data for compounds of the invention which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

Amphotericin B is commercially available in a number of formulations, including deoxycholate-based (sometimes referred to as desoxycholate-based) formulations and lipid-based (including liposomal) formulations. Amphotericin B derivative compounds of the invention similarly may be formulated, for example, and without limitation, as deoxycholate-based formulations and lipid-based (including liposomal) formulations.

For use in therapy, an effective amount of the compound of the invention can be administered to a subject by any mode that delivers the compound of the invention to the desired surface. Administering the pharmaceutical composition of the present invention may be accomplished by any means known to the skilled artisan. Routes of administration include but are not limited to oral, intravenous, intramuscular, intraperitoneal, subcutaneous, direct injection (for example, into a tumor or abscess), mucosal, pulmonary (e.g., inhalation), and topical.

For intravenous and other parenteral routes of administration, the compounds of the invention generally may be formulated similarly to AmB. For example, a compound of the invention can be formulated as a lyophilized preparation with deoxycholic acid, as a lyophilized preparation of liposome-intercalated or -encapsulated active compound, as a lipid complex in aqueous suspension, or as a cholesteryl sulfate complex. Lyophilized formulations are generally reconstituted in suitable aqueous solution, e.g., in sterile water or saline, shortly prior to administration.

For oral administration, the compounds (i.e., compounds of the invention, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4: 185-9 (1982). Other polymers that could be used are poly-1,3-dioxolane and poly-1,3,6-tioxocane. Preferred for pharmaceutical usage, as indicated above, are polyethylene glycol moieties.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds of the invention (or derivatives thereof). The compound of the invention (or derivative) is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins”, Proceedings of Symposium on Respiratory Drug Delivery II, Keystone, Colo., March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor). A method and composition for pulmonary delivery of drugs for systemic effect is described in U.S. Pat. No. 5,451,569, issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of compound of the invention (or derivative). Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise compound of the invention (or derivative) dissolved in water at a concentration of about 0.1 to 25 mg of biologically active compound of the invention per mL of solution. The formulation may also include a buffer and a simple sugar (e.g., for compound of the invention stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990), which is incorporated herein by reference.

The compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound of the invention and optionally at least one additional therapeutic agent included in a pharmaceutically acceptable carrier.

The therapeutic agent(s), including specifically but not limited to the compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

### INCORPORATION BY REFERENCE

All U.S. patent application publications and U.S. patents mentioned herein are hereby incorporated by reference in their entirety as if each individual publication or patent was specifically and individually indicated to be incorporated by reference. In case of conflict, the application, including any definitions herein, will control.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the embodiments described herein is not intended to be limited to the above Description, but rather is as set forth in the appended claims. Those of ordinary skill in the art will appreciate that various changes and modifications to this description may be made without departing from the spirit or scope of the invention, as defined in the following claims.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Novel Chemical Design with No Mammalian Toxicity

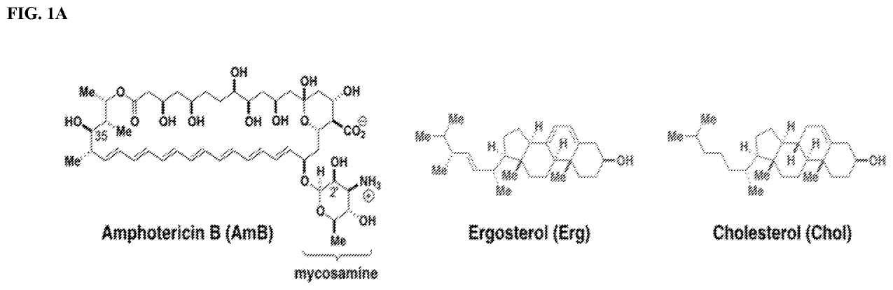

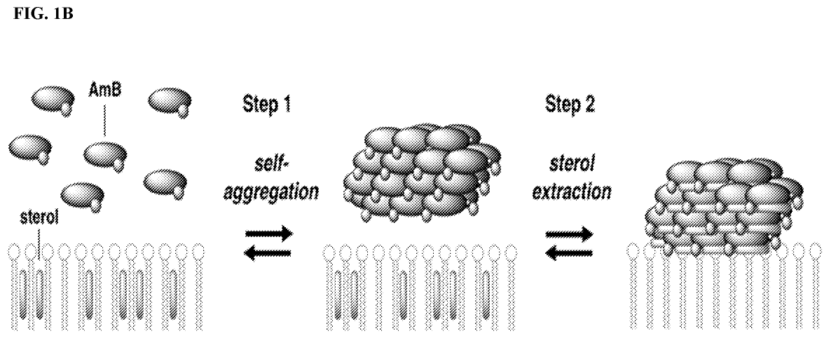

Enabled by the disclosed development of frontier synthesis methods for efficient modification of new AmB derivatives, it is alternatively discovered that AmB primarily kills fungal and human cells by binding ergosterol and cholesterol, respectively (FIG. 1A); channel formation is not required. All data are consistent with a “sterol sponge” model (FIG. 1B), whereby AmB self-assembles into a large extramembraneous aggregate and rapidly extracts physiologically vital sterols from fungal and human cells, thereby causing cell death. Frontier SSNMR studies further revealed key insights into the structure of AmB sponge-sterol complexes. Anderson, T. M. et al., Nat Chem Biol 2014, 10 (5), 400-6.

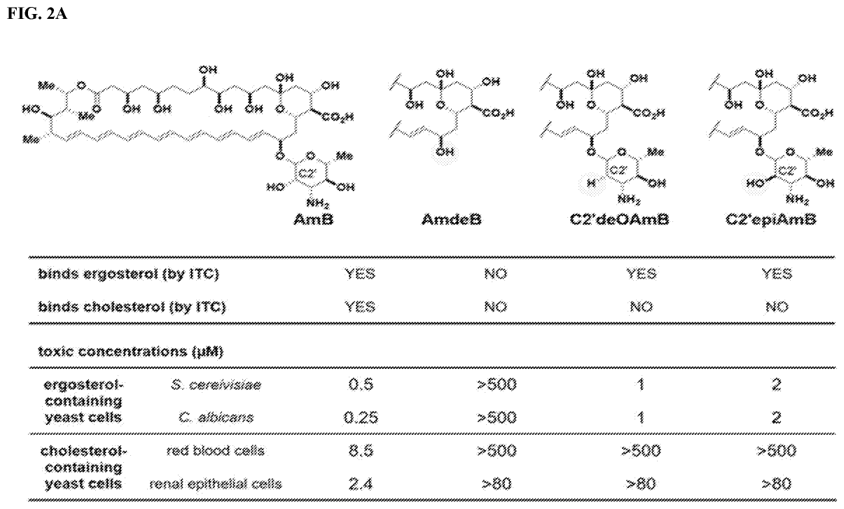

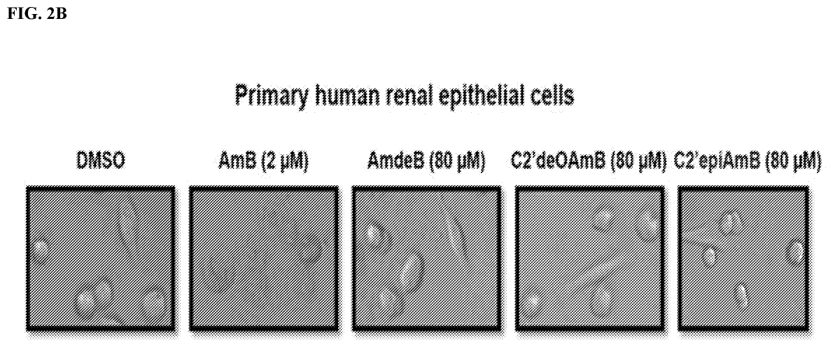

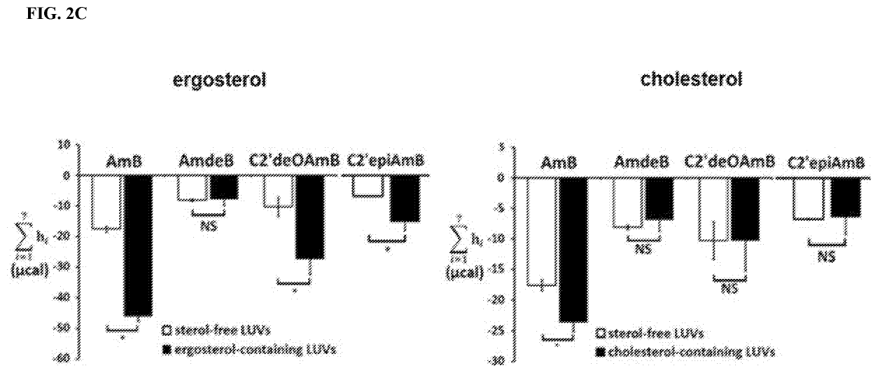

This key discovery opened a path to the rational development of a non-toxic AmB variant. To probe its predicted role in sterol binding, the hydroxyl group was synthetically deleted at the C2′ position on the mycosamine appendage. The resulting derivative, C2′deOAmB (FIG. 2A), was found to bind ergosterol but, within the detection limits of isothermal titration calorimetry (ITC), not cholesterol (FIG. 2C). Consistent with the sterol sponge model, this derivative retained good activity against yeast but, most importantly, was nontoxic to human red blood cells and primary (hREC) (FIG. 2B).

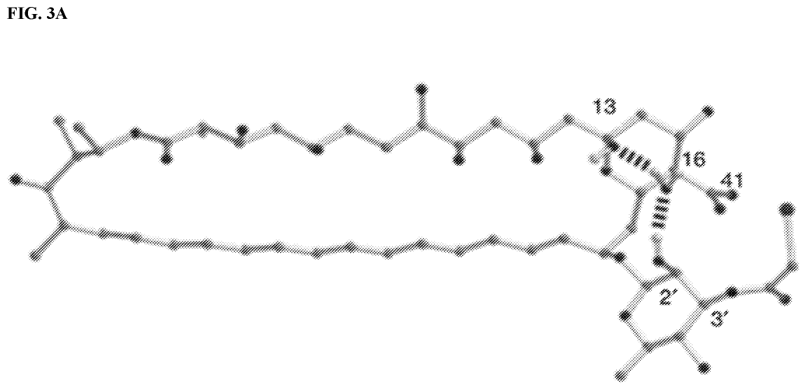

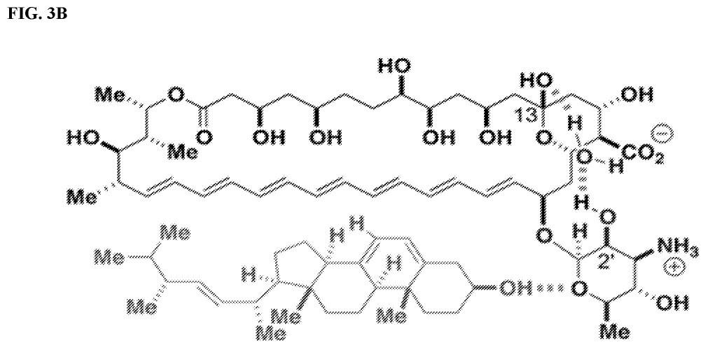

2-Deoxy glycosides are notoriously challenging to synthesize and lack of scalable access to C2′deOAmB has precluded its development. However, these findings led us to a predictive model for guiding the development of more synthetically accessible derivatives with similar selectivity profiles. Crich, D. et. al., The Journal of Organic Chemistry 2011, 76 (22), 9193-9209; Hou, D. et al., Carbohyd Res 2009, 344 (15), 1911-1940; Rodríguez, M. Á. Et al., The Journal of Organic Chemistry 2005, 70 (25), 10297-10310; and Hou, D., et al., Organic Letters 2007, 9 (22), 4487-4490. Specifically, to rationalize the selective toxicity of C2′deOAmB for fungal vs. human cells, a model was proposed in which the C2′-OH stabilizes a conformer of AmB that readily binds both ergosterol and cholesterol. The deletion of this hydroxyl group favors a shift to a different conformer or set of conformers which retain the capacity to bind ergosterol but not the more sterically bulky cholesterol. Alternatively, this model suggests that deletion of the C2′OH of AmB causes a small molecule-based allosteric effect that results in ligand-selective binding. Based on the high-resolution X-ray crystal structure of N-iodoacyl AmB (FIG. 3A), there is a prominent water-bridged hydrogen-bond between the hydroxyl groups at C2′ and C13 that may serve to stabilize a particular conformation of the mycosamine appendage relative to the polyene macrolide core. This observation, combined with our previous findings that the mycosamine appendage is critical for binding both ergosterol and cholesterol and observations by SSNMR of direct intermolecular contacts between the AmB polyene and the A/B rings of ergosterol, allowed us to propose a specific structural model for both AmB-sterol complexes consistent with all of our data (FIG. 3B). Woerly, E. M. et al, Nat Chem 2014, 6 (6), 484-91; Anderson, T. M. et al., Nat Chem Biol 2014, 10 (5), 400-6.

Guided by this model, a simple epimerization of the more synthetically accessible C2′ hydroxyl group, would likewise eliminate the water-bridged C2′OH—C13OH interaction and cause a shift in the orientation of the mycosamine appendage similar to that predicted in C2′deOAmB. The resulting derivative, C2′epiAmB (FIG. 2A), selectively binds ergosterol and exerts cytocidal action against fungal but not human cells. Notably, C2′epiAmB differs from AmB only in the stereochemistry at a single atom.

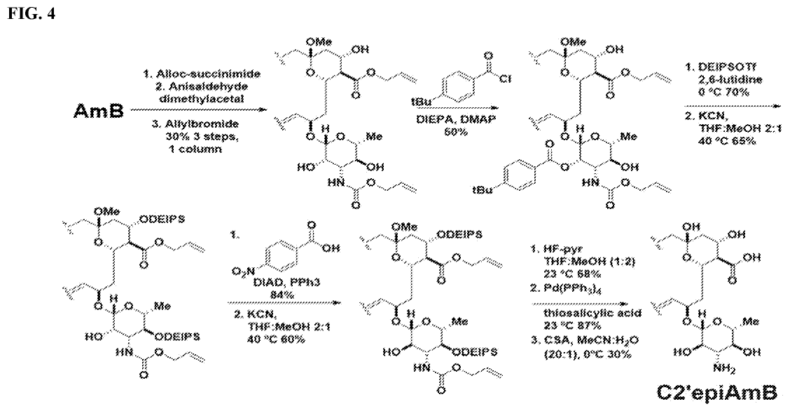

A practical 11-step synthesis of C2′epiAmB using a frontier site-selective acylation method was developed (FIG. 4). Wilcock, B. C. et al., Nat Chem 2012, 4 (12), 996-1003; Uno, B. E. A synthesis enabled understanding of Amphotericin B leading to derivatives with improved therapeutic indices. University of Illinois at Urbana-Champaign, 2014. The sterol binding and cell killing activities was then determined. As predicted, like C2′deOAmB, C2′epiAmB was found by ITC to bind ergosterol but not (detectably) cholesterol, and, most importantly, to kill fungal but not human cells (FIGS. 2A-2C).

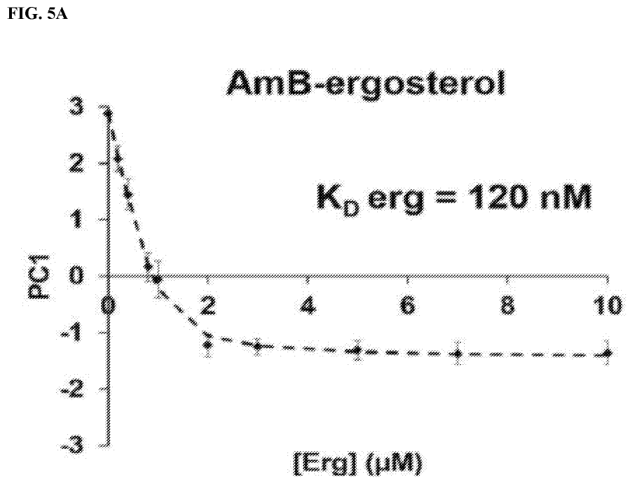

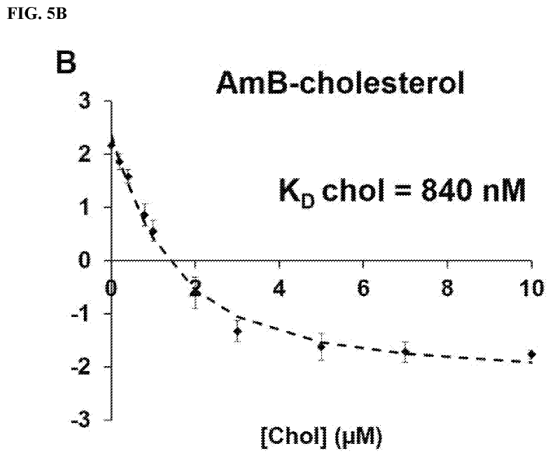

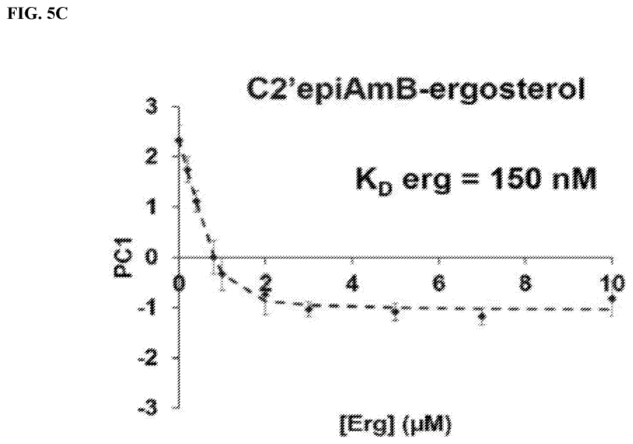

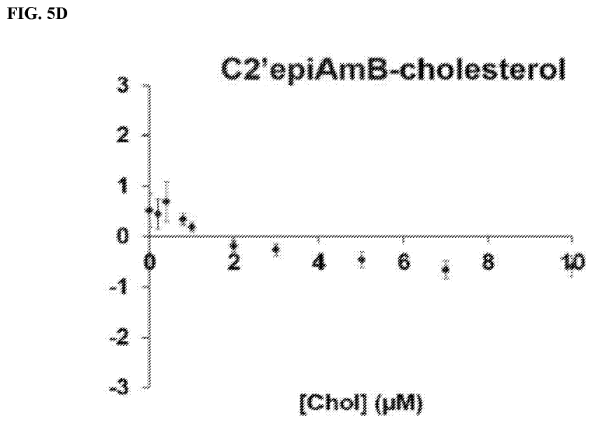

These ITC studies failed to yield S-shaped isotherms, precluding determination of binding constants and other thermodynamic parameters. However, an alternative method was developed for reproducible formation of homogenous AmB and C2′epiAmB sterol sponge aggregates in vitro. Using these preparations, a quantitative UV-Vis and Principle Component (PCA) based assay for determining the apparent K_{D}s for the binding of AmB and C2′epiAmB to ergosterol and cholesterol (FIGS. 5A-5D) was developed. Consistent with the small therapeutic index of this natural product, strong binding of AmB to both ergosterol (K_{D, erg}=120 nM) and cholesterol (K_{D,chol}=840 nM) was observed. Consistent with evaluating C2′epiAmB in vitro, strong binding for C2′epiAmB to ergosterol (K_{D,erg}=150 nM) (FIG. 5C), but little or no binding of cholesterol (FIG. 5D) was observed. The data does not permit confident assigning of a K_{D} for the latter interaction, but it was estimated that it is at least >2000 nM (which is the estimated K_{D,chol} if the data was fitted). Since C2′epiAmB shows no mammalian toxicity, these mechanistically grounded biophysical parameters can be used as benchmarks to prioritize new hybrid derivatives for further development.

### Example 2. AmB Derivatives with No Observed Animal Toxicity

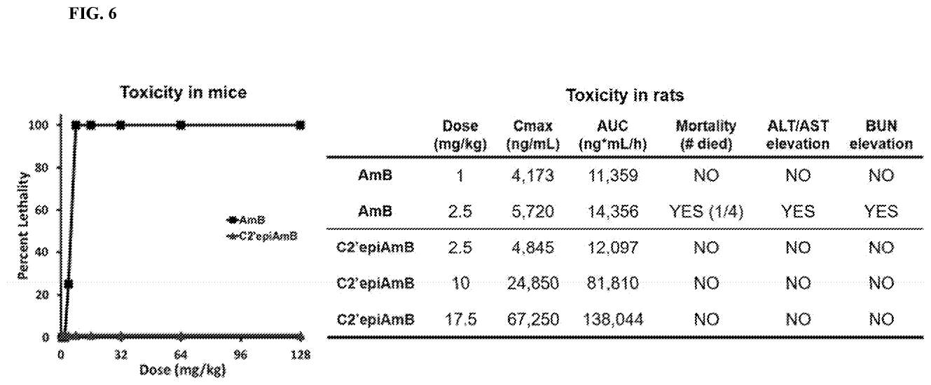

>100 mg of C2′epiAmB was prepared, formulated it as the corresponding deoxycholate complex, and evaluated this derivative head-to-head with AmB-deoxycholate for toxicity and efficacy in animal models. Intravenous (IV) administration of AmB-deoxycholate to mice was found to be lethal at 2-4 mg/kg (FIG. 6, Left). In contrast, no mortality was observed upon IV injection of C2′epiAmB-deoxycholate even at 128 mg/kg (the highest dose tested). IV administration of AmB-deoxycholate to rats (2.5 mg/kg) caused significant elevations in Blood Urea Nitrogen (BUN), Alanine transaminase/Aspartate transaminase (ALT/AST) and mortality (FIG. 6, Right). Alternatively, no elevations in BUN or ALT/AST and no mortality when rats were treated with IV injections of C2′epiAmB at doses of 2, 10, and 17.5 mg/kg (the highest dose that was tested) was observed. The Cₘₐₓ for C2′epiAmB at 17.5 mg/kg was >16-fold higher than the Cₘₐₓ for AmB at 1 mg/kg.

The toxicity of C2′epiAmB to AmBisome®, a liposomal formulation of AmB that is widely used clinically because it is somewhat less toxic than Fungizone® (AmB-deoxycholate) (FIG. 7) was directly compared. Consistent with literature precedent, we confirmed that AmBisome® shows significant toxicity in mice at 48 mg/kg as judged by state-of-the art renal genotoxicity biomarkers. Kondo, C. et al., J Toxicol Sci 2012, 37 (4), 723-37. Alternatively, mice were injected with the same high dose (48 mg/kg) of C2′epiAmB-deoxycholate and observed no significant elevations in these same biomarkers. Thus, C2′epiAmB is significantly less toxic than AmBisome® in mice.

In each case, C2′epiAmB is non-toxic to human red blood cells, primary hREC, mice, and rats up to the highest dose tested. These results are consistent with the finding that, within limits of detection of all of the experiments, C2′epiAmB does not bind cholesterol.

### Example 3. Partially Retained In Vitro Antifungal Activity

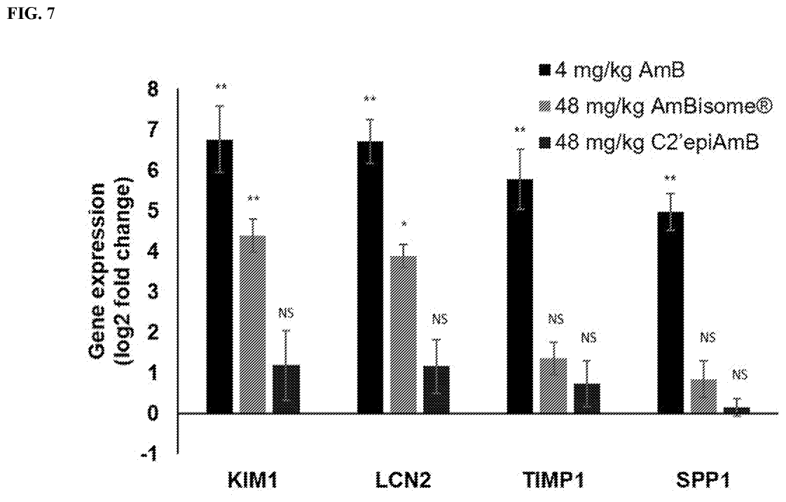

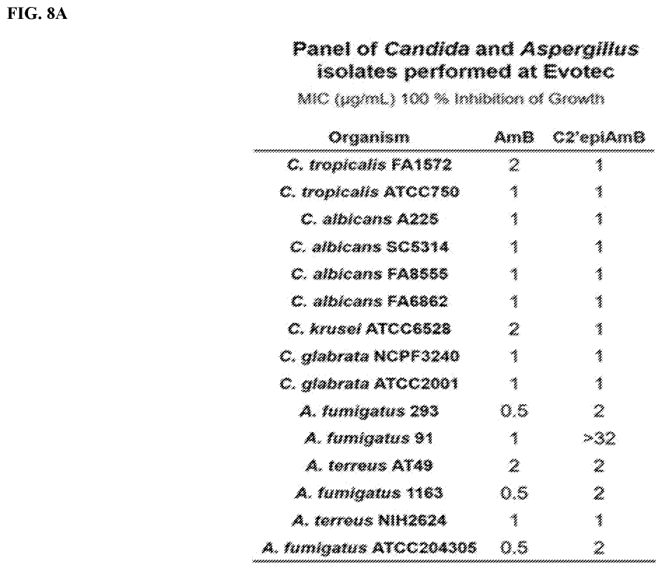

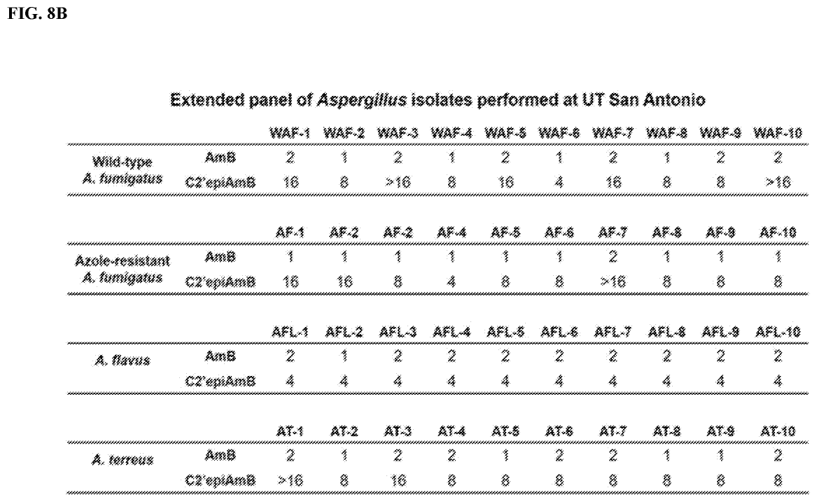

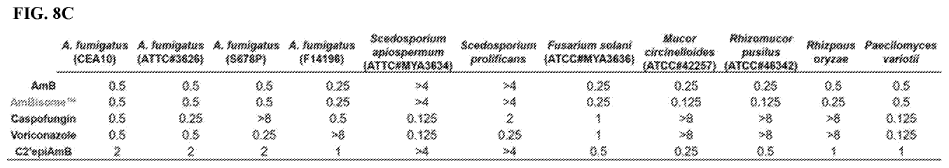

In vitro antifungal activity of C2′epiAmB was compared with that of AmB against an extensive series of Candida and Aspergillus clinical isolates (FIG. 8A) at Evotec (Oxfordshire, UK). C2′epiAmB showed good activity against many Candida and several Aspergillus strains. However, there were several strains of A. fumigatus (AF293, A1163, and ATC204305), for which C2′epiAmB was 4-fold less potent than AmB, and in one strain (AF91) C2′epiAmB was >32 times less potent. C2′epiAmB was also sent to the US national Fungus Testing Laboratory at UT-San Antonio for antifungal testing against an extended panel of especially challenging 40 Aspergillus clinical isolates, including azole-resistant A. fumigatus, A. flavus, and A. terreus (FIG. 8B). C2′epiAmB was found to be 2-16 times less potent than AmB (average 5.6-fold less potent across all 40 strains). Recently, Steinbach and Burke directly compared the activity of AmB, AmBisome®, caspofungin, voriconazole, and C2′epiAmB against an even broader panel of clinically relevant invasive molds (FIG. 8C). These studies again showed good antifungal potency for C2′epiAmB against many strains, including a pan-azole resistant strain (F14196), but also important opportunities for improved activity against Aspergillus.

### Example 4. Retained Primary Mechanism of In Vitro Antifungal Activity

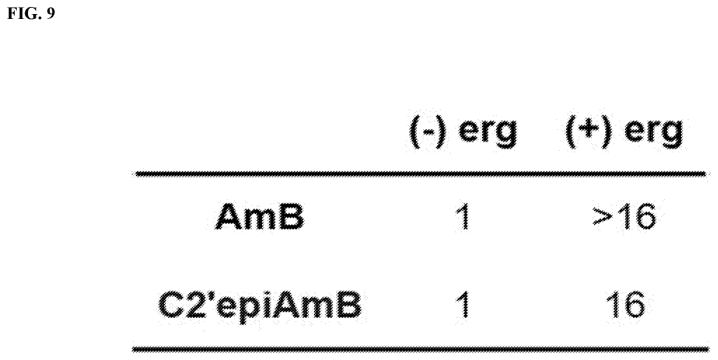

Providing strong evidence for the sterol sponge mechanism, it was previously demonstrated that the antifungal activity of AmB is mitigated via pre-complexing the AmB sterol sponge with ergosterol, thus blocking its ability to extract ergosterol from yeast cells. Anderson, T. M. et al., Nat Chem Biol 2014, 10 (5), 400-6. In a follow-up study performed in collaboration with Susan Lindquist at MIT, this mechanism also showed that it is inherently evasive to clinical resistance, because mutating the ergosterol target causes loss of pathogenicity. Davis, S. A., et al., Nat Chem Biol 2015, 11 (7), 481-7. To test whether C2′epiAmB primarily kills cells via the same sterol sponge mechanism, the C2′epiAmB sponge was similarly pre-complexed with ergosterol (FIG. 9). The same reduction in potency for AmB and C2′epiAmB upon ergosterol pre-complexation was observed. Thus, C2′epiAmB similarly kills yeast primarily via sterol binding, and, by extension, the new compounds targeted in this application are expected to have a similar barrier to fungal resistance that has been observed for the past 50+ years with AmB.

### Example 5. Non-Toxic Dose-Dependent Efficacy in Murine Invasive Candidiasis

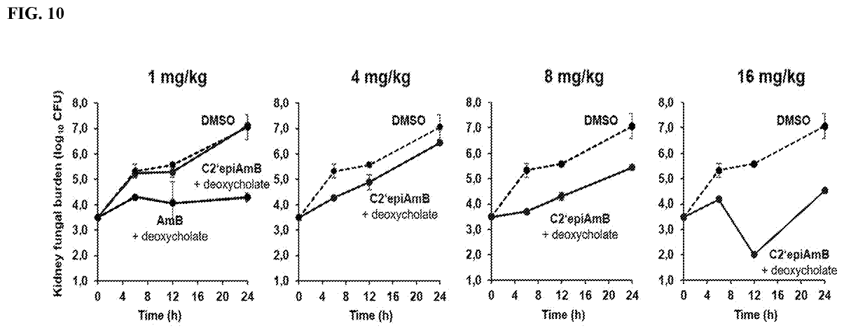

Finally, the dose-dependent efficacy of C2′epiAmB-deoxycholate complex in a murine model of invasive candidiasis was tested (FIG. 10). Neutropenic ICR/Swiss mice were injected via lateral tail vein with a lethal inoculum of C. albicans and then treated via single IP injection of AmB-deoxycholate (1 or 4 mg/kg) or C2′epiAmB-deoxycholate (1, 4, 8, or 16 mg/kg). Previous work from the Andes lab shows dose-dependent efficacy for AmB-deoxycholate. Andes, D. et al., Antimicrobial agents and chemotherapy 2001, 45 (3), 922-6. In fact, the PD parameter that best correlates with outcome is Cₘₐₓ-/MIC. The same was subsequently observed in a pulmonary model of invasive aspergillosis. Wiederhold, N. P. et al., Antimicrobial agents and chemotherapy 2006, 50 (2), 469-73. As shown in FIG. 10, C2′epiAmB also showed dose-dependent efficacy, with outstanding reductions in fungal burden at the 16 mg/kg dose.

These results show that C2′epiAmB is a unique antifungal agent with potent fungicidal activity against several Candida and Aspergillus strains and no detectable mammalian toxicity, a first for an amphotericin derivative. However, C2′epiAmB also has some important limitations with respect to potency and pathogen scope. Thus, the next plan is to develop a new series of “hybrid” derivatives designed to improve the antifungal potency and pathogen scope of C2′epiAmB while maintaining its lack of toxicity.

### Part I. C16 Amide AmB

### Example 6. General Synthetic Procedure and HPLC Method for C16 Amide AmB

General Synthetic Procedure:

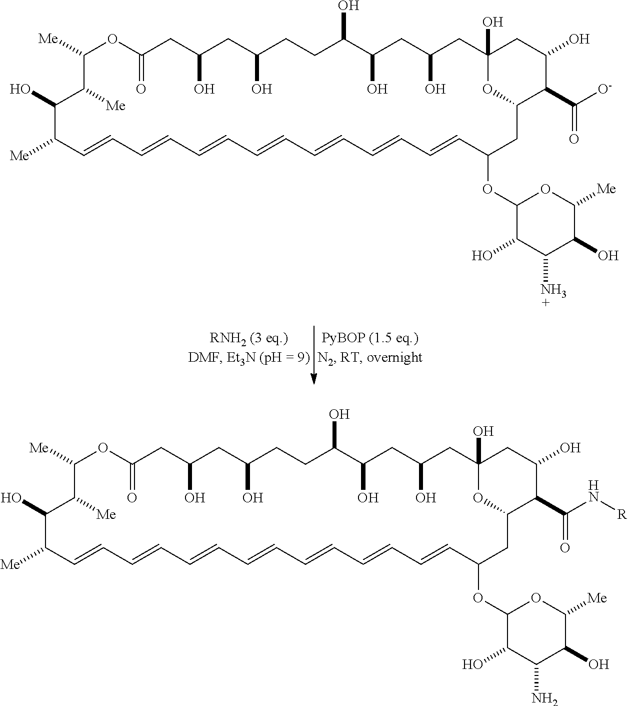

Freshly distilled Et₃N was added drop wise to a solution of Amphotericin B (10 mg; 0.01 mmol) and amine (3 eq) in DMF (500 □L) until pH=9 is reached (by pH paper). The reaction mixture was stirred for 15 minutes at room temperature. Solid PyBOP (1.5 eq; 8.4 mg) was added under nitrogen atmosphere, and the sealed vial was stirred overnight at rt. The progress of the reaction was monitored by analytical HPLC traces.

Once completed, the product was precipitated and washed with anhydrous diethyl ether (10 mL). The suspension was centrifuged at 3000 g for 5 minutes. The solvent was decanted out and the pellet was dissolved in DMSO and filtered through 0.2 micron syringe filter for purification on C18 Prep HPLC system. The pure product was dried on lyophilizer as yellowish powder and stored at −80° C. under nitrogen atmosphere.

HPLC method:

Analytical Column: C18 Agilent column (Catalogue number: 993967-902)

| | | 10 mM | |
|---|---|---|---|
| | | NH₄OAc/ | |
| | | 0.1% Formic | Flow rate |
| Time(min) | Acetonitrile | Acid buffer | (mL/min) |
| 0 | 5 | 95 | 1.2 |
| 8 | 95 | 5 | 1.2 |
| 8.5 | 95 | 5 | 1.2 |
| 9.5 | 5 | 95 | 1.2 |
| 10.5 | 5 | 95 | 1.2 |

Prep Column: C18 Agilent column (Catalogue number: 410910-502)

| | | 10 mM | |
|---|---|---|---|
| | | NH₄OAc | Flow rate |
| Time(min) | Acetonitrile | buffer | (mL/min) |
| 0 | 5 | 95 | 30 |
| 1 | 5 | 95 | 50 |
| 15 | 95 | 5 | 50 |
| 16 | 95 | 5 | 50 |
| 17 | 5 | 95 | 50 |
| 18 | 5 | 95 | 30 |

### Example 7. Characterization Data for Exemplary C16 Amide AmB Derivatives

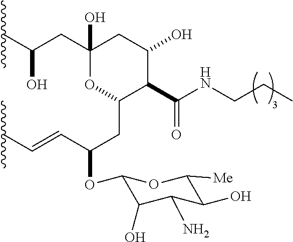

NMR: ¹H NMR (500 MHz, PyrMeOD) δ 6.55 (m, 2H), 6.42 (m, 1H), 6.30 (m, 7H), 6.20 (m, 3H), 5.56 (m, 1H), 5.42-5.37 (m, 1H), 4.88-4.81 (m, 1H), 4.72 (s, 1H), 4.70-4.63 (m, 2H), 4.56 (s, 1H), 4.41-4.33 (m, 1H), 4.18 (d, J=3.0 Hz, 1H), 3.87 (s, 1H), 3.78 (d, J=11.0 Hz, 1H), 3.49-3.42 (m, 1H), 3.25 (d, J=9.5 Hz, 1H), 3.20 (m, 1H), 2.87 (m, 1H), 2.47 (d, J=6.5 Hz, 1H), 2.42-2.27 (m, 4H), 2.26-2.19 (m, 1H), 2.11-2.03 (m, 1H), 2.02 (s, 1H), 1.95-1.86 (m, 2H), 1.84-1.66 (m, 4H), 1.63-1.46 (m, 6H), 1.46-1.40 (m, 2H), 1.36 (d, J=6.0 Hz, 3H), 1.26 (d, J=6.5 Hz, 3H), 1.25-1.16 (m, 4H), 1.14 (d, J=6.5 Hz, 3H), 1.07 (d, J=7.0 Hz, 3H), 0.76 (t, J=7.0 Hz, 3H).

LCMS: [M+H]⁺ Calculated C₅₂H₈₄N₂O₁₆, 992.5821, found 993.5899.

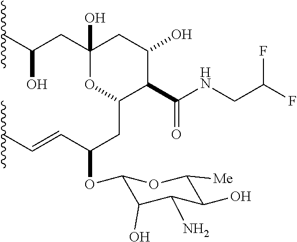 [SMILES: */C=C/[C@@H](C[C@@H]1O[C@](O)(C[C@H](*)O)C[C@H](O)[C@H]1C(=O)NCC(F)F)OC1OC(C)C(O)C(N)C1O]

NMR: ¹H NMR (500 MHz, PyrMeOD) δ 6.61-6.50 (m, 3H), 6.46-6.38 (m, 2H), 6.31 (m, 7.1 Hz, 8H), 6.19 (m, 4H), 6.09-6.05 (m, 1H), 5.98-5.94 (m, 1H), 5.56 (d, J=6.7 Hz, 3H), 5.08 (s, 2H), 4.87-4.82 (m, 2H), 4.70 (s, 1H), 4.69-4.60 (m, 3H), 4.58-4.52 (m, 2H), 4.41-4.35 (m, 2H), 4.19 (d, J=3.1 Hz, 1H), 3.95-3.84 (m, 3H), 3.77 (d, J=11.0 Hz, 2H), 3.54 (d, J=12.8 Hz, 3H), 3.52-3.42 (m, 3H), 3.25 (d, J=8.0 Hz, 1H), 3.21-3.17 (m, 1H), 2.86 (m, 1H), 2.47 (m, 2H), 2.39 (m, 3H), 2.33 (d, J=4.5 Hz, 1H), 2.28 (d, J=2.7 Hz, 1H), 2.26-2.19 (m, 2H), 2.06 (d, J=11.0 Hz, 1H), 2.01 (s, 1H), 1.95-1.86 (m, 3H), 1.81-1.50 (m, 10H), 1.47-1.40 (m, 3H), 1.35 (d, J=6.0 Hz, 3H), 1.26 (d, J=6.5 Hz, 3H), 1.14 (d, J=6.5 Hz, 3H), 1.07 (d, J=7.0 Hz, 3H).

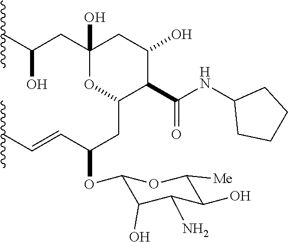 [SMILES: */C=C/[C@@H](C[C@@H]1O[C@](O)(C[C@H](*)O)C[C@H](O)[C@H]1C(=O)NC1CCCC1)OC1OC(C)C(O)C(N)C1O]

NMR: ¹H NMR (500 MHz, PyrMeOD) δ 6.55 (td, J=14.7, 11.0 Hz, 2H), 6.42 (d, J=14.2 Hz, 1H), 6.30 (ddd, J=14.6, 10.3, 4.9 Hz, 6H), 6.20 (ddd, J=15.3, 13.3, 5.5 Hz, 2H), 4.86-4.80 (m, 1H), 4.72 (s, 1H), 4.71-4.66 (m, 1H), 4.64 (dd, J=9.5, 6.1 Hz, 1H), 4.55 (t, J=10.4 Hz, 1H), 4.40-4.34 (m, 1H), 4.29-4.23 (m, 1H), 4.19 (d, J=3.1 Hz, 1H), 3.87 (s, 1H), 3.78 (d, J=10.9 Hz, 1H), 3.45 (t, J=9.1 Hz, 1H), 3.42-3.37 (m, 1H), 3.25 (d, J=9.4 Hz, 1H), 2.87 (dd, J=9.5, 3.0 Hz, 1H), 2.50-2.44 (m, 1H), 2.43-2.37 (m, 1H), 2.37-2.31 (m, 1H), 2.28 (dd, J=6.5, 3.8 Hz, 1H), 2.26-2.19 (m, 1H), 2.05 (d, J=5.7 Hz, 1H), 2.02 (s, 1H), 1.98-1.86 (m, 3H), 1.82-1.64 (m, 4H), 1.64-1.40 (m, 10H), 1.37 (d, J=6.0 Hz, 3H), 1.28-1.21 (m, 3H), 1.14 (t, J=7.9 Hz, 3H), 1.07 (d, J=7.2 Hz, 3H).

LCMS: [M+H]⁺ Calculated C₅₂H₈₂N₂O₁₆, 990.5821, found 991.5743.

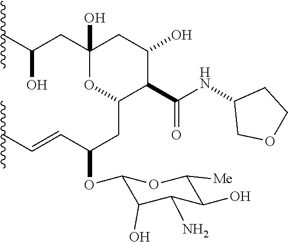

NMR: ¹H NMR (500 MHz, PyrMeOD) δ 6.54 (m, 3H), 6.41 (m, 2H), 6.37-6.26 (m, 9H), 6.25-6.13 (m, 4H), 4.83 (dd, J=16.1, 9.6 Hz, 2H), 4.73-4.52 (m, 7H), 4.48 (dd, J=7.8, 5.3 Hz, 1H), 4.37 (t, J=9.7 Hz, 2H), 4.22-4.17 (m, 1H), 3.93-3.61 (m, 10H), 3.44 (dd, J=9.5, 2.7 Hz, 1H), 2.91-2.84 (m, 1H), 2.40 (m, 2H), 2.36-2.30 (m, 2H), 2.28 (d, J=2.7 Hz, 1H), 2.22 (m, 3H), 1.98-1.64 (m, 11H), 1.63-1.39 (m, 9H), 1.36 (m, 4H), 1.26 (d, J=6.4 Hz, 3H), 1.14 (d, J=6.5 Hz, 3H), 1.07 (d, J=7.0 Hz, 3H).

LCMS: [M+H]⁺ Calculated C₅₁H₈₀N₂O₁₇, 993.5644, found 993.5535.

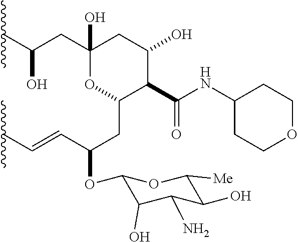 [SMILES: */C=C/[C@@H](C[C@@H]1O[C@](O)(C[C@H](*)O)C[C@H](O)[C@H]1C(=O)NC1CCOCC1)OC1OC(C)C(O)C(N)C1O]

NMR: ¹H NMR (500 MHz, PyrMeOD) δ 6.55 (m, 2H), 6.47-6.39 (m, 2H), 6.31 (m, 7H), 6.26-6.15 (m, 3H), 4.87-4.81 (m, 1H), 4.71 (s, 1H), 4.68 (dd, J=10.6, 4.6 Hz, 1H), 4.64 (d, J=8.0 Hz, 1H), 4.56 (t, J=10.4 Hz, 1H), 4.37 (t, J=9.8 Hz, 1H), 4.18 (d, J=3.0 Hz, 1H), 4.04 (m, 2H), 3.94-3.76 (m, 5H), 2.83 (m, 1H), 2.47 (m, 1H), 2.43-2.19 (m, 6H), 2.10-2.02 (m, 1H), 2.01 (s, 1H), 1.98-1.87 (m, 3H), 1.85-1.76 (m, 2H), 1.76-1.71 (m, 2H), 1.70-1.60 (m, 3H), 1.53 (m, 5H), 1.45-1.39 (m, 2H), 1.35 (d, J=6.1 Hz, 3H), 1.26 (d, J=6.4 Hz, 3H), 1.14 (d, J=6.4 Hz, 3H), 1.06 (t, J=11.7 Hz, 3H).

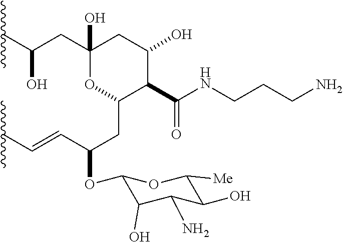 [SMILES: */C=C/[C@@H](C[C@@H]1O[C@](O)(C[C@H](*)O)C[C@H](O)[C@H]1C(=O)NCCCN)OC1OC(C)C(O)C(N)C1O]

NMR: ¹H NMR (500 MHz, 1:1 Pyridine d-5:Methanol d-4) δ 6.55 (ddd, J=17.9, 14.7, 10.7 Hz, 2H), 6.46-6.39 (m, 1H), 6.38-6.27 (m, 7H), 6.26-6.17 (m, 3H), 5.56 (dd, J=6.6, 2.2 Hz, 1H), 5.39 (dd, J=15.0, 10.1 Hz, 1H), 4.84-4.79 (m, 1H), 4.70 (s, 1H), 4.65-4.52 (m, 3H), 4.35 (ddt, J=9.9, 6.1, 2.9 Hz, 1H), 4.26 (d, J=3.3 Hz, 1H), 3.86 (td, J=10.9, 9.9, 2.8 Hz, 1H), 3.76 (dt, J=11.0, 2.2 Hz, 1H), 3.60-3.51 (m, 2H), 3.42-3.34 (m, 3H), 3.25 (dd, J=9.5, 2.2 Hz, 1H), 3.16-3.06 (m, 3H), 2.47 (qd, J=9.9, 8.3, 5.2 Hz, 1H), 2.39 (dd, J=16.9, 9.6 Hz, 1H), 2.33-2.19 (m, 4H), 2.08-2.00 (m, 1H), 1.97-1.86 (m, 4H), 1.83-1.65 (m, 5H), 1.62-1.48 (m, 4H), 1.43 (ddd, J=11.7, 6.3, 3.3 Hz, 2H), 1.33 (d, J=6.1 Hz, 3H), 1.26 (d, J=6.4 Hz, 3H), 1.14 (d, J=6.4 Hz, 3H), 1.07 (d, J=7.1 Hz, 3H).

LCMS: [M+H]⁺ Calculated (C₅₀H₈₁N₃O₁₆+H)⁺, 980.5625, Observed 980.5695.

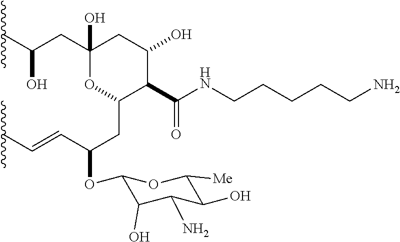 [SMILES: */C=C/[C@@H](C[C@@H]1O[C@](O)(C[C@H](*)O)C[C@H](O)[C@H]1C(=O)NCCCCCN)OC1OC(C)C(O)C(N)C1O]

NMR: ¹H NMR (500 MHz, 1:1 Pyridine d-5:Methanol d-4) δ 6.55 (ddd, J=17.5, 14.7, 10.9 Hz, 2H), 6.42 (d, J=11.5 Hz, 1H), 6.31 (dtd, J=19.0, 14.8, 12.7, 8.4 Hz, 7H), 6.20 (td, J=15.2, 14.1, 8.7 Hz, 3H), 5.56 (d, J=6.9 Hz, 1H), 5.39 (dd, J=15.0, 10.1 Hz, 1H), 4.80 (t, J=9.8 Hz, 1H), 4.70 (s, 1H), 4.66-4.52 (m, 3H), 4.35 (tt, J=9.8, 3.0 Hz, 1H), 4.24 (d, J=3.2 Hz, 1H), 3.86 (t, J=9.8 Hz, 1H), 3.77 (dt, J=11.2, 2.4 Hz, 1H), 3.56 (t, J=9.5 Hz, 1H), 3.42-3.35 (m, 2H), 3.30-3.22 (m, 3H), 3.07-3.02 (m, 1H), 2.94 (t, J=7.4 Hz, 2H), 2.47 (td, J=9.8, 6.3 Hz, 1H), 2.43-2.36 (m, 1H), 2.33-2.25 (m, 3H), 2.24-2.19 (m, 1H), 2.04 (dd, J=11.0, 6.1 Hz, 1H), 1.89 (ddd, J=11.7, 8.6, 6.1 Hz, 2H), 1.83-1.74 (m, 2H), 1.69 (qd, J=8.8, 8.1, 4.5 Hz, 3H), 1.60-1.53 (m, 4H), 1.43 (ddd, J=11.6, 8.0, 3.7 Hz, 4H), 1.35 (d, J=6.1 Hz, 3H), 1.26 (d, J=6.3 Hz, 3H), 1.15 (d, J=6.4 Hz, 3H), 1.07 (d, J=7.0 Hz, 3H).

LCMS: [M+H]⁺ Calculated (C₅₂H₈₅N₃O₁₆+H)⁺, 1008.5990 Observed 1008.6008.

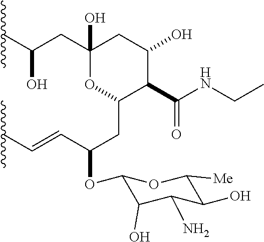 [SMILES: */C=C/[C@@H](C[C@@H]1O[C@](O)(C[C@H](*)O)C[C@H](O)[C@H]1C(=O)NCC)OC1OC(C)C(O)C(N)C1O]

NMR: ¹H NMR (500 MHz, 1:1 Pyridine d-5:Methanol d-4) δ 6.54 (td, J=15.0, 10.9 Hz, 2H), 6.45-6.38 (m, 1H), 6.31 (ddd, J=17.9, 9.7, 5.7 Hz, 7H), 6.19 (ddd, J=19.4, 14.5, 9.4 Hz, 3H), 5.58-5.53 (m, 1H), 5.39 (dd, J=15.0, 10.1 Hz, 1H), 4.80 (t, J=9.8 Hz, 1H), 4.70-4.60 (m, 3H), 4.55 (t, J=10.4 Hz, 1H), 4.36 (tt, J=9.8, 3.0 Hz, 1H), 4.14 (d, J=3.1 Hz, 1H), 3.86 (t, J=10.0 Hz, 1H), 3.77 (dt, J=11.0, 2.3 Hz, 1H), 3.41 (t, J=9.3 Hz, 1H), 3.37-3.35 (m, 1H), 3.27-3.18 (m, 2H), 2.81 (dd, J=9.2, 2.9 Hz, 1H), 2.46 (dd, J=10.0, 6.2 Hz, 1H), 2.42-2.36 (m, 1H), 2.32 (dd, J=14.8, 4.9 Hz, 1H), 2.28-2.18 (m, 3H), 2.09-2.00 (m, 1H), 1.94-1.85 (m, 2H), 1.81-1.66 (m, 4H), 1.59-1.50 (m, 3H), 1.46-1.40 (m, 2H), 1.37-1.34 (m, 3H), 1.25 (dd, J=6.8, 3.3 Hz, 3H), 1.14 (dd, J=6.4, 2.9 Hz, 3H), 1.10 (t, J=7.3 Hz, 3H), 1.07 (d, J=7.2 Hz, 3H).

LCMS: [M+H]⁺ Calculated C₄₉H₇₈N₂O₁₆, 950.5351 Observed 951.6876.

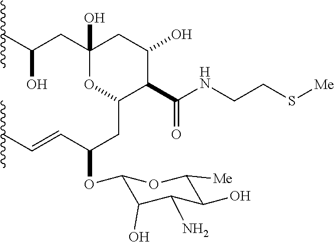 [SMILES: */C=C/[C@@H](C[C@@H]1O[C@](O)(C[C@H](*)O)C[C@H](O)[C@H]1C(=O)NCCSC)OC1OC(C)C(O)C(N)C1O]

NMR: ¹H NMR (500 MHz, 1:1 Pyridine d-5:Methanol d-4) δ 6.54 (ddd, J=18.2, 14.8, 10.9 Hz, 2H), 6.43 (dd, J=14.3, 10.8 Hz, 1H), 6.31 (ddt, J=14.4, 9.1, 5.0 Hz, 7H), 6.19 (ddd, J=20.6, 14.7, 10.0 Hz, 3H), 5.60-5.53 (m, 1H), 5.38 (dd, J=15.1, 10.1 Hz, 1H), 4.82 (t, J=9.9 Hz, 1H), 4.76 (s, 1H), 4.68-4.59 (m, 2H), 4.59-4.51 (m, 1H), 4.36 (tt, J=9.8, 2.9 Hz, 1H), 4.27 (d, J=3.2 Hz, 1H), 3.90-3.83 (m, 1H), 3.77 (dt, J=11.2, 2.3 Hz, 1H), 3.64-3.54 (m, 2H), 3.44 (dq, J=9.1, 6.1 Hz, 1H), 3.38-3.35 (m, 1H), 3.25 (dd, J=9.7, 2.2 Hz, 1H), 3.10 (dd, J=9.9, 3.2 Hz, 1H), 2.71 (td, J=12.7, 12.0, 6.1 Hz, 1H), 2.63 (dt, J=13.5, 6.9 Hz, 1H), 2.46 (ddd, J=13.6, 8.3, 3.7 Hz, 1H), 2.42-2.35 (m, 2H), 2.31-2.26 (m, 2H), 2.25-2.19 (m, 1H), 2.04 (s, 3H), 1.94-1.86 (m, 2H), 1.81-1.64 (m, 4H), 1.60-1.49 (m, 3H), 1.46-1.39 (m, 2H), 1.36 (d, J=6.0 Hz, 3H), 1.26 (d, J=6.5 Hz, 3H), 1.14 (d, J=6.4 Hz, 3H), 1.07 (d, J=7.2 Hz, 3H).

LCMS: [M+H]⁺ Calculated C₅₀H₈₀N₂O₁₆S, 996.5229 Observed 997.5206.

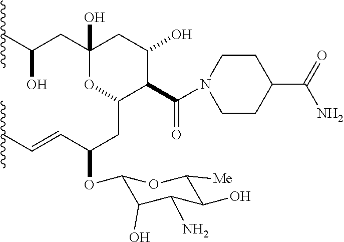 [SMILES: */C=C/[C@@H](C[C@@H]1O[C@](O)(C[C@H](*)O)C[C@H](O)[C@H]1C(=O)N1CCC(C(N)=O)CC1)OC1OC(C)C(O)C(N)C1O]

NMR: ¹H NMR (500 MHz, 1:1 Pyridine d-5:Methanol d-4) δ 6.61-6.49 (m, 2H), 6.42 (d, J=11.8 Hz, 1H), 6.31 (dt, J=15.3, 5.8 Hz, 8H), 6.25-6.16 (m, 3H), 5.59-5.53 (m, 1H), 5.40 (d, J=10.1 Hz, 1H), 4.86 (d, J=8.9 Hz, 1H), 4.74 (d, J=32.3 Hz, 2H), 4.60 (dt, J=15.2, 10.4 Hz, 4H), 4.35 (dt, J=9.5, 2.7 Hz, 4H), 3.87 (t, J=9.8 Hz, 1H), 3.80-3.75 (m, 1H), 3.69-3.63 (m, 1H), 3.47 (d, J=3.2 Hz, 1H), 3.25 (dt, J=10.1, 2.8 Hz, 2H), 3.23-3.11 (m, 2H), 2.84 (d, J=10.2 Hz, 1H), 2.68 (s, 3H), 2.43-2.35 (m, 2H), 2.31-2.17 (m, 3H), 1.98-1.86 (m, 5H), 1.84-1.73 (m, 5H), 1.65 (t, J=5.7 Hz, 5H), 1.56-1.41 (m, 5H), 1.38 (d, J=6.2 Hz, 3H), 1.26 (d, J=6.4 Hz, 3H), 1.15 (d, J=6.4 Hz, 3H), 1.07 (d, J=7.1 Hz, 3H).

LCMS: [M+H]⁺ Calculated C₅₃H₈₃N₃O₁₇, 996.5229 Observed 1034.7296.

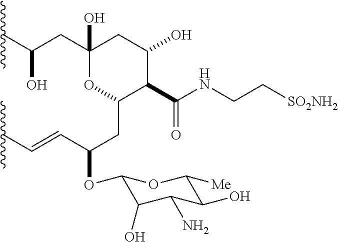

NMR: ¹H NMR (500 MHz, 1:1 Pyridine d-5:Methanol d-4) δ 6.54 (ddd, J=21.8, 14.8, 10.9 Hz, 2H), 6.42 (dd, J=14.0, 11.1 Hz, 1H), 6.36-6.25 (m, 7H), 6.25-6.13 (m, 3H), 5.59-5.52 (m, 1H), 5.42-5.36 (m, 1H), 4.86-4.79 (m, 2H), 4.65-4.51 (m, 3H), 4.39-4.32 (m, 2H), 3.96 (dt, J=13.8, 6.7 Hz, 1H), 3.86 (t, J=9.7 Hz, 1H), 3.81-3.75 (m, 2H), 3.61 (t, J=9.5 Hz, 1H), 3.53-3.46 (m, 3H), 3.27-3.23 (m, 1H), 2.47 (td, J=9.7, 6.2 Hz, 1H), 2.43-2.35 (m, 2H), 2.31-2.18 (m, 3H), 2.08-1.99 (m, 1H), 1.94-1.85 (m, 2H), 1.79-1.67 (m, 4H), 1.60-1.49 (m, 3H), 1.45-1.38 (m, 2H), 1.34 (d, J=6.2 Hz, 3H), 1.26 (d, J=6.5 Hz, 3H), 1.15 (d, J=6.3 Hz, 3H), 1.07 (d, J=7.1 Hz, 2H).

LCMS: [M+H]⁺ Calculated C₄₉H₇₉N₃O₁₈S, 1029.5079 Observed 1030.6719.

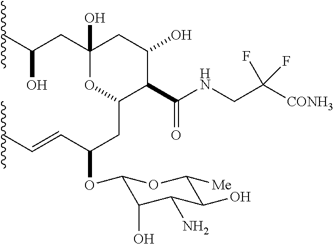

NMR: ¹H NMR (500 MHz, 1:1 Pyridine d-5:Methanol d-4) δ 6.60-6.48 (m, 2H), 6.41 (d, J=12.9 Hz, 1H), 6.36-6.20 (m, 8H), 6.17 (s, 1H), 5.61-5.53 (m, 1H), 5.40 (d, J=10.1 Hz, 1H), 4.85 (d, J=13.1 Hz, 2H), 4.67-4.59 (m, 2H), 4.58-4.44 (m, 2H), 4.42 (d, J=3.1 Hz, 1H), 4.36 (t, J=2.9 Hz, 1H), 3.87 (d, J=9.7 Hz, 1H), 3.81-3.74 (m, 1H), 3.66 (d, J=9.9 Hz, 1H), 3.58 (d, J=6.3 Hz, 2H), 3.42-3.35 (m, 1H), 3.25 (dd, J=9.7, 2.2 Hz, 1H), 3.03 (td, J=6.6, 3.9 Hz, 1H), 2.39 (ddd, J=12.1, 9.7, 4.6 Hz, 4H), 2.28-2.19 (m, 2H), 2.04 (d, J=5.0 Hz, 1H), 1.94-1.86 (m, 2H), 1.79-1.70 (m, 3H), 1.69-1.64 (m, 2H), 1.61-1.49 (m, 3H), 1.45-1.39 (m, 2H), 1.35 (d, J=6.1 Hz, 3H), 1.26 (d, J=6.4 Hz, 3H), 1.14 (d, J=6.4 Hz, 3H), 1.07 (d, J=7.1 Hz, 2H).

LCMS: [M+H]⁺ Calculated C₅₀H₇₇F₂N₃O₁₇, 1029.5221 Observed 1030.6863.

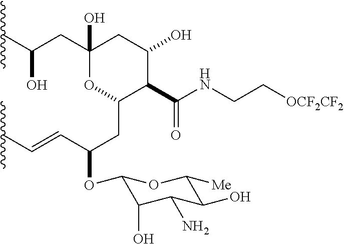

NMR: ¹H NMR (500 MHz, 1:1 Pyridine d-5:Methanol d-4) δ 6.55 (ddd, J=18.3, 14.8, 10.9 Hz, 2H), 6.48-6.39 (m, 1H), 6.32 (ddt, J=14.8, 10.7, 4.4 Hz, 8H), 6.19 (td, J=15.9, 15.3, 10.5 Hz, 2H), 5.59-5.53 (m, 1H), 5.39 (dd, J=15.1, 10.1 Hz, 1H), 4.82 (t, J=10.0 Hz, 1H), 4.73 (s, 1H), 4.66-4.51 (m, 3H), 4.39-4.30 (m, 2H), 4.25-4.19 (m, 1H), 4.16 (ddd, J=10.1, 6.1, 4.7 Hz, 1H), 3.89-3.82 (m, 1H), 3.77 (dt, J=11.1, 2.2 Hz, 1H), 3.70-3.62 (m, 2H), 3.54-3.47 (m, 1H), 3.42 (dq, J=9.1, 6.1 Hz, 1H), 3.36-3.34 (m, 1H), 3.24 (dt, J=10.1, 3.2 Hz, 2H), 2.50-2.43 (m, 1H), 2.43-2.37 (m, 1H), 2.35-2.27 (m, 3H), 2.25-2.19 (m, 1H), 2.04 (dq, J=11.1, 3.8, 3.1 Hz, 1H), 1.89 (ddd, J=9.4, 4.6, 2.8 Hz, 2H), 1.80-1.66 (m, 3H), 1.62-1.46 (m, 4H), 1.45-1.38 (m, 2H), 1.36 (d, J=6.1 Hz, 3H), 1.26 (d, J=6.4 Hz, 3H), 1.14 (d, J=6.4 Hz, 3H), 1.07 (d, J=7.1 Hz, 3H).

LCMS: [M+H]⁺ Calculated C₅₁H₇₇F₅N₂O₁₇, 1084.5142 Observed 1085.6813.

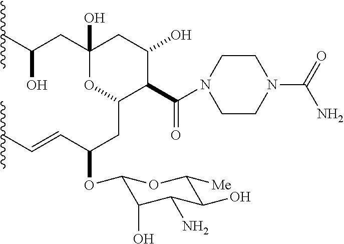

NMR: ¹H NMR (500 MHz, 1:1 Pyridine d-5:Methanol d-4) δ 6.61-6.49 (m, 2H), 6.43 (s, 1H), 6.32 (dtt, J=10.8, 7.6, 4.1 Hz, 7H), 6.26-6.16 (m, 3H), 5.57 (dd, J=6.8, 2.1 Hz, 1H), 5.39 (dd, J=15.1, 10.2 Hz, 1H), 4.89 (t, J=9.6 Hz, 1H), 4.75 (s, 1H), 4.68-4.56 (m, 3H), 4.38 (dd, J=12.6, 3.1 Hz, 2H), 3.98-3.76 (m, 5H), 3.73-3.66 (m, 3H), 3.52 (d, J=8.3 Hz, 3H), 3.45 (ddd, J=9.1, 5.6, 2.3 Hz, 2H), 3.38-3.34 (m, 1H), 3.27 (ddd, J=19.7, 9.8, 2.5 Hz, 3H), 2.83 (t, J=10.0 Hz, 1H), 2.47 (d, J=6.5 Hz, 1H), 2.43-2.37 (m, 1H), 2.29-2.22 (m, 2H), 2.14 (dd, J=14.9, 4.7 Hz, 1H), 1.97-1.89 (m, 2H), 1.81-1.71 (m, 3H), 1.65-1.55 (m, 3H), 1.53-1.47 (m, 2H), 1.46-1.39 (m, 2H), 1.37 (d, J=6.1 Hz, 3H), 1.26 (d, J=6.4 Hz, 3H), 1.15 (d, J=6.4 Hz, 3H), 1.07 (d, J=7.1 Hz, 3H).

LCMS: [M+H]⁺ Calculated C₅₂H₈₂N₄O₁₇, 1034.5675 Observed 1035.7306.

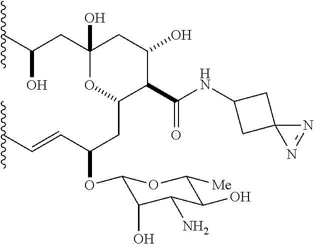

NMR: ¹H NMR (500 MHz, 1:1 Pyridine d-5:Methanol d-4) δ 6.55 (ddd, J=21.3, 14.7, 10.9 Hz, 2H), 6.48-6.40 (m, 1H), 6.36-6.26 (m, 8H), 6.20 (td, J=15.5, 10.5 Hz, 2H), 5.56 (dt, J=8.1, 5.7 Hz, 1H), 5.41-5.35 (m, 1H), 4.81 (t, J=9.8 Hz, 1H), 4.73 (s, 1H), 4.69-4.45 (m, 5H), 4.35 (dd, J=7.8, 3.2 Hz, 2H), 3.89-3.83 (m, 1H), 3.78 (dt, J=10.9, 2.3 Hz, 1H), 3.69 (t, J=9.6 Hz, 1H), 3.44-3.35 (m, 2H), 3.27 (ddd, J=15.4, 9.9, 2.7 Hz, 2H), 2.53-2.45 (m, 2H), 2.43-2.28 (m, 7H), 2.25-2.21 (m, 1H), 2.10-2.00 (m, 1H), 1.90 (tdd, J=9.7, 6.6, 3.1 Hz, 2H), 1.83-1.66 (m, 4H), 1.62-1.52 (m, 3H), 1.46-1.40 (m, 2H), 1.34 (d, J=6.0 Hz, 3H), 1.26 (d, J=6.5 Hz, 3H), 1.15 (d, J=6.4 Hz, 3H), 1.07 (d, J=7.2 Hz, 3H).

LCMS: [M+H]⁺ Calculated C₅₁H₇₈N₄O₁₆, 1002.5413 Observed 1003.6988.

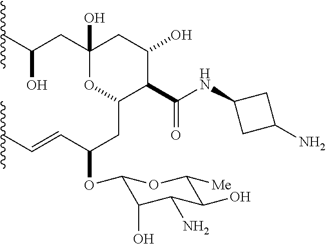

NMR: ¹H NMR (500 MHz, 1:1 Pyridine d-5:Methanol d-4) δ 6.60-6.50 (m, 2H), 6.47-6.39 (m, 1H), 6.38-6.14 (m, 10H), 5.56 (dd, J=6.5, 2.1 Hz, 1H), 5.39 (dd, J=15.0, 10.1 Hz, 1H), 4.82-4.74 (m, 1H), 4.72 (s, 1H), 4.69-4.52 (m, 4H), 4.35 (qd, J=6.8, 5.9, 2.9 Hz, 2H), 3.86 (qd, J=10.5, 8.8, 4.5 Hz, 2H), 3.76 (dt, J=10.9, 2.3 Hz, 1H), 3.70 (t, J=9.6 Hz, 1H), 3.48-3.41 (m, 1H), 3.35 (d, J=3.0 Hz, 1H), 3.25 (dd, J=9.6, 2.1 Hz, 1H), 2.53-2.43 (m, 4H), 2.42-2.35 (m, 2H), 2.35-2.26 (m, 3H), 2.25-2.19 (m, 1H), 2.09-2.00 (m, 1H), 1.94-1.85 (m, 2H), 1.82-1.74 (m, 2H), 1.73-1.62 (m, 2H), 1.60-1.47 (m, 4H), 1.45-1.39 (m, 2H), 1.36 (d, J=6.1 Hz, 3H), 1.26 (d, J=6.4 Hz, 3H), 1.14 (d, J=6.3 Hz, 3H), 1.07 (d, J=7.1 Hz, 3H).

LCMS: [M+H]⁺ Calculated C₅₁H₈₁N₃O₁₆, 991.5617 Observed 992.7187.

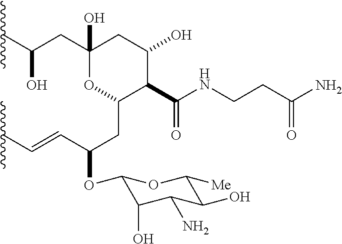

NMR: ¹H NMR (500 MHz, PyrMeOD) δ 6.53 (ddd, J=19.4, 14.7, 10.9 Hz, 2H), 6.45-6.37 (m, 1H), 6.36-6.12 (m, 10H), 5.55 (dd, J=6.4, 2.2 Hz, 1H), 5.39 (dd, J=15.0, 10.1 Hz, 1H), 4.83-4.76 (m, 2H), 4.67-4.51 (m, 3H), 4.36 (tt, J=9.8, 2.9 Hz, 1H), 4.28 (d, J=3.2 Hz, 1H), 3.86 (dd, J=11.1, 8.3 Hz, 1H), 3.76 (dt, J=10.8, 2.3 Hz, 1H), 3.69 (dt, J=13.6, 6.8 Hz, 1H), 3.59-3.44 (m, 3H), 3.25 (dd, J=9.5, 2.2 Hz, 1H), 3.14 (dd, J=10.0, 3.1 Hz, 1H), 2.59 (dd, J=14.9, 7.7 Hz, 2H), 2.47 (td, J=10.0, 6.5 Hz, 1H), 2.43-2.18 (m, 5H), 2.04 (dt, J=13.8, 6.3 Hz, 1H), 1.94-1.84 (m, 2H), 1.80-1.65 (m, 4H), 1.61-1.49 (m, 3H), 1.46-1.38 (m, 2H), 1.35 (d, J=6.0 Hz, 3H), 1.26 (d, J=6.4 Hz, 3H), 1.14 (d, J=6.4 Hz, 3H), 1.07 (d, J=7.2 Hz, 3H).

LCMS: [M+H]⁺ Calculated C₅₀H₇₉N₃O₁₇, 993.5409 Observed 994.6960.

### Example 8. C16 Amide AmB (AmBHEA) Shows Comparable Potency as AmB

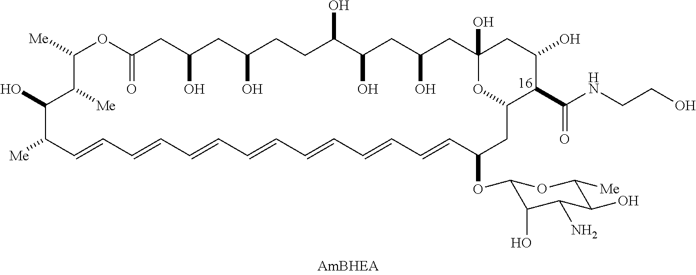

TABLE 1
Minimum Inhibition Concentrations for AmB
and AmBHEA Against Different Fungas.

| MIC (μM) | AmB | AmBHEA |
|---|---|---|
| C. albicans SN250 | 0.225 | 0.25 |
| C. albicans | 0.25 | 0.225 |
| C. krusei | 0.7 | 0.45 |
| C. glabrata | 0.14 | 0.145 |
| C. tropicalis | 0.325 | 0.225 |
| A. fumigatus 91 | 1.5 | 1 |
| A. fumigatus 1163 | 1 | 0.525 |
| A. fumigatus 1100 | 1 | 0.7 |
| Average MIC | 0.642 | 0.44 |

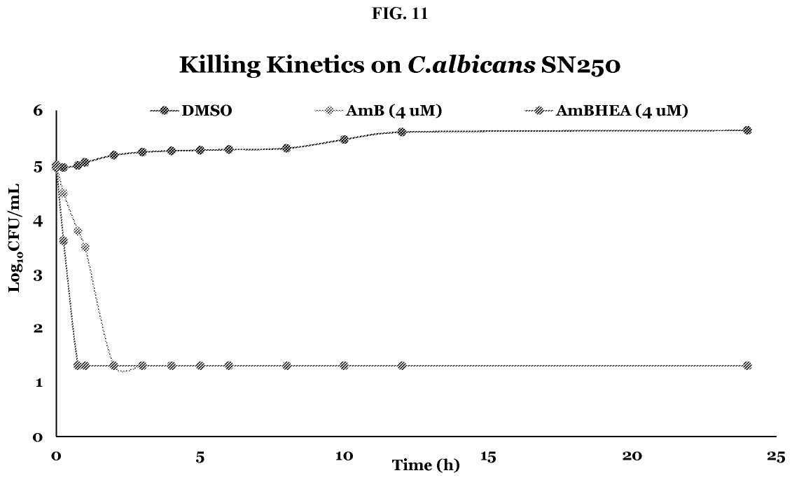

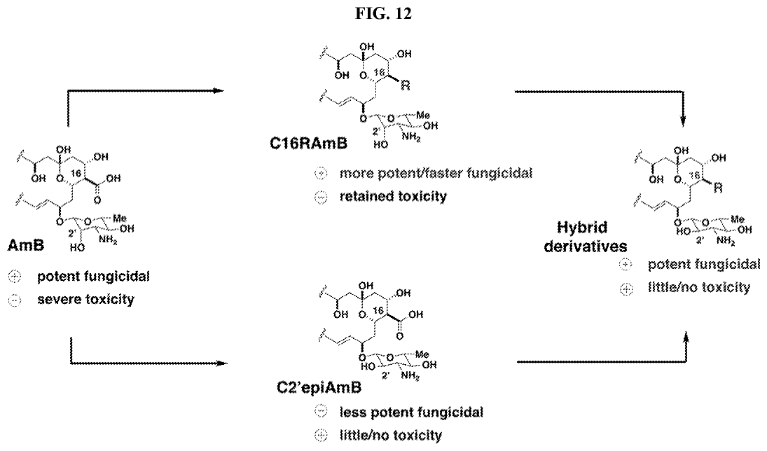

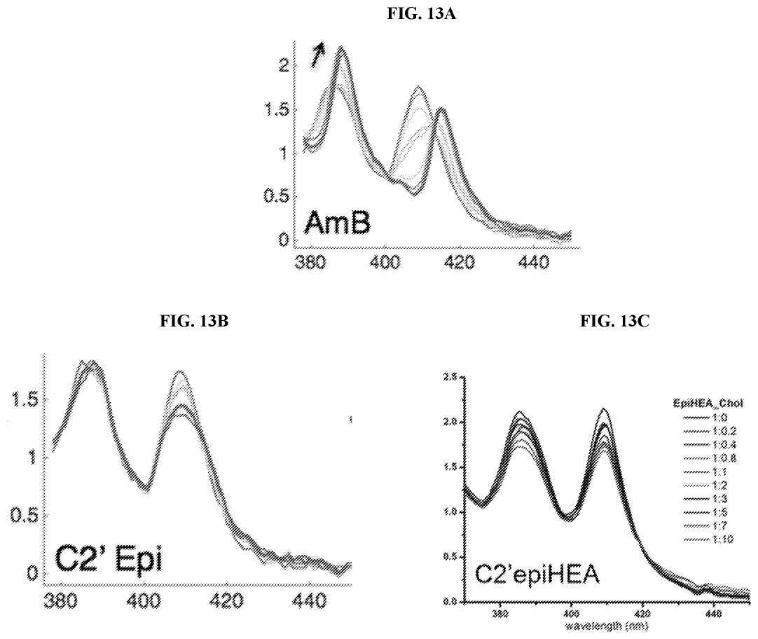

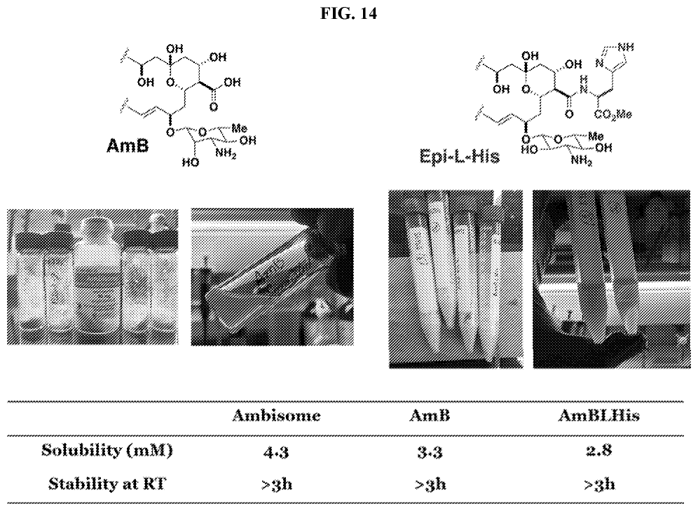

Killing kinetics for a C16 amide AmB (AmBHEA; Compound AA) as compared to AmB or DMSO (FIG. 11).

Colonies of Candida albicans SN250 from SDA plate was suspended in RPMI media and the inoculum density was maintained to 10⁵ CFU/ml. 990 μL aliquots of the dilute cell suspension were added to a sterile 1.7 mL eppendorf tubes followed by 10 uL of 400 uM solution of the compound (in DMSO). The concentration of DMSO in each eppendorf tube was 1% and a control sample to confirm viability using only 1% DMSO was also performed. At predetermined time points (0, 0.5, 1, 2, 3, 4, 5, 6, 8, 10, and 24 h), a 10 μL sample was removed from each tube and serially diluted 10 fold with RPMI, and a 10 μL aliquot was plated onto a SDA plate for colony count determination. When colony counts were expected to be less than 1,000 CFU/mL, a 50 μL aliquot was taken directly from the test solution and plated onto a SDA plate without dilution. Plates were incubated at 37° C. (C. albicans) for 24 prior to examination. All experiments were conducted in duplicate.

### Example 9. Additional Compounds Similar to AmBHEA (Compound AA)

TABLE 2
Exemplary Compounds Synthesized.

| Structure | Compound # |
|---|---|
| 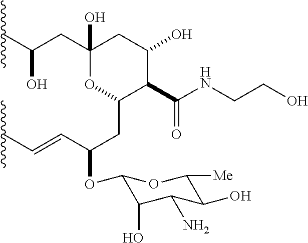 | AA |
| 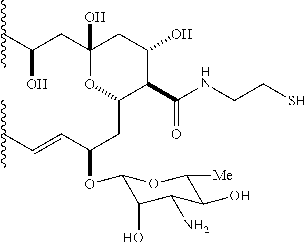 | AB |
| 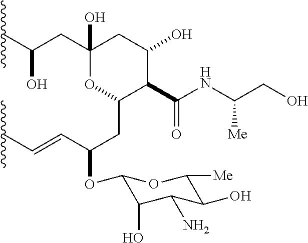 | AC |
| 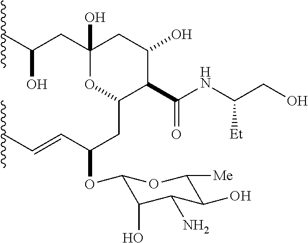 | AD |
| 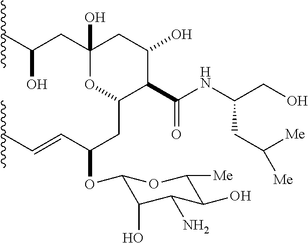 | AE |
| 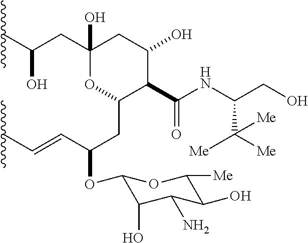 | AF |
| 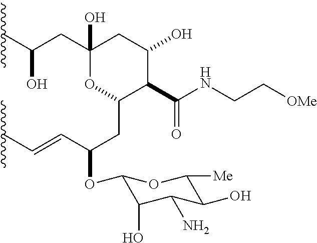 | AG |
| 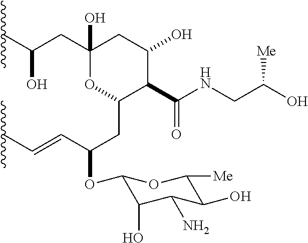 | AH |
| 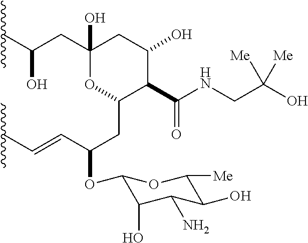 | AI |
| 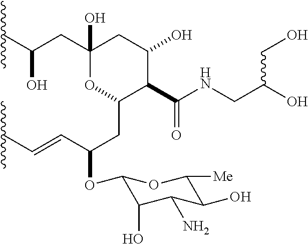 | AJ |
| 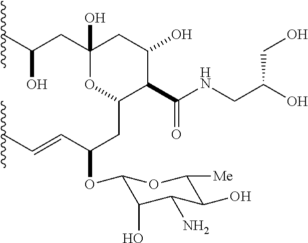 | AK |
| 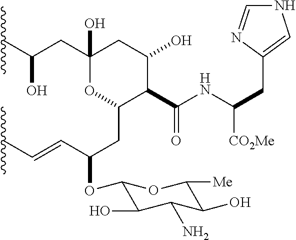 | AL |
| 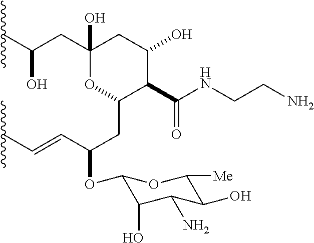 | AM |
| 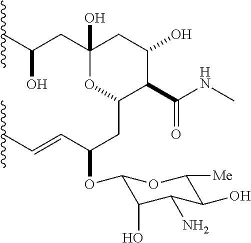 | AN |

### Example 10. Anti-Fungal Potency for AmBHEA Analogues

TABLE 3
Minimum Inhibition Concentrations (MIC) for Representative heteroatom and α-
substituted variations on AmBHEA (compound AA).

| | AmB | AA | AB | AC | AD | AE | AF | AG |
|---|---|---|---|---|---|---|---|---|
| | (μM) | (μM) | (μM) | (μM) | (μM) | (μM) | (μM) | (μM) |
| C. albicans SN250 | 0.225 | 0.25 | 0.4 | 0.3 | 0.3 | 1 | 0.4 | 0.4 |
| C. albicans | 0.25 | 0.225 | 0.45 | 0.3 | 0.35 | 0.75 | 0.5 | 035 |
| C. krusei | 0.7 | 0.45 | 1 | 0.45 | 0.5 | 1 | 1 | 1 |
| C. glabrata | 0.14 | 0.145 | 0.35 | 0.3 | 0.3 | 0.4 | 0.4 | 0.3 |
| C. tropicalis | 0.325 | 0.225 | 0.45 | 0.35 | 0.4 | 0.5 | 0.75 | 0.4 |
| A. fumigatus 91 | 1.5 | 1 | 1 | 2 | 2 | 2 | 2 | 2 |
| A. fumigatus 1163 | 1 | 0.525 | 1 | 1 | 1 | 1 | 2 | 1 |
| A. fumigatus 1100 | 1 | 0.7 | 1 | 1 | 1 | 1 | 2 | 1 |
| Average MIC | 0.642 | 0.44 | 0.706 | 0.713 | 0.731 | 0.956 | 1.131 | 0.806 |

TABLE 4
Minimum Inhibition Concentrations (MIC) for Representative β-substituted
variants of AmBHEA (compound AA).

| | AmB | AA | AH | AI | AJ | AK |
|---|---|---|---|---|---|---|
| | (μM) | (μM) | (μM) | (μM) | (μM) | (μM) |
| C. albicans SN250 | 0.225 | 0.25 | 0.35 | 0.3 | 0.15 | 0.15 |
| C. albicans | 0.25 | 0.225 | 0.35 | 0.4 | 0.20 | 0.25 |
| C. krusei | 0.7 | 0.45 | 0.75 | 1 | 0.3 | 0.4 |
| C. glabrata | 0.14 | 0.145 | 0.2 | 0.25 | 0.08 | 0.1 |
| C. tropicalis | 0.325 | 0.225 | 0.45 | 0.45 | 0.2 | 0.3 |
| A. fumigatus 91 | 1.5 | 1 | 2 | 1 | 1 | 1 |
| A. fumigatus 1163 | 1 | 0.525 | 1 | 1 | 0.3 | 0.5 |
| A. fumigatus 1100 | 1 | 0.7 | 1 | 1 | 0.4 | 0.75 |
| Average MIC | 0.642 | 0.44 | 0.763 | 0.675 | 0.329 | 0.431 |

### Example 11. Plasma Stability

TABLE 5
Plasma Stability in Rat and Human
for various C16 Amide AmB variants.

| | | % Remaining | Half Life |
|---|---|---|---|
| Compound ID | Species/Matrix | at 120 min | (min) |
| AmB | Rat Plasma | 88.3 | >289 |
| | Human Plasma | 97.7 | >289 |
| AL | Rat Plasma | 111 | >289 |
| | Human Plasma | 107 | >289 |
| AA | Rat Plasma | 108 | >289 |
| | Human Plasma | 119 | >289 |
| AM | Rat Plasma | 118 | >289 |
| | Human Plasma | 102 | >289 |
| AN | Rat Plasma | 118 | >289 |
| | Human Plasma | 106 | >289 |

### Example 12. In Vitro Metabolism

TABLE 6
In Vitro Metabolism Different Species for Various C16 Amide AmB variants.

| Compound | | | T_{1/2} | CL | CLᵢₙₜ | Remaining | Remaining |
|---|---|---|---|---|---|---|---|
| ID | Species | R² | (min) | (μl/min/mg) | (mL/min/kg) | (T = 60 min) | (T = NCF60 min)* |
| AmB | Mouse | 0.2588 | >145 | <9.6 | <38 | 106% | 93.1% |
| | Rat | 0.2532 | >145 | <9.6 | <17 | 121% | 111% |
| | Dog | 0.0152 | >145 | <9.6 | <14 | 106% | 94.7% |
| | Monkey | 0.2374 | >145 | <9.6 | <13 | 142% | 105% |
| | Human | 0.0079 | >145 | <9.6 | <8.6 | 100% | 104% |
| AL | Mouse | 0.1944 | >145 | <9.6 | <38 | 127% | 139% |
| | Rat | 0.8249 | >145 | <9.6 | <17 | 132% | 75.4% |
| | Dog | 0.0552 | >145 | <9.6 | <14 | 101% | 87.1% |
| | Monkey | 0.0605 | >145 | <9.6 | <14 | 107% | 106% |
| | Human | 0.0009 | >145 | <9.6 | <8.6 | 113% | 96.8% |
| AA | Mouse | 0.0010 | >145 | <9.6 | <38 | 118% | 140% |
| | Rat | 0.9714 | >145 | <9.6 | <17 | 130% | 113% |
| | Dog | 0.7355 | >145 | <9.6 | <14 | 88.5% | 97.4% |
| | Monkey | 0.0004 | >145 | <9.6 | <13 | 113% | 103% |
| | Human | 0.0013 | >145 | <9.6 | <8.6 | 111% | 142% |
| AM | Mouse | 0.0023 | >145 | <9.6 | <38 | 109% | 176% |
| | Rat | 0.0000 | >145 | <9.6 | <17 | 113% | 83.1% |
| | Dog | 0.0144 | >145 | <9.6 | <14 | 107% | 109% |
| | Monkey | 0.0130 | >145 | <9.6 | <13 | 113% | 121% |
| | Human | 0.0011 | >145 | <9.6 | <8.6 | 101% | 115% |
| AN | Mouse | 0.0229 | >145 | <9.6 | <38 | 102% | 131% |
| | Rat | 0.8534 | >145 | <9.6 | <17 | 106% | 93% |
| | Dog | 0.3099 | >145 | <9.6 | <14 | 92.4% | 113% |
| | Monkey | 0.2201 | >145 | <9.6 | <13 | 139% | 135% |
| | Human | 0.0435 | >145 | <9.6 | <8.6 | 104% | 127% |

### Example 13. Additional C16 Amide AmB Variants Synthesized

TABLE 7
Additional C16 Amide AmB Variants Synthesized and Their Average Minimum
Inhibition Concentrations (MICs).

| Structure | MIC (uM) | Compound # |
|---|---|---|
| 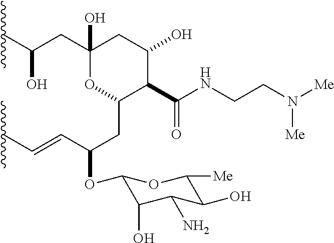 | 0.352 | DA |
| 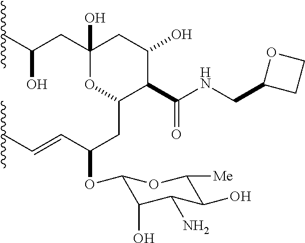 | 0.359 | DB |
| 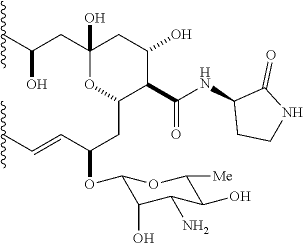 | 0.367 | DC |
| 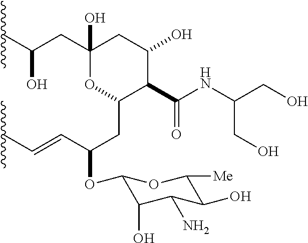 | 0.375 | DD |
| 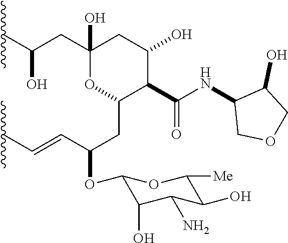 | 0.375 | DE |
| 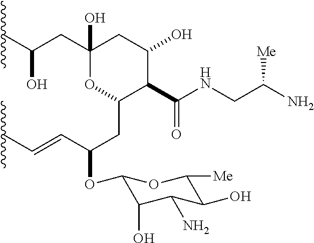 | 0.375 | DF |
| 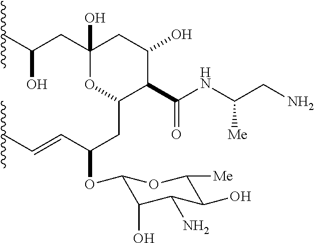 | 0.375 | DG |
| 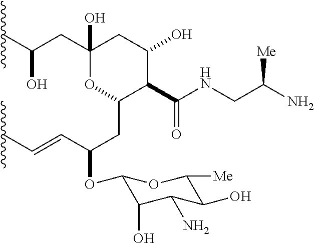 | 0.375 | DH |
| 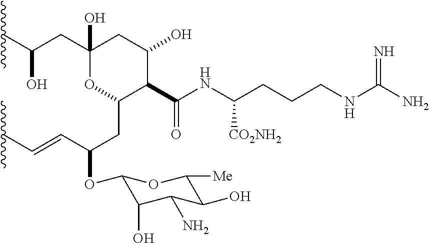 | 0.375 | DI |
| 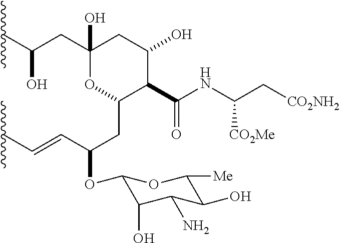 | 0.375 | DJ |
| 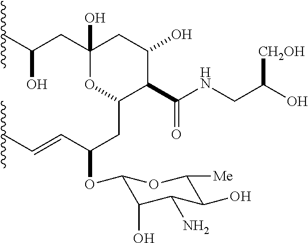 | 0.381 | DK |
| 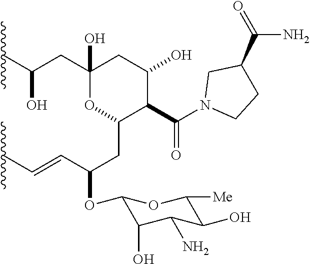 | 0.383 | DL |
| 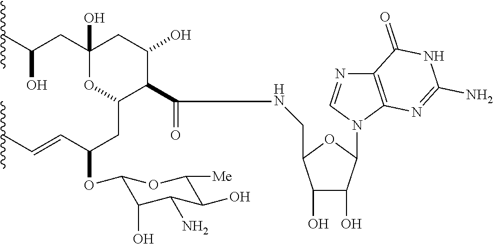 | 0.391 | DM |
| 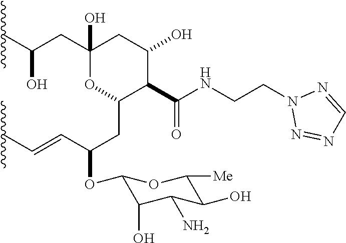 | 0.391 | DN |
| 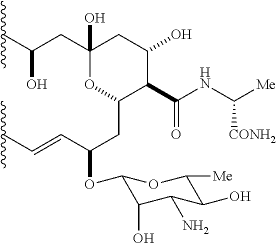 | 0.391 | DO |
| 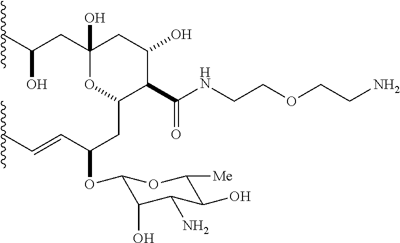 | 0.391 | DP |
| 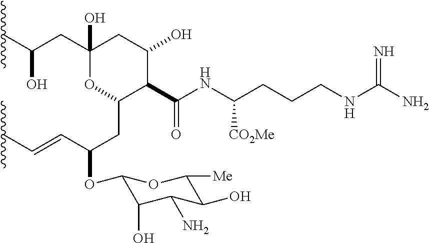 | 0.406 | DQ |
| 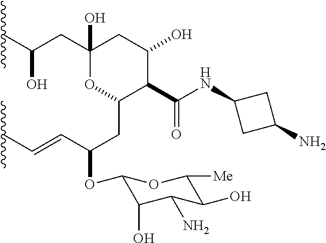 | 0.406 | DR |
| 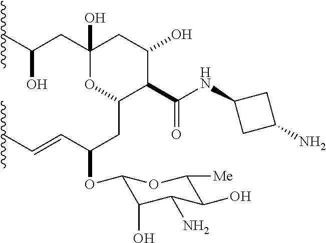 | 0.406 | DS |
| 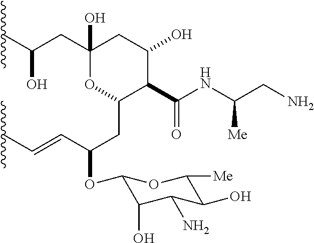 | 0.406 | DT |
| 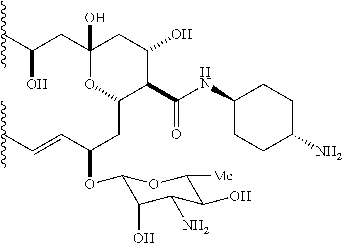 | 0.406 | DU |
| 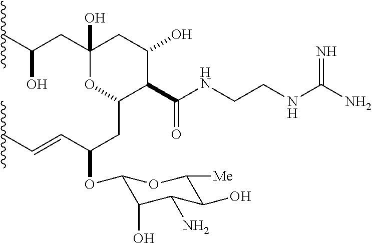 | 0.406 | DV |
| 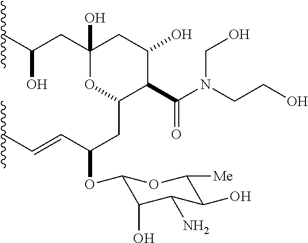 | 0.419 | DW |
| 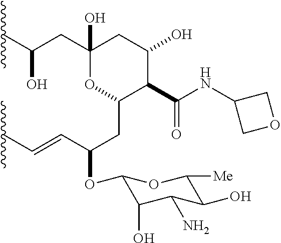 | 0.422 | DX |
| 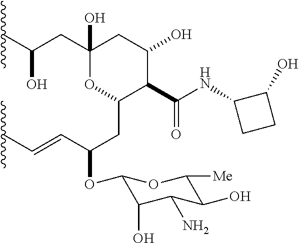 | 0.422 | DY |
| 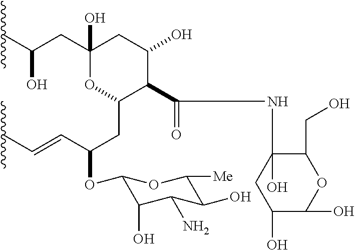 | 0.422 | DZ |
| 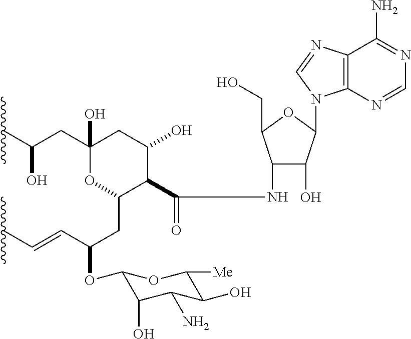 | 0.422 | EA |
| 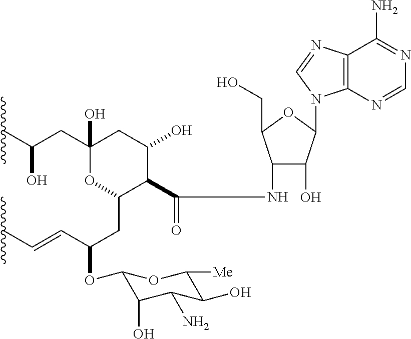 | 0.422 | EB |
| 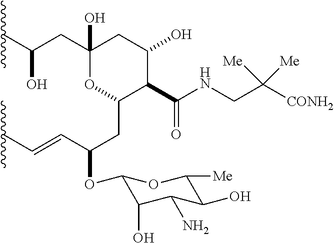 | 0.422 | EC |
| 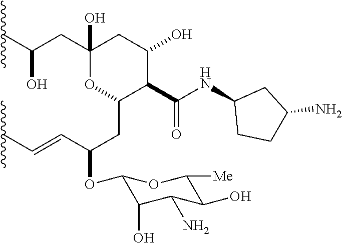 | 0.422 | ED |
| 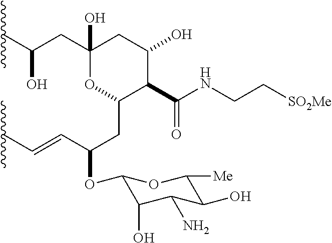 | 0.422 | EE |
| 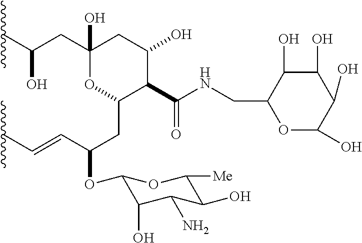 | 0.430 | EF |
| 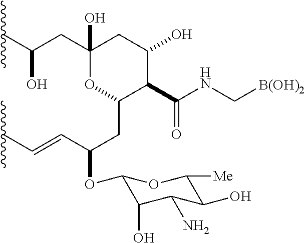 | 0.430 | EG |
| 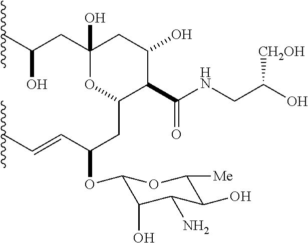 | 0.431 | EH |
| 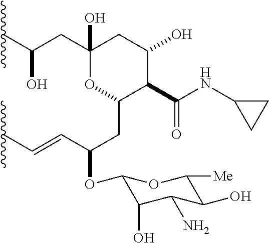 | 0.438 | EI |
| 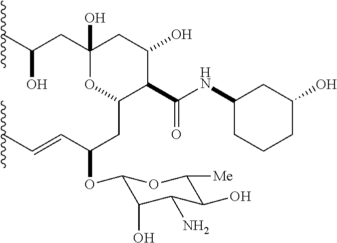 | 0.438 | EJ |
| 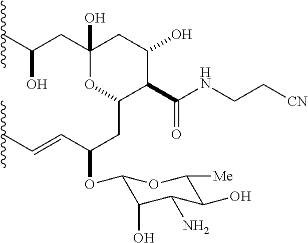 | 0.438 | EK |
| 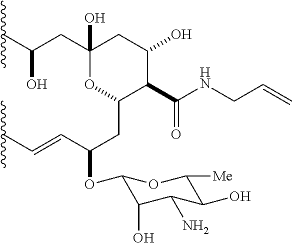 | 0.453 | EL |
| 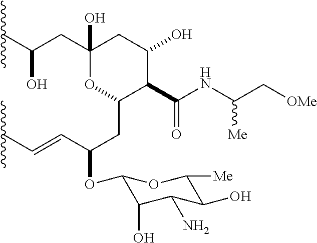 | 0.469 | EM |
| 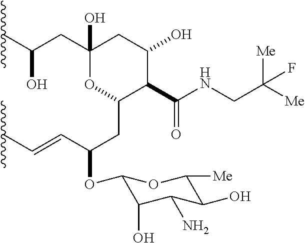 | 0.469 | EN |
| 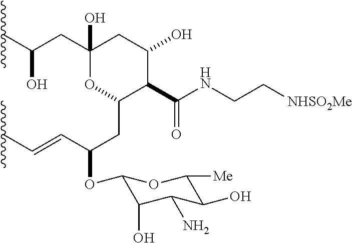 | 0.469 | EO |
| 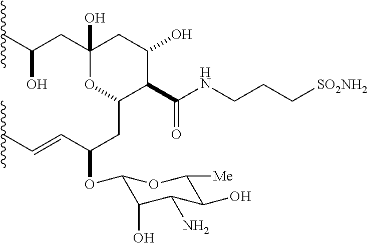 | 0.484 | EP |
| 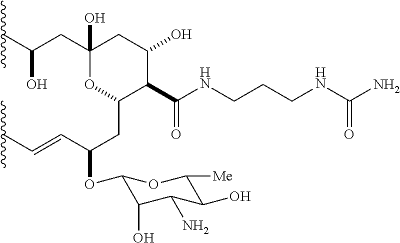 | 0.484 | EQ |
| 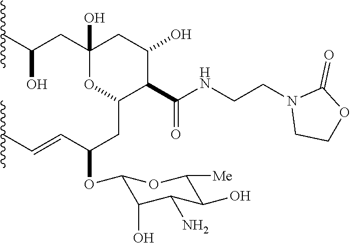 | 0.484 | ER |
| 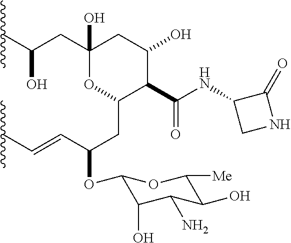 | 0.484 | ES |
| 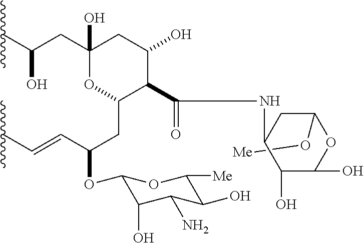 | 0.492 | ET |
| 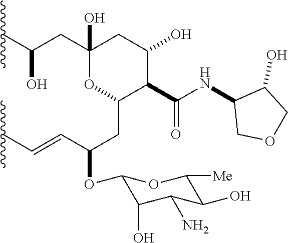 | 0.500 | EU |
| 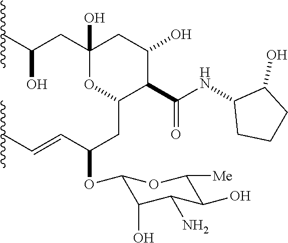 | 0.500 | EV |
| 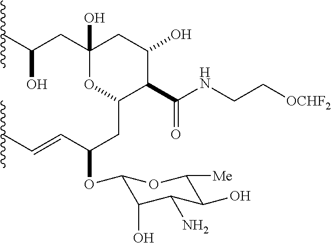 | 0.500 | EW |
| 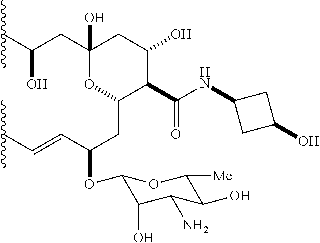 | 0.500 | EX |
| 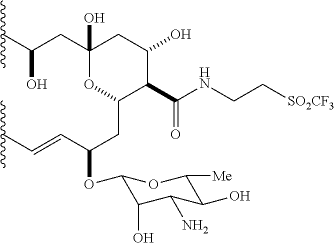 | 0.500 | EX |
| 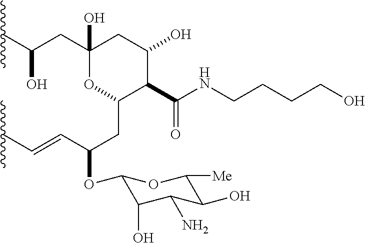 | 0.500 | EY |
| 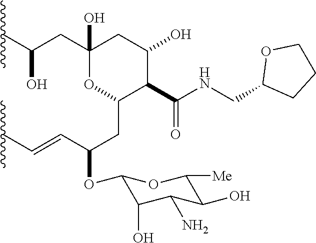 | 0.516 | EZ |
| 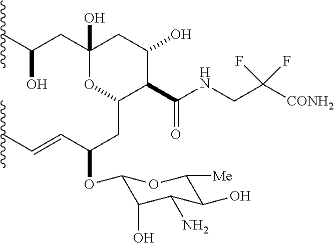 | 0.516 | FA |
| 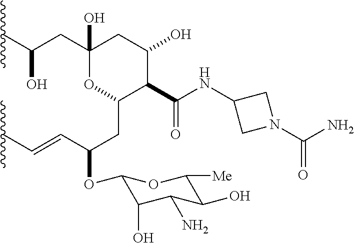 | 0.516 | FB |
| 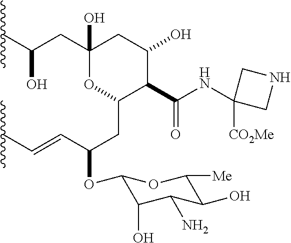 | 0.516 | FC |
| 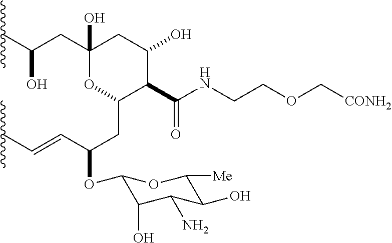 | 0.516 | FD |
| 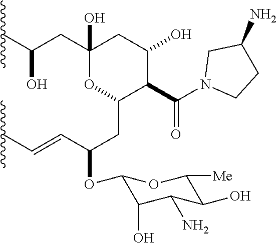 | 0.531 | FE |
| 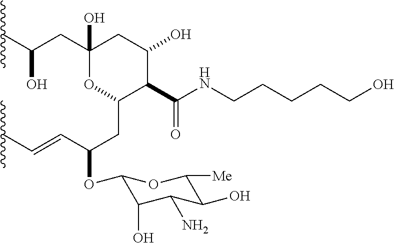 | 0.531 | FF |
| 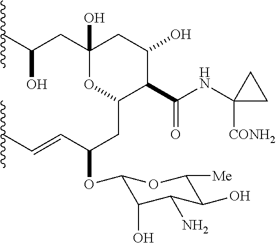 | 0.531 | FG |
| 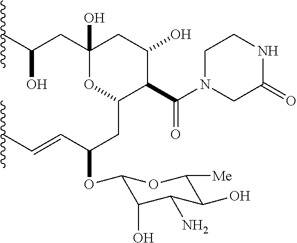 | 0.531 | FH |
| 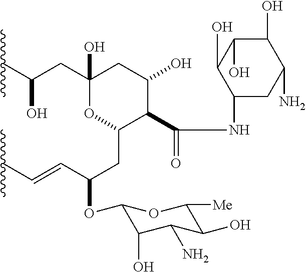 | 0.172 | FI |
| 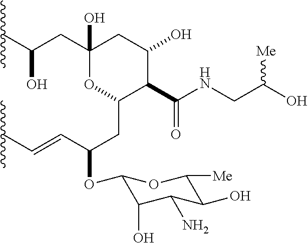 | 0.195 | FJ |
| 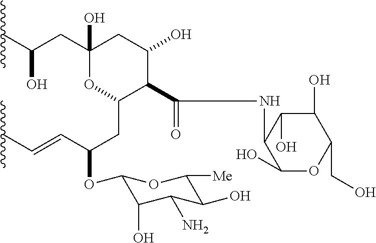 | 0.211 | FK |
| 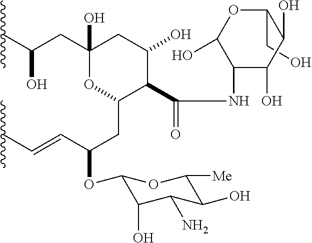 | 0.227 | FL |
| 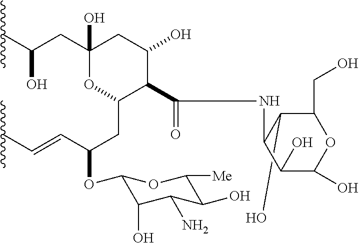 | 0.234 | FM |
| 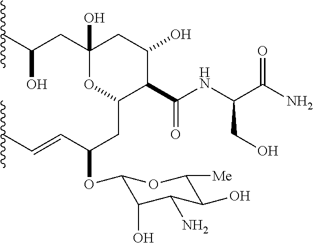 | 0.238 | FN |
| 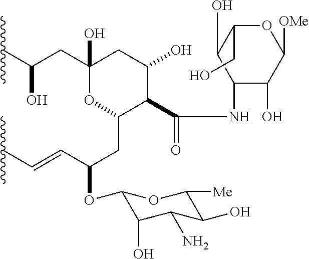 | 0.258 | FO |
| 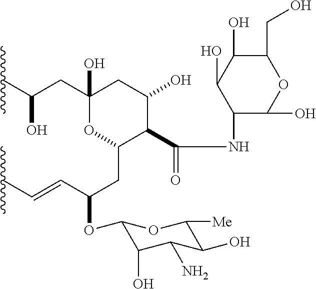 | 0.262 | FP |
| 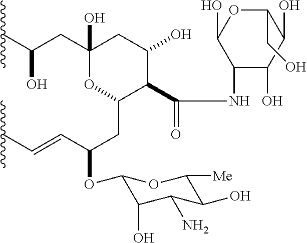 | 0.270 | FQ |
| 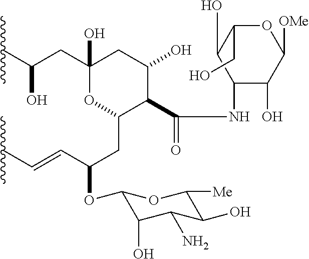 | 0.270 | FR |
| 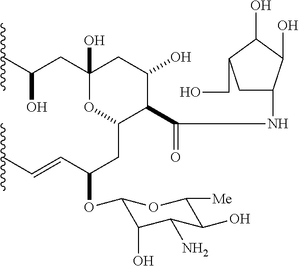 | 2.81 | FS |
| 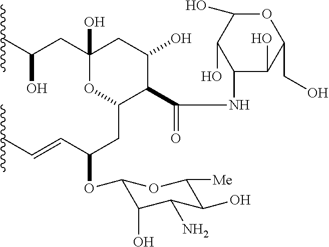 | 0.289 | FT |
| 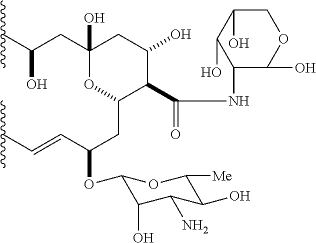 | 0.289 | FU |
| 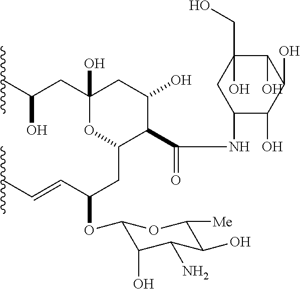 | 0.305 | FV |
| 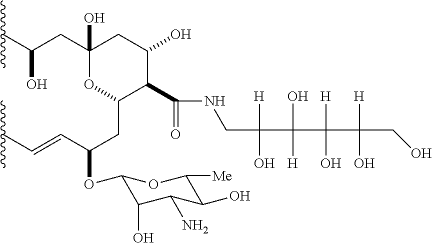 | 0.313 | FW |
| 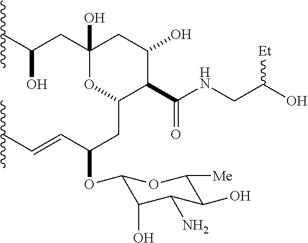 | 0.313 | FX |
| 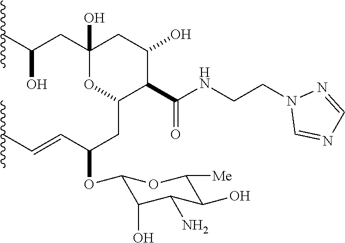 | 0.316 | FY |
| 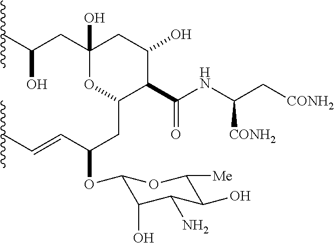 | 0.316 | FZ |
| 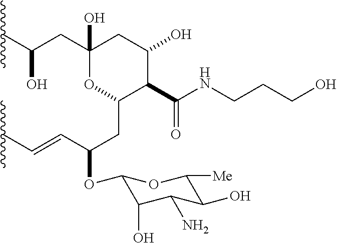 | 0.320 | GA |
| 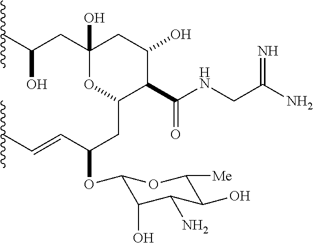 | 0.320 | GB |
| 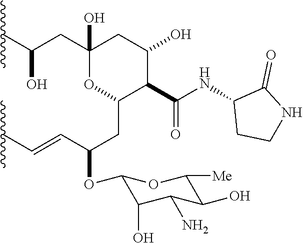 | 0.320 | GC |
| 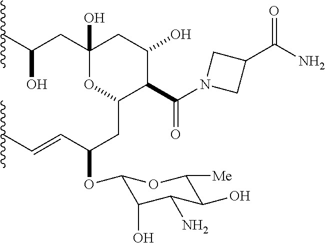 | 0.320 | GD |
| 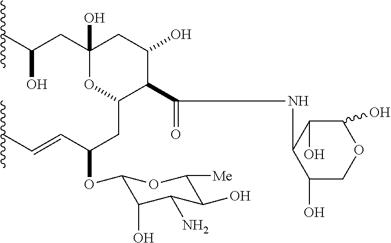 | 0.320 | GE |
| 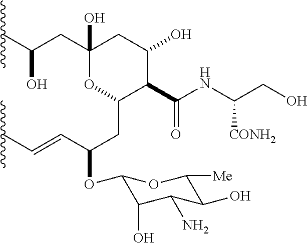 | 0.324 | GF |
| 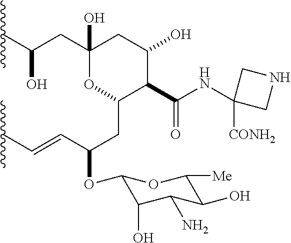 | 0.328 | GG |
| 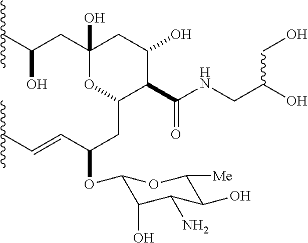 | 0.329 | GH |
| 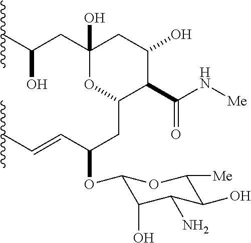 | 0.336 | GI |
| 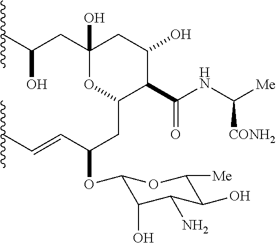 | 0.336 | GJ |
| 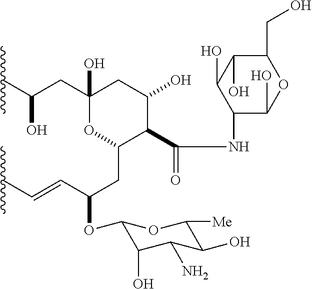 | 0.340 | GK |
| 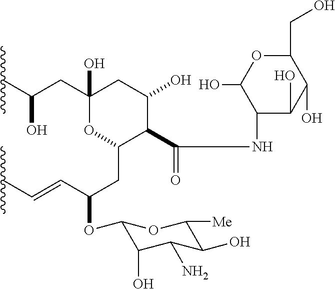 | 0.344 | GL |
| 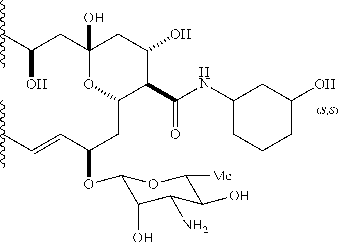 | 0.344 | GM |
| 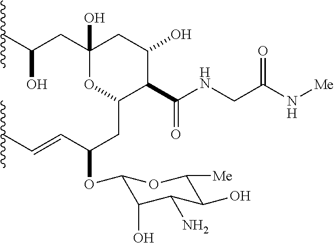 | 0.344 | GN |

### Example 14. Structure-Activity Relationship

### Structure-Activity Relationship—Aliphatic Ring.

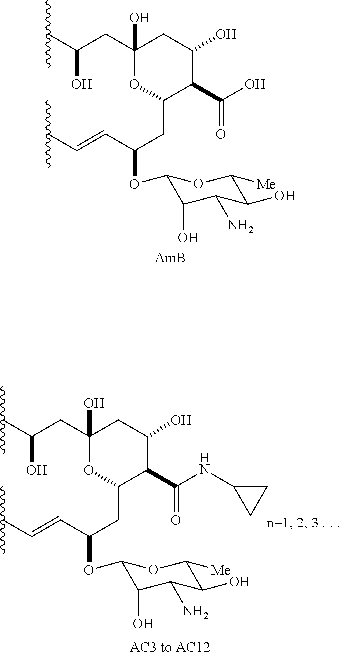

TABLE 8
Structure-Activity Relationship - Aliphatic Ring

| MIC | | | | | | | | |
|---|---|---|---|---|---|---|---|---|
| (mM) | AmB | AR3 | AR4 | AR5 | AR6 | AR7 | AR-10 | AR12 |
| # Carbons (n + 2) | | 3 | 4 | 5 | 6 | 7 | 10 | 12 |
| C. albicans SN250 | 0.218 | 0.25 | 0.5 | 0.5 | 0.5 | 0.75 | 1 | 2 |
| C. albicans | 0.215 | 0.25 | 0.5 | 0.5 | 0.5 | 1 | 1 | 2 |
| C. glabrata | 0.510 | 0.5 | 1 | 1 | 0.75 | 2 | 2 | 1 |
| C. krusei | 0.139 | 0.25 | 0.55 | 0.5 | 0.375 | 1 | 1 | 8 |
| C. tropicalis | 0.285 | 0.25 | 0.5 | 0.5 | 0.5 | 1 | 1 | 3 |
| A. fumigatus 91 | 1.311 | 1 | 1 | 2 | 2 | 2 | 8 | 8 |
| A. fumigatus 1100 | 0.967 | 0.5 | 1 | 1.5 | 1.5 | 2 | 2 | 8 |
| A. fumigatus 1163 | 0.8 | 0.5 | 1 | 2 | 1 | 2 | 2 | 8 |
| Average MIC | 0.556 | 0.438 | 0.750 | 1.063 | 0.891 | 1.469 | 2.25 | 5.00 |

### Structure-Activity Relationship—Aliphatic Acyclic Chain.

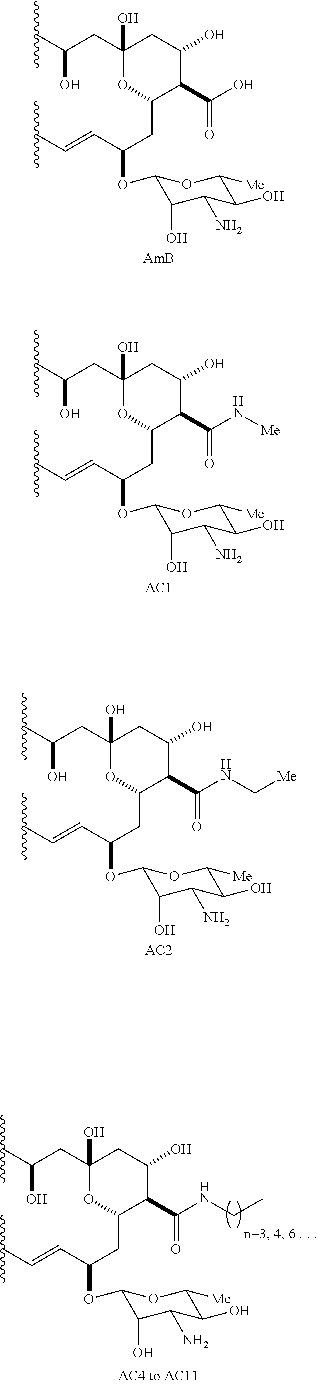

TABLE 9
Structure-Activity Relationship - Aliphatic Acyclic Chain

| MIC | | | | | | | | | |
|---|---|---|---|---|---|---|---|---|---|
| (mM) | AmB | AC1 | AC2 | AC4 | AC5 | AC7 | AC8 | AC9 | AC-11 |
| # of Carbons | | 1 | 2 | 4 | 5 | 7 | 8 | 9 | 11 |
| C. albicans SN250 | 0.218 | 0.1875 | 0.25 | 1 | 1 | 1 | 1 | 1 | 4 |
| C. albicans | 0.215 | 0.125 | 0.25 | 1 | 1 | 1 | 1 | 2 | 4 |
| C. glabrata | 0.139 | 0.125 | 0.375 | 1 | 1 | 1 | 1 | 1 | 1 |
| C. krusei | 0.510 | 0.5 | 0.5 | 1 | 1 | 2 | 2 | 2 | 8 |
| C. tropicalis | 0.285 | 0.25 | 0.25 | 1 | 1 | 1 | 1 | 2 | 8 |
| A. fumigatus 91 | 1.311 | 0.5 | 1 | 2 | 2 | 2 | 3 | 2 | 4 |
| A. fumigatus 1100 | 0.967 | 0.5 | 1 | 1 | 1.5 | 2 | 2 | 2 | 4 |
| A. fumigatus 1163 | 0.8 | 0.5 | 0.75 | 1 | 1.5 | 2 | 2 | 2 | 4 |
| Average MIC | 0.556 | 0.336 | 0.547 | 1.125 | 1.250 | 1.500 | 1.625 | 1.750 | 4.625 |

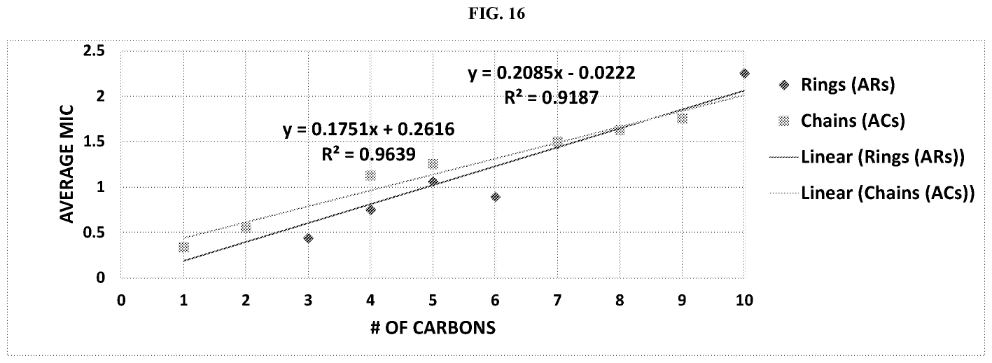

Impact of aliphatic rings and aliphatic acyclic chains is summarized in FIG. 16,

### Structure-Activity Relationship—Impact of Polar Functional Groups.

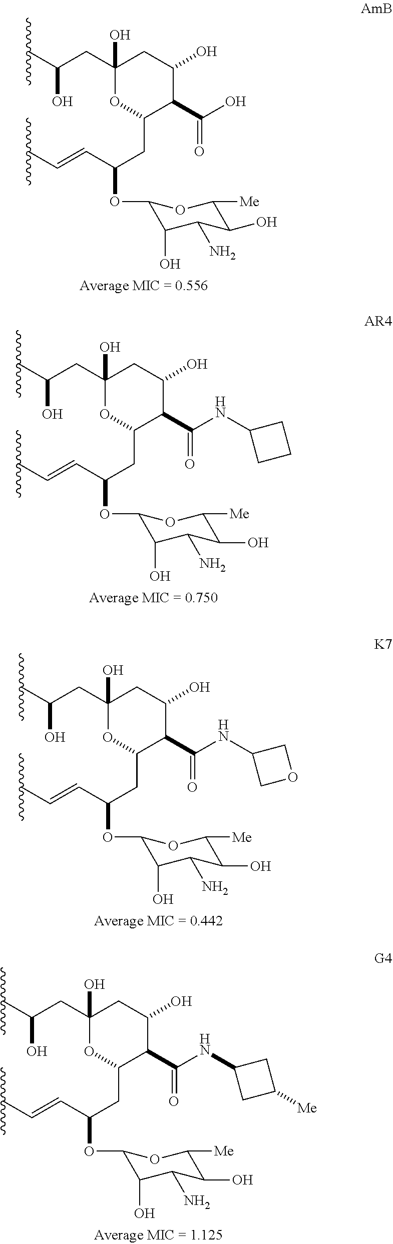

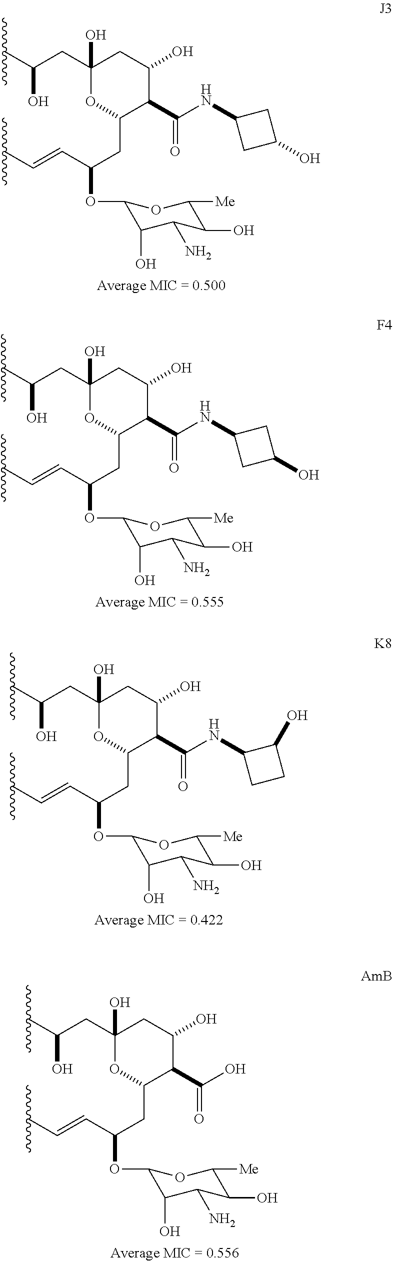

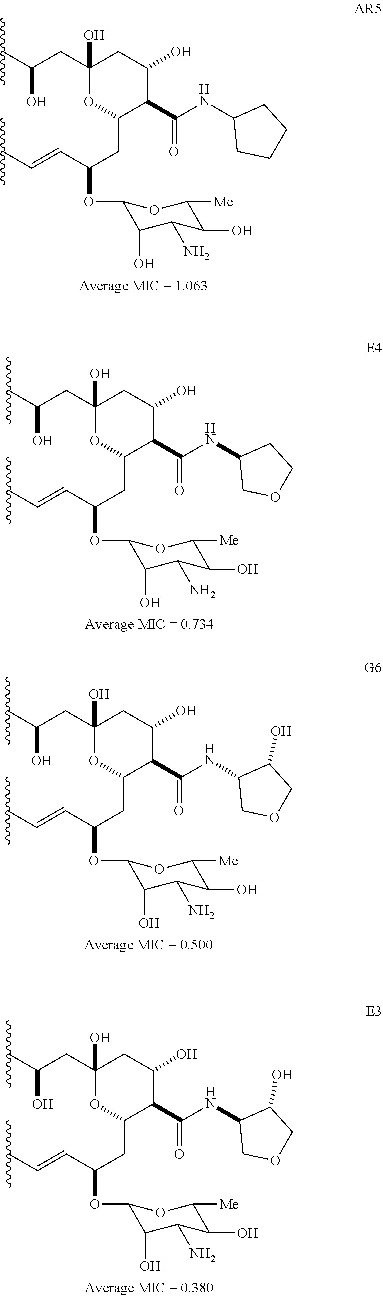

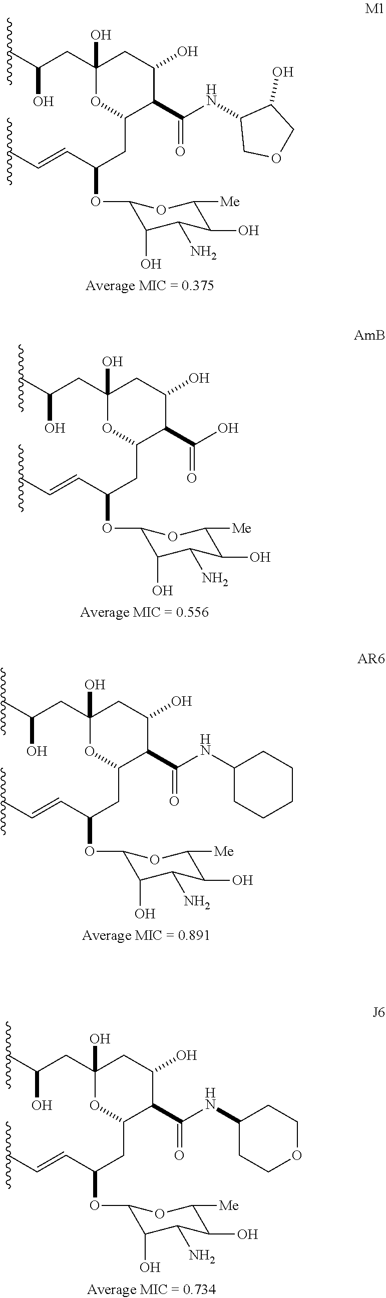

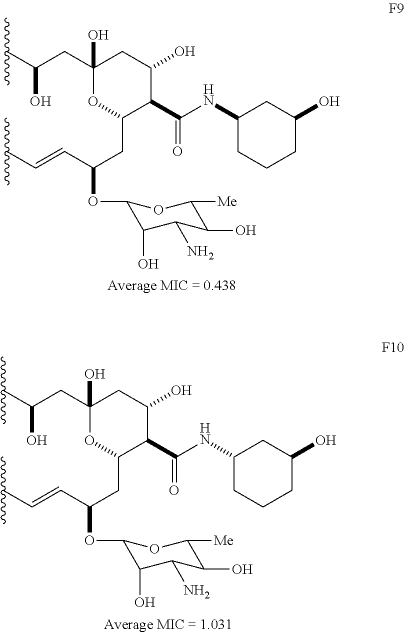

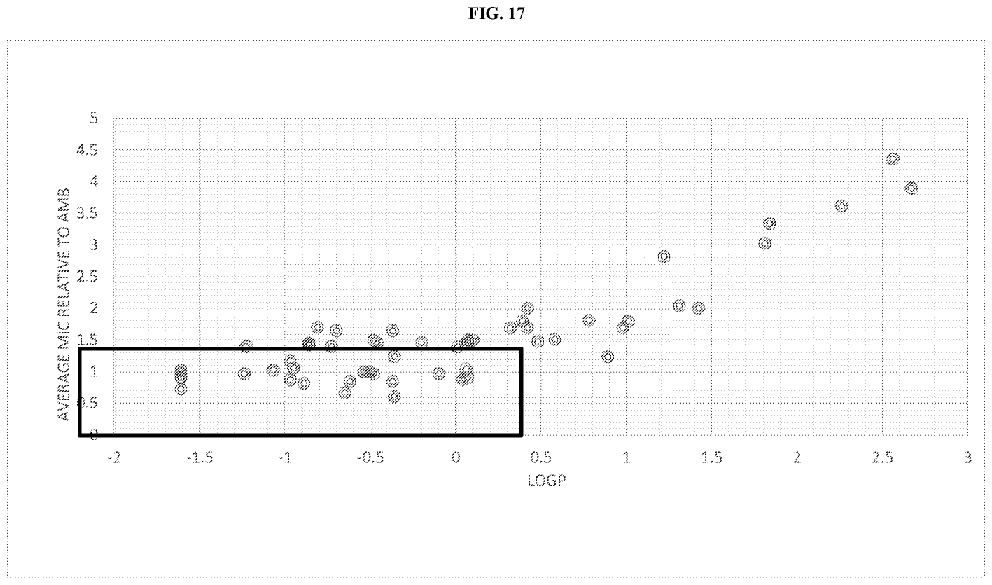

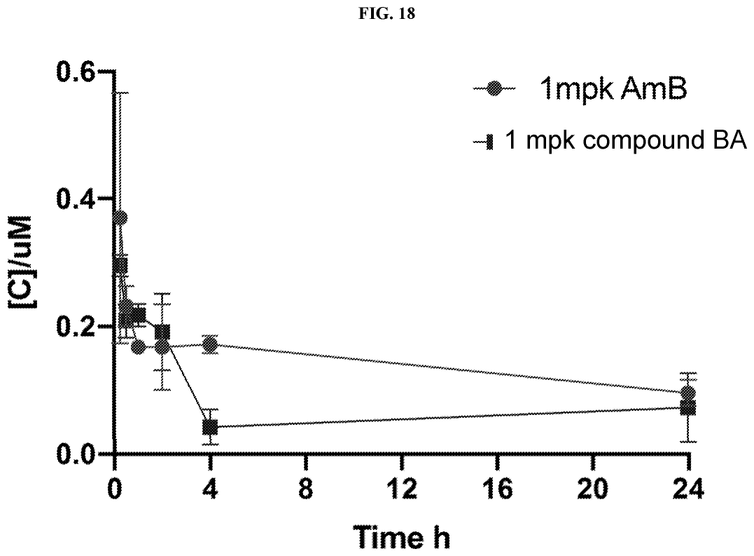

Impact of various polar functional groups are summarized in FIG. 17.

### Part II. C16 Amide C2′epiAmB Derivatives

### Example 15, General Synthetic Procedure and HPLC Method for C16 Amides of C2′epiAmB

Synthetic Procedure

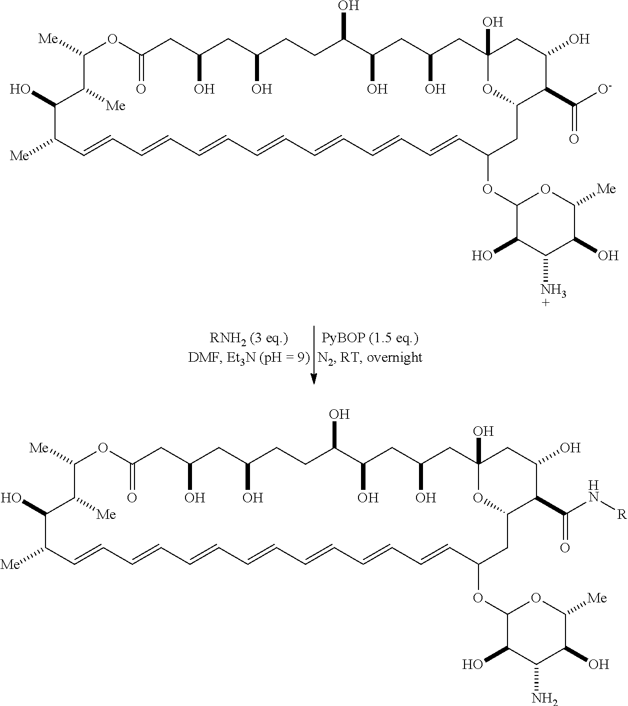

Freshly distilled Et₃N was added drop wise to a solution of C2′epi-Amphotericin B (10 mg; 0.01 mmol) and amine (3 eq.) in DMF (500 μL) until pH=9 is reached (by pH paper). The reaction mixture was stirred for 15 minutes at room temperature. Solid PyBOP (1.5 eq; 8.4 mg) was added under nitrogen atmosphere, and the sealed vial was stirred overnight at rt. The progress of the reaction was monitored by analytical HPLC traces.

Once completed, the product was precipitated and washed with anhydrous diethyl ether (10 mL). The suspension was centrifuged at 3000 g for 5 minutes. The solvent was decanted out and the pellet was dissolved in DMSO and filtered through 0.2 micron syringe filter for purification on C18 Prep HPLC system. The pure product was dried on lyophilizer as yellowish powder and stored at −80° C. under nitrogen atmosphere.

HPLC Method:

Analytical Column: C18 Agilent column (Catalogue number: 993967-902)

| | | 10 mM NH₄OAc | Flow rate |
|---|---|---|---|
| Time (min) | Acetonitrile | buffer | (mL/min) |
| 0 | 5 | 95 | 1.2 |
| 8 | 95 | 5 | 1.2 |
| 8.5 | 95 | 5 | 1.2 |
| 9.5 | 5 | 95 | 1.2 |
| 10.5 | 5 | 95 | 1.2 |

Prep Column: C18 Agilent column (Catalogue number: 410910-502)

| | | 10 mM NH₄OAc | Flow rate |
|---|---|---|---|
| Time (min) | Acetonitrile | buffer | (mL/min) |
| 0 | 5 | 95 | 30 |
| 1 | 5 | 95 | 50 |
| 15 | 95 | 5 | 50 |
| 16 | 95 | 5 | 50 |
| 17 | 5 | 95 | 50 |
| 18 | 5 | 95 | 30 |

### Example 16. Synthesis of Representative C16 Amide C2′epiAmB Derivatives

Synthesis of C2′epiAmBMethyl Amide (C2′epiAmBMA)

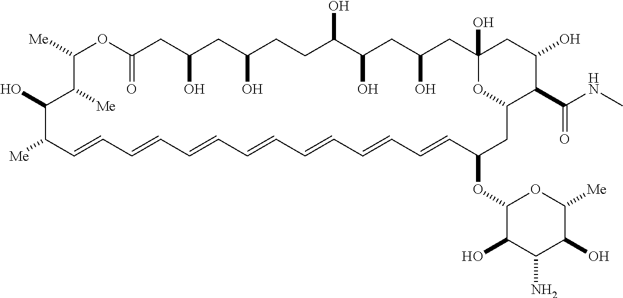

A 7 mL reaction vial was charged with C2′epiAmB (5 mg, 1 equiv) and Fmoc-succinimide (3 mg, 1.5 eq) which were dissolved in a 2:1 mixture of DMF:MeOH (150 μL) at room temperature. Pyridine (3 μL, 6 equiv.) was subsequently added and the reaction was stirred overnight at room temperature. The reaction mixture was then poured into diethyl ether (5 mL) and the yellow solid was collected as pellet through centrifugation. The solid was dried under N₂ flow for 2 mins and used for the next step without any purification.

To a stirred solution of Fmoc-epiAmB (A) in DMF (250 μL) in 7 mL oven dried clean vial at 23° C., PyBOP (9 mg; 1 equiv.) and DIPEA (1.25 μL; 2 equiv.) were added and stirred for 5 mins. A solution of MeNH₂ in THF (3.5 μL; 1(M) in THF; 1.5 equiv.) was added sequentially and the mixture was stirred for 2 h. The progress of the reaction was monitored by HPLC.

Upon complete consumption of substrate (2 h), piperidine (5 μL; 2 equiv.) was added to the reaction and stirred for another 1 h. The completion of the reaction was monitored by HPLC. The mixture was poured in 5 mL diethylether and the resulting yellow solid was collected as pellet through centrifugation (5 mins; 3000 g). The solid was resuspended in DMSO and purified by singleprep HPLC (C18, 5-μm, 50×250 mm, 75 mL/min, 95:5 to 5:95 15 mM NH₄OAc (aq):MeCN over 22 minutes). Following the HPLC, the solvent was removed under reduced pressure and the compound was re-dissolved in DMSO and lyophilized resulting yellow white solid. The compound was stored at −80° C. in air-tight vial. Overall Yield=35% Mass: Observed [M+H⁺]=937.5249; Calculated [M+H⁺]=937.5268; Observed [M+Na⁺]=959.5065; Calculated [M+Na⁺]=950.5087.

Synthesis of C2′epiAmBAminoethyl Amide (C2′epiAmBAEA)

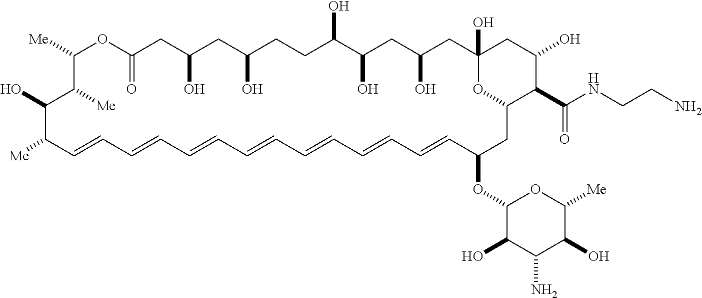

A 7 mL reaction vial was charged with C2′epiAmB (5 mg, 1 equiv) and Fmoc-succinimide (3 mg, 1.5 eq) which were dissolved in a 2:1 mixture of DMF:MeOH (150 μL) at room temperature. Pyridine (3 μL, 6 equiv.) was subsequently added and the reaction was stirred overnight at room temperature. The reaction mixture was then poured into diethyl ether (5 mL) and the yellow solid was collected as pellet through centrifugation. The solid was dried under N₂ flow for 2 mins and used for the next step without any purification.

To a stirred solution of Fmoc-epiAmB in DMF (250 μL) in 7 mL oven dried clean vial at 23° C., PyBOP (2.25 mg; 1 equiv.) and DIPEA (1.25 μL; 2 equiv.) were added and stirred for 5 mins. Solid Fmoc-ethylamine, HCl salt (2.5 mg; 1.5 equiv.) was added sequentially and the mixture was stirred for 2 h. The progress of the reaction was monitored by HPLC.

Upon complete consumption of substrate (2 h), piperidine (5 μL; 2 equiv.) was added to the reaction and stirred for another 1 h. The completion of the reaction was monitored by HPLC. The mixture was poured in 5 mL diethylether and the resulting yellow solid was collected as pellet through centrifugation (5 mins; 3000 g). The solid was resuspended in DMSO and purified by singleprep HPLC (C18, 5-μm, 50×250 mm, 75 mL/min, 95:5 to 5:95 15 mM NH₄OAc (aq):MeCN over 22 minutes). Following the HPLC, the solvent was removed under reduced pressure and the compound was re-dissolved in DMSO and lyophilized resulting yellow white solid. The compound was stored at −80° C. in air-tight vial. Overall Yield=29%; Mass: Observed [M+Na⁺]=988.53; Calculated [M+Na⁺]=988.5353.

### Example 17. Exemplary C16 Amide C2′epiAmB Synthesized

TABLE 10
Exemplary C16 Amides of C2'epiAmB Sythesized

| Structure | Designation | LCMS |
|---|---|---|
| 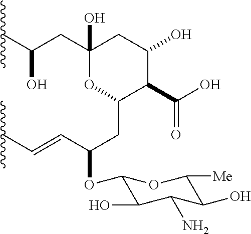 | C2'epiAmB | — |
| 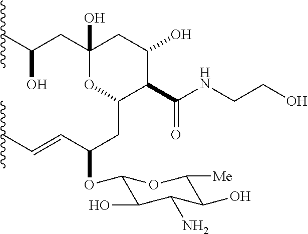 | BA | [M + H]⁺ Calculated C₄₉H₇₈N₂O₁₇, 949.5273, found 949.5278. |
| 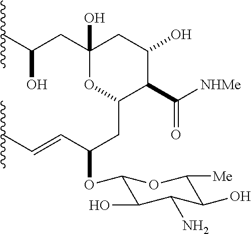 | BB | |
| 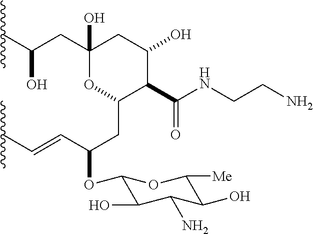 | BC | |
| 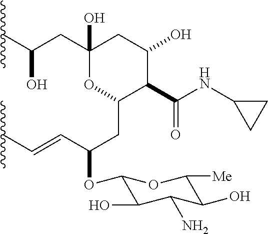 | BD | [M + H]⁺ Calculated C₅₀H₇₈N₂O₁₆, 963.5351, Observed 963.5405. |
| 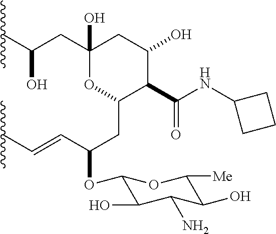 | BE | [M + H]⁺ Calculated C₅₁H₈₀N₂O₁₆, 977.5508, found 977.5593. |
| 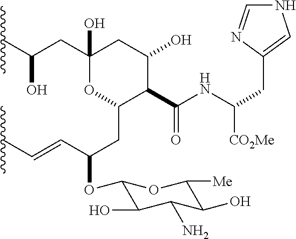 | BF | |
| 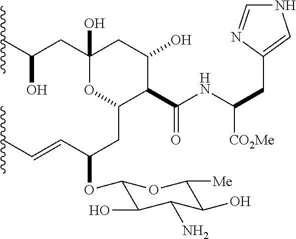 | BG | [M + H]⁺ Calculated C₅₂H₈₂N₄O₁₈, 1075.5702, found 1075.5735. |
| 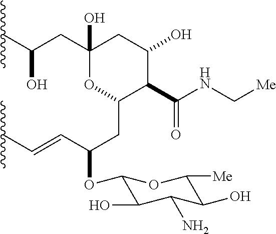 | BH | [M + H]⁺ Calculated C₄₉H₇₈N₂O₁₆, 951.5424, Observed 951.5622 |
| 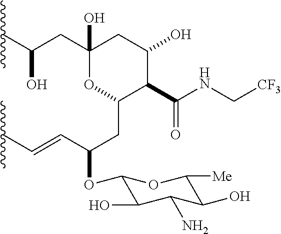 | BI | |
| 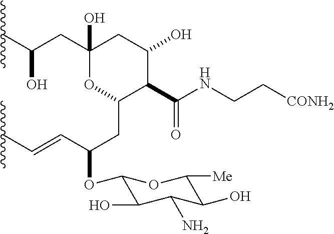 | BJ | [M + H]⁺ Calculated C₅₀H₇₉N₃O₁₇, 994.5482, observed 994.5382 |
| 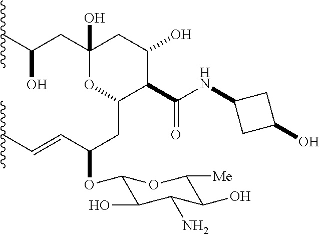 | BK | [M + H]⁺ Calculated C₅₁H₈₀N₂O₁₇, Observed 993.4768. |
| 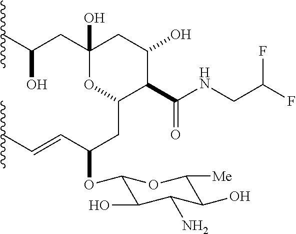 | BL | [M + H]⁺ Calculated C₄₉H₇₆F₂N₂O₁₆, 987.5163, found 987.5248. |

### Example 18. C16 Amide C2′epiAmB Derivatives Show Strong Inhibition Against A. fumigatus

TABLE 11
Minimum Inhibition Concentrations (MIC) for Representative C16 amides of C2’epiAmB.

| | | C2’epi | | | | | | |
|---|---|---|---|---|---|---|---|---|
| | AmB | AmB | BA | BB | BC | BD | BE | BF |
| | (μM) | (μM) | (μM) | (μM) | (μM) | (μM) | (μM) | (μM) |
| C. albicans SN250 | 0.15 | 0.5 | 0.25 | 0.5 | 0.5 | 0.5 | 1 | 0.5 |
| C. albicans | 0.24 | 0.5 | 0.25 | 0.5 | 0.5 | 1 | 2 | 0.5 |
| C. krusei | 0.28 | 1 | 1 | 2 | 4 | 4 | 4 | 4 |
| C. glabrata | 0.11 | 0.25 | 0.125 | 0.375 | 0.5 | 0.5 | 0.5 | 0.25 |
| C. tropicalis | 0.21 | 0.5 | 0.1875 | 0.5 | 0.5 | 0.5 | 1 | 0.375 |
| A. fumigatus 91 | 1.1 | >32 | 2 | 4 | 4 | 8 | 8 | >32 |
| A. fumigatus 1163 | 0.67 | 4 | 1 | 4 | 2 | 6 | 8 | 32 |
| A. fumigatus 1100 | 0.75 | 16 | 1 | 2 | 2 | 4 | 4 | 8 |
| Average MIC | 0.44 | >6.844 | 0.727 | 1.734 | 1.750 | 3.063 | 3.563 | >9.703 |
| Average MIC | | | 0.328 | 0.336 | 0.563 | 0.500 | 1.281 | 1.047 |
| for AmB analogue | | | | | | | | |
| MHC | 8.4 | >500 | >500 | >500 | 250 | ND | ND | >500 |
| Cholesterol Binding | Yes | No | No | No | Yes? | ND | ND | No |

TABLE 11A
Minimum Inhibition Concentrations for AmB and the
Disclosed Compounds Against Different Moulds.

| MIC (μM) | # of Isolates | AmB | BA | BM |
|---|---|---|---|---|
| A. fumigatus | 5 | 0.71 | 2 | 1.83 |
| A. flavus | 6 | 1.16 | 2.67 | 2.5 |
| A. niger | 6 | 0.21 | 0.58 | 3 |
| A. terreus | 6 | 3 | 3.33 | 1.08 |
| A. calldoustus | 6 | 1.5 | 1.67 | 1.5 |
| A. lentulus | 6 | 3.75 | 3 | 2.83 |
| A. thermomutatus | 6 | 0.6 | 1.6 | 1.7 |
| A. tubingensis | 6 | 0.125 | 0.92 | 0.92 |
| Mucor circinelloides | 6 | 0.125 | 1 | 1 |
| Mucor janssenii | 6 | 0.06 | 0.92 | 0.92 |
| Mucor velutinosus | 6 | 0.08 | 1.25 | 0.92 |
| Histoplasma capsulatum | 6 | 0.06 | 0.06 | 0.06 |
| Coccidioides immitis | 6 | 0.125 | 0.29 | 0.23 |
| Coccidioides posadasii | 6 | 0.06 | 0.46 | 0.42 |
| Fusarium oxysporum | 6 | 1.5 | 11.3 | 9.3 |
| Fusarium solani | 6 | 1.33 | 8.67 | 8.8 |
| P. variotii MYA-3630 (QC) | 5 | 1 | 2.7 | 2.67 |
| Cunninghamella sp. | 6 | 2 | 7.3 | 6 |
| Licht. Corymbifera | 6 | 0.23 | 1 | 1 |
| Licht. Ramosa | 6 | 0.15 | 1.1 | 1 |
| Syncephalastrum sp. | 6 | 0.06 | 1 | 1 |
| Claophialophora bantiana | 6 | 0.375 | 2 | 1 |
| Blastomyces dermatitidis | 5 | <0.03 | 0.6 | 0.5 |
| Fonsecaea sp. | 6 | <0.03 | 0.06 | <0.03 |
| Talaromyces marneffei | 6 | 0.09 | 0.15 | 0.2 |
| Apophysomyces sp. | 3 | 0.75 | 16 | 8.3 |
| Saksenaea sp. | 4 | 0.06 | 0.34 | 0.34 |
| Average MIC | 157 | 0.9 | 2.3 | 2.3 |

### Example 19. Additional C16 Amides of C2′epiAmB Synthesized and their Average Minimum Inhibition Concentrations (MICs)

TABLE 12
Additional C16 Amides of C2'epiAmB Synthesized and their
Average Minimum Inhibition Concentrations (MICs).

| | Average | Com- |
|---|---|---|
| | MIC | pound |
| Structure | (uM) | # |
| 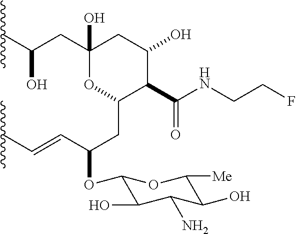 | 0.844 | BM |
| 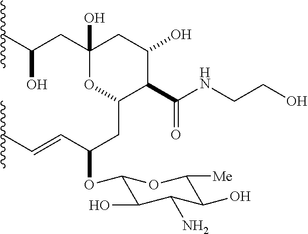 | 1.031 | BA |
| 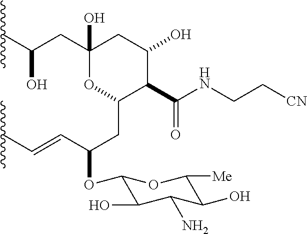 | 1.188 | BO |
| 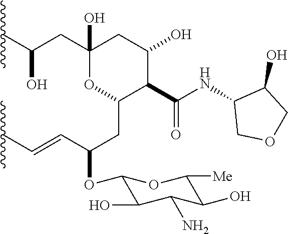 | 1.250 | BP |
| 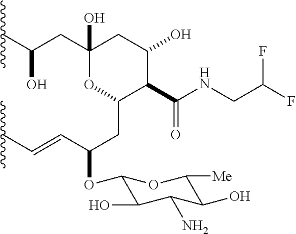 | 1.625 | BQ |
| 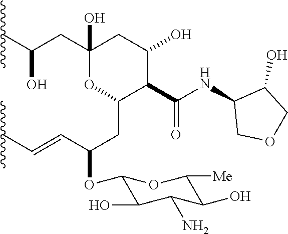 | 1.688 | BR |
| 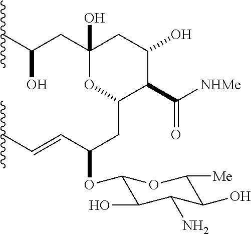 | 1.734 | BS |
| 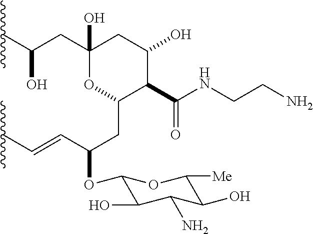 | 1.750 | BT |
| 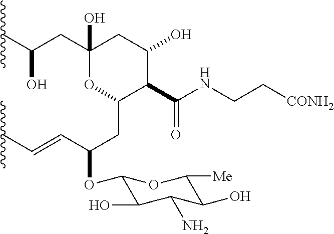 | 2.563 | BU |
| 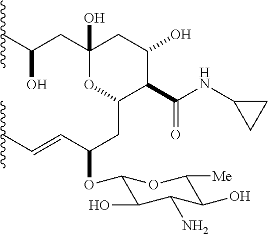 | 3.063 | BV |
| 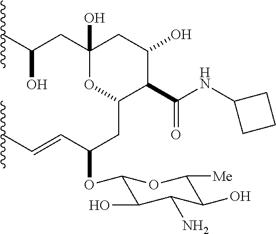 | 5.563 | BW |
| 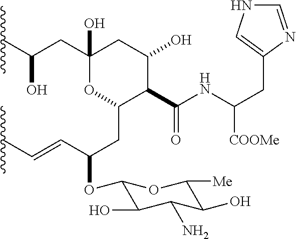 | 9.073 | BX |

TABLE 13
Minimum Inhibition Concentrations (MIC) for Representative C16 amides of
C2’epiAmB Against Various Fugal Species.

| MIC (mM) | C2’epiAmB | BR | BM | BQ | BO | BA |
|---|---|---|---|---|---|---|
| C. albicans | 0.5 | 0.5 | 0.5 | 1 | 0.5 | 0.5 |
| C. glabrata | 0.25 | 0.5 | 0.25 | 0.5 | 0.5 | 0.25 |
| C. krusei | 0.5 | 2 | 1 | 2 | 2 | 2 |
| C. tropicalis | 0.5 | 0.5 | 0.5 | 1 | 0.5 | 0.5 |
| A. fumigatus 91 | 64 | 2 | 2 | 4 | 2 | 2 |
| A. fumigatus 1100 | 4 | 2 | 1 | 2 | 2 | 1.5 |
| A. fumigatus 1163 | 2 | 2 | 1 | 2 | 1.5 | 1 |
| Average MIC | 9.03 | 1.25 | 0.84 | 1.625 | 1.19 | 1.03 |
| Avg. MIC of AmB amide | | 0.38 | 0.39 | 0.30 | 0.42 | 0.18 |

TABLE 13A
Minimum Inhibition Concentrations for AmB and the
Disclosed Compounds Against Different Yeasts.

| MIC (μM) | # of Isolates | AmB | BA | BM |
|---|---|---|---|---|
| Candida auris | 10 | 1.2 | 2.8 | 2.4 |
| Candida krusei | 2 | 1 | 4 | 2 |
| Candida parapsilosis | 8 | 1.1 | 2.5 | 2.4 |
| Cryptococcus neoformans | 10 | 0.55 | 2 | 2 |
| Cryptococcus gattii | 10 | 0.75 | 2 | 2 |
| Rhodotorula sp. | 6 | 1 | 3.3 | 3.3 |
| Average MIC | 46 | 0.9 | 2.5 | 2.3 |

TABLE 13B
Minimum Inhibition Concentrations for AmB and the Disclosed
Compounds Against Different Rare Moulds Resistant to AmB.

| MIC (μM) | # of Isolates | AmB | BA | BM |
|---|---|---|---|---|
| Sporothrix schenckii | 6 | 4 | 6 | 6.67 |
| Purpureocillium lilacinum | 6 | >16 | >16 | >16 |
| Scedosporium aurantiacum | 4 | >16 | >16 | >16 |
| Scedosporium boydii | 6 | >16 | >16 | >16 |
| Lomentospora prolificans | 6 | >16 | >16 | >16 |
| Average MIC | 28 | | | |

### Example 20. Mice Study for AmB, C2′epiAmB, and C2′epiAmB-L-his in AmBisome®-Like Formulation

Procedure: The experiment was performed using the AmB, C2′epiAmB, and C2′epiAmB-L-His in Ambisome like formulation. All the compounds were dissolved in D5W (5% dextrose in water) for IV injection. Male CD-1 mice were (3 per group; body weight 30 g each) injected with the drugs/compounds and monitored for 24 h for death or distress signs. After 24 h mice were sacrificed and the kidneys were harvested, homogenized and analyzed for biomarkers of renal injury by RTPCR.

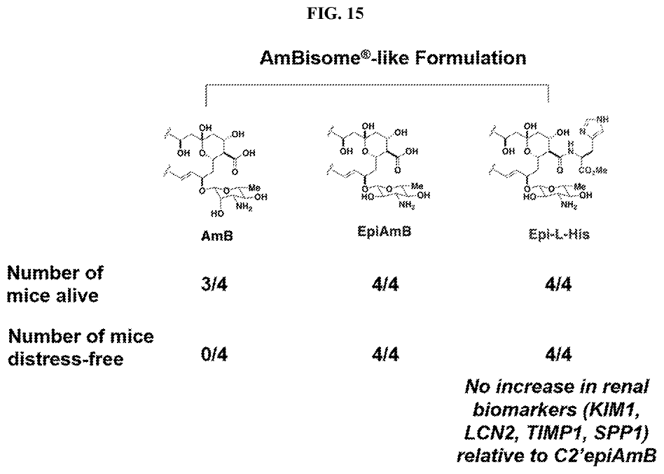

Results are summarized in FIG. 15.

### Example 21. Stability Study of Compound BA In Vitro

Liver Microsome:

Working Compound solution: 10 uM in 50 mM potassium phosphate buffer with 9% MeOH and 1% DMSO

Microsome solution: working concentration 0.625 mg/ml

Stopping solution: Cold ACN with 200 ng/ml Tolbutamide and 200 ng/ml Labetalol (4° C.)

Procedure: In the well plates (T5, T10, T20, T30, T60, NCF60 (no cofactor 60)) 10 uL of the compound/control working solution was added (except T0 and matric blank) followed by microsome solution (80 uL) and the mixture was incubated at 37 C for about 10 mins. A solution of potassium phosphate (10 ul of 50 mM) was added to NCF60 well, incubated at 37 C and time counting started. After prewarming 10 ul of NADPH regenerating solution was added to each plate to start the reaction. At different time point 300 uL/well of stopping solution to terminate the reaction. The sampling plates were shaken for 10 mins and the centrifuged for 20 mins at 4000 rpm. The supernatant was half diluted with ultra pure water and the analyzed using LCMS.

Plasma Stability:

Procedure: In order to run the experiment 25 uM working solution of the compound for TA analysis and 50 uM working solution for PC analysis was prepared (2.5% DMSO, 20% MeOH, water). Plasma was warmed to 37° C. prior to the experiment and centrifuged for 5 mins at 4000 rpm. The pH of the sample was adjusted to 7.4±0.1. 96 uL/well of blank plasma from each species was transferred and pre-incubated for 5 min. Each time point was monitored in triplicate (0, 10, 30, 60 and 120 min). 4 uL/well aliquot of the working solution was mixed to the blank plasma and incubated at 37° C. AT each time point samples were quenched with ACN stop solution containing 200 ng/ml Tolbutamide & 200 ng/ml Labetalol at 1:3 ratio and mix well on shaker for 10 min then centrifuge at 4000 rpm for 15 min. A 100 ul aliquot of supernatant was mixed with 100 μL of ultra-pure and submitted for LC-MS/MS analysis.

CYP Inhibition:

The working solutions of the test compounds and the standard inhibitors were prepared in DMSO (100× final concentration). 20 ul of substrate solution (5 in 1 cocktail) was added to the wells followed by 2 ul test compound solution was added. 158 ul of human liver microsome (HLM) was added to all the wells of the incubated plate and warmed to 37° C. for 10 mins. 20 ul of NADPH cofactor solution was then added to all the wells of incubated plate and incubated at 37° C. for 10 mins. At the time point the reaction was quenched by adding 400 uL of cold stop solution. The samples were centrifuged at 4000 rpm for 20 mins to precipitate protein. 100 ul supernatant was diluted with 100 ul of ultra pure water and analyzed using LCMS.

TABLE 14
Plasma Stability

| Species/Matrix | % Remaining at 120 min | Half Life (min) |
|---|---|---|
| Mouse Plasma | 100 | >289 |
| Human Plasma | 118 | >289 |

TABLE 15
Liver Microsomes in Vitro

| | | | CL | Remaining |
|---|---|---|---|---|
| Species | R² | T_{1/2} (min) | (μl/min/mg) | (T = 60) |
| Mouse | 0.1300 | >145 | <9.6 | 97.7% |
| Rat | 0.2828 | >145 | <9.6 | 115% |
| Dog | 0.5805 | >145 | <9.6 | 111% |
| Monkey | 0.4737 | >145 | <9.6 | 108% |
| Human | 0.1410 | >145 | <9.6 | 99.9% |

TABLE 16
P450 inhibition at 10 um

| 1A2 | 2C9 | 2C19 | 2D6 | 3A4 |
|---|---|---|---|---|
| % Inhibition | % Inhibition | % Inhibition | % Inhibition | % Inhibition |
| 0.00 | 18.7 | 26.0 | 2.74 | 15.7 |

TABLE 17
Positive Controls at 3 μM on P450 inhibition

| CYP Isozyme | Standard Inhibitor | % Inhibition |
|---|---|---|
| 1A2 | α-Naphthoflavone | 92.7 |
| 2C9 | Sulfaphenazole | 69.7 |
| 2C19 | N-3-Benzylnirvanol | 91.8 |
| 2D6 | Quinidine | 95.3 |
| 3A4 | Ketoconazole | 97.8 |

### Example 22. Compound BA is Well-Tolerated in Mice

Procedure: The experiment was performed using the commercial Fungizone and Ambisome. Compound BA was directly used after purification without any special treatment. All the compounds were dissolved in D5W (5% dextrose in water) for IV injection. Female CD-1 mice were (3 per group; body weight 30 g each) injected with the drugs/compounds and monitored for 24 h for death or distress signs. After 24 h mice were sacrificed and the kidneys were harvested, homogenized and analyzed for biomarkers of renal injury by RTPCR.

TABLE 18
Death or Signs of Distress After Administering
Fungizone, AmBisome, and Compound BA.

| | Fungizone | Fungizone | Ambisome | BA |
|---|---|---|---|---|
| | 2 mg/kg | 4 mg/kg | 40 mg/kg | 40 mg/kg |
| Number of Mice | 3/3 | 0/3 | 3/3 | 3/3 |
| Alive | | | | |
| Number of Mice | 0/3 | 0/3 | 0/3 | 3/3 |
| Distress-Fee | | | | |

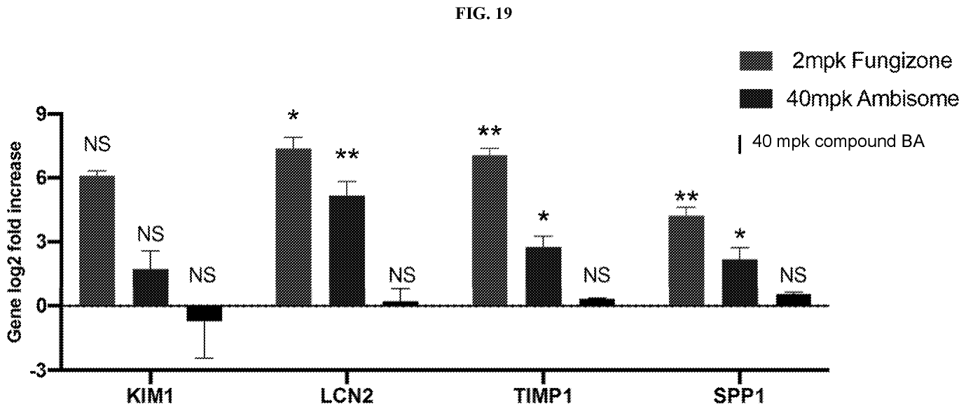

Biomarks of renal injury was analyzed and summarized in FIG. 19.

### Example 23. Compound BA is Highly Efficacious in a Mouse Model of Invasive Candidiasis, but Less Potent than Ambisome®

Procedure: The experiment was performed using commercial Ambisome, C2′epiAmB-deoxycholate (1:2) and BA. Female CD-1 mice (n=3 or 4/group) was infected with Candida albicans SN250 through IV injection. After 2 hours of inoculation, mice were treated with the compound and monitored. After 24 h post treatment mice were sacrificed and the kidneys were harvested and homogenized. The serum was plated in order to count CFU/mL of C. albicans in kidney homogenates (detection limit ˜10²)

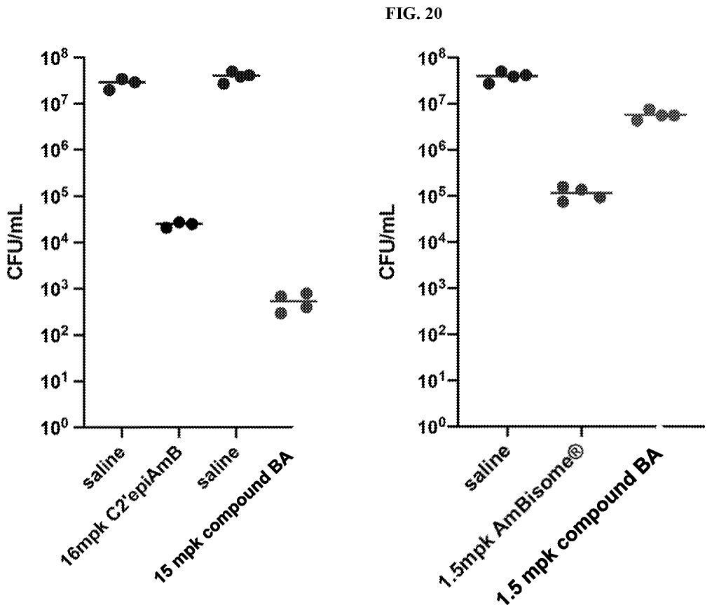

Results of mice study are summarized in FIG. 20.

### Example 24. In Vitro and In Vivo Safety of Compound BA

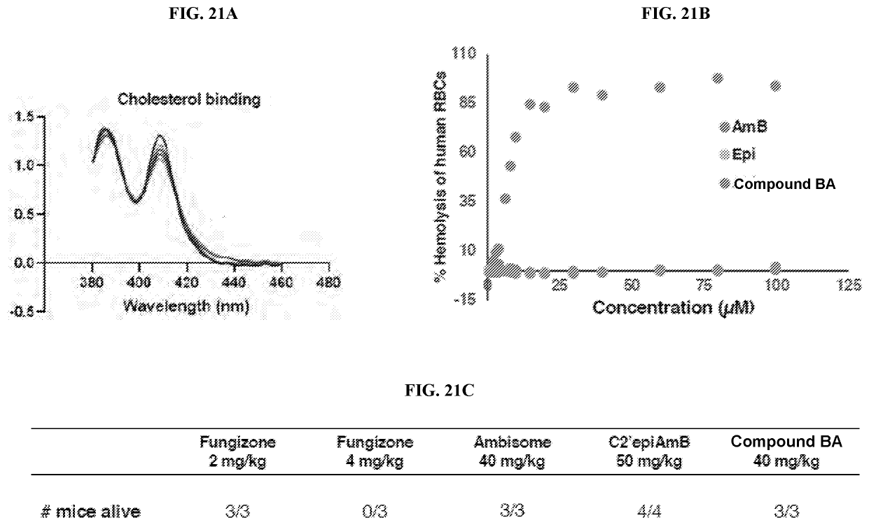

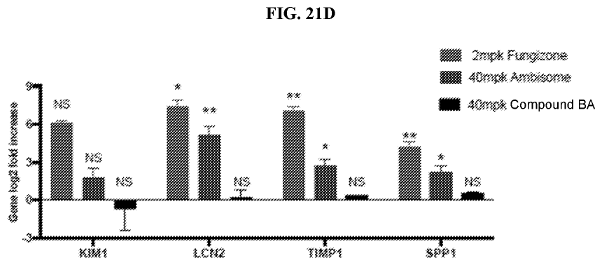

UV-Vis Binding Assay (FIG. 21A): The protocol for the sterol binding assay (UV-Vis) was developed in our lab. Compounds were dissolved in DMSO at a final concentration of 1 mM. Sterol were first dissolved in CHCl₃ (>200 mM) and then diluted to 1 mM concentration with DMSO. To synthesize the complex 1 ul of compound solution was taken in a clean eppendorf tube (2 ml) and sterol solution (volume depends on the stoichiometry) was added to it and the volume was made up to 20 ul with DMSO. 0.98 ml of PBS buffer was added to the Eppendorf tube and mixed properly. The absorbance of the solution was measured after 30 mins of incubation.

MHC (FIG. 21B): The protocol for the hemolysis assay was adapted from the report of Paquet and coworkers (Chem. Eur. J. 2008, 14, 2465-2481). Whole human blood (sodium heparin) was purchased from Bioreclamation LLC (Westbury, N.Y.) and stored at 4° C. and used within two days of receipt. To a 2.0 mL eppendorf tube, 1 mL of whole human blood was added and centrifuged at 10,000 g for 2 minutes. The supernatant was removed and the erythrocyte pellet was washed with 1 mL of sterile saline and centrifuged at 10,000 g for 2 minutes. The saline wash was repeated for a total of three washes. The erythrocyte pellet was suspended in 1 mL of RBC buffer (10 mM NaH2PO4, 150 mM NaCl, 1 mM MgCl2, pH 7.4) to form the erythrocyte stock suspension.

Compounds were prepared as >15 mM stock solutions in DMSO and serially diluted to the following concentrations with DMSO: 7689, 5126, 2563, 2050, 1538, 1025, 769, 513, 384, 256, 205, 154, 103, 77, 51, 26 μM. To a 0.2 mL PCR tube, 24 μL of RBC buffer and 1 μL of compound stock solution were added, which gave final concentrations of 500, 300, 200, 100, 80, 60, 40, 30, 20, 15, 10, 8, 6, 4, 3, 2, 1 μM. Positive and negative controls were prepared by adding 1 μL of DMSO to MilliQ water or RBC buffer, respectively to 0.2 mL PCR tube. To each PCR tube, 0.63 μL of the erythrocyte stock suspension was added and mixed by inversion. The samples were incubated at 37° C. for 2 hours. The samples were mixed by inversion and centrifuged at 10,000 g for 2 minutes. 15 μL of the supernatant from each sample was added to a 384-well place. Absorbances were read at 540 nm using a Biotek H1 Synergy Hybrid Reader (Wanooski, Vt.). Experiments were performed in triplicate and the reported MHC represents an average of three experiments.

In vivo Toxicity (FIG. 21C and FIG. 21D): The experiment was performed using the commercial Fungizone and Ambisome. Compound BA was directly used after purification without any special treatment. All the compounds were dissolved in D5W (5% dextrose in water) for IV injection. Female CD-1 mice were (3 per group; body weight 30 g each) injected with the drugs/compounds and monitored for 24 h for death or distress signs. After 24 h mice were sacrificed and the kidneys were harvested, homogenized and analyzed for biomarkers of renal injury by RTPCR.

### Example 25. In Vivo Mouse Pharmacokinetic Experiments with Compound BA

The experiment was performed using the compounds synthesized in lab and purified by preparative HPLC (>91%). All the compounds were dissolved in D5W (5% dextrose in water) at for IV injection. Female CD-1 mice were (3 per group; body weight approx. 30 g each) injected with the compounds (as per planned dosage) and the blood samples were collected at different time points and the compound content was analysed using the following procedure.

### Instrument: Triple Quad 6500+

Matrix Male CD-1 mouse plasma (EDTA-K2)

### Analyte(s): Compound BA

Internal standard(s): 100 ng/mL Labetalol & 100 ng/mL Tolbutamide in ACN
MS conditions ESI: positive

### SRM Detection

### Compound BA: [M+H]+m/z 949.8>732.6

### Labelatol (IS): [M+H]+m/z 329.2>162.1

### UPLC Conditions

### Mobile Phase A: 0.1% FA in Water

### Mobile Phase B: 0.1% FA in ACN

| Time (min) | Mobile Phase B (%) |
|---|---|
| 1.10 | 98 |
| 1.50 | 98 |
| 1.51 | 15 |
| 2.10 | Stop |

### Column: Waters ACQUITY UPLC HSS T3 1.8 μm 2.1×50 mm

Flow rate: 0.6000 mL/min
Retention time: Labelatol (IS): 0.998 min

### Sample Preparation:

An aliquot of 244, sample was protein precipitated with 1204, IS solution (100 ng/mL Labetalol & 100 ng/mL Tolbutamide in ACN), the mixture was vortex-mixed well and centrifuged at 3900 rpm for 10 min, 4□. An aliquot of 90 μL supernatant was transferred to sample plate and mixed with 60 μL water, then the plate was shaken at 800 rpm for 10 min. 15.0 μL supernatant was injected for LC-MS/MS analysis.

### Calibration Curve:

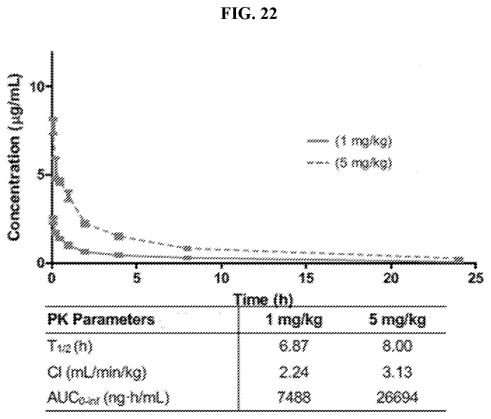

1.00-3000 ng/mL for Compound BA in female CD-1 mouse plasma (EDTA-K2) The results of this experiment are shown in FIG. 22.

### Example 26. Solubility of Disclosed Compounds

Test Article 1: Synthesized in lab (purity >95%)

### D5W: Braun USA (Product No: L5101)

### Instrument Details:

Sonicator: Branson Ultrasonics 2800; Vortex-Genie 2 lab mixer
UV-Vis: Thermo Fisher Nanodrop oneC
Steps:* *amounts are based on the solution prepared for 50 mg/kg dosage invivo toxicity experiment
Take 6 mg of sample (measured by UV-Vis) in a clean oven-dried 7 mL vial
Add 1.65 mL sterile D5W (at room temperature)
Vortex it for 2 min.
Water bath sonication: 2 min×2
Repeat step 3 and 4 until the solution is clear
Transferred in a 2 mL Eppendorf tube and centrifuged (3000 g×2 min) to ensure compound is dissolved completely and there is no insoluble part. (optional step)
Concentration measured by UV (abs at 406 nm) (optional step)

TABLE 19
Solubility of Disclosed Compounds and AmB in D5W.

| | AmB | Compound BA | Compound BM |
|---|---|---|---|
| Solubility (mM) | 0.023 | >2.6 | 0.15 |
| Fold of Increase w.r.t. | — | >113 | 6.5 |
| AmB | | | |

### Example 27. Summary Data for Compounds BA and BM Compared to AmB

| | | |
|---|---|---|
| AmB | BA | BM |

EFFICACY

| | | | |
|---|---|---|---|
| MIC_{avg} yeast (5 strains) | 0.23 | 0.59 | 0.58 |
| MIC_{avg} moulds (3 strains) | 0.92 | 1.3 | 1.7 |
| MIC_{avg} yeast (46 strains) | 0.9 | 2.5 | 2.3 |
| MIC_{avg} moulds (159 strains) | 0.9 | 2.3 | 2.2 |
| Mouse candidiasis model | 5.2 | 6.3 | 5.7 |
| 5 mpk; Log (CFU/mL) | | | |

SAFETY

| | | | |
|---|---|---|---|
| Binds Cholesterol | Yes | No | No |
| MHC | 8.4 | >500 | >100 |
| Mouse single IV injection: | 0/3 | 0/4 | ND |
| mortality (40 mpk) | | | |
| Renal toxicity biomarkers | elevated | not | ND |
| KIM1, LCN2, TIMP1, SPP1 | | elevated | |

DMPK

| | | | |
|---|---|---|---|
| Liver microsome (T_{1/2}, min) | >145 | >145 | >145 |
| Mouse, rat, dog, monkey, | All species | All species | All species |
| human | | | |
| Blood Plasma Stability | >289; >289 | >289; >289 | >289; >289 |
| (T_{1/2}, min) Mouse; Human | | | |
| P450 Inhibition (%) | 23.1; 4.1; | 0.0; 18.7; | 7.8; 89.1; |
| 1A2; 2C9; 2C19; 2D6; 3A4 | 7.3; 2.3; | 26.0; 2.77; | 19.7; 2.1; |
| | 8.1 | 15.7 | 12.4 |
| In vivo PK: [1 mg/kg]; | ND | [6.87; 2.24; | ND |
| 5 mg/kg T_{1/2}, (h), | | 7488] | |
| Cl (mL/min/kg), | | 8.00; 3.13; | |
| AUC_{0-inf} (ng*hr/mL) | | 26694 | |

SOLUBILITY

| | | | |
|---|---|---|---|
| In D5W (mM) | 0.023 | >2.6 | 0.15 |

### REFERENCES CITED

1. Eggimann, P.; Garbino, J.; Pittet, D., Management of Candida species infections in critically ill patients. The Lancet. Infectious diseases 2003, 3 (12), 772-85.
2. Pfaller, M. A.; Diekema, D. J.; Colombo, A. L.; Kibbler, C.; Ng, K. P.; Gibbs, D. L.; Newell, V. A., Candida rugosa, an emerging fungal pathogen with resistance to azoles: Geographic and temporal trends from the ARTEMIS DISK antifungal surveillance program. J Clin Microbiol 2006, 44 (10), 3578-3582.
3. Martin, C. A., Invasive Fungal Infections in the Critically Ill Patient. Journal of Pharmacy Practice 2005, 18 (1), 9-17.
4. Howard, S. J.; Webster, I.; Moore, C. B.; Gardiner, R. E.; Park, S.; Perlin, D. S.; Denning, D. W., Multi-azole resistance in Aspergillus fumigatus. Int J Antimicrob Ag 2006, 28 (5), 450-453.
5. Hakki, M.; Staab, J. F.; Marr, M. A., Emergence of a Candida krusei isolate with reduced susceptibility to caspofungin during therapy. Antimicrobial agents and chemotherapy 2006, 50 (7), 2522-2524.
6. Cannon, R. D.; Lamping, E.; Holmes, A. R.; Niimi, K.; Tanabe, K.; Niimi, M.; Monk, B. C., Candida albicans drug resistance—another way to cope with stress. Microbiol-Sgm 2007, 153, 3211-3217.
7. Barker, K.; Rogers, P., Recent insights into the mechanisms of antifungal resistance. Current Infectious Disease Reports 2006, 8 (6), 449-456.
8. Agarwal, R.; Singh, N., Amphotericin B is still the drug of choice for invasive aspergillosis. Am J Resp Crit Care 2006, 174 (1), 102-102.
9. May, R. C.; Stone, N. R. H.; Wiesner, D. L.; Bicanic, T.; Nielsen, K., Cryptococcus: from environmental saprophyte to global pathogen. Nat Rev Micro 2016, 14 (2), 106-117.
10. Chen, S. C.; Meyer, W.; Sorrell, T. C., Cryptococcus gattii infections. Clinical microbiology reviews 2014, 27 (4), 980-1024.
11. Morens, D. M.; Fauci, A. S., Emerging Infectious Diseases: Threats to Human Health and Global Stability. PLOS Pathogens 2013, 9 (7), e1003467.
12. Andes, D.; Stamsted, T.; Conklin, R., Pharmacodynamics of amphotericin B in a neutropenicmouse disseminated-candidiasis model. Antimicrobial agents and chemotherapy 2001, 45 (3), 922-6.
13. Edmond, M. B.; Wallace, S. E.; McClish, D. K.; Pfaller, M. A.; Jones, R. N.; Wenzel, R. P., Nosocomial bloodstream infections in United States hospitals: a three-year analysis. Clinical infectious diseases: an official publication of the Infectious Diseases Society of America 1999, 29 (2), 239-44.
14. Marr, K. A.; Carter, R. A.; Crippa, F.; Wald, A.; Corey, L., Epidemiology and outcome of mould infections in hematopoietic stem cell transplant recipients. Clinical infectious diseases: an official publication of the Infectious Diseases Society of America 2002, 34 (7), 909-17.
15. Jolliffe, I. T., Principal Component Analysis and Factor Analysis. In Principal component analysis, Springer: 1986; pp 115-128.
16. Smith, P. B.; Alexander, B. D.; Perfect, J. R.; Johnson, M. D. In Mortality rates in candidemia over the last decade, 49th Annual Interscience Conference on Antimicrobial Agents and Chemotherapy, San Francisco, American Society of Microbiology: San Francisco, 2009; p Abstract M1009.
17. McNeil, M. M.; Nash, S. L.; Hajj eh, R. A.; Phelan, M. A.; Conn, L. A.; Plikaytis, B. D.; Warnock, D. W., Trends in mortality due to invasive mycotic diseases in the United States, 1980-1997. Clinical infectious diseases: an official publication of the Infectious Diseases Society of America 2001, 33 (5), 641-7.
18. Herbrecht, R.; Denning, D. W.; Patterson, T. F.; Bennett, J. E.; Greene, R. E.; Oestmann, J. W.; Kern, W. V.; Marr, K. A.; Ribaud, P.; Lortholary, O.; Sylvester, R.; Rubin, R. H.; Wingard, J. R.; Stark, P.; Durand, C.; Caillot, D.; Thiel, E.; Chandrasekar, P. H.; Hodges, M. R.; Schlamm, H. T.; Troke, P. F.; de Pauw, B., Voriconazole versus amphotericin B for primary therapy of invasive aspergillosis. The New England journal of medicine 2002, 347 (6), 408-15.
19. Maertens, J.; Raad, I.; Petrikkos, G.; Boogaerts, M.; Selleslag, D.; Petersen, F. B.; Sable, C. A.; Kartsonis, N. A.; Ngai, A.; Taylor, A.; Patterson, T. F.; Denning, D. W.; Walsh, T. J., Efficacy and safety of caspofungin for treatment of invasive aspergillosis in patients refractory to or intolerant of conventional antifungal therapy. Clinical infectious diseases: an official publication of the Infectious Diseases Society of America 2004, 39 (11), 1563-71.
20. Kontoyiannis, D. P.; Marr, K. A.; Park, B. J.; Alexander, B. D.; Anaissie, E. J.; Walsh, T. J.; Ito, J.; Andes, D. R.; Baddley, J. W.; Brown, J. M.; Brumble, L. M.; Freifeld, A. G.; Hadley, S.; Herwaldt, L. A.; Kauffman, C. A.; Knapp, K.; Lyon, G. M.; Morrison, V. A.; Papanicolaou, G.; Patterson, T. F.; Perl, T. M.; Schuster, M. G.; Walker, R.; Wannemuehler, K. A.; Wingard, J. R.; Chiller, T. M.; Pappas, P. G., Prospective surveillance for invasive fungal infections in hematopoietic stem cell transplant recipients, 2001-2006: overview of the Transplant-Associated Infection Surveillance Network (TRANSNET) Database. Clinical infectious diseases: an official publication of the Infectious Diseases Society of America 2010, 50 (8), 1091-100.
21. Neofytos, D.; Horn, D.; Anaissie, E.; Steinbach, W.; Olyaei, A.; Fishman, J.; Pfaller, M.; Chang, C.; Webster, K.; Marr, K., Epidemiology and outcome of invasive fungal infection in adult hematopoietic stem cell transplant recipients: analysis of Multicenter Prospective Antifungal Therapy (PATH) Alliance registry. Clinical infectious diseases: an official publication of the Infectious Diseases Society of America 2009, 48 (3), 265-73.
22. Pagano, L.; Caira, M.; Candoni, A.; Offidani, M.; Fianchi, L.; Martino, B.; Pastore, D.; Picardi, M.; Bonini, A.; Chierichini, A.; Fanci, R.; Caramatti, C.; Invernizzi, R.; Mattei, D.; Mitra, M. E.; Melillo, L.; Aversa, F.; Van Lint, M. T.; Falcucci, P.; Valentini, C. G.; Girmenia, C.; Nosari, A., The epidemiology of fungal infections in patients with hematologic malignancies: the SEIFEM-2004 study. Haematologica 2006, 91 (8), 1068-75.
23. Pappas, P. G.; Alexander, B. D.; Andes, D. R.; Hadley, S.; Kauffman, C. A.; Freifeld, A.; Anaissie, E. J.; Brumble, L. M.; Herwaldt, L.; Ito, J.; Kontoyiannis, D. P.; Lyon, G. M.; Marr, K. A.; Morrison, V. A.; Park, B. J.; Patterson, T. F.; Perl, T. M.; Oster, R. A.; Schuster, M. G.; Walker, R.; Walsh, T. J.; Wannemuehler, K. A.; Chiller, T. M., Invasive fungal infections among organ transplant recipients: results of the Transplant-Associated Infection Surveillance Network (TRANSNET). Clinical infectious diseases: an official publication of the Infectious Diseases Society of America 2010, 50 (8), 1101-11.
24. Patterson, T. F.; Thompson, G. R., 3rd; Denning, D. W.; Fishman, J. A.; Hadley, S.; Herbrecht, R.; Kontoyiannis, D. P.; Marr, K. A.; Morrison, V. A.; Nguyen, M. H.; Segal, B. H.; Steinbach, W. J.; Stevens, D. A.; Walsh, T. J.; Wingard, J. R.; Young, J. A.; Bennett, J. E., PracticeGuidelines for the Diagnosis and Management of Aspergillosis: 2016 Update by the Infectious Diseases Society of America. Clinical infectious diseases: an official publication of the Infectious Diseases Society of America 2016, 63 (4), e1-e60.
25. Tortorano, A. M.; Richardson, M.; Roilides, E.; van Diepeningen, A.; Caira, M.; Munoz, P.; Johnson, E.; Meletiadis, J.; Pana, Z. D.; Lackner, M.; Verweij, P.; Freiberger, T.; Comely, O. A.; Arikan-Akdagli, S.; Dannaoui, E.; Groll, A. H.; Lagrou, K.; Chakrabarti, A.; Lanternier, F.; Pagano, L.; Skiada, A.; Akova, M.; Arendrup, M. C.; Boekhout, T.; Chowdhary, A.; Cuenca-Estrella, M.; Guinea, J.; Guarro, J.; de Hoog, S.; Hope, W.; Kathuria, S.; Lortholary, O.; Meis, J. F.; Ullmann, A. J.; Petrikkos, G.; Lass-Florl, C., ESCMID and ECMM joint guidelines on diagnosis and management of hyalohyphomycosis: Fusarium spp., Scedosporium spp. and others. Clinical microbiology and infection: the official publication of the European Society of Clinical Microbiology and Infectious Diseases 2014, 20 Suppl 3, 27-46.
26. Fuhren, J.; Voskuil, W. S.; Boel, C. H.; Haas, P. J.; Hagen, F.; Meis, J. F.; Kusters, J. G., High prevalence of azole resistance in Aspergillus fumigatus isolates from high-risk patients. The Journal of antimicrobial chemotherapy 2015, 70 (10), 2894-8.
27. Talbot, G. H.; Bradley, J.; Edwards, J. E., Jr.; Gilbert, D.; Scheld, M.; Bartlett, J. G., Bad bugs need drugs: an update on the development pipeline from the Antimicrobial Availability Task Force of the Infectious Diseases Society of America. Clinical infectious diseases: an official publication of the Infectious Diseases Society of America 2006, 42 (5), 657-68.
28. Wiederhold, N. P.; Tam, V. H.; Chi, J.; Prince, R. A.; Kontoyiannis, D. P.; Lewis, R. E., Pharmacodynamic activity of amphotericin B deoxycholate is associated with peak plasma concentrations in a neutropenic murine model of invasive pulmonary aspergillosis. Antimicrobial agents and chemotherapy 2006, 50 (2), 469-73.
29. Anderson, T. M.; Clay, M. C.; Cioffi, A. G.; Diaz, K. A.; Hisao, G. S.; Tuttle, M. D.; Nieuwkoop, A. J.; Comellas, G.; Maryum, N.; Wang, S.; Uno, B. E.; Wildeman, E. L.; Gonen, T.; Rienstra, C. M.; Burke, M. D., Amphotericin forms an extramembranous and fungicidal sterol sponge. Nat Chem Biol 2014, 10 (5), 400-6.
30. Verweij, P. E.; Ananda-Rajah, M.; Andes, D.; Arendrup, M. C.; Bruggemann, R. J.; Chowdhary, A.; Comely, 0. A.; Denning, D. W.; Groll, A. H.; Izumikawa, K.; Kullberg, B. J.; Lagrou, K.; Maertens, J.; Meis, J. F.; Newton, P.; Page, I.; Seyedmousavi, S.; Sheppard, D. C., Viscoli, C.; Warris, A.; Donnelly, J. P., International expert opinion on the management of infection caused by azole-resistant Aspergillus fumigatus. Drug resistance updates: reviews and commentaries in antimicrobial and anticancer chemotherapy 2015, 21-22, 30-40.
31. Taubes, G., The bacteria fight back. Science 2008, 321 (5887), 356-361.
32. Monk, B. C.; Goffeau, A., Outwitting multidrug resistance to antifungals. Science 2008, 321 (5887), 367-369.
33. Walsh, T. J.; Anaissie, E. J.; Denning, D. W.; Herbrecht, R.; Kontoyiannis, D. P.; Marr, K. A.; Morrison, V. A.; Segal, B. H.; Steinbach, W. J.; Stevens, D. A.; van Burik, J. A.; Wingard, J. R.; Patterson, T. F., Treatment of aspergillosis: clinical practice guidelines of the Infectious Diseases Society of America. Clinical infectious diseases: an official publication of the Infectious Diseases Society of America 2008, 46 (3), 327-60.
34. Pappas, P. G.; Kauffman, C. A.; Andes, D.; Benjamin, D. K., Jr.; Calandra, T. F.; Edwards, J. E., Jr.; Filler, S. G.; Fisher, J. F.; Kullberg, B. J.; Ostrosky-Zeichner, L.; Reboli, A. C.; Rex, J. H.; Walsh, T. J.; Sobel, J. D., Clinical practice guidelines for the management of candidiasis: 2009 update by the Infectious Diseases Society of America. Clinical infectious diseases: an official publication of the Infectious Diseases Society of America 2009, 48 (5), 503-35.
35. Pappas, P. G.; Kauffman, C. A.; Andes, D. R.; Clancy, C. J.; Marr, K. A.; Ostrosky-Zeichner, L.; Reboli, A. C.; Schuster, M. G.; Vazquez, J. A.; Walsh, T. J.; Zaoutis, T. E.; Sobel, J. D., Clinical Practice Guideline for the Management of Candidiasis: 2016 Update by the Infectious Diseases Society of America. Clinical infectious diseases: an official publication of the Infectious Diseases Society of America 2016, 62 (4), e1-50.
36. Andes, D. R.; Safdar, N.; Baddley, J. W.; Alexander, B.; Brumble, L.; Freifeld, A.; Hadley, S.; Herwaldt, L.; Kauffman, C.; Lyon, G. M.; Morrison, V.; Patterson, T.; Perl, T.; Walker, R.; Hess, T.; Chiller, T.; Pappas, P. G., The epidemiology and outcomes of invasive Candida infections among organ transplant recipients in the United States: results of the Transplant-Associated InfectionSurveillance Network (TRANSNET). Transplant infectious disease: an official journal of the Transplantation Society 2016, 18 (6), 921-931.
37. Davis, S. A.; Vincent, B. M.; Endo, M. M.; Whitesell, L.; Marchillo, K.; Andes, D. R.; Lindquist, S.; Burke, M. D., Nontoxic antimicrobials that evade drug resistance. Nat Chem Biol 2015, 11 (7), 481-7.
38. Gray, K. C.; Palacios, D. S.; Dailey, I.; Endo, M. M.; Uno, B. E.; Wilcock, B. C.; Burke, M. D., Amphotericin primarily kills yeast by simply binding ergosterol. Proc Natl Acad Sci USA 2012, 109 (7), 2234-9.
39. Wilcock, B. C.; Endo, M. M.; Uno, B. E.; Burke, M. D., CT-OH of amphotericin B plays an important role in binding the primary sterol of human cells but not yeast cells. J Am Chem Soc 2013, 135 (23), 8488-91.

We claim:

1. A compound of Formula (I): 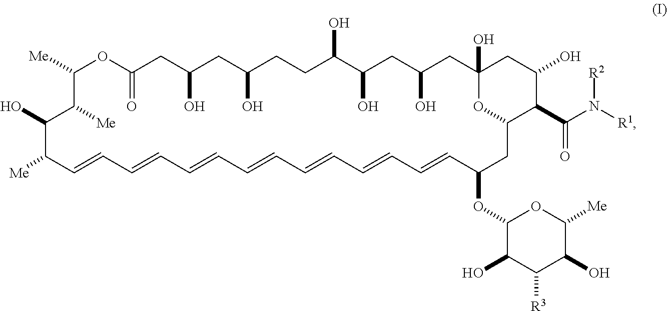
or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

2. The compound of claim 1, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

3. The compound of claim 2, wherein
R¹ and R² independently are hydrogen, unsubstituted C₁₋₆ alkyl, alkoxy C₁₋₆ alkyl, halo C₁₋₆ alkyl, amino C₁₋₆ alkyl, heterocyclyl C₁₋₆ alkyl, unsubstituted C₂₋₆ alkynyl, unsubstituted C₃₋₁₀ carbocyclyl, amino C₃₋₁₀ carbocyclyl, unsubstituted 3- to 10-membered heterocyclyl, or hydroxyl 3- to 10-membered heterocyclyl.

4. The compound of any one of claims 1-3, wherein at least one of R¹ and R² is hydrogen.

5. The compound of any one of claims 1-4, wherein at least one of R¹ and R² is hydrogen; and R¹ and R² are not both hydrogen.

6. The compound of claim 1, wherein
R¹ and R², together with the nitrogen to which they are attached, form a substituted or unsubstituted 3- to 10-membered heterocyclyl.

7. The compound of claim 6, wherein
R¹ and R², together with the nitrogen to which they are attached, form an unsubstituted 3- to 10-membered heterocyclyl, amino 3- to 10-membered heterocyclyl, hydroxyl 3- to 10-membered heterocyclyl, or heterocyclyl 3- to 10-membered heterocyclyl.

8. The compound of any one of claims 1-7, wherein R³ is —NR⁵R⁶, wherein
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein
R^{f} is selected from the group consisting of 2-alken-1-yl, tert-butyl, benzyl and fluorenylmethyl; or
R⁵ and R⁶, together with the nitrogen to which they are attached, form a substituted or unsubstituted 3- to 10-membered heterocyclyl.

9. The compound of claim 8, wherein
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein

10. The compound of claim 9, wherein R⁵ and R⁶ independently are hydrogen or C(O)OR^{f}.

11. The compound of claim 10, wherein R^{f} is fluorenylmethyl.

12. The compound of any one of claims 8-11, wherein at least one of R⁵ and R⁶ is hydrogen.

13. The compound of any one of claims 8-12, wherein R⁵ and R⁶ are both hydrogen.

14. The compound of any one of claims 1-13, wherein R⁴ is hydrogen, substituted or unsubstituted C₁₋₆ alkyl, or substituted or unsubstituted C₂₋₆ alkenyl.

15. The compound of claim 14, wherein R⁴ is hydrogen, halo C₁₋₆ alkyl, or unsubstituted C₂₋₆ alkenyl.

16. The compound of claim 15, wherein R⁴ is hydrogen.

17. A compound of Formula (II): 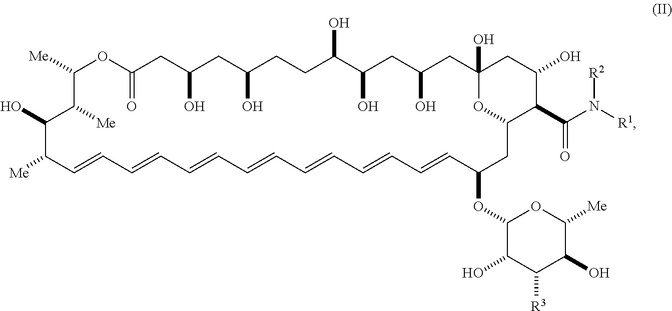
or a pharmaceutically acceptable salt thereof, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or
R¹ and R², together with the nitrogen to which they are attached, form a substituted or unsubstituted 3- to 10-membered heterocyclyl;
R³ is substituted or unsubstituted amino, substituted or unsubstituted urea, substituted or unsubstituted carbamate or substituted or unsubstituted guanidinyl; and
R⁴ is hydrogen or substituted or unsubstituted C₁₋₆ alkyl.

18. The compound of claim 17, wherein
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl.

19. The compound of claim 18, wherein
R¹ and R² independently are hydrogen, unsubstituted C₁₋₆ alkyl, hydroxyl C₁₋₆ alkyl, amino C₁₋₆ alkyl, unsubstituted C₃₋₁₀ carbocyclyl.

20. The compound of any one of claims 17-19, wherein at least one of R¹ and R² is hydrogen.

21. The compound of any one of claims 17-20, wherein at least one of R¹ and R² is hydrogen; and R¹ and R² are not both hydrogen.

22. The compound of any one of claims 17-21, wherein R³ is —NR⁵R⁶, wherein
R⁵ and R⁶ independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; or
R⁵ and R⁶, together with the nitrogen to which they are attached, form a substituted or unsubstituted 3- to 10-membered heterocyclyl;

23. The compound of claim 22, wherein
R⁵ and R⁶ independently are hydrogen, C(O)OR^{f}, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, or substituted or unsubstituted 5- to 10-membered heteroaryl; wherein
R^{f} is selected from the group consisting of 2-alken-1-yl, tert-butyl, benzyl and fluorenylmethyl.

24. The compound of claim 23, wherein R⁵ and R⁶ independently are hydrogen or C(O)OR^{f}.

25. The compound of claim 24, wherein R^{f} is fluorenylmethyl.

26. The compound of any one of claims 22-25, wherein at least one of R⁵ and R⁶ is hydrogen.

27. The compound of any one of claims 22-26, wherein R⁵ and R⁶ are both hydrogen.

28. The compound of any one of claims 17-27, wherein R⁴ is hydrogen, substituted or unsubstituted C₁₋₆ alkyl, or substituted or unsubstituted C₂₋₆ alkenyl.

29. The compound of claim 28, wherein R⁴ is hydrogen, halo C₁₋₆ alkyl, or unsubstituted C₂₋₆ alkenyl.

30. The compound of claim 29, wherein R⁴ is hydrogen.

31. A compound selected from the group consisting of:

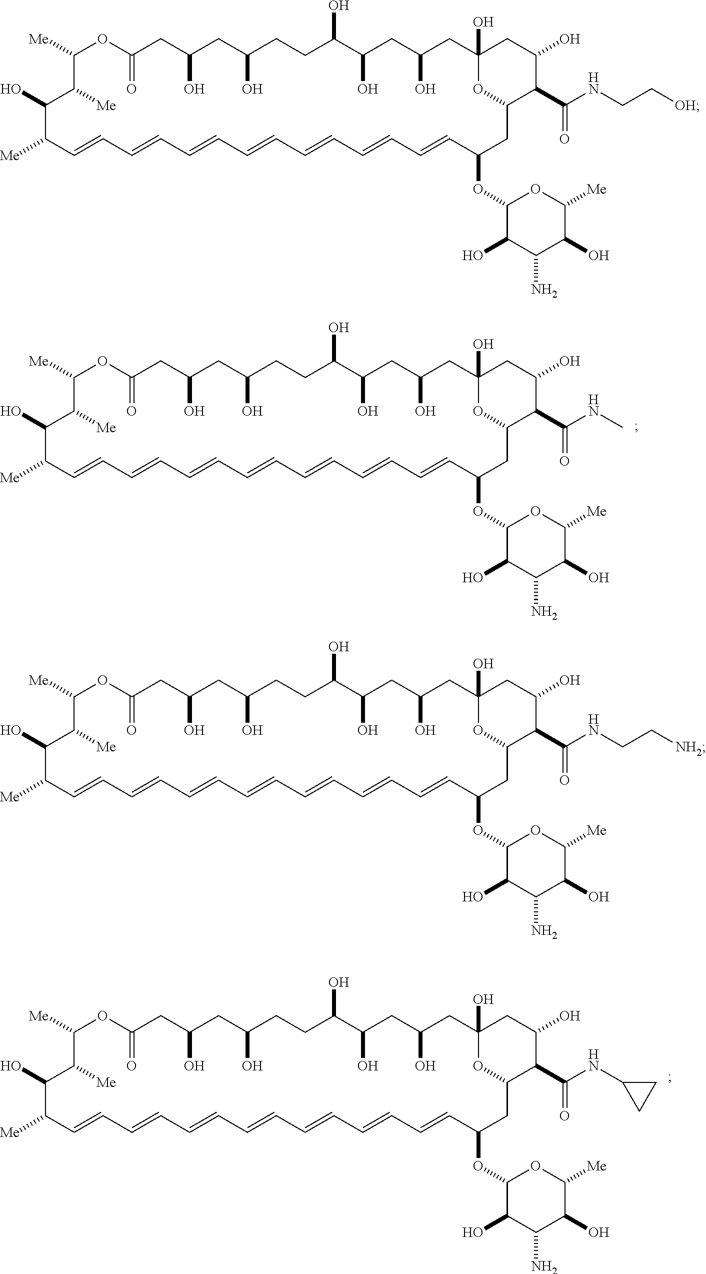

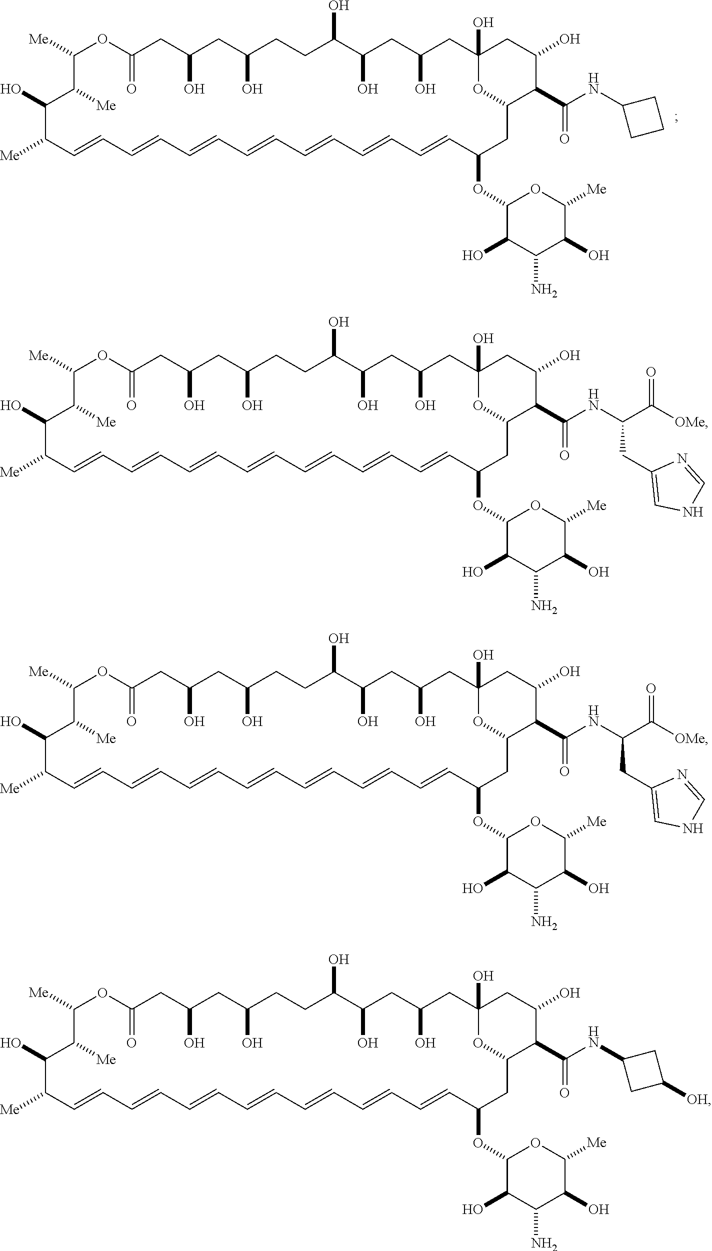

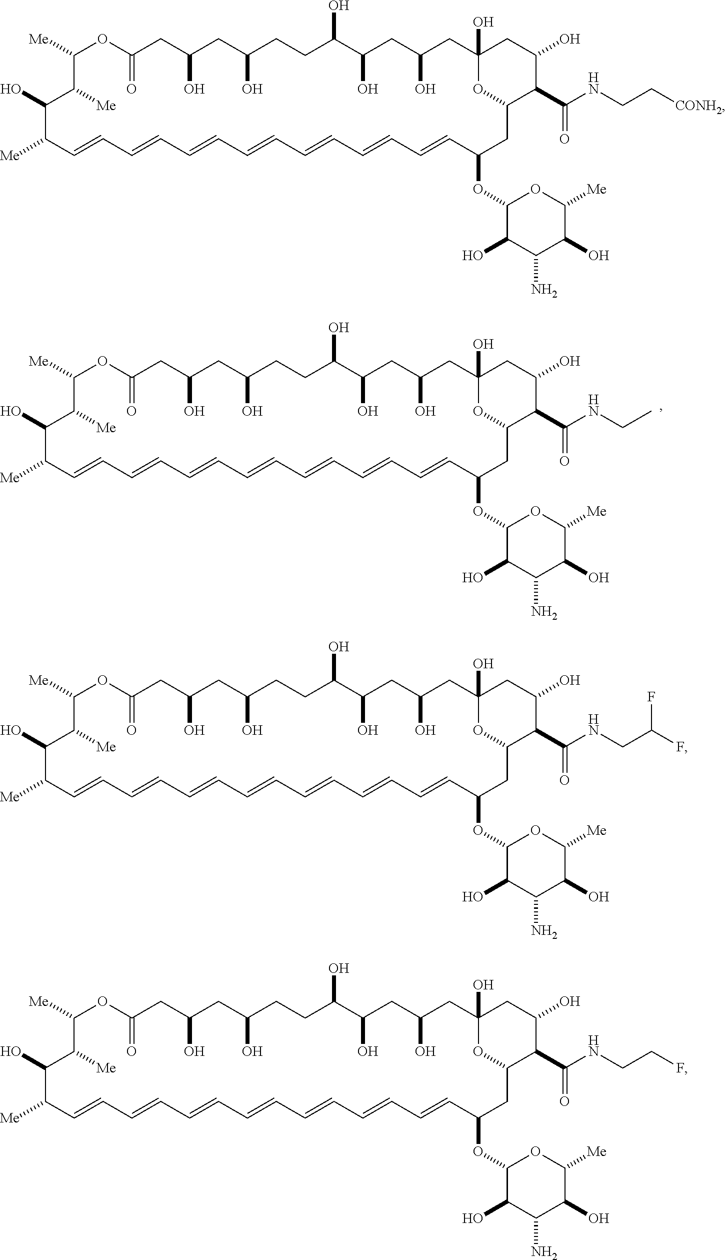

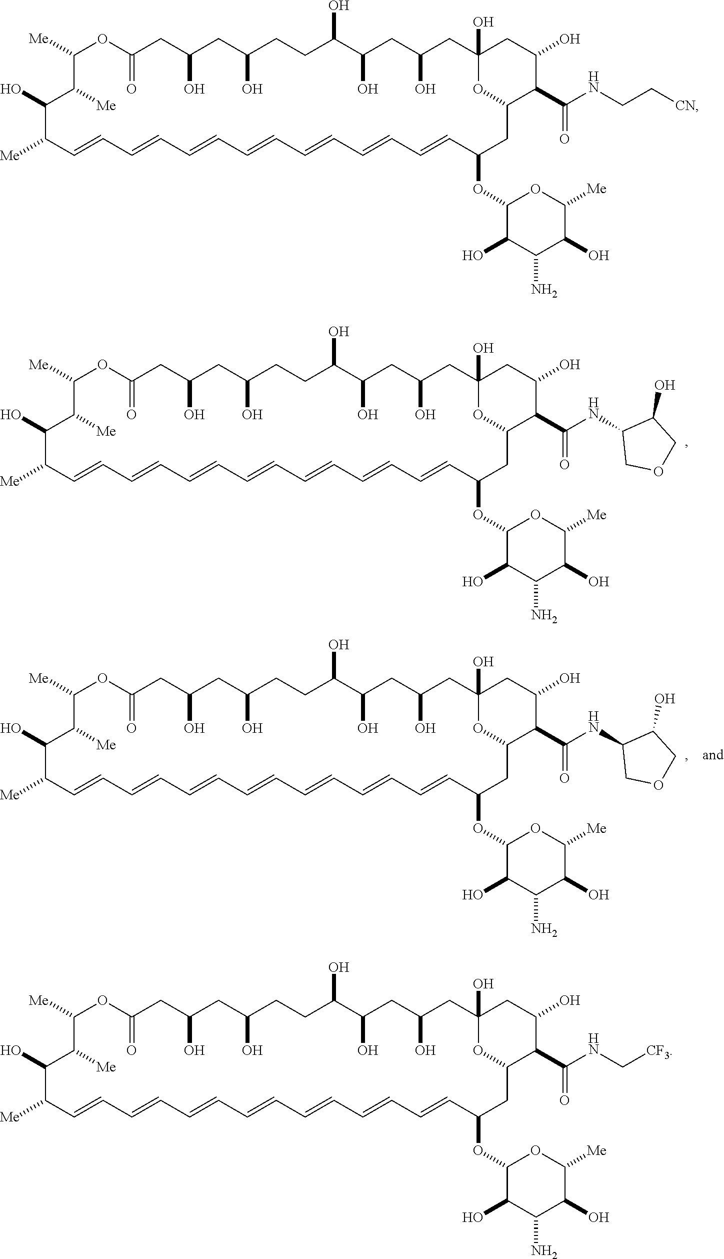

32. A compound selected from the group consisting of:

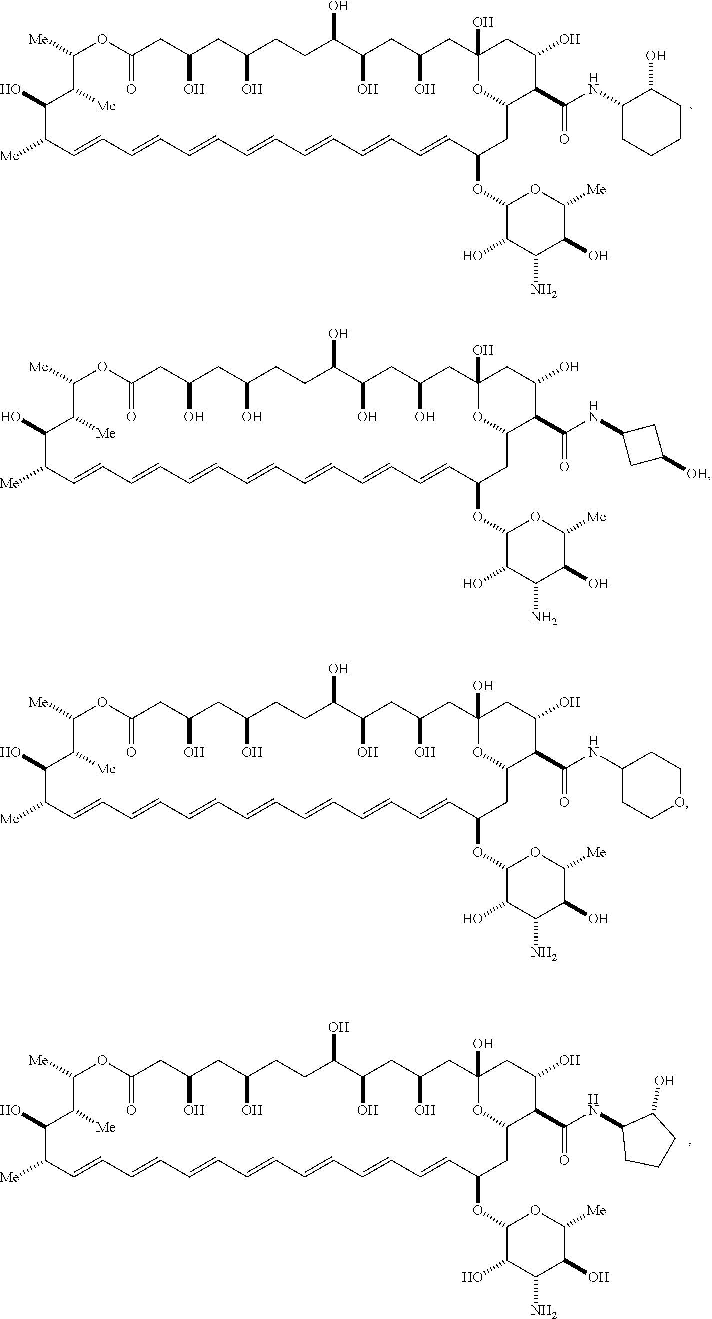

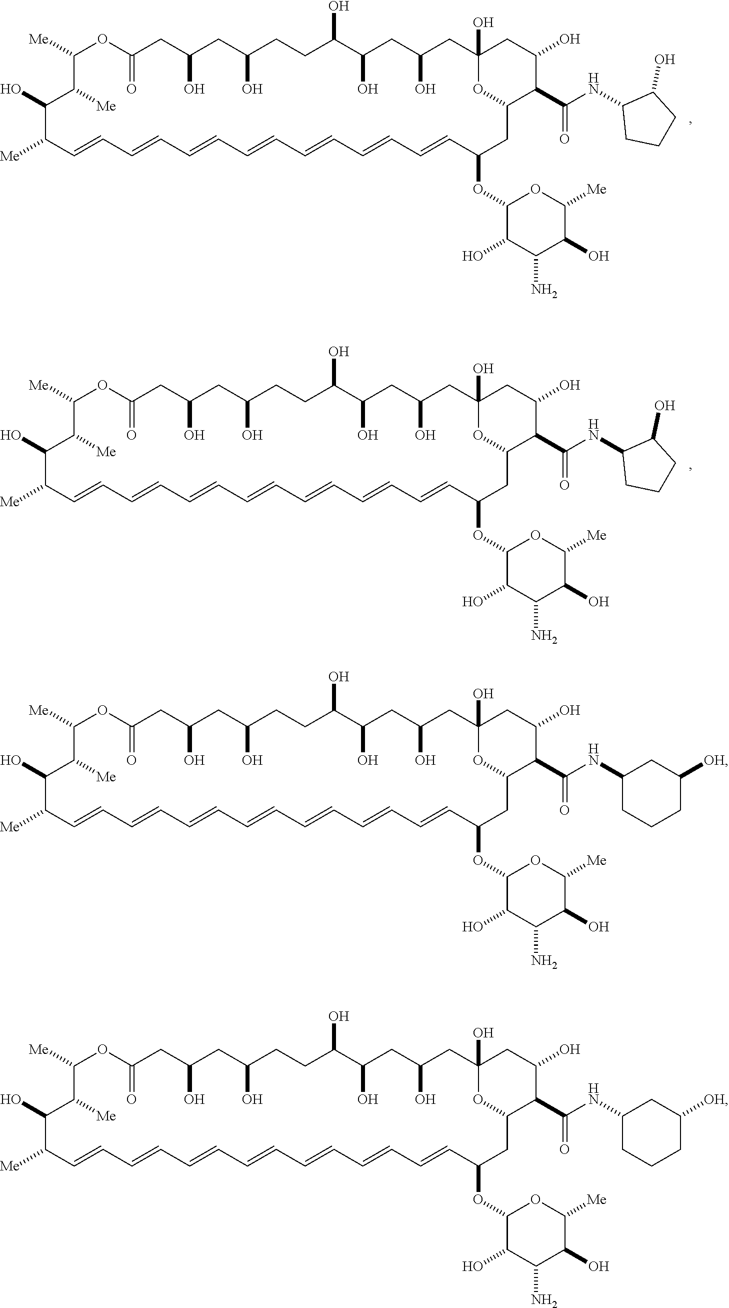

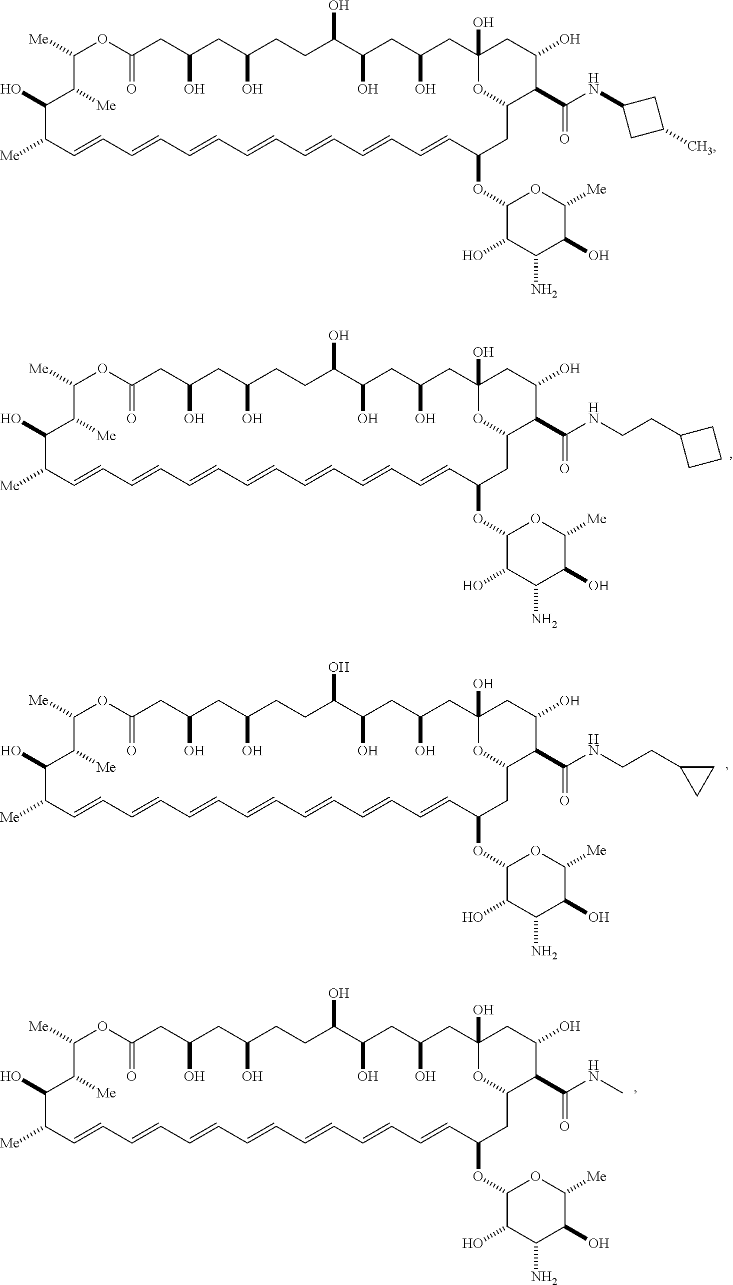

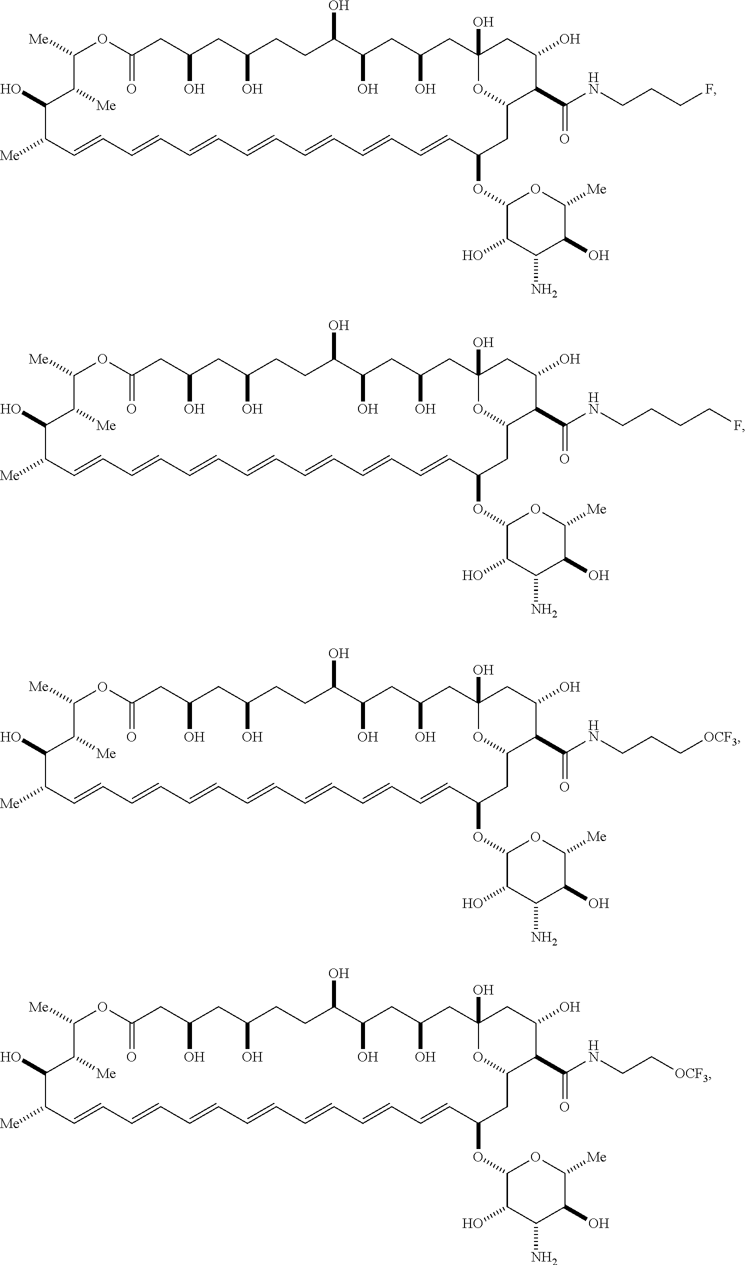

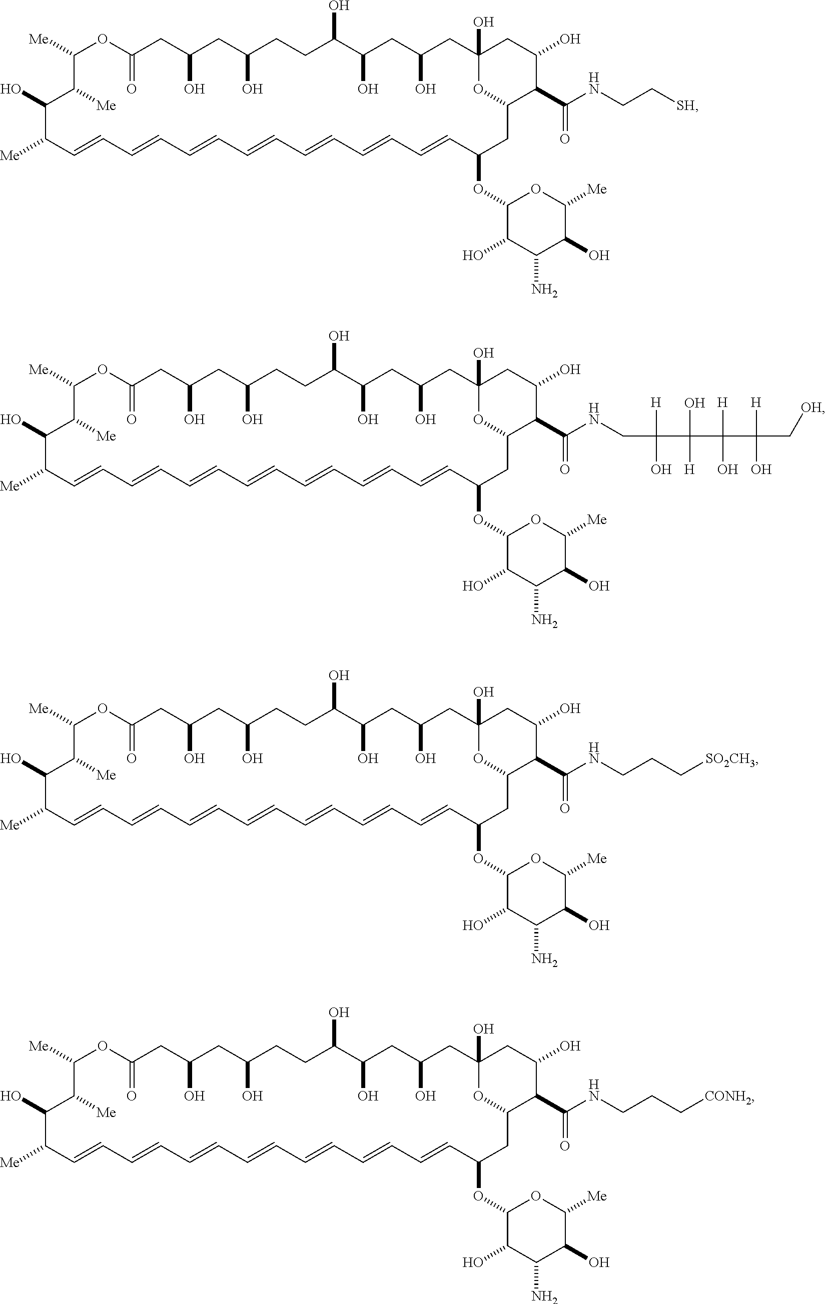

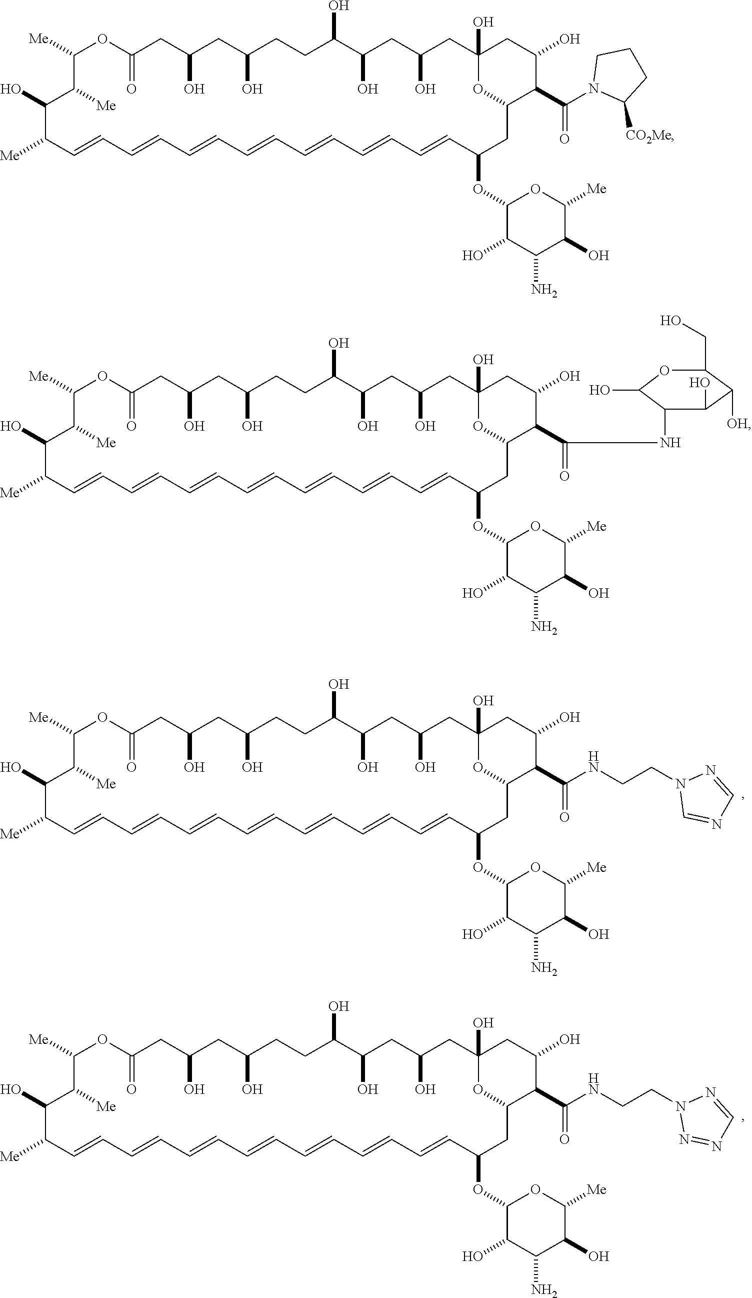

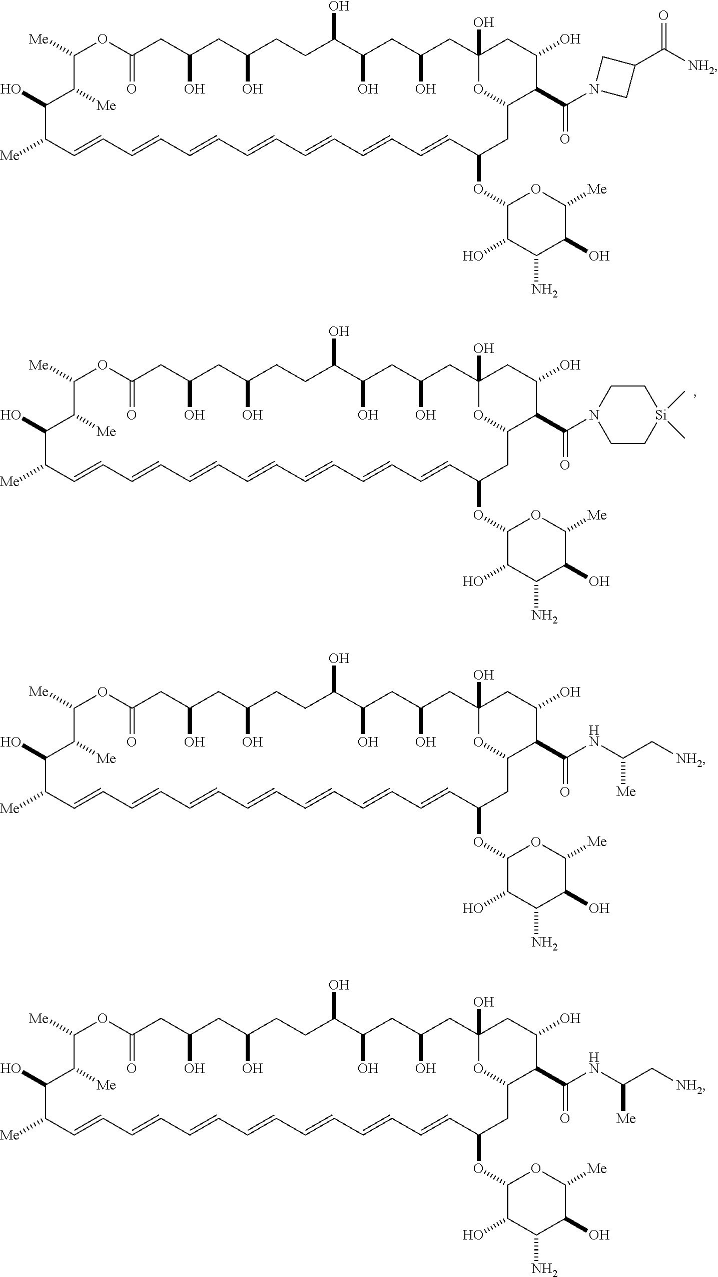

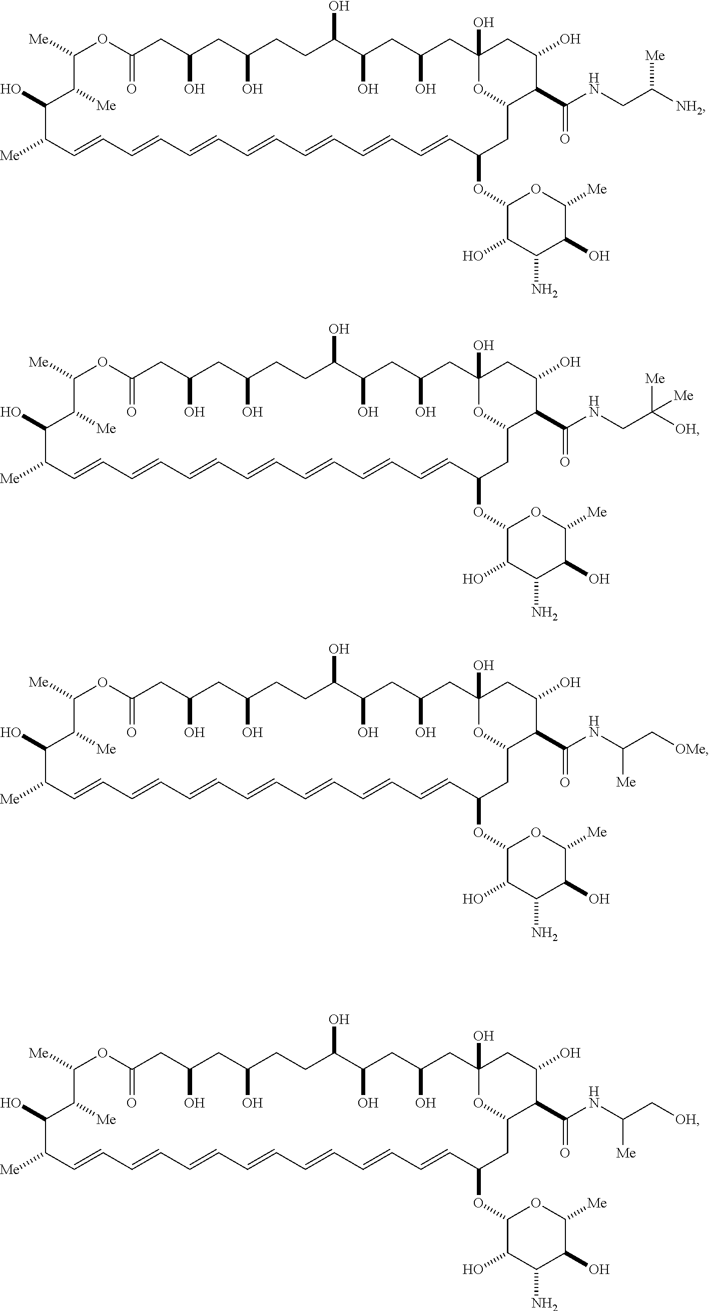

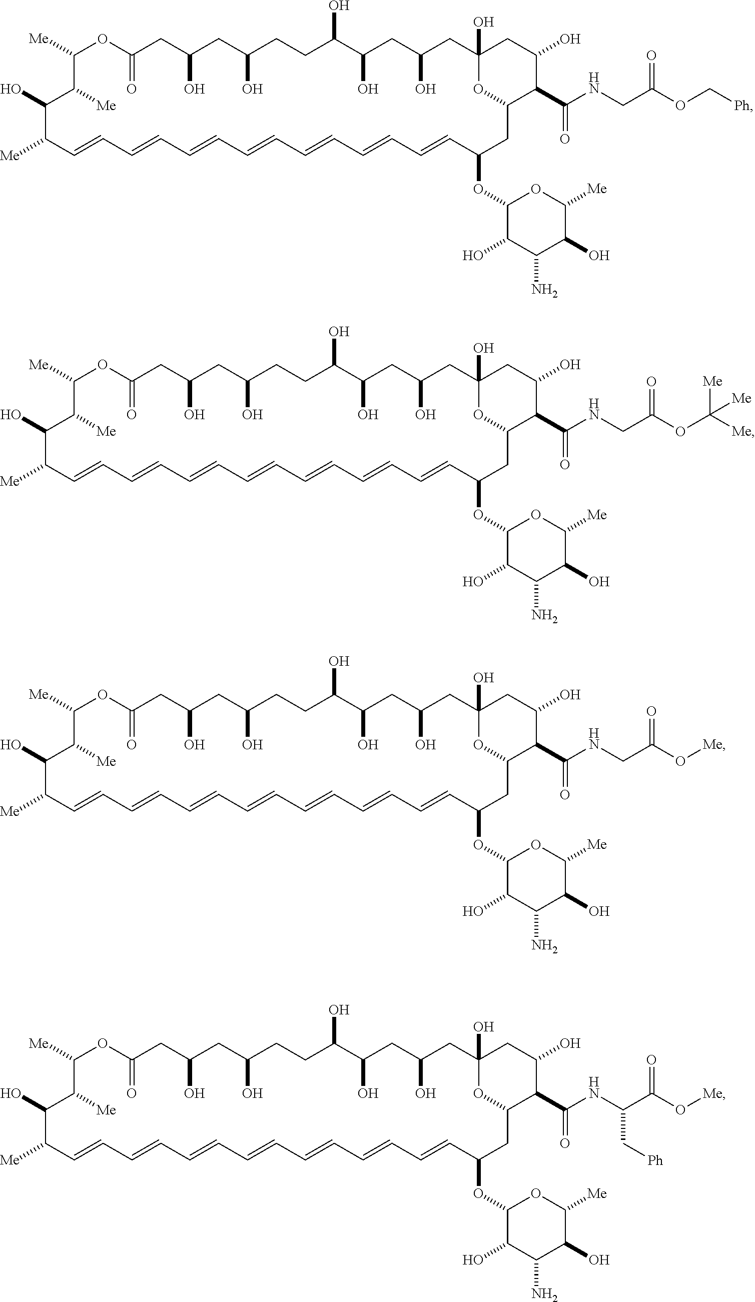

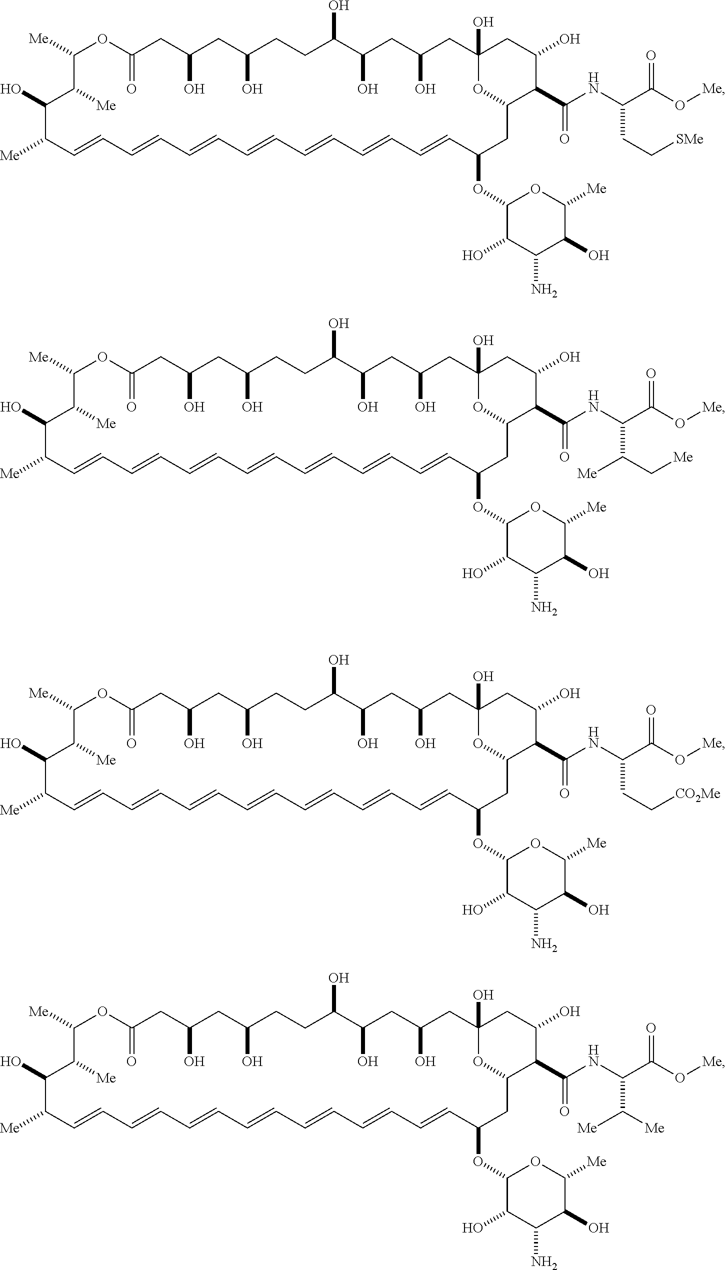

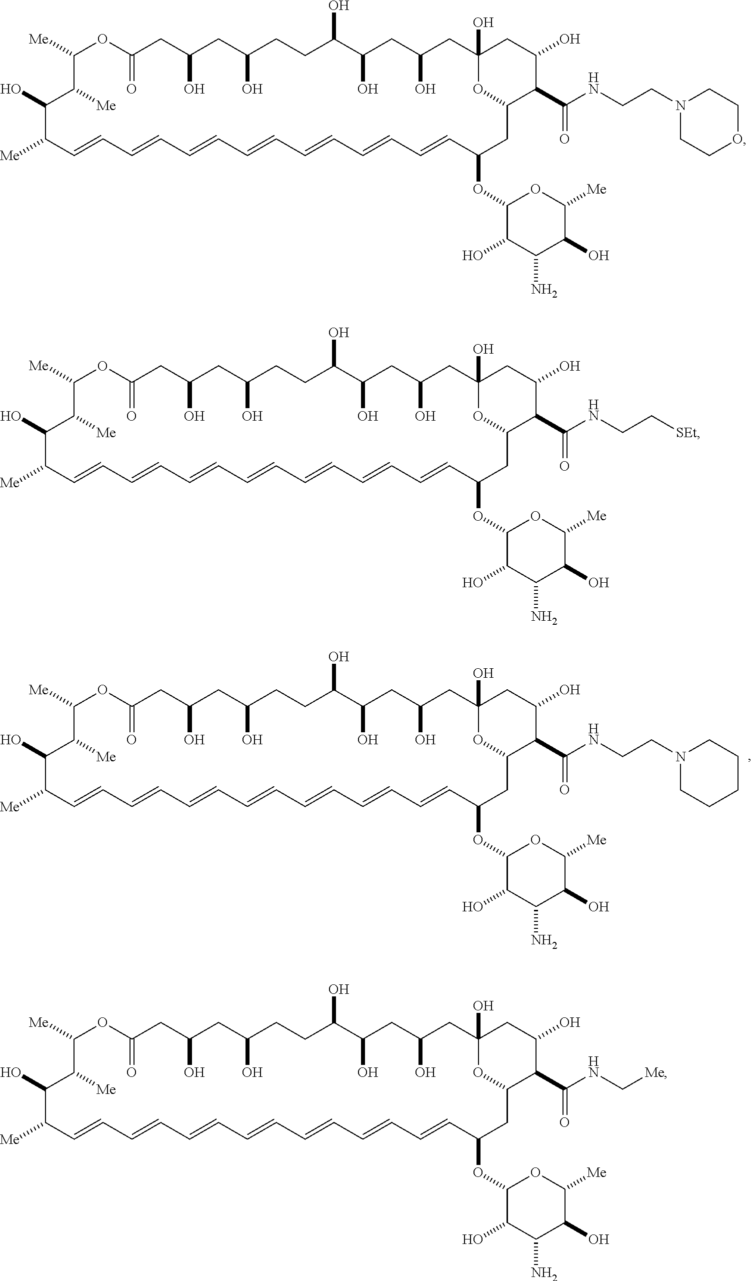

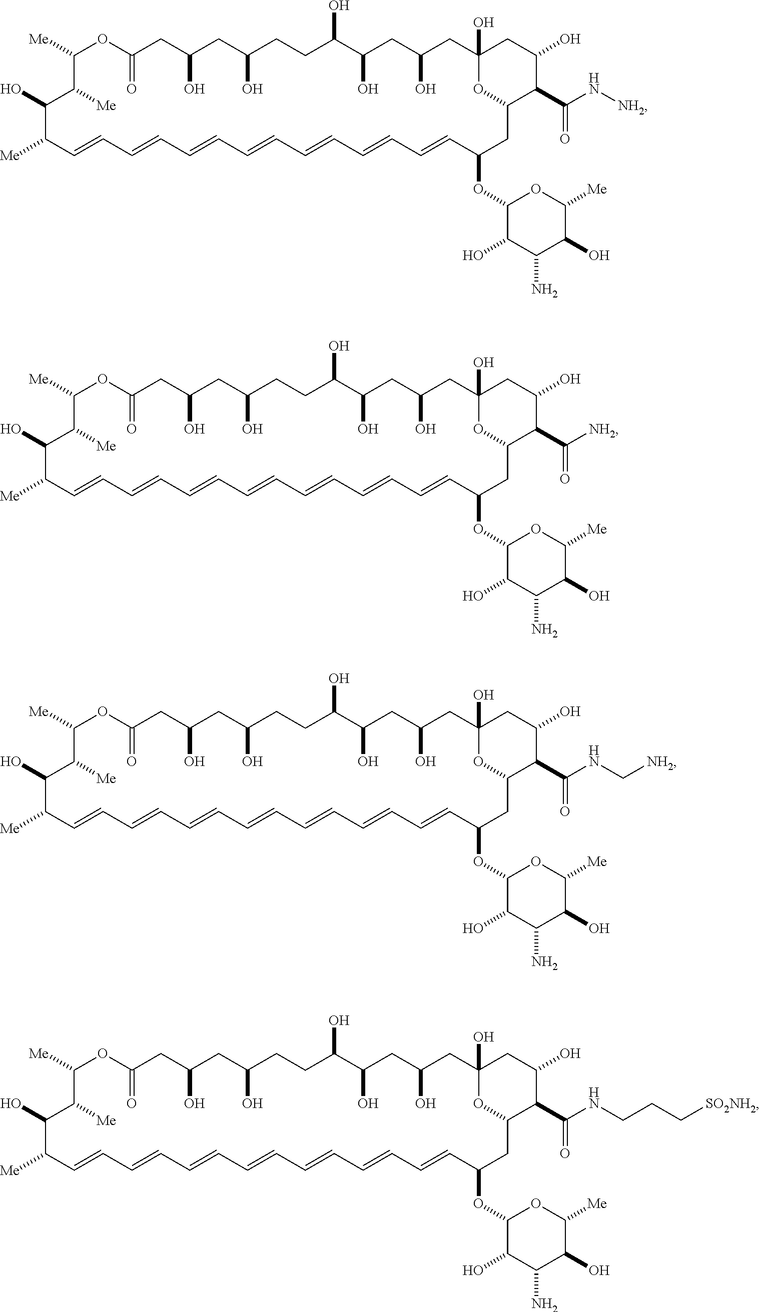

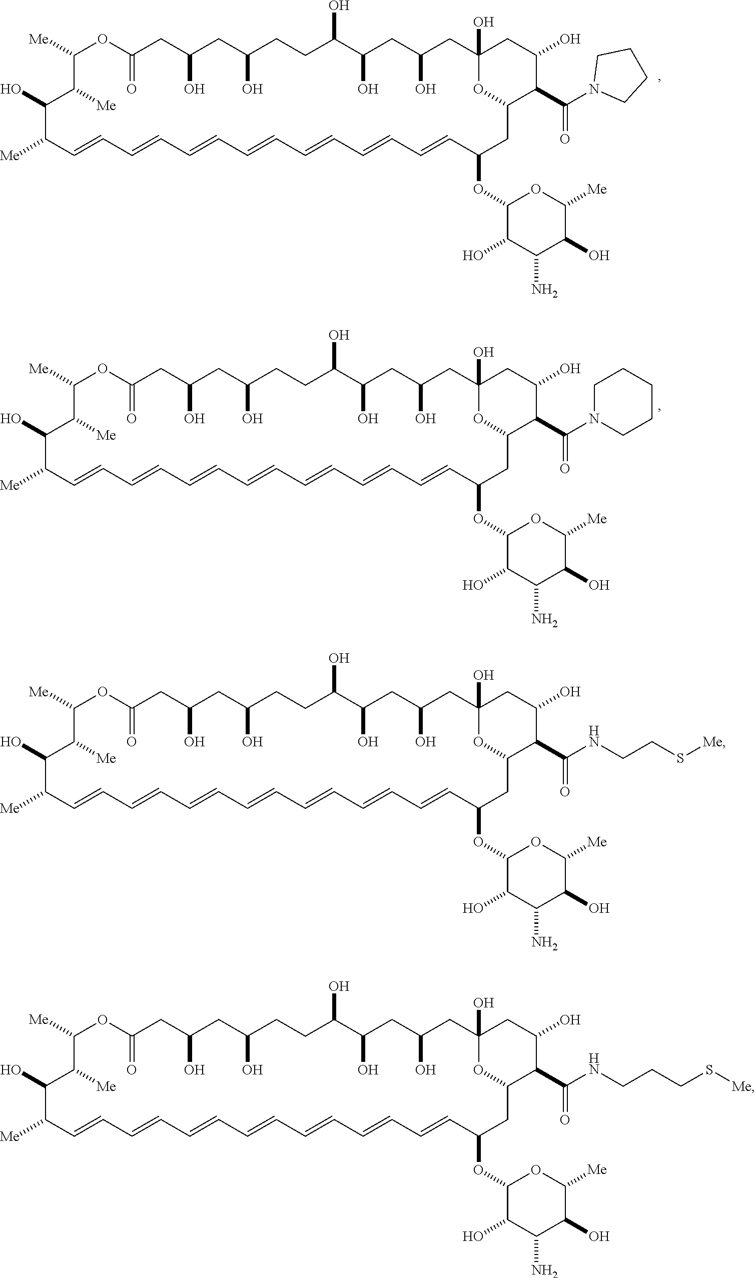

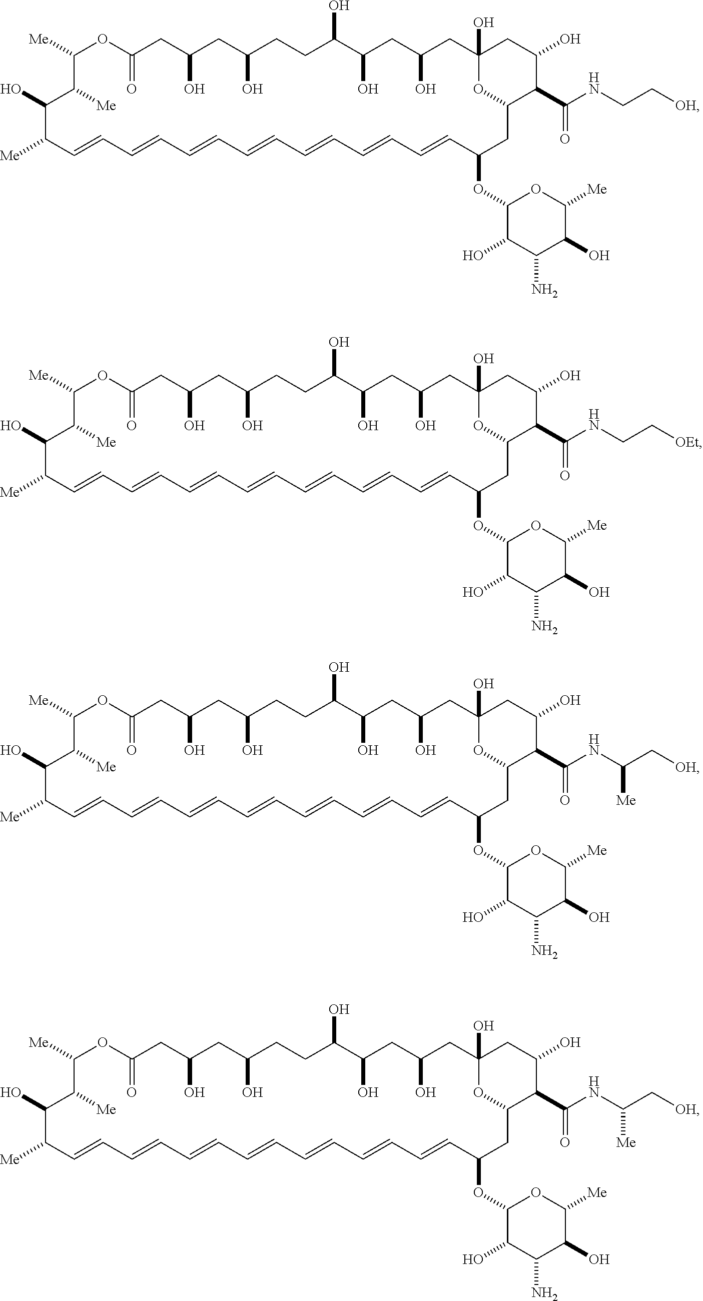

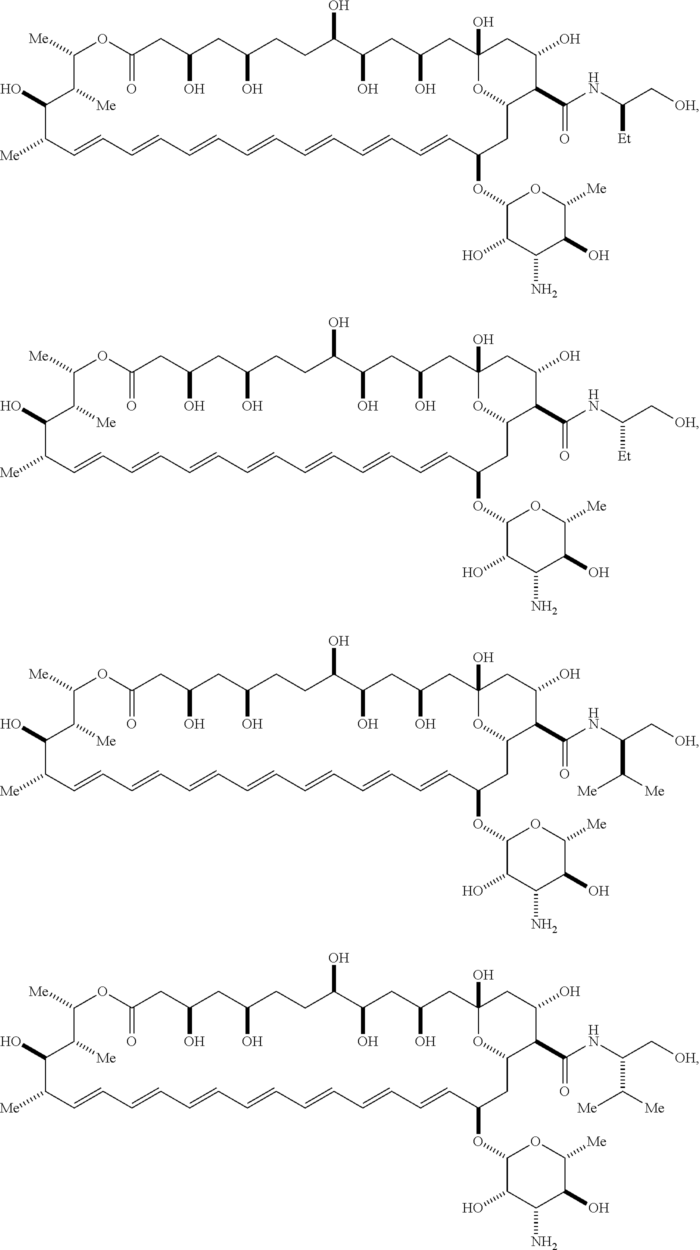

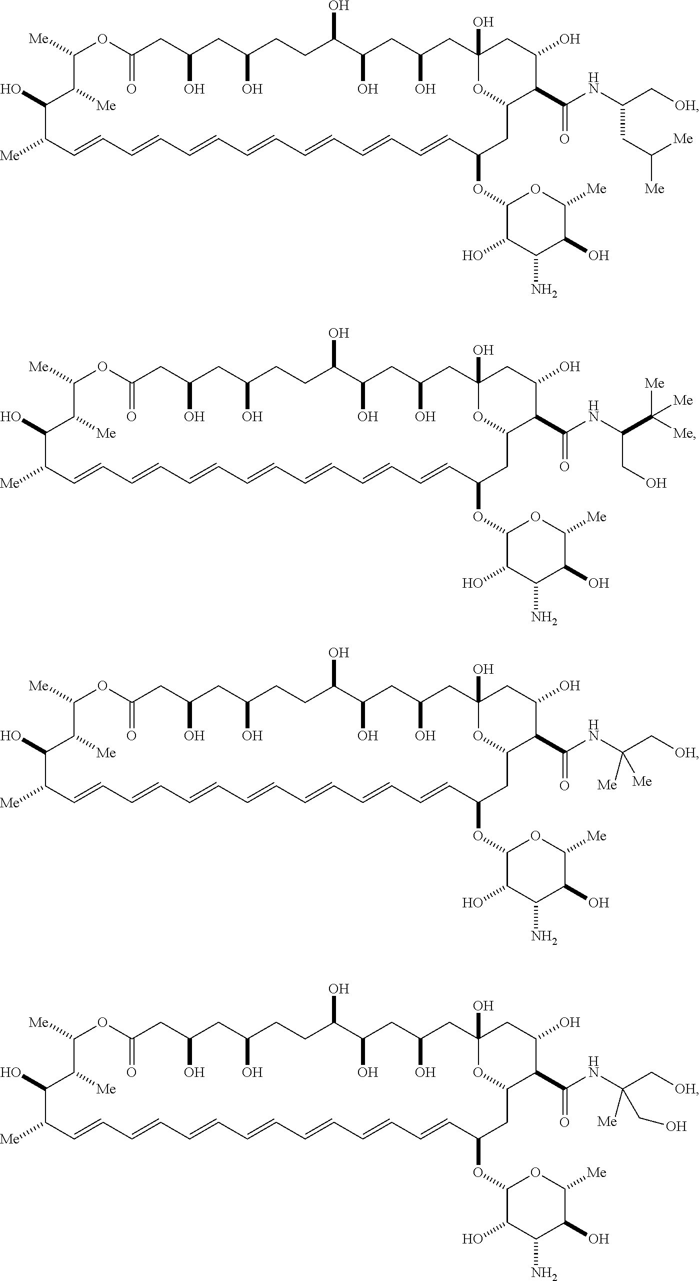

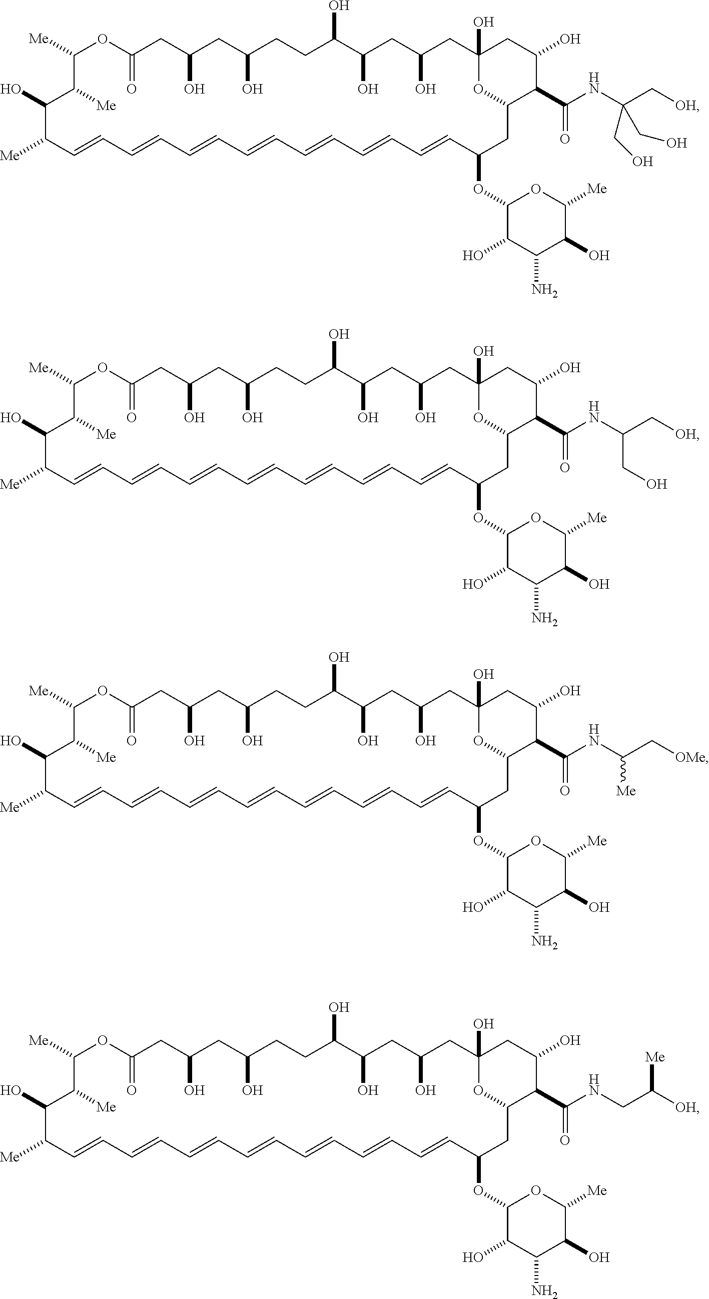

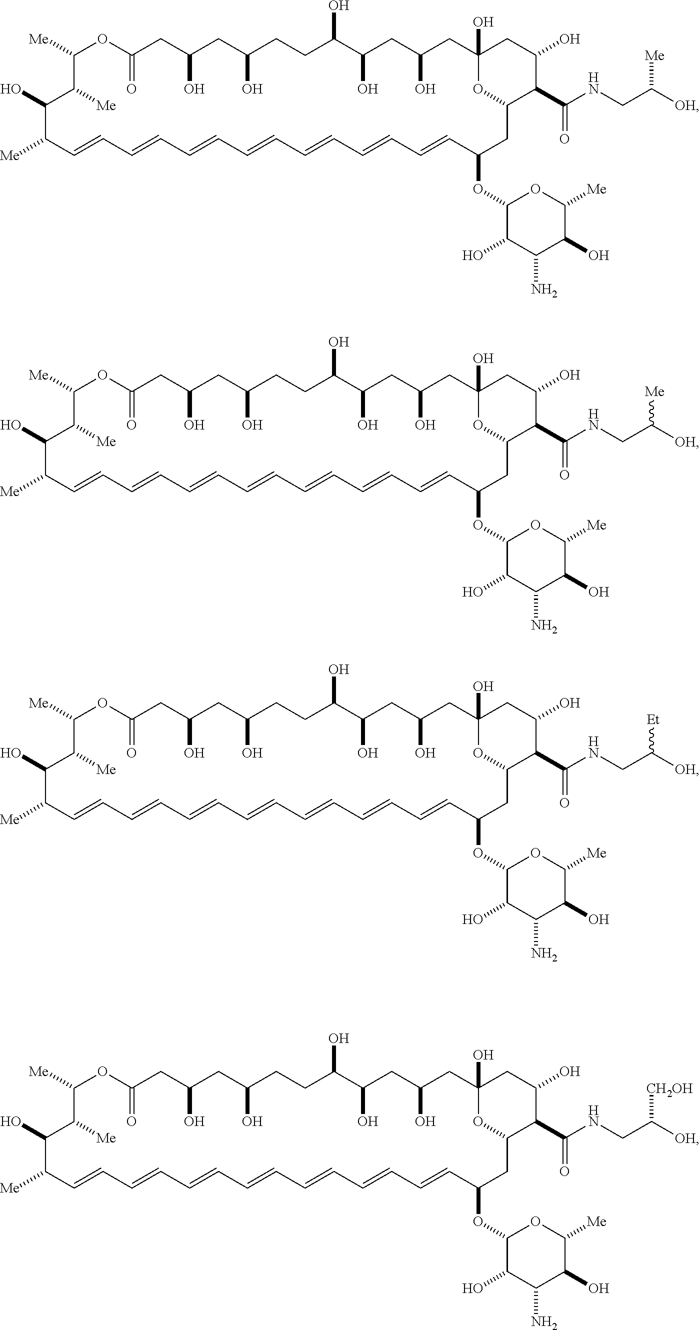

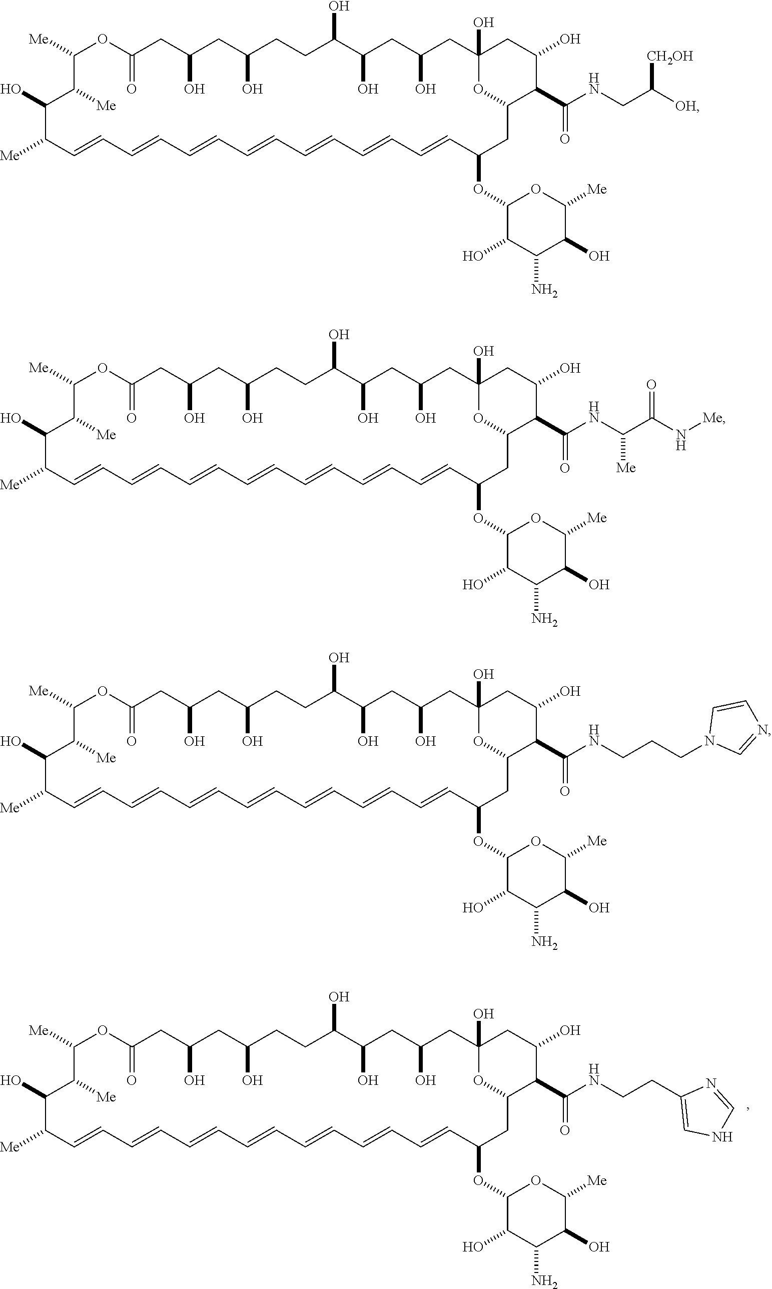

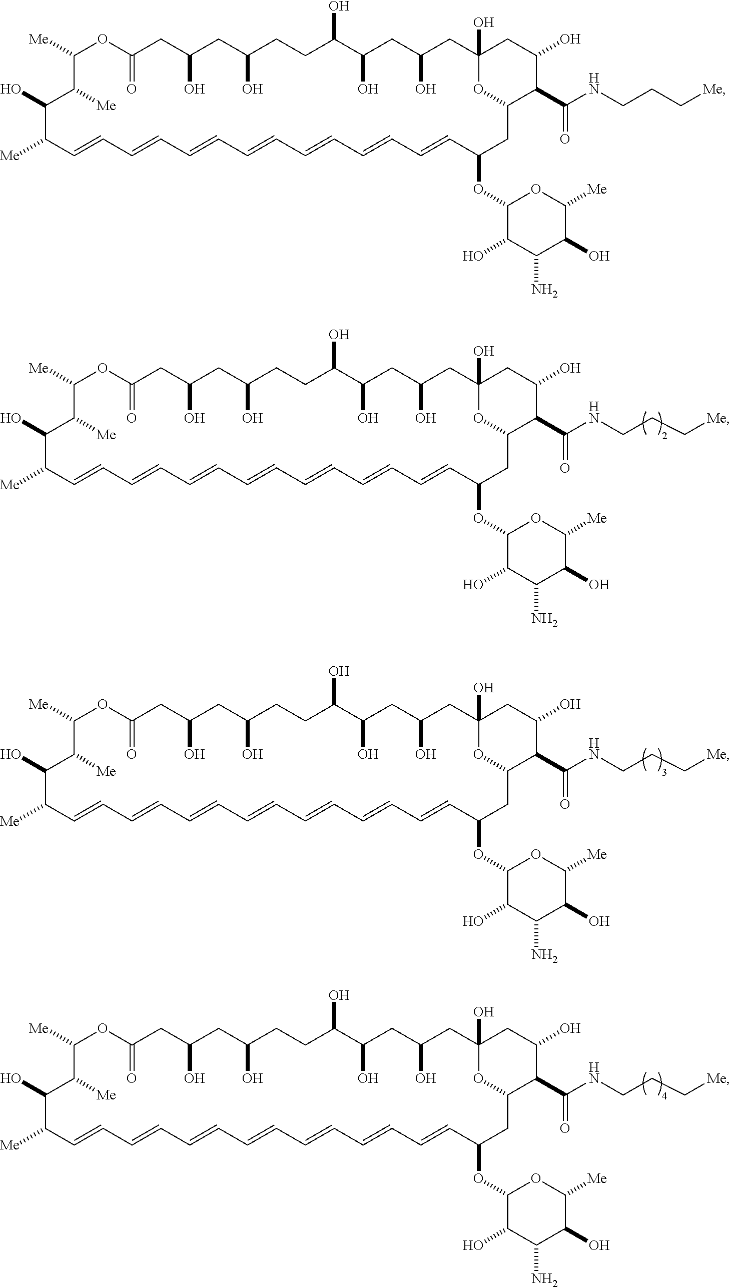

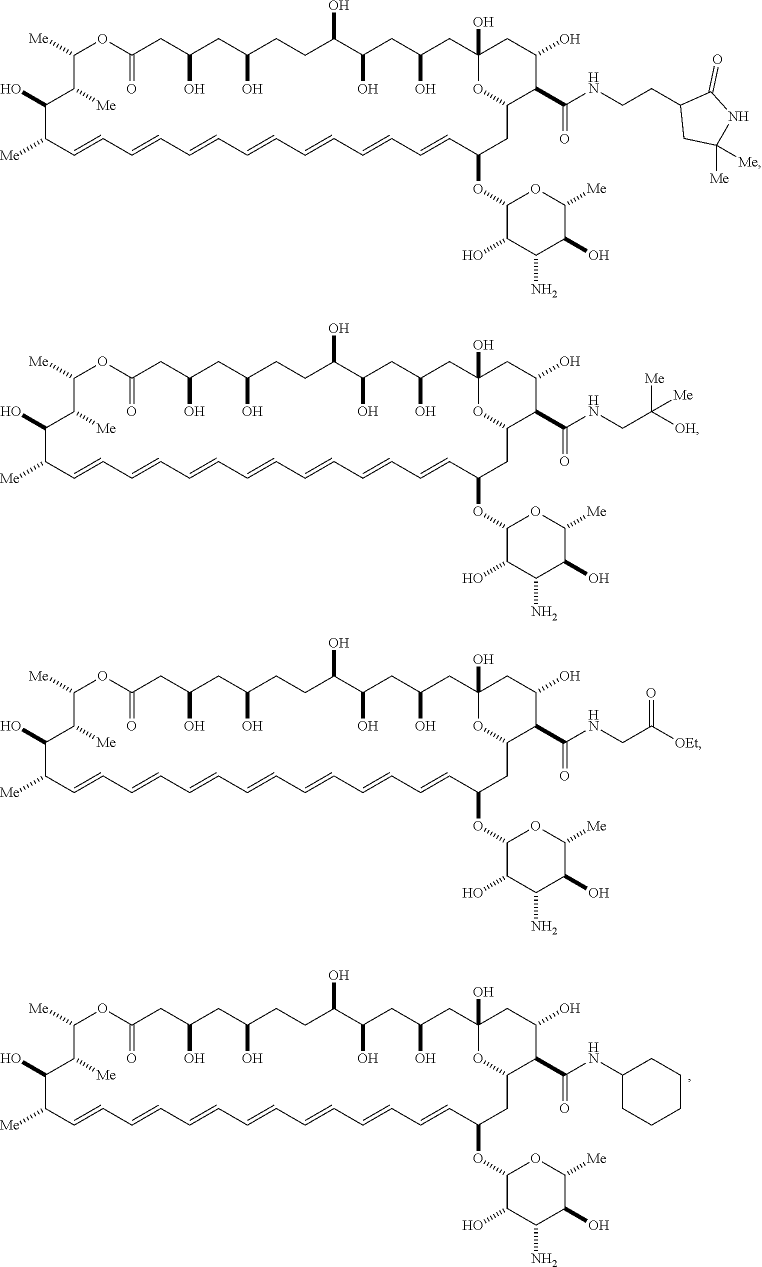

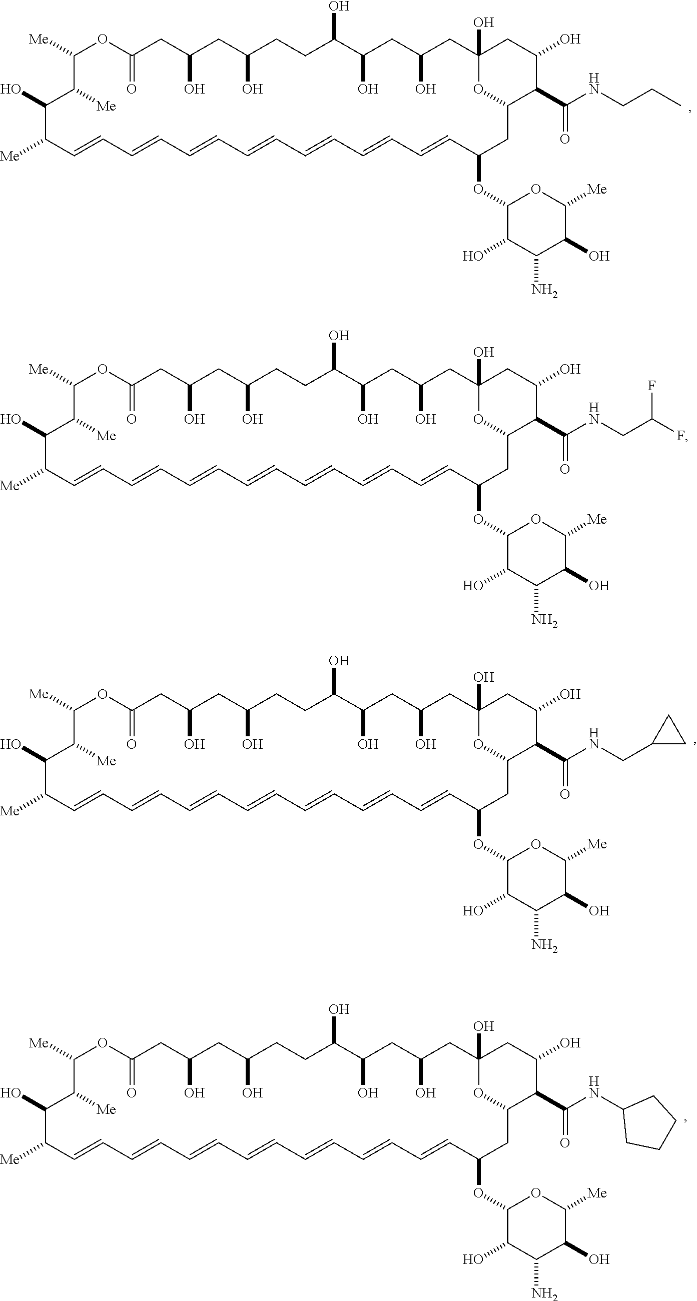

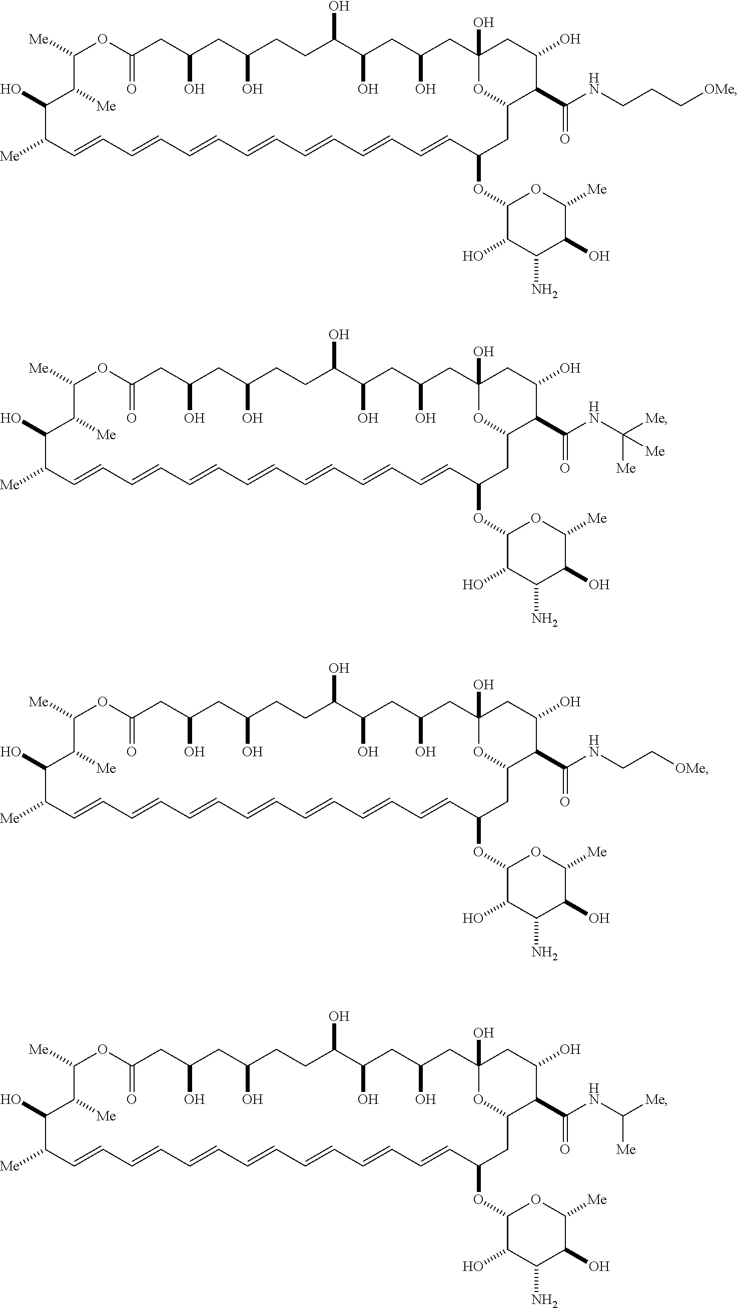

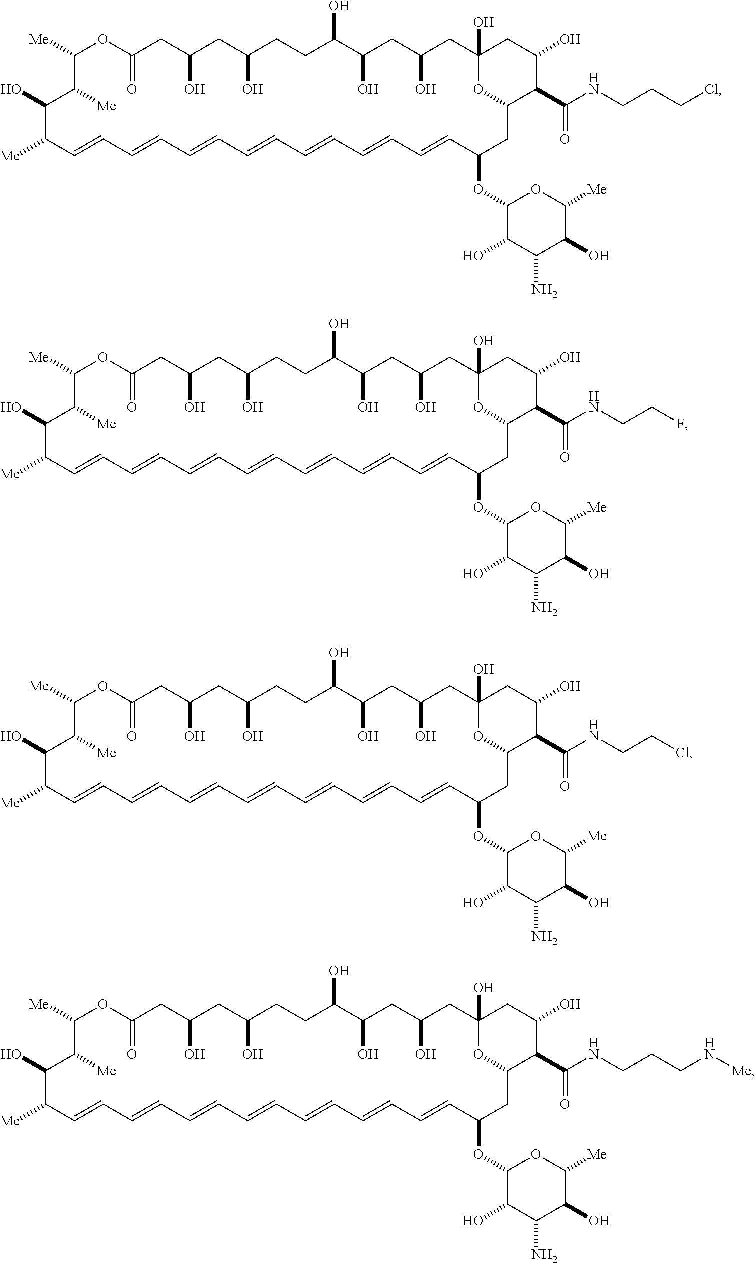

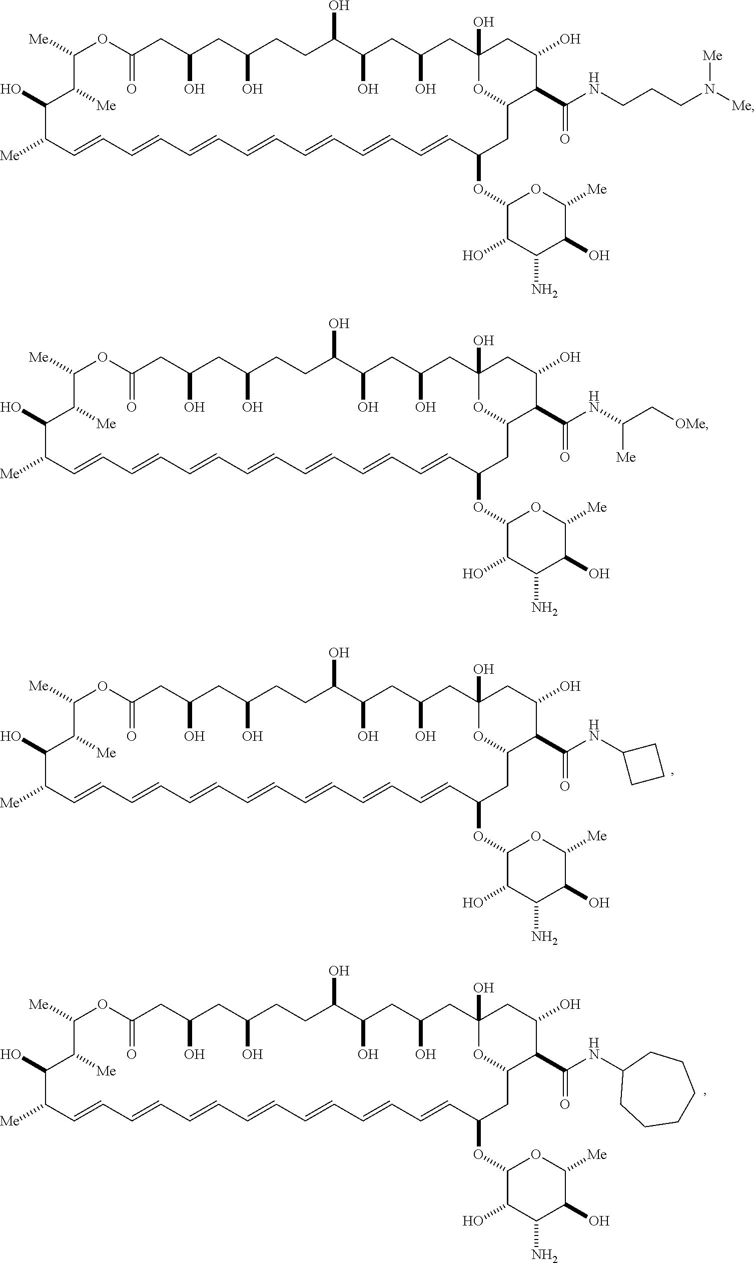

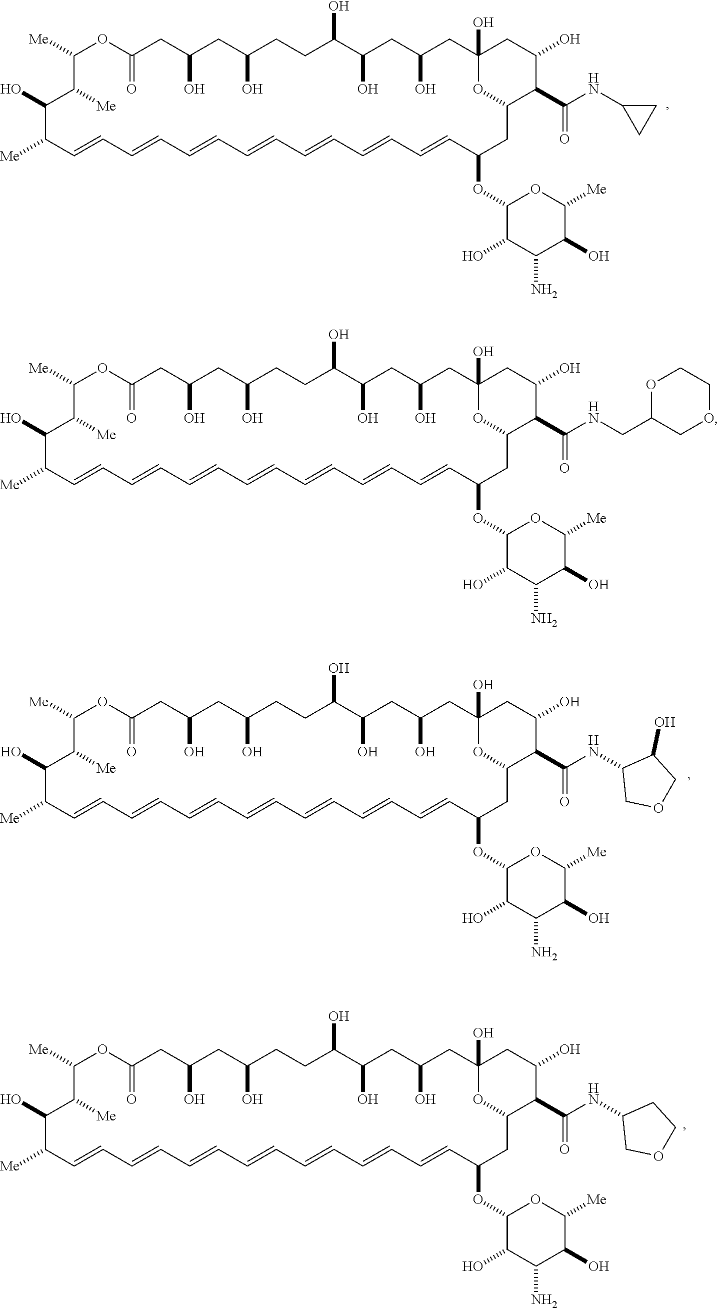

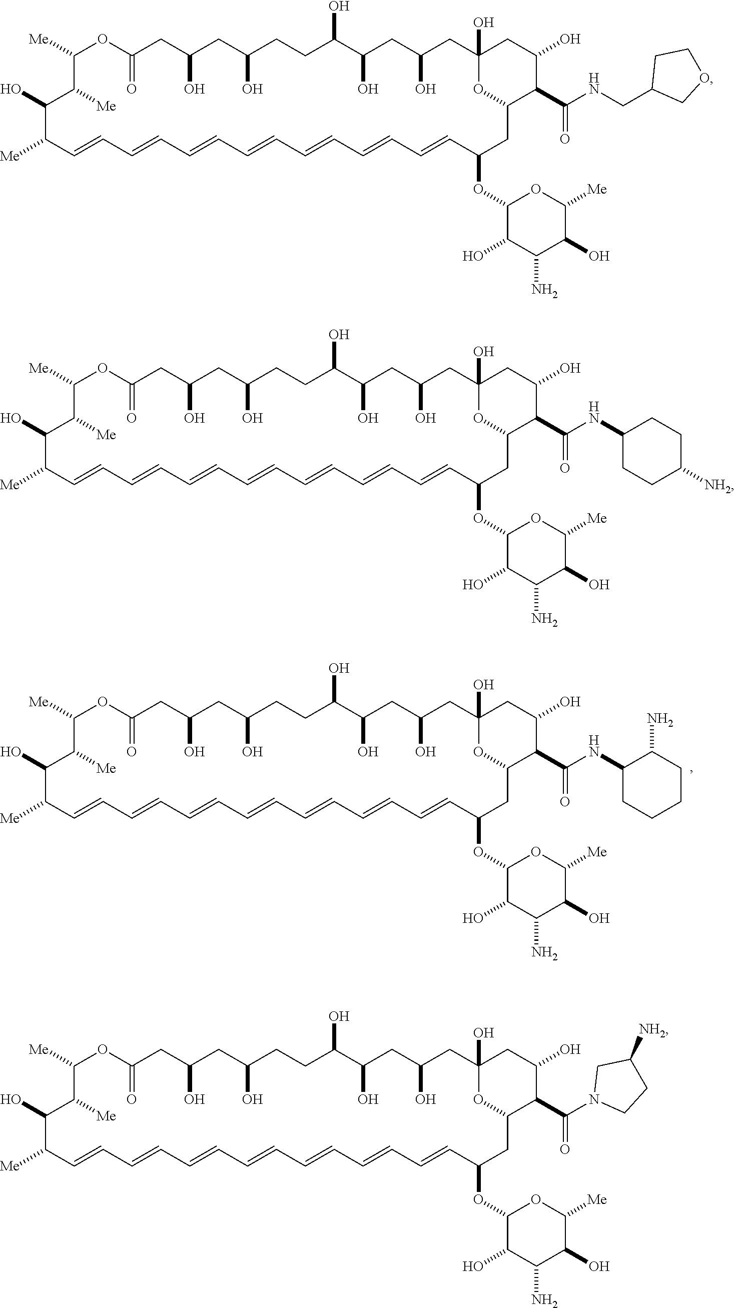

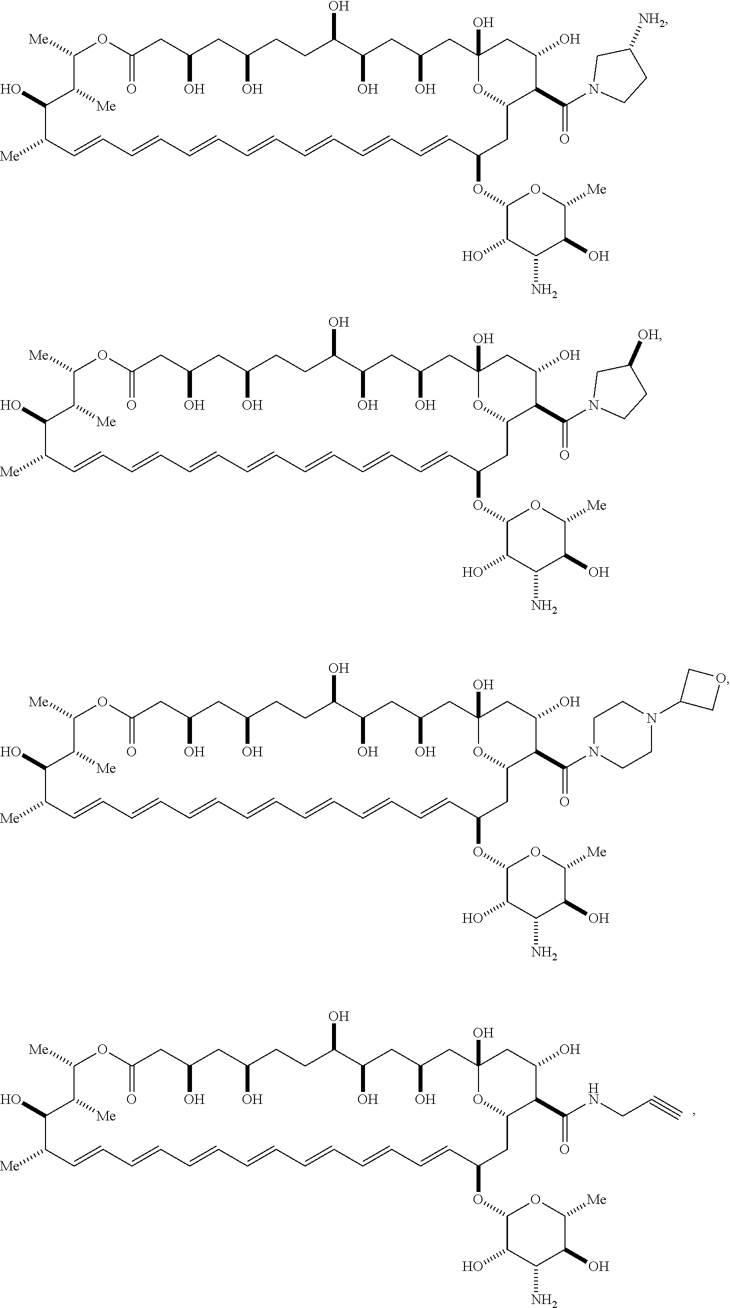

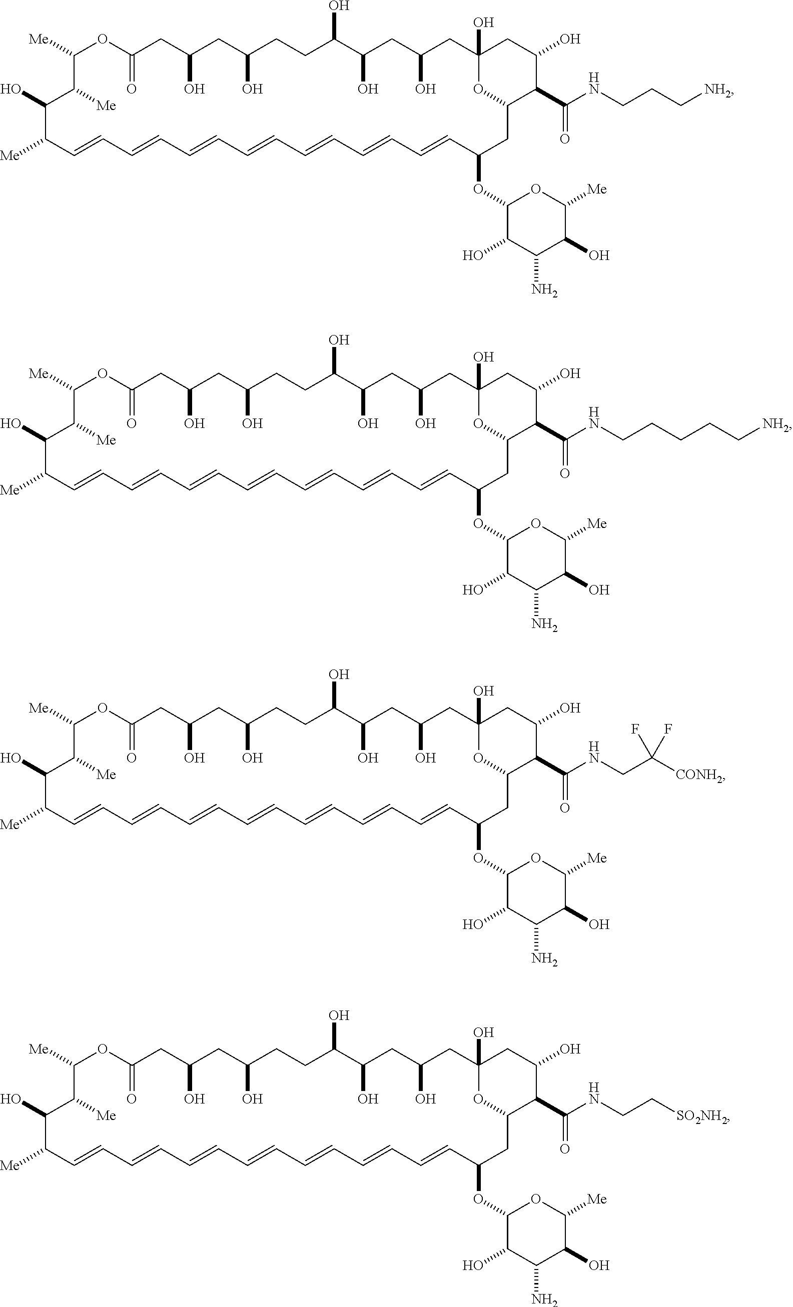

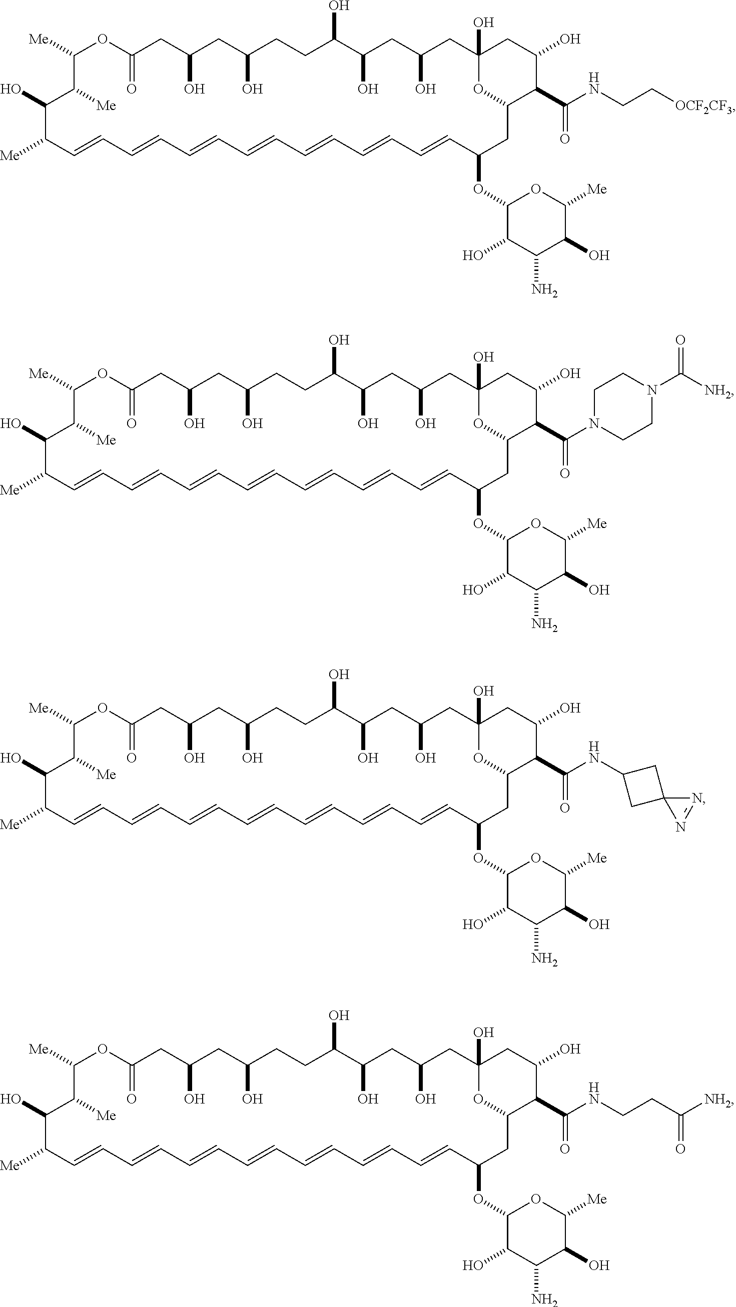

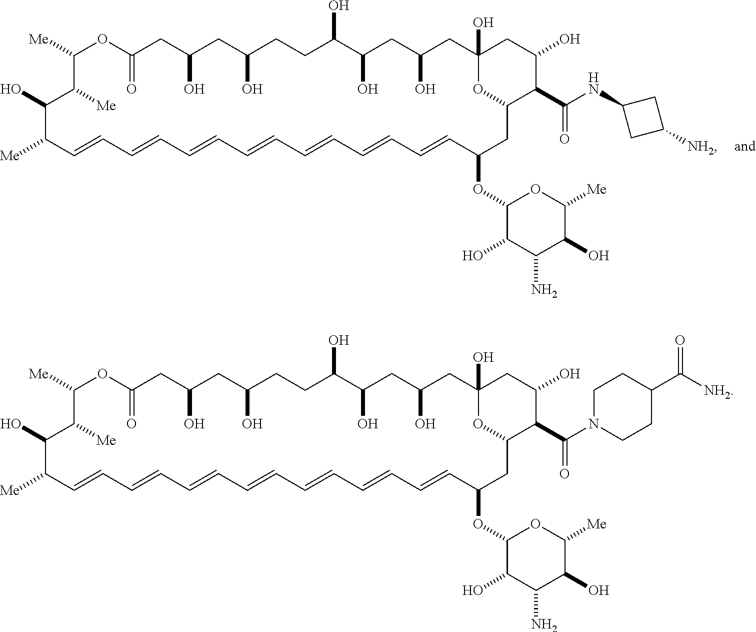

33. A pharmaceutical composition, comprising a compound of any one of claims 1-32; and a pharmaceutically acceptable carrier.

34. The pharmaceutical composition of claim 33, wherein the pharmaceutical composition is an intravenous dosage form.

35. The pharmaceutical composition of claim 33, wherein the pharmaceutical composition is an oral dosage form.

36. A method of treating a fungal infection, comprising administering to a subject in need thereof a therapeutically effective amount of a compound of any one of claims 1-32, thereby treating the fungal infection.

37. The method of claim 36, wherein the compound is administered intravenously.

38. The method of claim 36, wherein the compound is administered orally.

39. A method of making a C16 amide of C2′-epi-amphotericin B according to the transformation shown in Scheme 1: 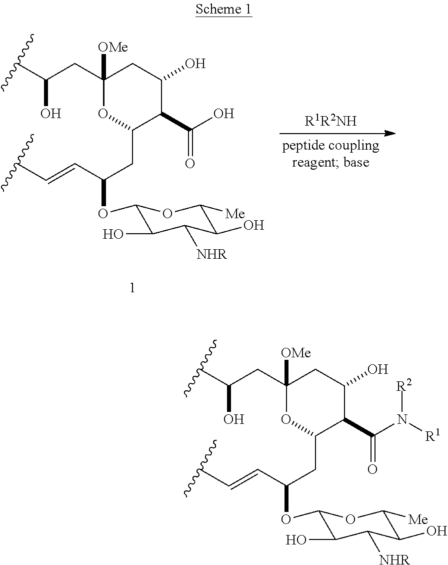
wherein:
1 represents 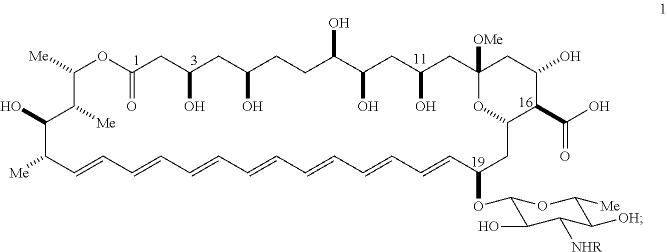
base is a tertiary amine (e.g., a trialkylamine [such as Et₃N]);
peptide coupling reagent is a peptide coupling reagent used in solid phase peptide synthesis (e.g., PyBOP, BOP, HATU, HBTU, DEPBT, DCC, or EDCI);
R is H or an amine protecting group (e.g., a carbamate protecting group selected from the group consisting of Fmoc, t-Boc, alloc, and Cbz); and
R¹ and R² independently are hydrogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocyclyl, substituted or unsubstituted 3- to 10-membered heterocyclyl, substituted or unsubstituted C₅₋₁₀ aryl, substituted or unsubstituted 5- to 10-membered heteroaryl; or R¹ and R², together with the nitrogen to which they are attached, form a substituted or unsubstituted 3- to 10-membered heterocyclyl.

40. The method of claim 39, wherein R is H.

41. The method of claim 39, wherein R is a carbamate protecting group selected from the group consisting of Fmoc, t-Boc, alloc, and Cbz.

42. The method of any one of claims 39-41, wherein base is a trialkylamine.

43. The method of claim 42, wherein base is Et₃N.

44. The method of any one of claims 39-43, wherein peptide coupling reagent is PyBOP, BOP, HATU, HBTU, DEPBT, DCC, or EDCI.
